(19) United States
(12) Patent Application Publication
(10) Pub. No.: US 2024/0200089 A1
(43) Pub. Date: Jun. 20, 2024

(54) IMMUNOMODULATING TRANSGENIC PLANTS AND RELATED METHODS

(71) Applicant: AGRIVIDA, INC., Woburn, MA (US)

(72) Inventors: R. Michael RAAB, Arlington, MA (US); Oleg Bougri, Winchester, MA (US); Matthew Parker, Cambridge, MA (US); Philip A. Lessard, Framingham, MA (US)

(73) Assignee: AGRIVIDA, INC., Woburn, MA (US)

(21) Appl. No.: 18/421,496

(22) Filed: Jan. 24, 2024

Related U.S. Application Data
(63) Continuation of application No. 17/748,507, filed on May 19, 2022, now Pat. No. 11,932,864.
Application No. 17/748,507 is a continuation of application No. 16/609,633, filed on Oct. 30, 2019, now Pat. No. 11,390,879.
(60) Provisional application No. 62/512,444, filed on May 30, 2017.

(51) Int. Cl. C12N 15/82; A61K 39/00; A61P 33/00; C07K 14/54; C07K 16/24

(52) U.S. Cl. CPC C12N 15/8258; A61P 33/00; C07K 14/5428; C07K 16/244; A61K 2039/505; A61K 2039/542; C07K 2317/13; C07K 2317/22; C07K 2317/23; C07K 2317/569; C07K 2317/76; C07K 2317/92; C07K 2317/94

(57) ABSTRACT

The transgenic plants expressing one or more antagonist IL-10R peptides and anti-IL-10 single domain antibodies that stimulate or modulate the immune system and improve gastrointestinal physiology of an animal fed the transgenic plants or tissues thereof and the genes encoding the antagonist IL-10R peptides and anti-IL-10 single domain antibodies are described. The animal feed additives and animal feed incorporating the transgenic plants or tissues thereof are also described. Methods of stimulating or modulating an animal's immune system, improving an animal's gastrointestinal physiology, improving animal performance by using the transgenic plants or tissues thereof, and treating animals infected with a gastrointestinal pathogen are provided.

### CROSS REFERENCE TO RELATED APPLICATION

This application is a continuation of U.S. patent application Ser. No. 17/748,507 filed on May 19, 2022. The U.S. patent application Ser. No. 17/748,507 is a continuation of U.S. patent application Ser. No. 16/609,633 filed on Oct. 30, 2019 as U.S. National Stage of International Patent Application No. PCT/US2018/034856, which was filed on May 29, 2018, and issued as U.S. Pat. No. 11,390,879 on Jul. 19, 2022. The International Patent Application No. PCT/US2018/034856 claimed the benefit of U.S. Provisional Application No. 62/512,444 filed May 30, 2017. All of these applications are incorporated herein by reference as if fully set forth.

The sequence listing electronically filed with this application titled “Sequence Listing,” which was created on Jan. 24, 2024 and had a size of 594,991 bytes is incorporated by reference herein as if fully set forth.

### FIELD OF INVENTION

This disclosure relates to antagonist IL-10 receptor (IL-10R) peptides and anti-IL-10 antibodies, including anti-IL-10 single domain antibodies. This disclosure relates to transgenic plants that express and accumulate antagonist IL-10R peptides and anti-IL-10 single domain antibodies that stimulate or modulate the immune system and improve gastrointestinal physiology of an animal fed the transgenic plants or tissues thereof. This disclosure also relates to the genes encoding these peptides and antibodies.

This disclosure relates to animal feed additives and animal feed that incorporates the transgenic plants or tissues thereof including the peptides and antibodies. This disclosure also relates to animal feed additives and animal feed that incorporates the peptides and antibodies.

This disclosure relates to methods of treating animals infected with a gastrointestinal pathogen by administering to them antagonist IL-10R peptides and anti-IL-10 single domain antibodies, transgenic plants expressing the peptides and antibodies disclosed herein, or feeding animals with animal feed that includes these transgenic plants, peptides or antibodies. The disclosure also relates to methods of stimulating or modulating an animal's immune system, methods of improving an animal's gastrointestinal physiology, methods of improving animal performance by using the disclosed transgenic plants or tissues thereof, antagonist IL-10R peptides, or anti-IL-10 single domain antibodies.

This disclosure relates to methods of making antagonist IL-10R peptides and anti-IL-10 single domain antibodies, and methods of making transgenic plants expressing the peptides and antibodies disclosed herein.

The sequence listing electronically filed with this application titled “Sequence Listing,” created on May 30, 2017, and having a file size of 210,215 bytes is incorporated herein by reference as if fully set forth.

### BACKGROUND

Coccidiosis is a common poultry disease caused by protozoan parasites that infect the gastrointestinal tract (Cervantes, H., 2002; Cervantes, H., 2006).

The disease spreads from one animal to another by contact with infected feces or ingestion of infected tissue. Coccidiosis in chickens is caused by infection of the intestinal lining cells by parasitic protozoa of the genus Eimeria, and commonly by Eimeria tenella. The most common medications used to treat coccidial infections are anti-Coccidial drugs, antibiotics, and vaccines.

Anti-Coccidial drugs and Coccidiostats are used in poultry production to control Coccidiosis and maintain animal productivity, which generally decreases when animals are infected by Eimeria, and develop subclinical or clinical Coccidiosis. Clinical Coccidiosis results in disruption of the digestive tract, and symptoms include weight loss, growth suppression, diarrhea, bloody droppings and increased mortality. Subclinical Coccidiosis is common in poultry production, even when employing current Coccidiostats or vaccines, and does not present many of the same symptoms as clinical Coccidiosis, but still decreases animal productivity. The reduced animal productivity from Coccidiosis results in significant losses for the poultry industry, estimated at over one billion US dollars per year.

Anti-Coccidial drugs, antibiotics, and vaccines are important for efficient poultry production, but are being phased out in many countries due to consumer concerns over their use and safety. Vaccine use is challenged by incomplete immunity within the flock, and anti-Coccidial drugs are costly, need to be administered at the right time and dose, and can lead to the development of resistant Eimeria strains. Industry has witnessed a rise in the number of drug-resistant strains, which lowers the value of these products and necessitates the development of other methods for controlling Coccidiosis.

Eimeria stimulates production of an anti-inflammatory cytokine interleukin 10 (IL-10). IL-10 interacts with its receptor IL-10R in the intestinal lining to suppress the immune response. In turn, this allows Eimeria infection to proceed without interference from the immune system. IL-10 is a potent anti-inflammatory cytokine that helps animals control inflammation responses. IL-10 also controls the immune system to prevent hyper immune responses. Blocking IL-10 to prevent its interaction with IL-10R would prevent immune suppression, and thus, helps the animal's normal immune response to reduce and clear Eimeria infection. In contrast to other prophylactic or therapeutic approaches to controlling Coccidiosis, blocking IL-10 suppression of the immune system should not lead to the development of resistant Eimeria strains because such intervention focuses on stimulating the host's immune response and not on attenuating or killing the infectious agent itself.

As previously described, this approach currently suffers from significant limitations that have prevented widespread commercial adoption and industrial use. First, the antibodies used thus far have been generated by inoculating either a maternal hen, or eggs, with the target peptide. In the case of the former, only chicks from the inoculated hen may be used, requiring the inoculation of many hens for chick production, and full protection is not guaranteed due to inadequate immunity, an ineffective peptide (stimulating antigen), or an unprolonged response. Peptide effectiveness may also be challenged since it is well known that small peptides often do not mobilize an effective immune response, and because IL-10 (or IL-10 homologs) is produced by the host, it may be difficult to generate adequate antibodies without the use of adjuvants or conjugates, which further increases the cost and complexity of this approach. Furthermore, because IL-10 is known to dimerize in vivo, selected peptides may generate antibodies to epitopes that are not normally exposed by the IL-10 dimer and therefore may be ineffective in binding IL-10 in the host animal. Likewise, inoculating eggs (or collecting eggs from inoculated hens) is cumbersome and increases costs, suffers from many of the same issues that challenge hen inoculation, and adds additional costs when the antibodies must be harvested from the yolks. In the case where the antibodies are harvested from the eggs, the material must be dried, stabilized, and then mixed into feed to deliver to chicks. While the added processing steps (including harvesting the eggs, drying, formulating and packaging for feed addition), add extra cost, it is unclear how consistent this production method will be, how susceptible it is to contamination by other infectious agents, or whether the antibodies generated in this manner will be thermally stable enough to survive the pelleting processes used in preparing animal feed. Many full-length antibodies do not possess the thermal stability required to maintain their solubility, structure, and affinity for IL-10, when combined with animal feed and processed through a pellet mill. Antibodies delivered in pelleted feed will be exposed to pelleting temperatures that may be 65° C., 70° C., 75° C., 80° C., 82° C., 85° C., 90° C., 95° C., or even greater. For these reasons, using eggs to produce antibodies for animal feed is a very challenging, high-cost practice, and because the antibodies are never fully sequenced or characterized, this production method precludes the use of biotechnology to improve antibody properties and the cost, efficiency, and efficacy of production. Therefore, new technologies are greatly needed if modulation of the IL-10 signaling pathway is to achieve any market relevance in the animal production industry. To address these shortcomings, there exists a need for a novel, low-cost feed additive that ideally is delivered in existing diet ingredients, that has increased thermal stability to endure the feed pelleting process, and that can more effectively inhibit the IL-10 signaling process.

### SUMMARY

In an aspect, the invention relates to a transgenic plant or tissues thereof comprising a synthetic polynucleotide encoding at least one antagonist IL-10R peptide, or an anti-IL-10 single domain antibody.

In an aspect, the invention relates to at least one antagonist IL-10R peptide. The at least one antagonist IL-10R peptide is one peptide comprising an amino acid sequence with at least 90% identity to a reference sequence selected from the group consisting of: SEQ ID NOS: 1-13. The at least one antagonist IL-10R peptide comprises concatenated peptides comprising an amino acid sequence with at least 90% identity to a reference sequence selected from the group consisting of: SEQ ID NOS: 32-40.

In an aspect, the invention relates to a synthetic polynucleotide encoding the at least one IL-10R antagonist peptide described herein.

In an aspect, the invention relates to an anti-IL-10 single domain antibody that binds to a polypeptide comprising an amino acid sequence of SEQ ID NO: 80.

In an aspect, the invention relates to a synthetic polynucleotide encoding any one of the anti-IL-10 single domain antibodies described herein.

In an aspect, the invention relates to an animal feed comprising any one of the transgenic plants or tissues thereof described herein.

In an aspect, the invention relates to an animal feed comprising any of the antagonist IL-10R peptides, or anti-IL-10 single domain antibodies described herein.

In an aspect, the invention relates to a method of treating or preventing a gastrointestinal infection in an animal comprising feeding the animal any one of the transgenic plants or tissues thereof, antagonist IL-10R peptides, anti-IL-10 single domain antibodies, or animal feed described herein.

In an aspect, the invention relates to a method of stimulating or modulating the immune system and improving gastrointestinal physiology of an animal comprising feeding the animal any one of the transgenic plants or tissues thereof, antagonist IL-10R peptides, anti-IL-10 single domain antibodies or the animal feed described herein.

In an aspect, the invention relates to a method of improving animal performance comprising feeding an animal any one of the transgenic plants or tissues thereof, antagonist IL-10R peptides, anti-IL-10 single domain antibodies or animal feed described herein.

In an aspect, the invention relates to a method of preparing an animal feed comprising mixing any one of the transgenic plants or tissues thereof described herein with plant material to form a mixture.

In an aspect, the invention relates to a method of preparing an animal feed comprising mixing any one of the antagonist IL-10R peptides or anti-IL-10 single domain antibodies described herein with plant material to form a mixture.

In an aspect, the invention relates to a method of preparing a transgenic plant or tissues thereof comprising any of the antagonist IL-10R peptides or anti-IL-10 single domain antibodies described herein.

### BRIEF DESCRIPTION OF THE DRAWINGS

The following detailed description of preferred embodiments of the present invention will be better understood when read in conjunction with the appended drawings. For the purpose of illustrating the invention, particular embodiments are shown in the drawings. It is understood, however, that the invention is not limited to the precise arrangements and instrumentalities shown. In the drawings:

FIGS. 1A-1G are schematic drawings of the vectors pAG4305 (FIG. 1A), pAG4306 (FIG. 1B), pAG4308 (FIG. 1C), pAG4310 (FIG. 1D), pAG4311 (FIG. 1E), pAG4312 (FIG. 1F), and pAG4313 (FIG. 1G).

FIGS. 2A-2D are schematic drawings of the vectors pAG4981 (FIG. 2A), pAG4982 (FIG. 2B), pAG4983 (FIG. 2C), and pAG4984 (FIG. 2D).

FIG. 3 illustrates the antibody response generated by a llama injected with full-length chicken IL-10. It demonstrates that specific antibodies are produced by the animal that increases the binding of chicken IL-10 post-injection, relative to the pre-immune (that is, pre-injection) state of the animal.

FIG. 4 illustrates the sequencing results from the anti-IL-10 antibody development and inter-relationship among the identified sequences. In this figure, sequences of sdAbs according to embodiments herein are aligned and compared to the sequence of sdAbs 40-IL-bR2-1H5 set forth as SEQ ID NO: 208. The amino acid (AA) positions replaced in the sequence of SEQ ID NO: 208 are as follows: for 68-IL-bR2-1D9, AA 26 to 34 (SEQ ID NO: 209); for 80-IL-bR2-1H10, AA 54 to 57 (SEQ ID NO: 210); for 03-IL-bR2-1C1, 04-IL-bR2-1D1, 81-IL-bR2-1A11, 63-IL-bR2-1G8, and 33-IL-bR2-1A5, AA 30 to 34 (SEQ ID NO: 211), AA 97 to 103 (SEQ ID NO: 212), and AA 105 to 112) SEQ ID NO: 213); for 35-IL-bR2-1C5, AA 30 to 34 (SEQ ID NO: 214), AA 50 to 54 (SEQ ID NO: 215), and AA 97 to 103 (SEQ ID NO: 212); for 48-IL-bR2-1H6, AA 27 to 34 (SEQ ID NO: 216), AA 75 to 79 (SEQ ID NO: 217), and AA 105 to 112 (SEQ ID NO: 218); for 01-IL-bR2-2A8 and 70-IL-bR2-1F9, AA 27 to 34 (SEQ ID NO: 216), AA 105 to 112 (SEQ ID NO: 218), and AA 76 to 79 (SEQ ID NO: 219); for 85-IL-bR2-1E11, AA 102 to 108 (SEQ ID NO: 220); for 44-IL-bR2-1D6, AA 26 to 34 (SEQ ID NO: 221), AA 99 to 102 (SEQ ID NO: 222), and AA 104 to 113 (SEQ ID NO: 223); for 27-IL-bR2-1C4, AA 27 to 31 (SEQ ID NO: 224), AA 46 to 50 (SEQ ID NO: 225), AA 53 to 61 (SEQ ID NO: 226), and AA 98 to 105 (SEQ ID NO: 227); for 32-IL-bR2-1H4, AA 52 to 57 (SEQ ID NO: 228), and AA 98 to 102 (SEQ ID NO: 229); for 86-IL-bR2-1F11, AA 100 to 104 (SEQ ID NO: 230), and AA 107 to 110 (SEQ ID NO: 231); for 20-IL-bR2-ID3, AA 44 to 47 (SEQ ID NO: 232), AA 52 to 55 (SEQ ID NO: 233), and AA 100 to 107 (SEQ ID NO: 234); for 49-IL-bR2-1A7, AA 27 to 35 (SEQ ID NO: 235), and AA 97 to 109 (SEQ ID NO: 236); for 24-IL-bR2-1H3, AA 24 to 37 (SEQ ID NO: 237), AA 98 to 101 (SEQ ID NO: 238), and AA 103 to 108 (SEQ ID NO: 239); for 58-IL-bR2-1B8, AA 26 to 35 (SEQ ID NO: 240), AA 46 to 61 (SEQ ID NO: 241), and AA 97 to 107 (SEQ ID NO: 242); for 10-IL-bR2-1B2, AA 27 to 32 (SEQ ID NO: 243), AA 52 to 59 (SEQ ID NO: 244), AA 75 to 80 (SEQ ID NO: 245), and AA 97 to 104 (SEQ ID NO: 246); for 12-IL-bR2-1D2, AA 30 to 34 (SEQ ID NO: 247), and AA 55 to 58 (SEQ ID NO: 248); and for 76-IL-bR2-1D10, AA 99 to 103 (SEQ ID NO: 249).

FIG. 5 illustrates apparent binding affinity of anti-IL-10 antibodies to chicken IL-10.

FIG. 6 illustrates results of the anti-IL-10 antibody digestion in the simulated gastric fluid (SGF) test.

FIG. 7 illustrates the apparent inhibition of IL-10 binding to the IL-10 receptor in the presence of anti-IL10 antibodies chIL10sdAB1A11 (SEQ ID NO: 135) and chIL10sdAB1F11 (SEQ ID NO: 146).

FIG. 8 illustrates IL-10 suppression of Concanavalin A-induced secretion of IFN-γ secretion in primary chicken spleen cells.

FIG. 9 illustrates recovery of IFN-γ secretion from primary chicken spleen cells treated with Concanavalin A and chicken IL-10, when also treated with the anti-IL-10 antibodies (chIL10sdAB1A11 (SEQ ID NO: 135), chIL10sdAB1B9 (SEQ ID NO: 111), and chIL10sdAB1F11 (SEQ ID NO: 146)).

FIG. 10A is a schematic drawing of the vector pAG4314.

FIG. 10B is a schematic drawing of the vector pAG4988.

FIG. 11 illustrates that single domain antibodies express at high level in individual corn grain. In both transgenic events, individual grain were genotyped and protein extracted. The presence of the sdAB band in the gel image correlates perfectly with the presence of the sdAB gene as represented at the top of the figure with a “+” if the sdAB gene is present and a “−” if the sdAB gene is absent.

FIG. 12 is a schematic drawing of the pLH1A11int expression cassette.

FIG. 13 is a schematic drawing of the pLH1A11 expression cassette.

FIG. 14 illustrates end point RT-PCR analysis of transiently expressed Nb1A11 in N. benthamiana leaves. Lanes 1-5 contain the following samples: lane 1—GV3101+pLH9000 (negative control); lane 2—GV3101+pLH1A11int; lane 3—GV3101+pLH1A11; lane 4—plasmid pLH1A11int; and lane 5—plasmid pLH1A11.

FIG. 15 illustrates sdAB1A11 expression in Agrobacterium infiltrated leaves of N. benthamiana. The Western blot shows detection of sdAB1A11 in samples 5 and 6.

### DETAILED DESCRIPTION OF THE PREFERRED EMBODIMENTS

Certain terminology is used in the following description for convenience only and is not limiting.

“Synthetic nucleic acid sequence,” “synthetic polynucleotide,” “synthetic oligonucleotide,” “synthetic DNA,” or “synthetic RNA” as used herein refers to a nucleic acid sequence, a polynucleotide, an oligonucleotide, DNA, or RNA that differs from one found in nature by having a different sequence that one found in nature or a chemical modification not found in nature. The definition of synthetic nucleic acid includes but is not limited to a DNA sequence created using biotechnology tools. Such tools include but are not limited to recombinant DNA technology, chemical synthesis, or directed use of nucleases (so called “genome editing” or “gene optimizing” technologies).

“Synthetic protein,” “synthetic polypeptide,” “synthetic oligopeptide,” or “synthetic peptide” as used herein refers to a protein, polypeptide, oligopeptide or peptide that was made through a synthetic process. The synthetic process includes but is not limited to chemical synthesis or recombinant technology.

As used herein, the terms “interleukin 10,” “IL10” and “IL-10” are used interchangeably, and refer to cytokine synthesis inhibitory factor, i.e., an anti-inflammatory cytokine. The terms “cIL-10,” “cIL10”, “chIL10”, and “chIL-10” refer to the chicken interleukin 10.

As used herein, the terms “antagonist IL-10R peptide,” “antagonist IL10R,” “IL10R anatagonist peptide,” and “IL-10R antagonist peptide” are used interchangeably, and refer to peptides that are inhibitors of IL-10 receptors (IL-10R). The IL-10R antagonist peptides may be fragments of IL-10, or may differ from the fragments of IL-10. The IL-10R antagonist peptide may be an antagonist derived from the IL-10R. The IL-10R antagonist peptide may be fusion of the peptides, concatenation of the peptides, or any other peptides that are capable of blocking or antagonizing IL-10 receptors. The IL-10R antagonist peptides can block or antagonize receptors in any way, e.g., by blocking the IL-10 binding pockets of the IL-10 receptors, preventing IL-10 from binding to the receptors, blocking IL-10 dimerization, or IL-10 receptor assembly, or allowing IL-10 binding to the receptors but blocking subsequent signal transduction. The IL-10R antagonist peptides can block or antagonize IL-10 receptors by any mechanism or mode of action.

“Antibody” as used herein refers to an immunoglobulin molecule which specifically binds with an antigen.

“Synthetic antibody” as used herein refers to an antibody which is generated using recombinant DNA technology, such as, for example, an antibody expressed by a host engineered to produce the antibody, such as a mammalian cell, microbial cell, or plant as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art. A “synthetic antibody” described herein may include fragments and hybrids of antibodies. A “synthetic antibody” described herein may be generated by an organism that is dosed with a specific antigen, and the antibody generated by the organism is isolated and propagated in a second organism.

A “single domain antibody,” or sdAB, refers to a synthetic antibody that is a small monomeric antigen-binding fragment of an antibody, i.e., the variable region of an antibody heavy or light chain. sdABs can be derived from antibodies that occur naturally or are generated in camelids, e.g., camels, and llamas, and may be produced by immunizing a camelid with a target antigen, isolating peripheral blood mononucleocytes, isolating their nucleic acids, and cloning sdAB coding regions from specific nucleic acid fragments. sdABs may be also produced in cell culture, by microbial hosts in a fermentation process, or by plants. An antibody described herein may be a sdAB comprising a VHH domain substantially as set out herein. A single domain antibody is a synthetic antibody.

“Antigen” as used herein is defined as a molecule that triggers an immune response. The immune response may involve either antibody production, or the activation of specific immunologically active cells, or both. The antigen may refer to any molecule capable of stimulating an immune response, including macromolecules such as proteins or peptides. The antigen may be synthesized, produced recombinantly in a mammalian, insect, microbial or plant cell, or may be derived from a biological sample, including but not limited to a tissue sample, a cell, or a biological fluid.

“Binding affinity” refers to the sum total noncovalent interaction between members of binding pairs, e.g., an antibody and antigen. The binding affinity of the antibody can be determined based on apparent binding EC50 value. As used herein, the term “EC50” or “EC₅₀” refers to the half maximal effective concentration, which includes the concentration of an antibody which induces a response halfway between the baseline and maximum after a specified exposure time. The EC₅₀ essentially represents the concentration of an antibody where 50% of its maximal effect is observed. In certain embodiments, the EC₅₀ value equals the concentration of an antibody of the invention that gives half-maximal binding to cells expressing chicken IL-10, as determined by, e.g., an ELISA assay. Thus, reduced or weaker binding is observed with an increased EC₅₀, or half maximal effective concentration value.

As used herein, “variant” refers to a protein or DNA molecule that has an amino acid or nucleic acid sequence that differs from the original sequence but retains a biological activity that is the same or substantially similar to that of the original sequence. The variant may be from the same or different species or be a synthetic sequence based on a natural or prior molecule.

The words “a” and “one,” as used in the claims and in the corresponding portions of the specification, are defined as including one or more of the referenced item unless specifically stated otherwise. This terminology includes the words above specifically mentioned, derivatives thereof, and words of similar import. The phrase “at least one” followed by a list of two or more items, such as “A, B, or C,” means any individual one of A, B or C as well as any combination thereof.

In an embodiment, one or more antagonist IL-10R peptides is provided. The antagonist IL-10R peptide may be expressed separately as one antagonist IL-10R peptide. The antagonist IL-10R peptide may include an amino acid sequence with at least 70, 72, 75, 80, 85, 90, 91, 92, 93, 94, 95, 96, 97, 98, 99 or 100% identity to a reference sequence selected from the group consisting of: SEQ ID NO: 1 [P21], SEQ ID NO: 2 [P22], SEQ ID NO: 3 [P23], SEQ ID NO: 4 [P24], SEQ ID NO: 5 [P25], SEQ ID NO: 6 [P26], SEQ ID NO: 7 [P27], SEQ ID NO: 8 [P28], SEQ ID NO: 9 [P29], SEQ ID NO: 10 [P11], SEQ ID NO: 11 [P30], SEQ ID NO: 12 [P31], and SEQ ID NO: 13 [P32].

An antagonist IL-10R peptide may be expressed in the form of concatenated antagonist IL-10R peptides. The concatenated antagonist IL-10R peptides may comprise a first antagonist IL-10R peptide having an amino acid sequence with at least 70, 72, 75, 80, 85, 90, 91, 92, 93, 94, 95, 96, 97, 98, 99 or 100% identity to a reference sequence selected from the group consisting of SEQ ID NOS: 1-13 fused to a second antagonist IL-10R peptide having an amino acid sequence with at least 70, 72, 75, 80, 85, 90, 91, 92, 93, 94, 95, 96, 97, 98, 99 or 100% identity to a reference sequence selected from the group consisting of SEQ ID NOS: 1-13. The first antagonist IL-10R peptide may differ from the second antagonist IL-10R peptide. The first antagonist IL-10R peptide may be similar to the second antagonist IL-10R peptide. The concatenated antagonist IL-10R peptides may have more than two antagonist IL-10R peptides. Each of the first antagonist IL-10R peptide and the second antagonist IL-10R peptide included in the concatenated antagonist IL-10R peptides may have an amino acid sequence with at least 70, 72, 75, 80, 85, 90, 91, 92, 93, 94, 95, 96, 97, 98, 99 or 100% identity to a reference sequence selected from the group consisting of SEQ ID NOS: 1-13. Subsequent antagonist IL-10R peptides may differ from the first and second antagonist IL-10R peptides and from each other. Subsequent antagonist IL-10R peptides may be similar to the first and the second antagonist IL-10R peptide and to each other. The concatenated antagonist IL-10R peptides may comprise an amino acid sequence with at least 70, 72, 75, 80, 85, 90, 91, 92, 93, 94, 95, 96, 97, 98, 99 or 100% identity to a reference sequence selected from the group consisting of SEQ ID NO: 32 [P2501], SEQ ID NO: 33 [P2502], SEQ ID NO: 34 [P2503], SEQ ID NO: 35 [P2504], SEQ ID NO: 36 [P2505], SEQ ID NO: 37 [P2506], SEQ ID NO: 38 [P2507], SEQ ID NO: 39 [P2508], and SEQ ID NO: 40 [P2509]. The first antagonist IL-10R peptide may be linked to the second antagonist IL-10R peptide by a linker. Each of the first, the second and the subsequent antagonist IL-10R peptides may be linked to each other by one or more linkers. The one or more linker may be selected from the group consisting of SEQ ID NOS: 41-44, and 65. The antagonist IL-10R peptide or the concatenated antagonist IL-10R peptides may comprise a signal peptide. The signal peptide may be but is not limited to an amyloplast targeting signal, a cell wall targeting peptide, a mitochondrial targeting peptide, a cytosol localization signal, a chloroplast targeting signal, a nuclear targeting peptide, or a vacuole targeting peptide. The signal peptide may an N-terminal signal peptide or a C-terminal signal peptide. The N-terminal signal peptide may be but is not limited to OsGluB4sp (rice Glu-B4 glutelin signal peptide), BAASS (barley alpha amylase signal sequence), PR1 (pathogenesis related protein), or zein 27 (xGZm27ss) signal peptide. The C-terminal signal peptide may be but is not limited to KDEL (SEQ ID NO: 29), HDEL (SEQ ID NO: 30), SEKDEL (SEQ ID NO: 31), HvVSD from barley polyamine oxidase, or HvAle from barley aleurone (thiol protease). The IL-10R antagonist peptide or the concatenated IL-10R antagonist peptides may be fused to the N-terminal signal peptide or C-terminal signal peptide, or both.

The antagonist IL-10R peptide, or the concatenated antagonist IL-10R peptides may be capable of reducing IL-10 binding to the IL-10R. The antagonist IL-10R peptide, or the concatenated antagonist IL-10R peptides may decrease the production of interferon gamma or nitric oxide when used in a cellular assay comprising cells that are stimulated by IL-10 to increase production of interferon gamma or nitric oxide.

In an embodiment, the antagonist IL-10R peptide having less than 100% identity to its corresponding amino acid sequence of SEQ ID NO: 1-13 or 32-40 may be a variant of the referenced peptide or amino acid. In an embodiment, an isolated peptide having a sequence with at least 70, 75, 80, 85, 90, 91, 92, 93, 94, 95, 96, 97, 98, 99, or 100% identity to a peptide having the sequence of any one of SEQ ID NOS: 1-13 and 32-40 along 7 to 10, 7 to 15, 7 to 30, 7 to 40, 7 to 50, or 7 to all amino acids of a protein having the sequence of any of one any one of SEQ ID NOS: 1-13 and 32-40 is provided. This list of sequence lengths encompasses every full length peptide in SEQ ID NOS: 1-13 and 32-40 and every smaller length within the list, even for peptides that do not include over 50 amino acids. For example, the lengths of 7 to 10, 7 to 20, 7 to 30, and 7 to all amino acids would apply to a sequence with 50 amino acids. A range of amino acid sequence lengths recited herein includes every length of amino sequence within the range, endpoints inclusive. The recited length of amino acids may start at any single position within a reference sequence where enough amino acids follow the single position to accommodate the recited length. The fragment of the antagonist IL-10R peptide may be a subsequence of the polypeptides herein that retain at least 40% of the antagonist IL-10R peptide EC₅₀ value when used in a cellular assay comprising cells that are inhibited by IL-10 (in the presence of ConA or PHA) to decrease production of interferon gamma or nitric oxide. The fragment may have 5, 7, 8, 9, 10, 15, 20, 25, 30, 35, 40, 45 or 50 amino acids. The fragments may include 5, 7, 8, 9, 10, 15, 20, 25, 30, 35, 40, 45 or 50 contiguous amino acids. Embodiments also include nucleic acids or polynucleotides, encoding said amino acid sequences. A less than full length amino acid sequence may be selected from any portion of one of the sequences of SEQ ID NOS: 1-13 and 32-40 corresponding to the recited length of amino acids. A less than full length amino acid sequence may be selected from a portion of any one of SEQ ID NOS: 1-13 and 32-40.

In an embodiment, an antagonist IL-10R peptide or concatenated antagonist IL-10R peptides may maintain or improve the body weight, and, or the feed conversion ratio, of poultry fed the peptides relative to poultry fed the same feed lacking the peptides. The antagonist IL-10R peptide or concatenated antagonist IL-10R peptides may be dosed at less than 500 mg per kg of pelleted feed, or more preferably at less than 50 mg per kg of pelleted feed, or even more preferably at less than 5 mg per kg of pelleted feed, or even more preferably at less than 1 mg per kg of pelleted feed. The antagonist IL-10R peptide or concatenated antagonist IL-10R peptides may also improve the body weight or feed conversion ratio of poultry when used in mash (non-pelleted) feed. The antagonist IL-10R peptide or concatenated antagonist IL-10R peptides may maintain their affinity for the IL-10R following incubation in liquid for at least 60 seconds at a temperature less than or equal to 65° C., or 70° C., or 75° C., or 80° C., or 85° C., or 90° C., or 95°C., or 100° C. The antagonist IL-10R peptide or concatenated antagonist IL-10R peptides may maintain their affinity for the IL-10R when heated to a temperature of 70° C. to 90° C. The antagonist IL-10R peptide or concatenated antagonist IL-10R peptides may maintain their affinity for the IL-10R when heated to a temperature in a range between any two of the following values: 70° C., 75°C., 80° C., 85° C., or 90° C.

In an embodiment, the antagonist IL-10R peptide or concatenated antagonist IL-10R peptides may be a peptide or concatenated peptides that are stable to pepsin digestion, may have an increased stability in the animal digestive tract, and may be produced by a microbial host. The antagonist IL-10R peptide or concatenated antagonist IL-10R peptides may be a peptide or concatenated peptides that are readily degradable by pepsin. The readily degradable peptide or concatenated peptides may completely degrade in a time period from 45 minutes to 40 minutes, from 40 minutes to 35 minutes, from 35 minutes to 30 minutes, from 30 minutes to 25 minutes, from 25 minutes to 20 minutes, from 20 minutes to 15 minutes, from 15 minutes to 10 minutes, from 10 minutes to 8 minutes, from 8 minutes to 6 minutes, from 6 minutes to 4 minutes, from 4 minutes to 2 minutes of the pepsin treatment. The time period for degradation may be in a range between any two integer value between 2 minutes and 45 minutes. The complete degradation of the peptide or concatenated peptides by pepsin may occur in 10 minutes.

An embodiment provides an antibody that binds to IL-10, and is referred herein as anti-IL-10 antibody. The anti-IL-10 antibody may bind to the Gallus gallus (chicken) IL-10, and is referred herein as an anti-chIL-10 antibody, or chII10AB. The anti-IL-10 antibody may be a single domain anti-IL-10 antibody (sdAB). Both “single domain anti-IL-10 antibody” and “anti-IL-10 single domain antibody” refer to the same type of antibody and may be used interchangeably herein. An anti-IL-10 single domain antibody may bind to a polypeptide comprising an amino acid sequence of SEQ ID NO: 80. The anti-IL-10 single domain antibody may be capable of reducing IL-10 binding to the IL-10 receptor (IL-10R).

In an embodiment, the anti-IL-10 single domain antibody may have a binding EC₅₀ for chicken II-10 of 30 nM, or less. The anti-IL-10 single domain antibody may have a binding EC50 for chicken IL-10 of about 30 nM, or less, about 25 nM, or less, about 20 nM, or less, about 15 nM, or less, about 10 nM, or less, about 5 nM, or less, or about 1 nM, or less. The anti-IL-10 single domain antibody may have a binding EC₅₀ for chicken II-10 in a range between any two of the following EC₅₀ values: 30, 20, 10, 5, or 1 nM. In an embodiment, the EC₅₀ of the anti-IL-10 single domain antibody provided herein may be measured by ELISA or any other assay known in the art. The EC₅₀ of the anti-IL-10 single domain antibody value may be measured by an ELISA assay described in Example 9 herein.

Without limitations, the anti-IL-10 single domain antibody may be a single domain antibody of any length and of any molecular mass that is capable of reducing IL-10 binding to the IL-10 receptor (IL-10R). The anti-IL-10 single domain antibody may have a molecular mass of 10, 11, 12, 13, 14, 15, 16, 17, 18, 19, or 20 kDa. The anti-IL-10 single domain antibody may have a molecular mass of 14, 15, 16, 17, 18, 19, or 20 kDa. The anti-IL-10 single domain antibody may have a molecular mass in a range between any two of the following molecular masses: 10, 11, 12, 13, 14, 15, 16, 17, 18, 19, or 20 kDa. The anti-II-10 single domain antibody may have a molecular mass in a range between any two of the following molecular masses: 14, 15, 16, 17, 18, 19, or 20 kDa.

The anti-IL-10 single domain antibody may include an amino acid sequence with at least 70, 72, 75, 80, 85, 90, 91, 92, 93, 94, 95, 96, 97, 98, 99 or 100% identity to a reference sequence selected from the group consisting of: SEQ ID NO: 87 [chIL10sdAB1H5], SEQ ID NO: 88 [chIL10sdAB1E9], SEQ ID NO: 89 [chIL10sdAB1H1], SEQ ID NO: 90 [chIL10sdAB1G6], SEQ ID NO: 91 [chIL10sdAB1C10], SEQ ID NO: 92 [chIL10sdAB1B6], SEQ ID NO: 93 [chIL10sdAB1D12], SEQ ID NO:947 [chIL10sdAB1C2], SEQ ID NO: 95 [chIL10sdAB1B5], SEQ ID NO: 96 [chIL10sdAB1E2], SEQ ID NO: 97 [chIL10sdAB1G7], SEQ ID NO: 98 [chIL10sdAB1G9], SEQ ID NO: 99 [chIL10sdAB1H12], SEQ ID NO: 100 [chIL10sdAB2A9], SEQ ID NO: 101 [chIL10sdAB1E12], SEQ ID NO: 102 [chIL10sdAB1E10], SEQ ID NO: 103 [chIL10sdAB1F12], SEQ ID NO: 104 [chIL10sdAB1A8], SEQ ID NO: 105 [chIL10sdAB1C8], SEQ ID NO: 106 [chIL10sdAB1C12], SEQ ID NO: 107 [chIL10sdAB1B1], SEQ ID NO: 108 [chIL10sdAB1F1], SEQ ID NO: 109 [chIL10sdAB1D11], SEQ ID NO: 110 [chIL10sdAB1E6], SEQ ID NO: 111 [chIL10sdAB1B9], SEQ ID NO: 112 [chIL10sdAB1B10], SEQ ID NO: 113 [chIL10sdAB1F5], SEQ ID NO: 114 [chIL10sdAB1A6], SEQ ID NO: 115 [chIL10sdAB1D5], SEQ ID NO: 116 [chIL10sdAB1D8], SEQ ID NO: 117 [chIL10sdAB1B4], SEQ ID NO: 118 [chIL10sdAB1C7], SEQ ID NO: 119 [chIL10sdAB1B3], SEQ ID NO: 120 [chIL10sdAB1D7], SEQ ID NO: 121 [chIL10sdAB1F7], SEQ ID NO: 122 [chIL10sdAB1F10], SEQ ID NO: 123 [chIL10sdAB1F2], SEQ ID NO: 124 [chIL10sdAB1F3], SEQ ID NO: 125 [chIL10sdAB1F8], SEQ ID NO: 126 [chIL10sdAB1C9], SEQ ID NO: 127 [chIL10sdAB1A12], SEQ ID NO: 128 [chIL10sdAB1C3], SEQ ID NO: 129 [chIL10sdAB1E7], SEQ ID NO: 130 [chIL10sdAB1D9], SEQ ID NO: 131 [chIL10sdAB1A9], SEQ ID NO: 132 [chIL10sdAB1H10], SEQ ID NO: 133 [chIL10sdAB1C1], SEQ ID NO: 134 [chIL10sdAB1D1], SEQ NO: 135 [chIL10sdAB1A11], SEQ ID NO: 136 [chIL10sdAB1G8], SEQ ID NO: 137 [chIL10sdAB1A5], SEQ ID NO: 138 [chIL10sdAB1C5], SEQ ID NO: 139 [chIL10sdAB1H6], SEQ ID NO: 140 [chIL10sdAB2A8], SEQ ID NO: 141 [chIL10sdAB1F9], SEQ ID NO: 142 [chIL10sdAB1E11], SEQ ID NO: 143 [chIL10sdAB1D6], SEQ ID NO: 144 [chIL10sdAB1C4], SEQ ID NO: 145 [chIL10sdAB1H4], SEQ ID NO: 146 [chIL10sdAB1F11], SEQ ID NO: 147 [chIL10sdAB1D3], SEQ ID NO: 148 [chIL10sdAB1A7], SEQ ID NO: 149 [chIL10sdAB1H8], SEQ ID NO: 150 [chIL 10sdAB1H3], SEQ ID NO: 151 [chIL10sdAB1B8], SEQ ID NO: 152 [chIL10sdAB1B2], SEQ ID NO: 153 [chIL10sdAB1D2], and SEQ ID NO: 154 [chIL10sdAB1D10]. The anti-IL-10 single domain antibody may be fused to a signal peptide. The signal peptide may be but is not limited to an amyloplast targeting signal, a cell wall targeting peptide, a mitochondrial targeting peptide, a cytosol localization signal, a chloroplast targeting signal, a nuclear targeting peptide, an endoplasmic reticulum retention signal, or a vacuole targeting peptide. The signal peptide may an N-terminal signal peptide or a C-terminal signal peptide. The N-terminal signal peptide may be but is not limited to OsGluB4sp (rice Glu-B4 glutelin signal peptide), BAASS (barley alpha amylase signal sequence), PR1 (pathogenesis related protein), or zein 27 (xGZm27ss) signal peptide. The C-terminal signal peptide may be but is not limited to KDEL (SEQ ID NO: 29), HDEL (SEQ ID NO: 30), SEKDEL (SEQ ID NO: 31), HvVSD from barley polyamine oxidase, or HvAle from barley aleurone (thiol protease). The anti-IL-10 single domain antibody may be fused to the N-terminal signal peptide or C-terminal signal peptide, or both.

In an embodiment, the anti-IL-10 single domain antibody having less than 100% identity to its corresponding amino acid sequence of one of SEQ ID NO: 87-154 may be a variant of the referenced peptide or amino acid. In an embodiment, an isolated peptide having a sequence with at least 70, 75, 80, 85, 90, 91, 92, 93, 94, 95, 96, 97, 98, 99, or 100% identity to a peptide having the sequence of any one of SEQ ID NOS: 87-154 along 10 to 20, 10 to 25, 10 to 30, 10 to 40, 10 to 50, 10 to 60, 10 to 70, 10 to 80, 10 to 90, 10 to 100, 10 to 110, 10 to 115, 10 to 116, 10 to 117, 10 to 118, 10 to 119, 10 to 120, 10 to 121, 10 to 122, 10 to 123, 10 to 124, 10 to 125, 10 to 126, or 10 to all amino acids of a protein having the sequence of any of one any one of SEQ ID NOS: 87-154 is provided. This list of sequence lengths encompasses every full length peptide in SEQ ID NOS: 87-154 and every smaller length within the list, even for peptides that do not include over 126 amino acids. For example, the lengths of 10 to 20, 10 to 25, 10 to 30, 10 to 40, 10 to 50, 10 to 60, 10 to 70, 10 to 80, 10 to 90, 10 to 100, 10 to 110, 10 to 115, 10 to 116, 10 to 117, 10 to 118, 10 to 119, 10 to 120, 10 to 121, 10 to 122, 10 to 123, 10 to 124, 10 to 125, 10 to 126, or 10 to all amino acids would apply to a sequence with 126 amino acids. A range of amino acid sequence lengths recited herein includes every length of amino sequence within the range, endpoints inclusive. The recited length of amino acids may start at any single position within a reference sequence where enough amino acids follow the single position to accommodate the recited length. The fragment of the anti-IL-10 single domain antibody may be a subsequence of the polypeptides herein that retain at least 40% of the anti-IL-10 single domain antibody's EC₅₀ value when used in a cellular assay comprising cells that are inhibited by IL-10 to decrease production of interferon gamma in the presence of ConA, which is described herein in Example 9. The fragment may have 10, 15, 20, 25, 30, 35, 40, 45, 50, 55, 60, 65 70, 75, 80, 85, 90, 95, 100, 115, 116, 117, 118, 19, 120, 121, 122, 123, 124, 124, or 126 amino acids. The fragments may include 10, 15, 20, 25, 30, 35, 40, 45, 50, 55, 60, 65 70, 75, 80, 85, 90, 95, 100, 115, 116, 117, 118, 19, 120, 121, 122, 123, 124, 124, or 126 contiguous amino acids. Embodiments also include nucleic acids or polynucleotides, encoding said amino acid sequences. A less than full length amino acid sequence may be selected from any portion of one of the sequences of SEQ ID NOS: 87-154 corresponding to the recited length of amino acids. A less than full length amino acid sequence may be selected from a portion of any one of SEQ ID NOS: 87-154.

The anti-IL-10 single domain antibody may increase the production of interferon gamma or nitric oxide when used in a cellular assay comprising cells that are inhibited by IL-10 to decrease production of interferon gamma or nitric oxide.

The anti-IL-10 single domain antibody may maintain or improve the body weight, and, or the feed conversion ratio, of poultry fed the antibodies relative to poultry fed the same feed lacking the antibodies. The anti-IL-10 single domain antibody may be dosed at less than 500 mg per kg of pelleted feed, or more preferably at less than 50 mg per kg of pelleted feed, or even more preferably at less than 5 mg per kg of pelleted feed, or even more preferably at less than 1 mg per kg of pelleted feed. The anti-IL-10 single domain antibody may also improve the body weight or feed conversion ratio of poultry when used in mash (non-pelleted) feed. The anti-IL-10 single domain antibody may maintain its affinity for IL-10 following exposure to pelleting process temperature less than or equal to 65° C., or 70° C., or 75° C., or 80° C., or 85° C., or 90° C., or 95° C., or 100° C. The anti-IL-10 single domain antibody may maintain its affinity for IL-10 following incubation in liquid for at least 60 seconds at a temperature less than or equal to 65° C., or 70° C., or 75° C., or 80° C., or 85° C., or 90° C., or 95° C., or 100° C. The anti-IL-10 single domain antibody may have activity when heated to a temperature of 70° C. to 90° C. The anti-IL-10 single domain antibody may have activity when heated to a temperature in a range between any two of the following values: 70° C., 75° C., 80° C., 85° C., or 90° ° C. The anti-IL-10 single domain antibody may be active following exposure of a temperature of 70° C. to 90° C., or any value in between the foregoing values. The anti-IL-10 single domain antibody may be an antibody stable to pepsin digestion, may have an increased stability in the animal digestive tract, and may be produced by a microbial host. The anti-IL-10 single domain antibody may be an antibody that is readily degradable by pepsin. The readily degradable antibody may completely degrade in a time period from 45 minutes to 40 minutes, from 40 minutes to 35 minutes, from 35 minutes to 30 minutes, from 30 minutes to 25 minutes, from 25 minutes to 20 minutes, from 20 minutes to 15 minutes, from 15 minutes to 10 minutes, from 10 minutes to 8 minutes, from 8 minutes to 6 minutes, from 6 minutes to 4 minutes, from 4 minutes to 2 minutes of the pepsin treatment. The time period for degradation may be in a range between any two integer value between 2 minutes and 45 minutes. The complete degradation of the antibody by pepsin may occur in 10 minutes.

An embodiment provides one or more synthetic polynucleotides encoding the anti-IL-10 single domain antibody or their variants described herein. The one or more synthetic polynucleotides may comprise, consist essentially of, or consist of a sequence with at least 70, 72, 75, 80, 85, 90, 91, 92, 93, 94, 95, 96, 97, 98, 99, or 100% identity to a reference sequence of SEQ ID NO: 173 [chIL101A11 coding seq], SEQ ID NO: 174 [chIL101A11B coding seq], SEQ ID NO: 175 [chIL101F11A coding seq], SEQ ID NO: 176 [chIL101F11B coding seq], SEQ ID NO: 177 [chIL101H1A coding seq], or SEQ ID NO: 178 [chIL101H1B coding seq]. The one or more synthetic polynucleotides may be included in the expression cassette to be expressed in a host. The host may be but is not limited to a microorganism, a plant cell, a phage, a virus, a mammalian cell, or an insect cell.

An embodiment provides an expression cassette. The expression cassette may comprise one or more synthetic polynucleotide encoding the antagonist IL-10R peptide, concatenated IL-10R antagonist peptides, anti-IL-10 single domain antibody or their variants described herein.

A polynucleotide sequence in an expression cassette, isolated nucleic acid, vector, or any other DNA construct herein, or utilized in a method herein may be operably connected to one or more regulatory elements. A regulatory element included may be a promoter. The promoter may be a constitutive promoter which provides transcription of the polynucleotide sequences throughout the plant in most cells, tissues and organs and during many but not necessarily all stages of development. The promoter may be an inducible promoter, which initiates transcription of the polynucleotide sequences only when exposed to a particular chemical or environmental stimulus. The promoter may be specific to a host. The promoter may be suitable for expression of the polynucleotide in a plant, a bacterium, yeast, a mammalian cell, or an insect cell. The promoter may be a plant specific promoter. The promoter may be specific to a particular developmental stage, organ or tissue. A tissue specific promoter may be capable of initiating transcription in a particular plant tissue. Plant tissue that may be targeted by a tissue specific promoter may be but is not limited to a stem, leaves, trichomes, anthers, seed, embryo, or endosperm. A constitutive promoter herein may be the maize Ubiquitin promoter, the rice Ubiquitin 3 promoter (OsUbi3P), the switchgrass ubiquitin promoter, the PEPC promoter, the maize Actin promoter, or the rice Actin 1 promoter. Other known constitutive promoters may be used, and include but are not limited to Cauliflower Mosaic Virus (CAMV) 35S promoter, the Cestrum Yellow Leaf Curling Virus promoter (CMP) or the CMP short version (CMPS), and the Rubisco small subunit promoter.

The tissue specific promoter may include the seed-specific promoter. The seed specific promoter may be but is not limited to the maize zein promoter, the rice glutelin (GluB4) promoter, the maize oleosin promoter, or the maize globulin promoter.

The promoter may be a promoter homolog to any one of the previously listed promoters derived from other species, or promoter variants to the previously listed promoters with greater than 80% identity.

The promoter may be suitable for expressing the one or more polynucleotides in a bacterium. The promoter may be the T7 RNA polymerase promoter, the LAC promoter or the arabinose promoter. The promoter may be suitable for expressing the polynucleotide in a yeast. The promoter may be the GAL promoter or the glucose promoter. The promoter may be any prokaryotic promoter. The prokaryotic promoter may be a bacterial promoter, or phage promoter that is active in bacteria. The prokaryotic promoter may be any inducible promoter that is active in bacteria, or any other promoter that is active in bacteria.

Another regulatory element that may be provided is a terminator sequence, which terminates transcription. A terminator sequence may be included at the 3′ end of a transcriptional unit of the expression cassette. The terminator may be derived from a variety of genes. The terminator may be from a eukaryote, such as a plant or mammalian cell, or a prokaryote. The terminator may be a terminator sequence from the nopaline synthase or octopine synthase genes of Agrobacterium tumefaciens. The terminator may be maize gamma zein 27 terminator. The terminator may be any other terminator sequence.

The one or more synthetic polynucleotide may further include one or more signal polynucleotide sequence encoding any one of the signal peptides described herein. The expression cassette may comprise, consist essentially of, or consist of a synthetic polynucleotide sequence with at least 70, 72, 75, 80, 85, 90, 91, 92, 93, 94, 95, 96, 97, 98, 99, or 100% identity to a reference sequence of SEQ ID NO: 84 [xGZein27ss: chIL10sdAB1A11:KDEL], SEQ ID NO: 85 [xGZein27ss:chIL10 sdAB 1B9:KDEL], SEQ ID NO: 86 [xGZein27ss:chIL10sdAB1F11:KDEL], or SEQ ID NO: 179 [xGZein27ss:chIL10sdAB1H1:KDEL].

The expression cassette including the one or more synthetic polynucleotides may be included in a vector.

An embodiment comprises a vector containing the expression cassette including one or more synthetic polynucleotides encoding the antagonist IL-10R peptide, the concatenated antagonist IL-10R peptides, or the anti-IL-10 single domain antibody of any of the above embodiments. The vector may contain any one of the expression cassettes described in any of the embodiments herein. The vector may be a vector used in plant transformation and that is capable of delivering its DNA into the genome of plant cells. The vector may be a vector used for yeast and fungal expression. The vector may be a vector for expression of the peptides, concatenated peptides or antibodies described herein used in bacterial expression. The vector may be a vector used for mammalian or insect cell expression.

The vector may comprise the expression cassette including synthetic polynucleotides encoding the antagonist IL-10R peptide or concatenated antagonist IL-10R peptides. The vector may comprise a nucleic acid sequence with at least 70, 72, 75, 80, 85, 90, 91, 92, 93, 94, 95, 96, 97, 98, 99, or 100% identity to a reference sequence of SEQ ID NO: 69 [pAG4305], SEQ ID NO: 70 [pAG4306], SEQ ID NO: 71 [pAG4308], SEQ IS NO: 72 [pAG4310], SEQ ID NO: 73 [pAG4311], SEQ ID NO: 74 [pAG4312], SEQ ID NO: 75 [pAG4313], SEQ ID NO: 76 [pAG4981], SEQ ID NO: 77 [pAG4982], SEQ ID NO: 78 [pAG4983], and SEQ ID NO: 79 [pAG4984].

The vector may comprise the expression cassette including the synthetic polynucleotide encoding an anti-IL-10 single domain antibody. The vector may comprise a nucleic acid sequence with at least 70, 72, 75, 80, 85, 90, 91, 92, 93, 94, 95, 96, 97, 98, 99, or 100% identity to a reference sequence of SEQ ID NO: 155 [pAG4314], SEQ ID NO: 156 [pAG4315], SEQ ID NO: 157 [pAG4316], SEQ ID NO: 158 [pAG4317], SEQ ID NO: 159 [pAG4985], SEQ ID NO: 160 [pAG4986], SEQ ID NO: 161 [pAG4987], SEQ ID NO: 162 [pAG4988], SEQ ID NO: 163 [pAG4989], SEQ ID NO: 164 [pAG4990], SEQ ID NO: 165 [pAG4991], SEQ ID NO: 166 [pAG4992], SEQ ID NO: 167 [pAG4993], SEQ ID NO: 168 [pAG4994], SEQ ID NO: 169 [pAG4995], SEQ ID NO: 170 [pAG4996], SEQ ID NO: 171 [pAG4997] , or SEQ ID NO: 172 [pAG4998].

An embodiment comprises a polynucleotide comprising, consisting essentially of, or consisting of a sequence that has at least 70, 72, 75, 80, 85, 90, 91, 92, 93, 94, 95, 96, 97, 98, 99, or 100% identity along its length to a contiguous portion of a polynucleotide having any one of the sequences set forth herein or the complements thereof. The contiguous portion may be any length up to the entire length of a sequence set forth herein or the complement thereof.

Determining percent identity of two amino acid sequences or two nucleic acid sequences may include aligning and comparing the amino acid residues or nucleotides at corresponding positions in the two sequences. If all positions in two sequences are occupied by identical amino acid residues or nucleotides then the sequences are said to be 100% identical. Percent identity is measured by the Smith Waterman algorithm (Smith T F, Waterman M S 1981 “Identification of Common Molecular Subsequences,” J Mol Biol 147: 195 -197, which is incorporated herein by reference as if fully set forth).

In an embodiment, a transgenic plant comprising any one of synthetic polynucleotides described herein and expressing any one of the antagonist IL-10R peptides, concatenated antagonist IL-10R peptides or anti-IL-10 single domain antibodies described herein is provided. As used herein, the term “transgenic plants” describes plants transformed with DNA that enables the plant containing the transformed DNA to perform a novel function; usually the transcription of the DNA, potentially at a level different from the level in wild-type plants, and potentially the translation of the transcript into a protein, which may be a novel protein to the plant. The transgenic plant may refer to a whole transgenic plant or tissues thereof. The tissues of transgenic plants may be any portion of a transgenic plant, including but not limited to leaves, stems, flowers, buds, petals, grain, seed, embryo, endosperm, leaves, stalks, roots, pollen, or anthers. The tissues may also refer to liquid extracts made by fractionating any portion of a transgenic plant in an organic or aqueous liquid (for example, extracting protein from transgenic seeds and using the extract as a source of the transgenic protein) and using the separated liquid to feed an animal, or in animal feed, or an animal feed additive. The tissue may be callus from a transgenic plant. The tissue may be seeds from a transgenic plant that accumulate peptides, antagonist IL-10R peptides, concatenated antagonist IL-10R peptides, or anti-IL-10 single domain antibodies described herein. A transgenic plant may be regenerated from tissues of a transgenic plant. A transgenic plant may be a product of sexual crossing of a first transgenic plant and a second transgenic plant or a non-transgenic plant where the product plant retains a synthetic nucleic acid introduced to the first transgenic plant. A transgenic plant may be a product of self-pollination of a first transgenic plant with itself.

An embodiment provides a progeny of any one of the transgenic plants described herein. The transgenic plant may express any one of the antibodies described herein. The antibodies may target endogenous molecules produced by the host animal ingesting the transgenic plant or tissues thereof. The targeted endogenous molecules may be but are not limited to interleukins, cytokines, hormones, peptides, cellular receptors, clusters of differentiation, or related molecules. The transgenic plants of the present disclosure may express other peptides or proteins that impact immune response of an animal fed with the transgenic plant or tissues thereof. The transgenic plant may contain at least one of the expression cassettes that are described herein. The transgenic plant may be produced using the vectors described herein. The transgenic plant may be capable of producing any one of the peptides, antagonist IL-10R peptides, concatenated antagonist IL-10R peptides, or anti-IL-10 single domain antibodies described herein. The transgenic plant expressing peptides, antagonist IL-10R peptides, concatenated antagonist IL-10R peptides, or anti-IL-10 single domain antibodies described herein, may be but is not limited to tobacco plant, corn plants, soy bean plants, or any other plant commonly eaten by animals.

The transgenic plants may express peptides and proteins that modulate, stimulate, or augment the immune system, or immune response of an animal fed the transgenic plants or tissues thereof. The transgenic plants may express antibodies targeting endogenous molecules produced by the host animal ingesting the transgenic plants or tissues thereof. The antibodies expressed by the transgenic plant may bind to molecules such as interleukins, cytokines, hormones, peptides, cellular receptors, clusters of differentiation, or similar molecules. The transgenic plants may express other peptides or proteins that modulate various endogenous immune system pathways, endocrine pathways, or other physiological systems. More specifically, the transgenic plants may express express one or more antibodies that bind to interleukin 10 (IL-10), or one or more peptide or protein antagonists that interfere or block the IL-10 receptor complex (IL-10R), or one or more peptide or protein molecules that otherwise inhibit IL-10 signaling pathways.

The transgenic plants, or tissue thereof may modulate, stimulate, or augment the immune system, or immune response of an animal fed the transgenic plant or tissues thereof. The transgenic plants or tissues thereof may improve the gastrointestinal physiology of an animal eating the plants. The transgenic plants or tissues thereof may decrease the binding of IL-10 with the IL-10 receptor (IL-10R) when fed to poultry. The transgenic plants or tissues thereof may maintain or improve the body weight, and, or the feed conversion ratio, of poultry fed the transgenic plants or tissues thereof, relative to poultry fed the same feed lacking the transgenic plants or tissues thereof. The transgenic plants or tissues thereof may be dosed at less than 700 kg per ton of pelleted feed, or more preferably at less than 5 kg per ton of pelleted feed, or more preferably at less than 1 kg per ton of pelleted feed, or even more preferably at less than 500 g per ton of pelleted feed, or even more preferably at less than 50 g per ton of pelleted feed, or yet even more preferably at less than 5 g per ton of pelleted feed. The transgenic plants and tissues thereof may also improve the body weight or feed conversion ratio of poultry when used in mash (non-pelleted) feed.

In an embodiment, a method of making any one of the transgenic plants described herein is provided. The method may comprise culturing explants from a target plant and contacting them with a vector that contains at least one expression cassette described herein. The target plant may be a corn or soy bean plant, or it may be wheat, rice, sorghum, tobacco, canola, cotton, switchgrass, or another plant. The method may include contacting the vector with the plant explant, for example, by using biolistic transformation or by using Agrobacterium transformation. Once the explant has been contacted by the vector, methods of selecting and regenerating whole plants may be used that are known in the art.

In an embodiment, any one of the antagonist IL-10R peptides, concatenated antagonist IL-10R peptides or anti-IL-10 single domain antibodies may be isolated from the transgenic plant or plant tissue.

In an embodiment, the specific recombinant, engineered or synthetic molecules described herein may be expressed by other hosts and may be isolated from the hosts.

In an embodiment, the transgenic plants or tissues thereof, or the isolated antagonist IL-10R peptides, concatenated antagonist IL-10R peptides or anti-IL-10 single domain antibodies may be included in an animal feed.

In an embodiment, an animal feed that includes any one of the transgenic plants, or tissues thereof described herein is provided. The term “animal feed” refers to any food, feed, feed composition, preparation, additive, supplement, or mixture suitable and intended for intake by animals for their nourishment and growth. The animal feed comprising transgenic plants, or plant tissues, may decrease the binding of IL-10 with the IL-10R when fed to poultry. The animal feed comprising transgenic plants, or plant tissues, may maintain or improve the body weight, and, or the feed conversion ratio, of poultry fed the transgenic plants or tissues thereof, relative to poultry fed the same feed lacking the transgenic plants or tissues thereof. The animal feed may comprise transgenic plants or tissues thereof at less than 700 kg per ton of pelleted feed, or more preferably at less than 5 kg per ton of pelleted feed, or more preferably at less than 1 kg per ton of pelleted feed, or even more preferably at less than 500 g per ton of pelleted feed, or even more preferably at less than 50 g per ton of pelleted feed, or yet even more preferably at less than 5 g per ton of pelleted feed. The animal feed or animal feed additives comprising transgenic plants and tissues thereof may also improve the body weight or feed conversion ratio of poultry when used in mash (non-pelleted) feed. The animal feed may include an isolated antagonist IL-10R peptide, concatenated antagonist IL-10R peptides or anti-IL-10 single domain antibody. The antagonist IL-10R peptide, concatenated antagonist IL-10R peptides or anti-IL-10 single domain antibodies included in the animal feed may be active in the gastrointestinal environment of animals. The antagonist IL-10R peptide, concatenated antagonist IL-10R peptides or anti-IL-10 single domain antibody included the animal feed may be a peptide or antibody that is stable to pepsin digestion. The antagonist IL-10R peptide, concatenated antagonist IL-10R peptides, anti-IL-10 single domain antibodies included the animal feed may be a peptide or antibody that is digested by pepsin. The animal may be a monogastric animal. The monogastric animal may be but is not limited to a chicken, a turkey, or a duck. The antagonist IL-10R peptide, concatenated antagonist IL-10R peptides or anti-IL-10 single domain antibody may be active after preparation of the animal feed. The temperatures which feeds are exposed to during preparation may be within the range of 20° C to 70° C, endpoints inclusive. The temperature may be 20° C., 25° C., 30° C., 35° C., 40° C., 45° C., 50° C., 55° C, 60° C., 65° C., 70° C., 20° C., to 25° C., 20° C., to 30° C., 2° C. to 35° C., 20° C. to 40° C., 20° C., to 45° C., 20° C. to 50° C., 20° C. to 55° C., 20° C. to 60° C., 20° C. to 65° C., 20° C. to 70° C., 30° C. to 70° C., 40° C. to 70° C., 50° C. to 70° C., 60° C. to 60° C., or less than 70° C. The temperatures which feeds are exposed to during pelleting may be within the range of 70° C. to 130° C., endpoints inclusive. The temperature may be 70° C., 75° C., 80° C., 85° C., 90° C., 95° C., 100° C., 105° C., 110° C., 115° C., 120° C., 125° C., 130° C., 0° C. to 75° C., 70° C. to 80° C., 70° C. to 85° C., 70° C. to 90° C., 70° C. to 95° C., 70° C. to 100° C., 70° C. to 105° C., 70° C. to 110° C., 70° C. to 115° C., 70° C. to 120° C., 70° C. to 125° C., 70° C. to 130° C., 80° C. to 130° C., 90° C. to 130° C., 100° C. to 130° C., 110° C. to 130° C., 120° C. to 130° C., or less than 130° C.

The antagonist IL-10R peptide, concatenated antagonist IL-10R peptides or anti-IL-10 single domain antibody may have improved thermal stability and may retain activity after being exposed to high temperatures during feed pelleting.

In an embodiment, the animal feed may further include a feed supplement. The feed supplement may be any plant material. The plant material may be a non-transgenic plant or a transgenic plant. The plant material may include a transgenic plant or a mutant plant. The plant material may be a grain that contains starch. The plant material may be a grain that contains fiber. The plant material may be achemically treated forage. The plant material may be a non-transgenic plant or part thereof. The plant material may include at least one component selected from the group consisting of: barley, wheat, rye, oat, corn, rice, triticale, beet, sugar beet, spinach, cabbage, quinoa, corn meal, corn pellets, corn oil, distillers grains, forage, wheat meal, wheat pellets, wheat grain, barley grain, barley pellets, soybean meal, soybean oilcake, lupin meal, rapeseed meal, sorghum grain, sorghum pellets, rapeseed, sunflower seed, and cotton seed.

The feed supplement may be a mineral. The mineral may be a trace mineral. The mineral may be a macro mineral. The mineral may be rock phosphate or a phosphate salt. The mineral may be calcium phosphate. The feed supplement may be at least one vitamin. The at least one vitamin may be a fat-soluble vitamin. The feed supplement may be an amino acid. The feed supplement may include one or more exogenous enzymes. The one or more exogenous enzymes may include a phytase enzyme. The one or more exogenous enzymes may include a hydrolytic enzyme. The hydrolytic enzyme may be an enzyme classified under EC3.4 as hydrolase. The hydrolytic enzymes may be, but are not limited to, xylanases, mannanases, carbohydrases, proteases, peptidases, glucanases, cellulases, lipases, phospholipases, pectinases, galactosidases, laccases, amylases, hemicellulases, or cellobiohydrolases. The enzymes may be expressed in the transgenic plants or parts thereof included in the feed supplement. The feed supplement may include purified enzymes. The feed supplements may be but are not limited to growth improving additives, coloring agents, flavorings, stabilizers, limestone, stearine, starch, saccharides, fatty acids, or a gum. The coloring agents may be carotenoids. The carotenoids may be but are not limited to cantaxanthin, beta-carotene, astaxanthin, or lutein. The fatty acids may be polyunsaturated fatty acids. The polyunsaturated fatty acids may include but are not limited to arachidonic acid, docosohexaenoic acid (DHA), eicosapentaenoic acid (EPA) or gamma-linoleic acid.

The feed supplement may include at least one component selected from the group consisting of: soluble solids, fat and vermiculite, limestone, plain salt, DL-methionine, L-lysine, L-threonine, COBAN®, vitamin premix, dicalcium phosphate, selenium premix, choline chloride, sodium chloride, and mineral premix. The feed supplement may include fish meal, fish oil, bone meal, feather meal and animal fat. The feed supplement may include yeast or yeast extract.

In an embodiment, a method of preparing an animal feed is provided. The method may include producing any one of the antagonist IL-10R peptides, concatenated antagonist IL-10R peptides or anti-IL-10 single domain antibodies described herein by any one of the methods described herein.

An embodiment provides a method of producing an animal feed. The method may include mixing any one of the transgenic plants or tissues thereof described herein, or the progeny thereof with plant material. The transgenic plant may be a progeny of the transgenic plant that include one or more synthetic polynucleotides encoding peptides and antibodies described herein. The one or more polynucleotides may be included in a genetic construct(s) or an expression cassette(s). The method may comprise making any transgenic plant herein. The transgenic plant or its progeny may be the plant expressing a peptide or protein produced by the method herein. The method may further include pelletizing the mixture. The method may further include adding a feed supplement to the mixture. The feed supplement may include at least one exogenous enzyme. The at least one exogenous enzyme may be selected from the group consisting of: phytase, xylanase, mannanase, protease, glucanase, and cellulase. Preparing the animal feed may include combining one or more transgenic plants described herein with any other feed supplement.

An expression cassette having one or more polynucleotides encoding an antagonist IL-10R peptide, concatenated antagonist IL-10R peptides or anti-IL-10 single domain antibody in a plant may be expressed prior to the step of step of mixing the plant, or prior to the step of pelletizing the plant. The expression may be constitutive or the expression may be induced. Upon the expression of the nucleic acid(s), the transgenic plant may have an increased level of the antagonist IL-10R peptide, concatenated antagonist IL-10R peptides or anti-IL-10 single domain antibodies compared to the level of antagonist IL-10R peptide, concatenated antagonist IL-10R peptides or anti-IL-10 single domain antibodies in a non-transgenic plant of the same genetic background but lacking the one or more expression cassettes.

The antagonist IL-10R peptide, concatenated antagonist IL-10R peptides or anti-IL-10 single domain antibodies may be isolated, purified and added to the animal feed as a pure antagonist IL-10R peptide, concatenated antagonist IL-10R peptides or anti-IL-10 single domain antibodies. The antagonist IL-10R peptide, concatenated antagonist IL-10R peptides or anti-IL-10 single domain antibodies may be isolated from the intact host organism and added to the animal feed as an antagonist IL-10R peptide, concatenated antagonist IL-10R peptides or anti-IL-10 single domain antibodies composition. The antagonist IL-10R peptide, concatenated antagonist IL-10R peptides or anti-IL-10 single domain antibodies may be added to the animal feed in admixture with other feed supplements. The transgenic plant including the antagonist IL-10R peptide, concatenated antagonist IL-10R antagonist peptides or anti-IL-10 single domain antibodies or the purified antagonist IL-10R peptide, concatenated antagonist IL-10R peptides or anti-IL-10 single domain antibodies may be combined with other feed supplements to form premixes.

An animal feed may be produced as mash feed. The animal feed may be produced as pelleted feed. The milled feed stuffs may be mixed with the premix that includes any one of the transgenic plants that include an antagonist IL-10R peptide, concatenated antagonist IL-10R peptides or anti-IL-10 single domain antibody. The antagonist IL-10R peptide, concatenated antagonist IL-10R peptides or anti-IL-10 single domain antibody may be an antagonist IL-10R peptide, concatenated antagonist IL-10R peptides or anti-IL-10 single domain antibody that is/are stable to pepsin digestion. The antagonist IL-10R peptide, concatenated antagonist IL-10R peptides or anti-IL-10 single domain antibody may be an antagonist IL-10R peptide, concatenated antagonist IL-10R peptides or anti-IL-10 single domain antibody that is/are digestable by pepsin. The milled feed stuffs may include the plant material and the feed supplements described herein. The feed supplements may include one or more exogenous enzymes described herein. Enzymes may be added as liquid or solid formulations. For mash feed, a solid or liquid peptide formulation may be added before or during the mixing step. For pelleted feed, the peptide preparation may be added before or after the pelleting step. The antagonist IL-10R peptide, concatenated antagonist IL-10R peptides or anti-IL-10 single domain antibody may be included in premix. The premix may also include vitamins and trace minerals. Macro minerals may be added separately to animal feedstock.

An embodiment comprises a method of treating or preventing a gastrointestinal infection in an animal. The gastrointestinal infection may be caused by a gastrointestinal pathogen. As used herein, a gastrointestinal pathogen may include a bacterium, yeast, fungi, archaea, virus, protozoa, or other infectious agent that is capable of replication inside or outside of the infected host animal, and causes irritation, necrosis, cellular disruption, or cellular damage within the infected host animal, or otherwise stimulates or modulates the immune system of the infected host animal. The gastrointestinal pathogen may belong to the genus Eimeria. The gastrointestinal pathogen may be but is not limited to Eimeria tenella, Eimeria acervulina, or Eimeria maxima. The method may include administering to an animal any one of the antagonist IL-10R peptides or anti-IL-10 single domain antibodies described herein. Administering may be performed by any known route, for example, by injection. Administering may be performed by feeding the infected animal with the transgenic plant expressing one or more antibodies that bind to IL-10, or one or more peptide or protein antagonists to the IL-10R, or plant tissues thereof, or animal feed or feed compositions containing the transgenic plants and tissues thereof. The method comprises administering any one of the antagonist IL-10R peptides or anti-IL-10 single domain antibodies described herein in a therapeutically effective amount. As used herein, a therapeutically effective amount of an antagonist IL-10 peptide, concatenated antagonist IL-10 peptides or anti-IL-10 single domain antibody is an amount effective to reduce the symptoms of the gastrointestinal disease in the animal when administered daily for a period of from one week to two months. The therapeutically effective amounts of the antagonist IL-10R peptide or concatenated antagonist IL-10R peptides may be a dose of less than 500 mg per kg of pelleted feed, or more preferably less than 50 mg per kg of pelleted feed, or even more preferably less than 5 mg per kg of pelleted feed, or even more preferably less than 1mg per kg of pelleted feed.

The therapeutically effective amounts of the anti-IL-10 single domain antibody may be at a dose of less than 500 mg per kg of pelleted feed, or more preferably at a dose of less than 50 mg per kg of pelleted feed, or even more preferably at a dose of less than 5 mg per kg of pelleted feed, or even more preferably at a dose of less than 1mg per kg of pelleted feed.

The therapeutically effective amounts of the transgenic plants or tissues thereof may be at a dose of less than 700 kg per ton of pelleted feed, or more preferably at a dose of less than 5 kg per ton of pelleted feed, or more preferably at a dose of less than 1 kg per ton of pelleted feed, or even more preferably at a dose of less than 500 g per ton of pelleted feed, or even more preferably at a dose of less than 50 g per ton of pelleted feed, or yet even more preferably at a dose less than 5 g per ton of pelleted feed.

The therapeutically effective amounts of the animal feed may comprise transgenic plants or tissues thereof at less than 700 kg per ton of pelleted feed, or more preferably at less than 5 kg per ton of pelleted feed, or more preferably at less than 1 kg per ton of pelleted feed, or even more preferably at less than 500 g per ton of pelleted feed, or even more preferably at less than 50 g per ton of pelleted feed, or yet even more preferably at less than 5 g per ton of pelleted feed.

An embodiment comprises a method of stimulating or modulating the immune system and improving gastrointestinal physiology of an animal comprising feeding the animal with the transgenic plants or tissues thereof. As used herein, the term “modulate” means to change, or respond to a stimulus. In this context “modulate” could mean to increase a response or decrease a response. With regards to modulating an immune response, it means to stimulate or to decrease an immune response. Words that are used synonymously with decrease as it relates to modulation of a response include blocking, interfering, antagonizing, lowering, alleviating, shutting down, or removing. The term gastrointestinal physiology describes the biological state of an animal's gastrointestinal tract, including the foregut, midgut, and hindgut. The actual anatomical features of the gastrointestinal tract may vary among animal species, but in poultry include the esophagus, crop, proventriculus, ventriculous, gizzard, duodenum, jejunum, ileum, small intestine, large intestine, cloaca, and ceca. The biological state of the gastrointestinal tract may be described as healthy or normal, lacking any abnormal visual or pathological observation, or aberrant histological evaluation. The biological state of the gastrointestinal tract may be described as inflamed, infected, or necrotic, all of which describe a physiological state that is impaired and could be improved to a normal or healthy state.

In an embodiment, a method of improving the gastrointestinal physiology of an animal is provided. The method may comprise feeding the animal any of the transgenic plants, or plant parts, described herein. In an embodiment, the method may comprise feeding the animal any of the anti-IL-10 single domain antibodies, peptides, or antagonist IL-10R s described herein.

In an embodiment, a method of improving animal performance or animal gastrointestinal physiology is provided. The method may comprise feeding the animals any of the transgenic plants expressing one or more antibodies that bind to IL-10, or one or more peptide or protein antagonists to the IL-10R, or plant tissues thereof, or feed, or feed compositions containing the transgenic plants or tissues thereof, or anti-IL-10 single domain antibodies, or the IL-10R antagonists. The method may comprise feeding the animals any one of the transgenic plants, or plant parts described herein. As used herein, animal performance is synonymous with animal growth or animal productivity, and each term can be used interchangeably. Animal performance relates to the weight gain of the animal over time, and to the animal's feed conversion ratio, which is defined as the mass of feed eaten by the animal divided by the weight gain of the animal. These terms may be used to describe either, or both, weight gain and feed conversion ratio, so an improvement in animal performance may indicate an increase in weight gain relative to control animals, and, or, a decrease (less feed eaten per mass of animal growth) in feed conversion ratio. In an embodiment, the method may comprise feeding an animal any of the animal feed or animal feed additives comprising any of the anti-IL-10 single domain antibodies, peptides, IL-10R antagonists, or transgenic plants or tissues thereof, described herein.

The following list includes particular embodiments of the present invention. But the list is not limiting and does not exclude alternate embodiments, or embodiments otherwise described herein. Percent identity described in the following embodiments list refers to the identity of the recited sequence along the entire length of the reference sequence.

### EMBODIMENTS

1. At least one antagonist IL-10R peptide, wherein (i) the at least one antagonist IL-10R peptide is one peptide comprising an amino acid sequence with at least 90% identity to a reference sequence selected from the group consisting of: SEQ ID NOS: 1-13, or (ii) the at least one antagonist IL-10R peptide comprises concatenated peptides comprising an amino acid sequence with at least 90% identity to a reference sequence selected from the group consisting of: SEQ ID NOS: 32-40.
2. The at least one antagonist IL-10R peptide of embodiment 1, wherein each of the concatenated peptides are linked to each other by one or more linkers.
3. The at least one antagonist peptide of any one or both of embodiments 1 and 2, wherein the one or more linkers comprise a sequence selected from the group consisting of SEQ ID NOS: 41-44, and 65.
4. The at least one antagonist IL-10R peptide of any one or more of embodiments 1-3, wherein the at least one peptide or each one of the concatenated peptides comprise an N-terminal signal peptide or C-terminal signal peptide, or both.
5. The at least one antagonist IL-10R peptide of any one or more of embodiments 1-4, wherein the N-terminal signal peptide is selected from a group consisting of: OsGluB4sp (rice Glu-B4 glutelin signal peptide), BAASS (barley alpha amylase signal sequence), PR1 (pathogenesis related protein), or zein 27 (xGZm27ss) signal peptide.
6. The at least one antagonist IL-10R peptide of any one or more of embodiments 1-5, wherein the peptide is stable at a temperature in a range from 70° C. to 90° C.
7. The at least one antagonist IL-10R peptide of any one or more of embodiments 1-6, wherein the peptide is digestible by pepsin.
8. The at least one antagonist IL-10R peptide of any one or more of embodiments 1-6, wherein the peptide is stable to digestion by pepsin.
9. The at least one antagonist IL-10R peptide of any one or more of embodiments 1-8, wherein the C-terminal signal peptide is selected from a group consisting of: KDEL (SEQ ID NO: 29), HDEL (SEQ ID NO: 30), SEKDEL (SEQ ID NO: 31), HvVSD from barley polyamine oxidase, or HvAle from barley aleurone (thiol protease).
10. A synthetic polynucleotide encoding the at least one antagonist IL-10R peptide of any one or more of embodiments 1-9.
11. The synthetic polynucleotide of embodiment 10, wherein the synthetic polynucleotide comprises a sequence with at least 90% identity to a reference sequence selected from the group consisting of: 16-28, and 56.
12. An anti-IL-10 single domain antibody that binds to a polypeptide comprising an amino acid sequence of SEQ ID NO: 80.
13. The anti-IL-10 single domain antibody of embodiment 12, wherein the antibody has a molecular mass in a range of 10 kDa to 20 kDa.
14. The anti-IL-10 single domain antibody of any one or both of embodiments 12 and 13, wherein the antibody is stable at a temperature in a range from 70° C to 90° C.
15. The anti-IL-10 single domain antibody of any one or more of embodiments 12-14, wherein the antibody binds to chicken II-10 with an EC₅₀ of 30 nM or less in a cell ELISA assay.
16. The anti-IL-10 single domain antibody of any one or more of embodiments 12-15, wherein the antibody comprises an amino acid sequence with at least 90% identity to a sequence selected from the group consisting of SEQ ID NOs: 87-154.
17. The anti-IL-10 single domain antibody of any one or more of embodiments 12 -16, wherein the antibody comprises the amino acid sequence with at least 90% identity to a sequence selected from the group consisting of SEQ ID NOs: 89, 135, and 146.
18. The anti-IL-10 single domain antibody of any one or more of embodiments 12-17, wherein the antibody is fused to an N-terminal signal peptide or C-terminal signal peptide, or both.
19. The anti-IL-10 single domain antibody of any one or more of embodiments 12-18, wherein the N-terminal signal peptide is selected from a group consisting of: OsGluB4sp (rice Glu-B4 glutelin signal peptide), BAASS (barley alpha amylase signal sequence), PR1 (pathogenesis related protein), or zein 27 (xGZm27ss) signal peptide.
20. The anti-IL-10 single domain antibody of any one or more of embodiments 12-19, wherein the C-terminal signal peptide is selected from a group consisting of: KDEL (SEQ ID NO: 29), HDEL (SEQ ID NO: 30), SEKDEL (SEQ ID NO: 31), HvVSD from barley polyamine oxidase, or HvAle from barley aleurone (thiol protease).
21. The anti-IL-10 single domain antibody of any one or more of embodiments 12-20, wherein the anti-IL-10 single domain antibody is digestible by pepsin.
22. The anti-IL-10 single domain antibody of any one or more of embodiments 12-20, wherein the anti-IL-10 single domain antibody is stable to digestion by pepsin.
23. The anti-IL-10 single domain antibody of any one or more of embodiments 12-22, wherein the anti-IL-10 single domain antibody is stable to a temperature exposure of greater than 70° C and less than 100° C.
24. A synthetic polynucleotide encoding the anti-IL-10 single domain antibody of any one or more of embodiments 12-23.
25. The synthetic polynucleotide of embodiment 24, wherein the synthetic polynucleotide comprises a sequence with at least 90% identity to a reference sequence selected from the group consisting of: SEQ ID NOs: 173-178.
26. A transgenic plant or tissues thereof comprising one or more polynucleotides encoding the at least one antagonist IL-10R peptide of any one or more of embodiments 1-9, or the anti-IL-10 single domain antibody of any one or more of embodiments 12-23.
27. A transgenic plant or tissues thereof comprising one or more polynucleotides encoding the at least one antagonist IL-10R peptide, or the anti-IL-10 single domain antibody.
28. The transgenic plant or tissues thereof of embodiment 27, wherein the antagonist IL-10R peptide is one peptide comprising an amino acid sequence with at least 90% identity to a reference sequence selected from the group consisting of: SEQ ID NOS: 1-13.
29. The transgenic plant or tissues thereof of any one or both embodiments 27 and 28, wherein the antagonist IL-10R peptide comprises concatenated antagonist IL-10R peptides comprising an amino acid with at least 90% identity to a reference sequence selected from the group consisting of SEQ ID NOS: 32-40.
30. The transgenic plant or tissues thereof of any one or more of embodiments 27-29, wherein the anti-IL-10 single domain antibody binds to a polypeptide comprising an amino acid sequence of SEQ ID NO: 80.
31. The transgenic plant or tissues thereof of any one or more of embodiments 27-30, wherein the anti-IL-10 single domain antibody has a molecular mass in a range of 10 kDa to 20 kDa.
32. The transgenic plant or tissues thereof of any one or more of embodiments 27-31, wherein the anti-IL-10 single domain antibody is stable at a temperature in a range from 70° C. to 90° C.
33. The transgenic plant or tissues thereof of any one or more of embodiments 27-32, wherein the anti-IL-10 single domain antibody binds to chicken II-10 with an EC₅₀ of 30 nM or less in a cell ELISA assay.
34. The transgenic plant or tissues thereof of any one or more of embodiments 27-33, wherein the anti-IL-10 single domain antibody comprises an amino acid sequence with at least 90% identity to a sequence selected from the group consisting of SEQ ID NOs: 87-154.
35. The transgenic plant or tissues thereof of any one or more of embodiments 27-34, wherein the anti-IL-10 single domain antibody comprises the amino acid sequence with at least 90% identity to a sequence selected from the group consisting of SEQ ID NOs: 89, 135, and 146.
36. The transgenic plant or tissues thereof of any one or more of embodiments 27-35, wherein the one or more polynucleotides comprise a sequence with at least 90% identity to a reference sequence selected from the group consisting of SEQ ID NOS: 16-28, and 56.
37. The transgenic plant or tissues thereof of any one or more of embodiments 27-36, wherein the one or more polynucleotides comprise a sequence with at least 90% identity to a reference sequence selected from the group consisting of SEQ ID NOS: 173-178.
38. The transgenic plant or tissues thereof of any one or more of embodiments 27-37, wherein the anti-IL-10 single domain antibody is digestible by pepsin.
39. The transgenic plant or tissues thereof of any one or more of embodiments 27-37, wherein the anti-IL-10 single domain antibody is stable to digestion by pepsin.
40. The transgenic plant or tissues thereof of any one or more of embodiments 27-39, wherein a plant is selected from the group consisting of: corn, soybean, wheat, rice, sorghum, canola, cotton, and switchgrass.
41. An animal feed comprising the transgenic plant or tissues thereof of any one or more of embodiments 26-40.
42. An animal feed comprising at least one antagonist IL-10R peptide of any one or more of embodiments 1-9, or an anti-IL-10 single domain antibody of any one or more of embodiments 12-23.
43. The animal feed of embodiment 42, wherein the at least one IL-10R antagonist IL-10R peptide, or the anti-IL-10 single domain antibody is active upon expression in the plant and exposure to a temperature in the range from 25° C. to 130° C.
44. The animal feed of any one or more of embodiments 41, or 42-43 further comprising a feed supplement.
45. The animal feed of any one or more of embodiments 41, or 42-44, wherein the feed supplement is plant material.
46. The animal feed of any one or more of embodiments 41, or 42-45, wherein the plant material is a non-transgenic plant or a transgenic plant.
47. The animal feed of any one or more of embodiments 41, or 42-46, wherein the plant material includes at least one component selected from the group consisting of: corn meal, corn pellets, wheat meal, wheat pellets, wheat grain, barley grain, barley pellets, soybean meal, soybean oilcake, sorghum grain and sorghum pellets.
48. The animal feed of any one or more of embodiments 41, or 42-47, wherein the feed supplement includes one or more exogenous enzymes.
49. The animal feed of any or more of embodiments 41, or 42-48, wherein the one or more exogenous enzymes includes a hydrolytic enzyme selected from the group consisting of: xylanase, endoglucanase, cellulase, protease, phytase, amylase and mannanase.
50. The animal feed of any one or more of embodiments 41, or 42-49, wherein the feed supplement includes at least one component selected from the group consisting of: soluble solids, fat and vermiculite, limestone, plain salt, DL-methionine, L-lysine, L-threonine, COBAN®, vitamin premix, dicalcium phosphate, selenium premix, choline chloride, sodium chloride, and mineral premix.
51. A method of treating or preventing a gastrointestinal infection in an animal comprising feeding the animal the at least one antagonist IL-10R peptide of any one or more of embodiments 1-9, the anti-IL-10 single domain antibody of any or more of embodiments 12-23, the transgenic plant or tissues thereof of any one or more of embodiments 26-40, or the animal feed of any one more of embodiments 41-50.
52. The method of embodiment 51, wherein the gastrointestinal infection is caused by a gastrointestinal pathogen selected from the group consisting of: bacteria, yeast, fungi, archae, virus, and protozoa.
53. The method of any one or both of embodiments 51 and 52, wherein the gastrointestinal pathogen belongs to the genus Eimeria.
54. The method any one or more of embodiments 51-53, wherein the gastrointestinal pathogen is selected from the group consisting of: Eimeria tenella, Eimeria acervulina, and Eimeria maxima.
55. A method of stimulating or modulating the immune system and improving gastrointestinal physiology of an animal comprising feeding the animal the at least one antagonist IL-10R peptide of any one or more of embodiments 1-9, the anti-IL-10 single domain antibody of any or more of embodiments 12-23, the transgenic plant or tissues thereof of any one or more of embodiments 26-40, or the animal feed of any one or more of embodiments 41-50.
56. A method of improving animal performance comprising feeding an animal the at least one antagonist IL-10R peptide of any one or more of embodiments 1-9, the anti-IL-10 single domain antibody of any one or more of embodiments 12-23, the transgenic plant or tissues thereof of any one or more of embodiments 26-40, or the animal feed of any one or more of embodiments 41-50.
57. A method of preparing an animal feed comprising mixing the antagonist IL-10R peptide of any one or more of embodiments 1-9, the anti-IL-10 single domain antibody of any one or more of embodiments 12-23, or the transgenic plant or tissues thereof of any one or more of embodiments 26-40 with plant material to form a mixture.
58. The method of embodiment 57, wherein the method further comprises pelletizing the mixture.
59. The method of any one or both of embodiments 57 and 58, wherein the method further comprises adding a feed supplement to the mixture.
60. The method of any one or more of embodiments 57-59, wherein the plant material is a non-transgenic plant or a transgenic plant.
61. The method of any one or more of embodiments 57-60, wherein the plant material includes at least one component selected from the group consisting of: corn meal, corn pellets, wheat meal, wheat pellets, wheat grain, barley grain, barley pellets, soybean meal, soybean oilcake, sorghum grain and sorghum pellets.
62. The method of any one or more of embodiments 57-61, wherein the feed supplement includes one or more exogenous enzymes.
63. The method of any or more of embodiments 57-62, wherein the one or more exogenous enzymes includes a hydrolytic enzyme selected from the group consisting of: xylanase, endoglucanase, cellulase, protease, phytase, amylase and mannanase.
64. The method of any one or more of embodiments 57-63, wherein the feed supplement includes at least one component selected from the group consisting of: soluble solids, fat and vermiculite, limestone, plain salt, DL-methionine, L-lysine, L-threonine, COBAN®, vitamin premix, dicalcium phosphate, selenium premix, choline chloride, sodium chloride, and mineral premix.

Further embodiments herein may be formed by supplementing an embodiment with one or more elements from any one or more other embodiments herein, and/or substituting one or more elements from one embodiment with one or more elements from one or more other embodiments

### EXAMPLES

The following non-limiting examples are provided to illustrate particular embodiments. The embodiments throughout may be supplemented with one or more details from one or more examples below, and/or one or more elements from an embodiment may be substituted with one or more details from one or more examples below.

### Example 1. Strategies for Engineering Peptides and Antibodies

While reducing IL-10 levels prior to and during Eimeria infection can help control the negative effects of Coccidiosis, the mechanisms known in the art that have been employed to reduce IL-10 levels are expensive and have questionable robustness to be employed broadly in industry. The shortcomings of the existing technologies (using isolated, or partially purified, peptides or antibodies) to control Coccidiosis can be addressed in several important ways using biotechnology to design novel products that target the IL-10 signaling pathway. First, by broadening antibody discovery and development beyond the common target chicken production system, as is currently done by inoculating maternal hens or eggs with conjugated IL-10 peptides, novel antibodies and peptides can be developed that have been specifically tailored to controlling Coccidiosis. The peptides and synthetic antibodies developed herein were engineered to have high affinity (thus reducing dosing levels), improved thermal stability (to survive pelleting when mixed into animal feed), and low molecular weight to promote high-levels of expression (to maximize production economics), properties that are not found in naturally occurring peptides and antibodies and that could not be simply selected for in nature, nor could these properties be efficiently replicated in a hen or egg production system without undue experimentation. Second, by engineering the genes encoding the peptides and antibodies developed herein into plants, their delivery can be made by directly feeding the plants or plant tissues without additional isolation, or purification, or formulation, into the diet. This greatly benefits production and animal administration economics. That such a combination of technologies works effectively in controlling Coccidiosis was unexpected. It was anticipated that antibodies and peptides delivered in whole grain or meal, with no isolation, would either not survive the pelleting process when being mixed in feed (which is often the case when using larger antibodies, such as IgG's or IgY's, and peptides), not diffuse adequately from the plant matrix and be readily available to the animal at sufficient concentrations to modulate the IL-10 signaling pathway, or would be rapidly degraded in the digestive tract. Unexpectedly, the combination of technologies used to make the products described herein, was able to overcome these challenges and address the challenges confronting current methods used in the art for controlling Coccidiosis.

Plant expression of heterologous peptides and proteins is one of the least expensive recombinant protein production systems on the planet. By engineering corn or soy beans to produce anti-IL-10 antibodies, IL-10R peptide antagonists, or other molecules that inhibit IL-10 signaling, these molecules can be made at high concentrations, e.g., a concentration in a range from 0.01 mg of heterologous protein (anti-IL-10 antibodies, IL-10R peptide antagonists, or other proteins or peptides that inhibit IL-10 signaling) per gram of milled grain up to as much as 20 mg of heterologous protein per gram of milled grain. Heterologous proteins and peptides produced in seed tissue are naturally stabilized in the seed as it progresses through its desiccation process following seed development. Antibodies and peptide antagonists produced in corn or soy beans can be delivered directly into poultry diets by simply milling the grain and mixing it with the other ingredients. While other processing and formulation steps may be used, there is no need for additional processing steps that would be required if these products were produced by fermentation or by inoculating eggs, both of which are more expensive processes. Further, because these molecules are encoded by DNA that is stably integrated into the plants' genome, there is the opportunity to further engineer these molecules and endow them with beneficial properties that cannot be implemented when generating antibodies in maternal hens or by inoculating eggs.

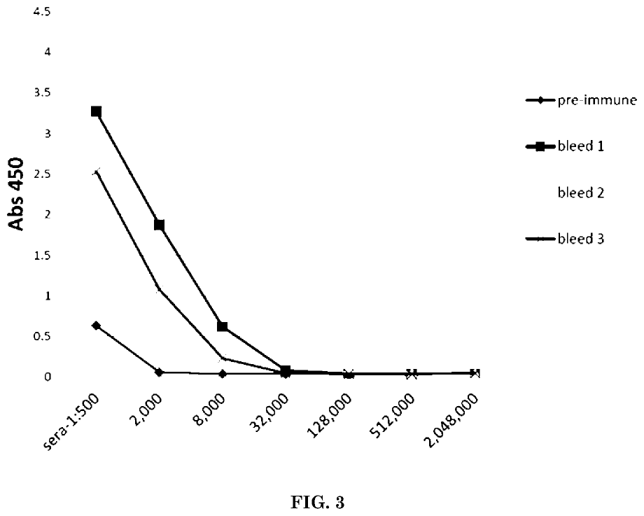

Engineering of antibodies, proteins, and peptides provides additional opportunities to address industry needs in modulating IL-10 signaling pathways. Anti-IL-10 antibodies can be generated by any number of hosts, including camels, calves, chickens, goats, horses, humans, llamas, mice, rabbits, rats, sharks, and many other species. The particular choice of host for generating an antibody may depend on a variety of considerations including the choice of antigen (will the antigen be recognized as self by the host or recognized as foreign and generate an appropriate immune response), the choice of antibody (IgG, IgY, a polyclonal, monoclonal, etc.), ease of working with the host, the amount of antibody desired, the intended species the antibody will be used in, and the type of antibody desired (full length (approximately 120-160 kDa), antibody fragments (Fab that can be approximately 50 kDa, or scFv that can be approximately 25 kDa), or single domain antibodies (that can be approximately 10-20 kDa)). Single domain antibodies are small synthetic antibodies abbreviated by sdAb, sdAB, V_{H}H, or V_{NAR}, and have specific features that make them suitable for targeting IL-10 when delivered through feed into an animal's digestive tract. In particular, sdAbs are generally recognized as having improved thermal stability relative to full length and other antibody fragments, and are very susceptible to molecular engineering, which provides the possibility of further improving their thermal stability and affinity, both of which may help in reducing the necessary dose required in the animal feed to modulate the IL-10 signaling pathway (E. R. Goldman, G. P. Anderson, J. L. Liu, J. B. Delehanty, L. J. Sherwood, L. E. Osborn, L. B. Cummins, and A. Hayhurst, 2006, Facile Generation of Heat-Stable Antiviral and Antitoxin Single Domain Antibodies from a Semisynthetic Llama Library, Annal. Chem., 78:8245-8255, which is incorporated herein by reference as if fully set forth). Chicken IL-10 (amino acid residues in positions 2 to 151 of SEQ ID NO: 80) was compared with llama IL-10 (SEQ ID NO: 207). Sequence alignment analysis showed the sequences to be only 48% identical. Based on this analysis, the full-length Gallus gallus IL-10 (chicken IL-10 or cIL-10) was selected as the target antigen for generating sdAbs in camels or llamas. Because llamas are not exposed to cIL-10, an endogenous chicken interleukin, and it's only through a biotechnology process that cIL-10 can be made, isolated and dosed into a llama, llamas are naive to cIL-10 and it was found that it's possible to generate a significant immune response as shown in FIG. 3. Further, given that IL-10 is known to dimerize in vivo (K. Asadullah, W. Sterry, H. D. Volk, “Interleukin-10 Theraphy—Review of a New Approach”, Pharmacological Reviews, 55 (2):241-269, 2003), using IL-10 peptides as target antigens is challenging because peptide epitopes may be selected that are not normally exposed in the IL-10 dimer in vivo. Thus, library generation and antibody screening may be more readily optimized, and synthetic antibodies developed, using full-length cIL-10. Another advantage of developing a single molecule for expression is that it can be selected from a diversity of molecules made by the inoculated host, allowing for screening and selection of a highly optimized sdAb that can be reproducibly made with great efficiency in corn or another host. In this way, many of the current challenges with modulating the IL-10 pathway in poultry can be addressed to bring this innovation into the marketplace.

### Example 2. Peptide Selection

Synthetic peptides were designed by analyzing the crystal structures of human IL-10/IL-10R1 complexes, determining portions of the sequences of the human proteins that contribute to binding, and finding the analogous sequences in the chicken proteins by aligning the sequences of human and chicken IL-10 and IL-10R1, respectively, with Clustal Omega (Diaz-Valdez et al., 2011, Josephson et al., 2001, Naiyer et al., 2013, Ni et al., 2016, Reineke et al., 1998, Yoon et al., 2005, Zdanov et al., 1996). Amino acid sequences were obtained from Pub Med; and the presumed signal peptide, transmembrane, and intracellular sequences were removed from the alignments. All peptide sequences were checked for known allergenic epitopes using the Allergen Online Database.

Human IL-10 residues involved in binding to its receptor as determined by peptide mapping are as follows:

| | |
|---|---|
| Chicken | ------ LEPTCLHFSELLPARLRELRVKFEEIKDYFQSRDD |
| Huma | SPGQGTQSENSCTHFPGNLPNMLRDLRDAFSRVKTFFQMKDQ |
| | . . * :* ** ** **:** *..:* :** :*: |
| Chicken | ELNIQLLSSELLDEFKGTFGCQSVSEMLRFYTDEVLPRAMQTSTSHQQSMGDLGNMLLGL |
| Human | -LDNLLLKESLLEDFKGYLGCQALSEMIQFYLEEVMPQAENQDPDIKAHVNSLGENLKTL |
| | *: **...**::*** :***::***::** :**:*:* : . . : :..**: * * |
| Chicken | KATMRRCHRFFTCEKRSKAIKQIKETFEKMDENGIYKAMGEFDIFINYIEEYLLMRRR- (SEQ ID NO: 80) |
| Human | RLRLRRCHRFLPCENKSKAVEQVKNAFNKLQEKGIYKAMSEFDIFINYIEAYMTMKIRN (SEQ ID NO: 81) |
| | : :******: **::***::*:*::*:*::*:******.********** *: *: * |

Human (AAA63207.1; SEQ ID NO: 81) and chicken (NP_001004414.2; SEQ ID NO: 80) sequences were obtained and edited as described above. Human IL-10 residues that are involved in binding to human IL-10R1 are in boldface and colored gray (Reineke et. al., 1998). Chicken IL-10 sequence alignment with the human IL-10 sequence is also shown.

Human IL-10 residues that bury >5 Å² surface area in the complex with the receptor (Yoon, et. al., 2005).

| | |
|---|---|
| Chicken | ------ LEPTCLHFSELLPARLRELRVKFEEIKDYFQSRDD |
| Human | SPGQGTQSENSCTHFPGNLPNMLRDLRDAFSRVKTFFQMKDQ |
| | . . * :* ** ** **:** *..:* :** :*: |
| Chicken | ELNIQLLSSELLDEFKGTFGCQSVSEMLRFYTDEVLPRAMQTSTSHQQSMGDLGNMLLGL |
| Human | -LDNLLLKESLLEDFKGYLGCQALSEMIQFYLEEVMPQAENQDPDIKAHVNSLGENLKTL |
| | *: **...**::*** :***::***::** :**:*:* : . . : :..**: * * |
| Chicken | KATMRRCHRFFTCEKRSKAIKQIKETFEKMDENGIYKAMGEFDIFINYIEEYLLMRRR- (SEQ ID NO: 80) |
| Human | RLRLRRCHRFLPCENKSKAVEQVKNAFNKLQEKGIYKAMSEFDIFINYIEAYMTMKIRN (SEQ ID NO: 81) |
| | : :******: **::***::*:*::*:*::*:******.********** *: *: * |

Chicken IL-10 sequence (SEQ ID NO: 80) alignment with the human IL-10 sequence (SEQ ID NO: 81) is shown, with the two segments of the human IL-10 sequence that contribute the majority of binding surface (Diaz-Valdez et al., 2011; Josephson et al., 2001; Naiyer et al., 2013; Ni et al., 2016; Reineke et al., 1998; Yoon et al., 2005; and Zdanov et al., 1996), are indicated in boldface and colored gray. Helical regions are underlined.

Examples of peptide design based on alignment with predicted sequence regions of binding interactions:

| | |
|---|---|
| Chicken | ------ LEPTCLHFSELLPARLRELRVKFEEIKDYFQSRDD |
| Human | SPGQGTQSENSCTHFPGNLPNMLRDLRDAFSRVKTFFQMKDQ |
| | . . * :* ** ** **:** *..:* :** :*: |
| Chicken | ELNIQLLSSELLDEFKGTFGCQSVSEMLRFYTDEVLPRAMQTSTSHQQSMGDLGNMLLGL |
| Human | -LDNLLLKESLLEDFKGYLGCQALSEMIQFYLEEVMPQAENQDPDIKAHVNSLGENLKTL |
| | *: **...**::*** :***::***::** :**:*:* : . . : :..**: * * |
| Chicken | KATMRRCHRFFTCEKRSKAIKQIKETFEKMDENGIYKAMGEFDIFINYIEEYLLMRRR- (SEQ ID NO: 80) |
| Human | RLRLRRCHRFLPCENKSKAVEQVKNAFNKLQEKGIYKAMSEFDIFINYIEAYMTMKIRN (SEQ ID NO: 81) |
| | : :******: **::***::*:*::*:*::*:******.********** *: *: * |

Binding hot spots and helical regions of human IL-10 are designated as shown previously. Sequences within the chicken IL-10 sequence that encompass peptides P25 (SEQ ID NO: 5) and P26 (SEQ ID NO: 6) are designated by boldface italics and colored gray. Peptide P21 (SEQ ID NO: 1) consists of P25, P26, and all of the chicken IL-10 residues between them in the sequence.

Examples of peptide design based on alignment with binding hot spots:

| | |
|---|---|
| Chicken | ------ LEPTCLHFSELLPARLRELRVKFEEIKDYFQSRDD |
| Human | SPGQGTQSENSCTHFPGNLPNMLRDLRDAFSRVKTFFQMKDQ |
| | . . * :* ** ** **:** *..:* :** :*: |
| Chicken | ELNIQLLSSELLDEFKGTFGCQSVSEMLRFYTDEVLPRAMQTSTSHQQSMGDLGNMLLGL |
| Human | -LDNLLLKESLLEDFKGYLGCQALSEMIQFYLEEVMPQAENQDPDIKAHVNSLGENLKTL |
| | *: **...**::*** :***::***::** :**:*:* : . . : :..**: * * |
| Chicken | 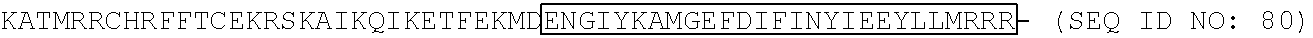 |
| Human | RLRLRRCHRFLPCENKSKAVEQVKNAFNKLQEKGIYKAMSEFDIFNYIEAYMTMKIRN (SEQ ID NO: 81) |
| | : :******: **::***::*:*::*:*::*:******.********** *: *: * |

Binding hot spots and helical regions of human IL-10 are designated as previously shown. Peptide P27 (SEQ ID NO: 7) is designated by boldface, italicized and is colored in gray. Peptide P22 (SEQ ID NO: 2) is designated in boldface and included in a frame. Peptide 27 is the region within the P22 sequence.

Alignment of human (SEQ ID NO: 83) and chicken (SEQ ID NO: 82) IL-10R1 soluble domain sequences:

| | |
|---|---|
| Human IL-10R1 | HGTELPSPPSVWFEAEFFHHILHWTPIPNQSESTCYEVALLRYGIE-SWNSISNCSQTLS |
| Chicken IL-10r1 | --ELRLKPTRVRFVAEMVYHLLQWEPGPDAPSDTRYDVEHKIYGTNSPWTAIPNCMKIHG |
| | .* * * **:.:*:*:* * : ..* *:* ** : *.:* ** : . |
| Human IL-10R1 | YDLTAVTLDLYHSNGYRARVRAVDGSRHSNWTVTNTRFSVDEVTLTVGSVNLEIHNGFIL |
| Chicken IL-10R1 | HSCDLTYYTLDPSLRYYARVRAVVGNHTSDWKRTNA-FSPQEASLRLSGHSLAVTDNSIH |
| | :. . * * * ****** *.: *:*. **: ** :*.:* :.. .* : :. * |
| Human IL-10R1 | GKIQLPRPKMAPANDTYESIFSHFREYEIAIRKVPGNFTFTHKKVKHENFSLLTSGEVGE |
| Chicken IL-10R1 | VQLQLLL-RAGNRTVKYDDIQKHARRYRVYIRRARDNQTYEVWETAS-EFYIRNLFWNTE |
| | ::** : . . .*:.* .* *.*.: **:. .* *: :. :* : . * |
| Human IL-10R1 | FCVQVKPSVASRSNKGMWSKEECISLTRQYFTVTN---VIIFFAFVLL---LSGALAYCL (SEQ ID NO: 83) |
| Chicken IL-10R1 | YCISVEPDVASRHIPAMRTAEQCVTIGHRDESAEL (SEQ ID NO: 82) |
| | :*:.*:*.**** .* : *:*::: :: :. |

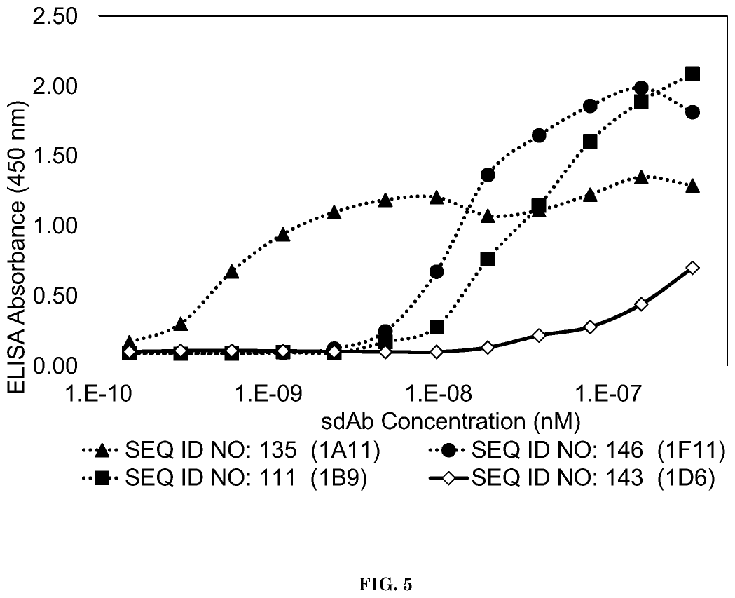

Human (NP_001549.2) and chicken (NP_001034686.1) sequences were obtained and edited as described above. Human IL-10R1 residues that are involved in binding to human IL-10 (FIG. 5 of Reineke et. al., 1998) are designated by boldface. Chicken IL-10R1 sequence alignment with the human IL-10R1 sequence is also shown; intracellular and transmembrane domains are omitted for clarity.

### Example 3. Peptide Screening

Peptides were chemically synthesized, desalted, and purified to >98% purity by Watsonbio, Inc., Houston TX. Peptide stock solutions were prepared at 10 mM in DMSO and diluted in assay buffer to 0.05 mM. Peptides were screened at Marin Biologic Laboratories, Inc., Novato, CA, by determining their effectiveness at blocking the inhibitory effect of chicken IL-10 on the induction of interferon gamma (IFN-Υ) induced by concanavalin A (ConA) (or alternatively phytohemagglutinin (PHA) may be used) in chicken spleen cells (Wu et al. (2016) and Rothwell et al. (2004)). Briefly, lymphocytes and mononuclear cells were isolated from chicken spleens by differential centrifugation on Ficoll-Hypaque. Cells were cultured at 5×10⁶ cells/mL in wells of a 96-well plate for 72 hours in the presence of peptide, 1.2 μg/mL ConA (or alternatively 12.5 μg/mL PHA), and with or without cIL-10. Spleen cells were also incubated without peptides and/or without ConA (or PHA) as controls. Levels of IFN-Υ in the supernatants were determined by ELISA.

The use of peptides derived from IL-10s of several species that can be added to feed to reduce respiratory and intestinal illness in these animals is known in the art. See U.S. Pat. No. 8,652,457 B2 and U.S. patent application publication No. US2016/0280778 A1, which are incorporated by reference herein as if fully set forth. These peptides were designed to elicit an immune response in the animals, which would lead to the production of anti-IL-10 antibodies. In contrast, peptides described herein were designed with the goal of directly interfering with binding of chicken IL-10 to its receptor (that is, as IL-10R antagonists). As a result, the peptides described here were designed in part to mimic sections of either cIL-10 or the R1 subunit of the cIL-10 receptor and to compete with either cIL-10 or cIL-10R for binding to the other species, as opposed to the peptides in the previous patents that were designed to incorporate antigenic features of IL-10.

TABLE 1
Amino acid sequences of peptides

| SEQ | | |
|---|---|---|
| ID | | |
| NO | Description | Sequence |
| 1 | P21 | PARLRELRVKFEEIKDYFQSRDDELNIQLLSSEL |
| | | LDEFKG |
| 2 | P22 | ENGIYKAMGEFDIFINYIEEYLLMRRR |
| 3 | P23 | PARLRELRVKFEEIKDYFQGGGSGGGSQQSMG |
| | | DLGNMLLGLKATMRR |
| 4 | P24 | GCQSVSEMLRFYTDEVLPRAMQGGGSGGGSKA |
| | | MGEFDIFINYIEEYLLMR |
| 5 | P25 | PARLRELR |
| 6 | P26 | LSSELLDEFKG |
| 7 | P27 | GEFDIFNYIE |
| 8 | P28 | SLRYYARVRA |
| 9 | P29 | TNAFSPQ |
| 10 | P11 | YDDIQKHARRYRVYIRRARDNQTYEVWET |
| 11 | P30 | IQKHARRY |
| 12 | P31 | NQTYEVWE |
| 13 | P32 | VASRHIPAM |
| 14 | P9* | FFKKFFKKFFKKFFKK |
| 15 | P6* | GTELPSPPSVWFEAEF |

*P9 and P6 are control peptides (Ni et al.)

### Example 4. Basic Plant Expression Constructs for Production of IL-10R Antagonist Peptides in Maize

The amino acid sequences for IL-10R antagonist peptides and all other sequences in this document have been back translated and codon optimized for expression in the desired host organism. As an example, for plant expression, maize codon usage was used to demonstrate codon optimization, expression cassette assembly, vector assembly, and plant transformation, but other host organisms could also be used. All IL-10R antagonist peptides and antibodies were back translated and codon optimized for expression in maize using the computer program Vector NTI (ThermoFisher Scientific, Waltham, MA). The resulting DNA sequences are presented in Table 2 and at the end of the document.

TABLE 2
IL-10R antagonist peptides and their maize codon optimized DNA coding
sequences

| Peptide | IL-10R antagonist | |
|---|---|---|
| name | peptide sequence | Maize codon optimized DNA sequence |
| P21 | PARLRELRVKFEEIKDY | CCGGCCAGGCTGAGGGAGCTGAGGGTGAAG |
| | FQSRDDELNIQLLSSEL | TTCGAGGAGATCAAGGACTACTTCCAGAGC |
| | LDEFKG (SEQ ID NO: 1) | AGGGACGACGAGCTGAACATCCAGCTGCTG |
| | | AGCAGCGAGCTGCTGGACGAGTTCAAGGGC |
| | | (SEQ ID NO: 16) |
| P22 | ENGIYKAMGEFDIFINY | GAGAACGGCATCTACAAGGCCATGGGCGAG |
| | IEEYLLMRRR (SEQ ID | TTCGACATCTTCATCAACTACATCGAGGAGT |
| | NO: 2) | ACCTGCTGATGAGGAGGAGG |
| | | (SEQ ID NO: 17) |
| P23 | PARLRELRVKFEEIKDY | CCGGCCAGGCTGAGGGAGCTGAGGGTGAAG |
| | FQGGGSGGGSQQSMGD | TTCGAGGAGATCAAGGACTACTTCCAGGGC |
| | LGNMLLGLKATMRR | GGCGGCAGCGGCGGCGGCAGCCAGCAGAG |
| | (SEQ ID NO: 3) | CATGGGCGACCTGGGCAACATGCTGCTGGG |
| | | CCTGAAGGCCACCATGAGGAGG |
| | | (SEQ ID NO: 18) |
| P24 | GCQSVSEMLRFYTDEV | GGCTGCCAGAGCGTGAGCGAGATGCTGAGG |
| | LPRAMQGGGSGGGSKA | TTCTACACCGACGAGGTGCTGCCGAGGGCC |
| | MGEFDIFINYIEEYLLM | ATGCAGGGCGGCGGCAGCGGCGGCGGCAG |
| | R (SEQ ID NO: 4) | CAAGGCCATGGGCGAGTTCGACATCTTCAT |
| | | CAACTACATCGAGGAGTACCTGCTGATGAG |
| | | G (SEQ ID NO: 19) |
| P25 | PARLRELR | CCGGCCAGGCTGAGGGAGCTGAGG |
| | (SEQ ID NO: 5) | (SEQ ID NO: 20) |
| P26 | LSSELLDEFKG | CTGAGCAGCGAGCTGCTGGACGAGTTCAAG |
| | (SEQ ID NO: 6) | GGC (SEQ ID NO: 21) |
| P27 | GEFDIFNYIE | GGCGAGTTCGACATCTTCAACTACATCGAG |
| | (SEQ ID NO: 7) | (SEQ ID NO: 22) |
| P28 | SLRYYARVRA | AGCCTGAGGTACTACGCCAGGGTGAGGGCC |
| | (SEQ ID NO: 8) | (SEQ ID NO: 23) |
| P29 | TNAFSPQ | ACCAACGCCTTCAGCCCGCAG |
| | (SEQ ID NO: 9) | (SEQ ID NO: 24) |
| P11 | YDDIQKHARRYRVYIRR | TACGACGACATCCAGAAGCACGCCAGGAGG |
| | ARDNQTYEVWET | TACAGGGTGTACATCAGGAGGGCCAGGGAC |
| | (SEQ ID NO: 10) | AACCAGACCTACGAGGTGTGGGAGACC |
| | | (SEQ ID NO: 25) |
| P30 | IQKHARRY | ATCCAGAAGCACGCCAGGAGGTAC |
| | (SEQ ID NO: 11) | (SEQ ID NO: 26) |
| P31 | NQTYEVWE (SEQ ID | AACCAGACCTACGAGGTGTGGGAG (SEQ ID |
| | NO: 12) | NO: 27) |
| P32 | VASRHIPAM (SEQ ID | GTGGCCAGCAGGCACATCCCGGCCATG |
| | NO: 13) | (SEQ ID NO: 28) |

Examples of basic cloning vectors containing individual expression cassettes for P24 IL-10R antagonist peptide are given in Table 3. Analogous vectors could be made for any of the other IL-10R antagonist peptides listed in Table 2, or antibodies, by substituting the P24 peptide transgene with a different peptide or antibody sequence.

TABLE 3
P24 basic expression vectors

| | | N-terminal | C-terminal |
|---|---|---|---|
| Vector | Promoter | signal | signal |
| pAG4305 | prOsGluB4 | xGZein27ss | KDEL (SEQ ID |
| | | | NO: 29) |
| pAG4981 | prZmgZ27 | xGZein27ss | KDEL (SEQ ID |
| | | | NO: 29) |
| pAG4982 | prGtl1 | xGZein27ss | KDEL (SEQ ID |
| | | | NO: 29) |
| pAG4983 | prZmGlb1 | xGZein27ss | KDEL (SEQ ID |
| | | | NO: 29) |
| pAG4984 | prZmOle16 | xGZein27ss | KDEL (SEQ ID |
| | | | NO: 29) |

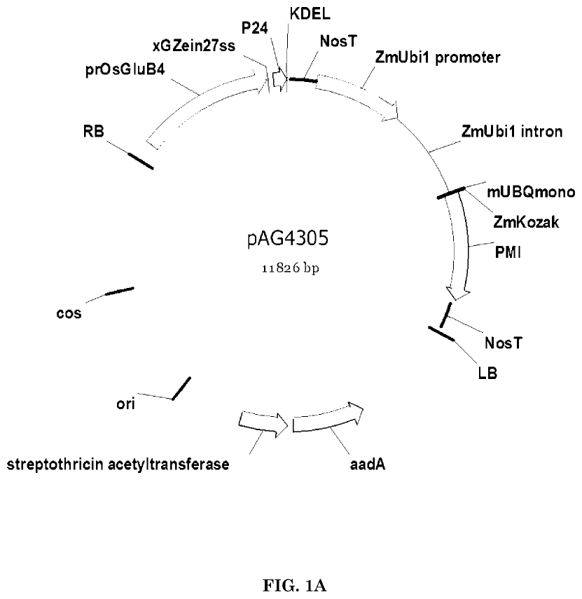

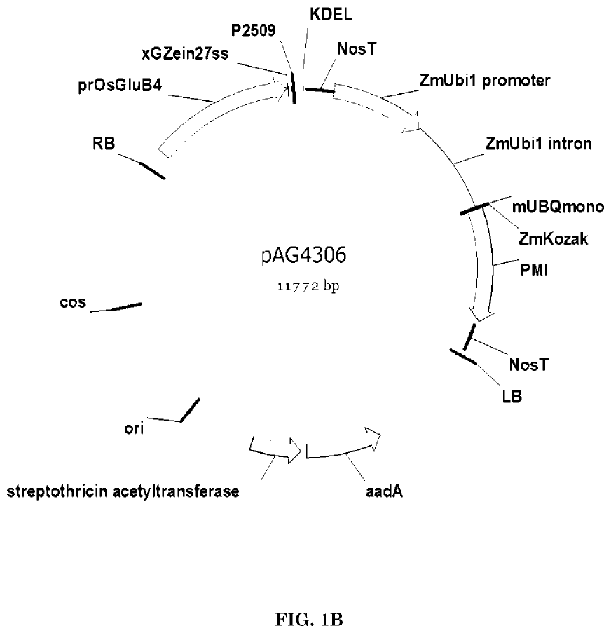

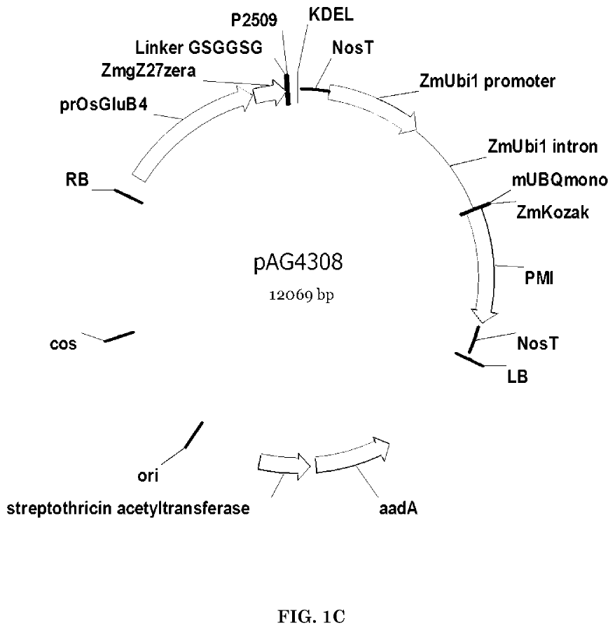

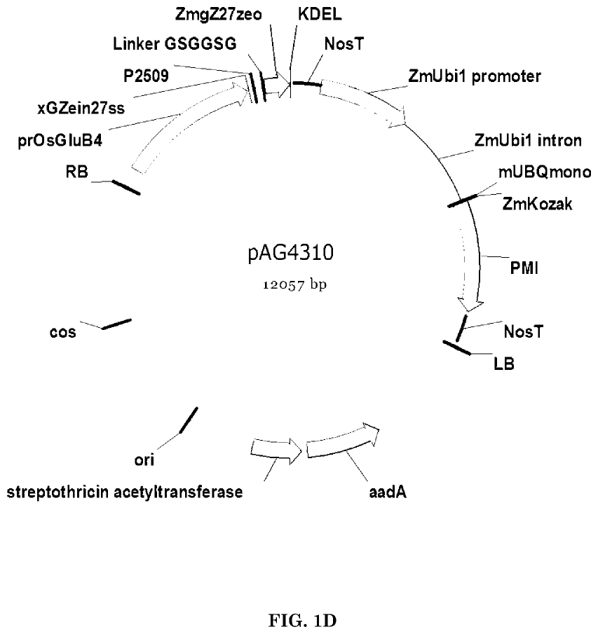

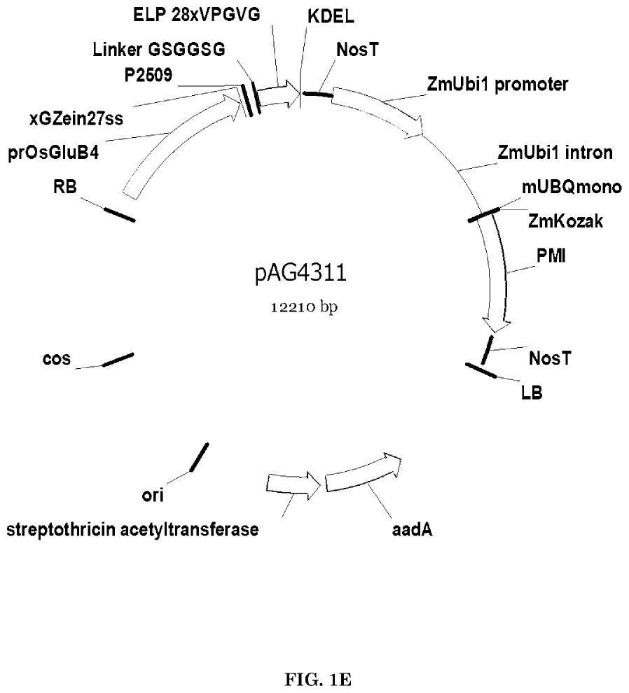

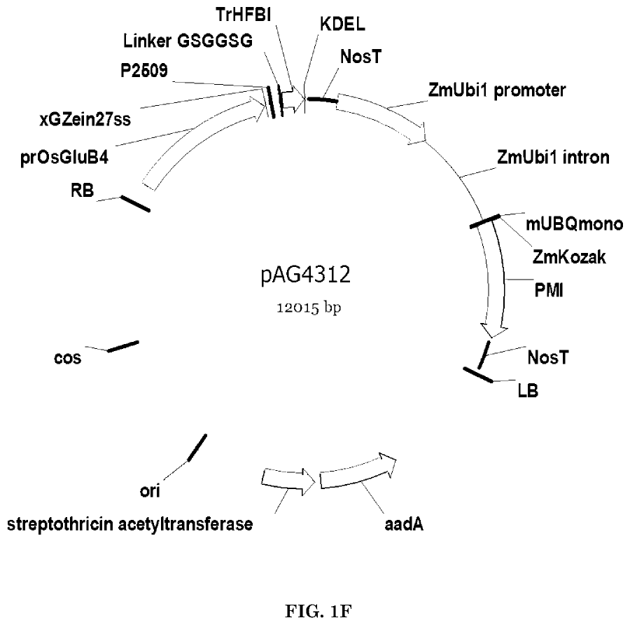

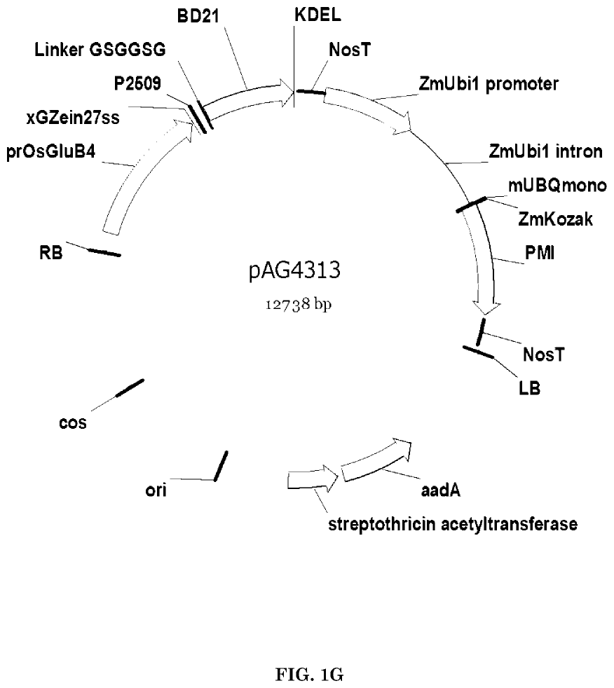

FIGS. 1A-1G illustrate the schematic drawings of the vectors pAG4305 (FIG. 1A), pAG4306 (FIG. 1B), pAG4308 (FIG. 1C), pAG4310 (FIG. 1D), pAG4311 (FIG. 1E), pAG4312 (FIG. 1F), and pAG4313 (FIG. 1G).

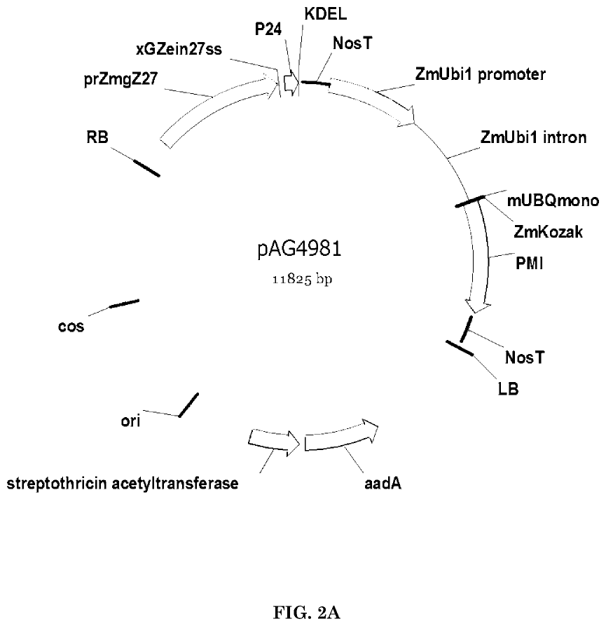

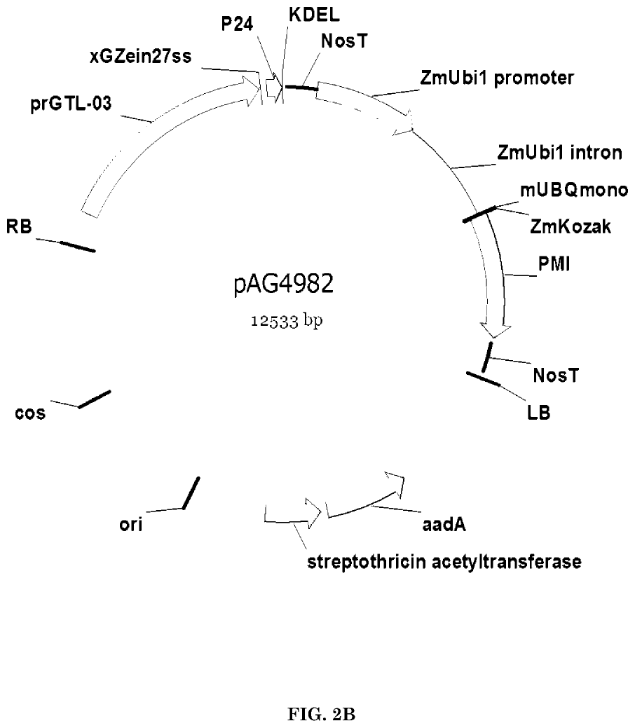

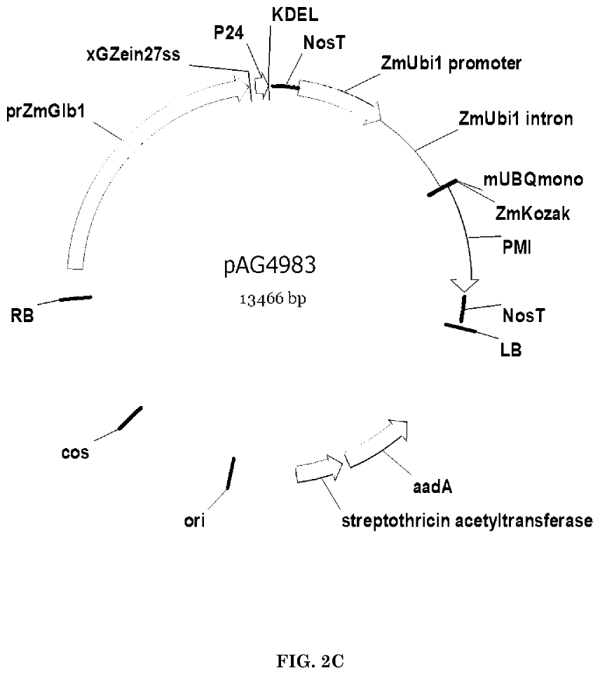

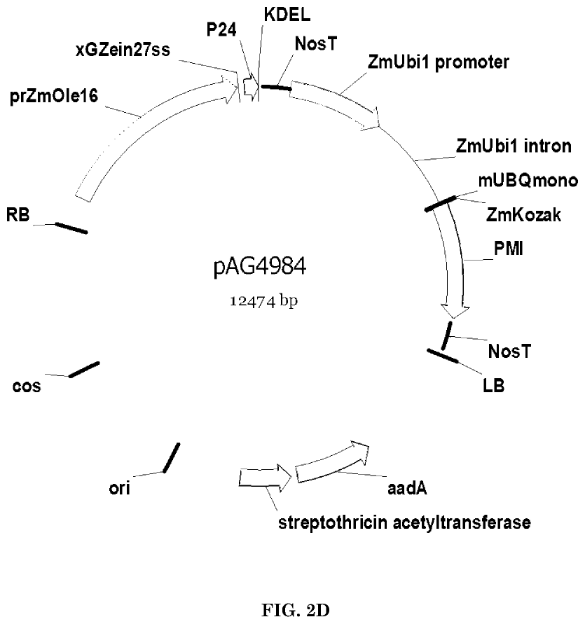

FIGS. 2A-2D illustrate the schematic drawings of the vectors pAG4981 (FIG. 2A), pAG4982 (FIG. 2B), pAG4983 (FIG. 2C), and pAG4984 (FIG. 2D).

Any DNA fragments encoding IL-10R antagonist peptides listed in Table 2, or anti-IL-10 single domain antibodies, can be cloned between desirable promoter and Nos terminator sequence in the basic P24 peptide expression vectors (Table 3), in order to generate required expression cassettes. In addition, amino terminal (N) signal sequences, such as xGZein27ss in maize expression vectors, can be replaced by other signal sequences in order to modulate specific expression and accumulation of IL-10R antagonist peptides or antibodies to desired levels. N-terminal signal sequences include, but not limited to, for example by OsGluB4sp (rice GluB-4 glutelin signal peptide), BAASS (barley alpha amylase signal sequence), or PR1 (pathogenesis related protein). The IL-10R antagonist peptides, or anti-IL-10 single domain antibodies, can be expressed to endoplasmic reticulum (ER) for improved accumulation and glycosylation using carboxyl terminal (C) retention signal sequences such KDEL (SEQ ID NO: 29), HDEL (SEQ ID NO: 30), or SEKDEL. Furthermore, IL-10R antagonist peptides, or anti-IL-10 single domain antibodies, can be expressed and directed to protein storage vacuoles with the help of signal sequences attached to either N-terminal or C-terminal part of the sequence. These storage vacuole signal sequences include HvAle from barley aleurone (thiol protease) or HvVSD from barley polyamine oxidase. If necessary, IL-10R antagonist peptides, or anti-IL-10 singe domain antibodies, can be also expressed from basic P24 vectors without signal sequences for accumulating expressed products in apoplast or cytoplasm. All those mentioned above and other signal sequences of similar functions can be added to or removed from the basic plant expression vectors. Signal sequences described above can be found in the “List of sequences” at the end of this document.

### Example 5. Additional Strategies for Expressing IL-10R Antagonist Peptides in Transgenic Maize

### Peptide Concatenation.

This strategy represents expression of a chimeric IL-10R antagonist sequence that contains multiple, contiguously linked, copies of DNA sequences encoding IL-10R antagonist peptides. The peptide coding sequences in a concatemer could be fused directly to one another or separated by intervening sequences such as AGPA hinges for stabilizing chimeric molecule for expression. Examples of possible variants of concatenated peptide sequences for the eight amino acid long peptide P25 are provided in Table 4. Each P25 concatemer can be synthesized as DNA molecule and cloned into any P24 basic expression vectors thus effectively replacing P24 coding sequence for subsequent expression in maize. In this way, new maize transformation vectors can be developed, such as for example pAG4306, where P2509 concatemer that is composed of three P25 units separated by AGPA hinges is expressed from the rice GluB4 promoter into ER. Nucleotide sequence for P2509 is available in the “List of sequences” section. A similar approach can be used for expressing all other short or all IL-10R antagonist peptides that are listed in Table 2.

TABLE 4
Examples of the P25 IL-10R antagonist peptide and
concatemers for expression in maize

| Peptide | Sequence for maize expression |
|---|---|
| P25 | PARLRELR |
| | (SEQ ID NO: 5) |
| P2501 | PARLRELRKDEL |
| | (SEQ ID NO: 32) |
| P2502 | PARLRELRPARLRELR |
| | (SEQ ID NO: 33) |
| P2503 | PARLRELRPARLRELRKDEL |
| | (SEQ ID NO: 34) |
| P2504 | PARLRELRAGPAPARLRELR |
| | (SEQ ID NO: 35) |
| P2505 | PARLRELRAGPAPARLRELRKDEL |
| | (SEQ ID NO: 36) |
| P2506 | PARLRELRPARLRELRPARLRELR |
| | (SEQ ID NO: 37) |
| P2507 | PARLRELRPARLRELRPARLRELRKDEL |
| | (SEQ ID NO: 38) |
| P2508 | PARLRELRAGPAPARLRELRAGPAPARLRELR |
| | (SEQ ID NO: 39) |
| P2509 | PARLRELRAGPAPARLRELRAGPAPARLRELRKDEL |
| | (SEQ ID NO: 40) |

### Gene Fusions.

Another strategy for expressing IL-10R antagonist peptides in plants can employ chimeric enlargement or gene fusion approach. In this strategy, target peptides can be expressed, for example, as chimeric fusions with parts of the maize gamma-zein 27kDa. This strategy was used for expressing zeolin and Zera fusion proteins (Mainieri et al., 2007; U.S. Pat. No. 8,802,825; Llop-Tous, 2010, all of which are incorporated herein by reference as if fully set forth). Co-expression of target sequences as gamma-zein fusions allows high level protein accumulation in ER-derived protein bodies. The IL-10R antagonist peptide sequences selected for expression can be fused to maize gamma-zein sequences with the help of linker sequences such as, for example linker GSGGSG (SEQ ID NO: 41). Additional linker sequences, for example linkers similar to those in zeolin fusion protein (GGGGS; SEQ ID NO: 42), Zera fusions (GGGGG; SEQ ID NO: 43), or other linkers can also be exploited (Table 5). All IL-10R antagonist peptides can be expressed with or without C-terminal sequence such as KDEL (SEQ ID NO: 29), HDEL (SEQ ID NO: 30), SEKDEL (SEQ ID NO: 31), HvVSD, or other such sequences. Furthermore, gamma-zein sequences as fusion components of chimeric protein enlargements can be substituted for other protein sequences such as, for example, elastin-like polypeptides (ELP) (Urry, 1992), which were used for expressing human IL-10 in tobacco (Patel et al., 2007) or hydrophobins that were used for transient protein expression in Nicotiana benthamiana (Joensuu et al., 2010; Jacquet et al., 2014). When either of the latter two approaches is used, expressed protein fusions form protein bodies. Two constructs that serve as examples for expressing P2509 concatemer fused with maize gamma-zein components are represented by the vectors pAG4308 and pAG4310. The pAG4311 vector is an example of expressing P2509 as a fusion with 28×VPGVG (SEQ ID NO: 44) elasting-like polypeptide (Conley et al., 2009). Variable number of repeats and sequences such as VPGXG in ELP fusion partner could be used for expressing IL-10R antagonist peptides. The ELP fusion proteins can be purified by nonchromatographic bioseparation of recombinant proteins (Lin et al., 2006). The pAG4312 construct provides an example of expressing P2509 peptide fused to the mature chain of Trichoderma reesei HFBI hydrophobin (GenBank Accession #P52754.1). Hydrophobins fusions can be purified by efficient surfactant-based aqueous two-phase system (ATPS) (Joensuu et al., 2010). Other fusion partners for IL-10R antagonist peptides could additionally be exploited such as, fusing P2509 to a thermal stable glucanase enzyme, which is presented in vector pAG4313.

TABLE 5
Linker sequences for developing protein fusions

| Linker | | |
|---|---|---|
| sequence | Nucleotide | Description |
| GSGGSG | ggcagcggcggcagcggc | Linker for expression |
| (SEQ ID | (SEQ ID NO: 45) | Phy02opt: BD21 |
| NO: 41) | | |
| GGGGS | ggcggcggcggcagc | Linker used for |
| (SEQ ID | (SEQ ID NO: 46) | Zeolin expression |
| NO: 42) | | (Mainieri |
| | | et al., 2004) |
| GGGGG | ggcggcggcggcggc | Linker used for ex- |
| (SEQ ID | (SEQ ID NO: 47) | pressing Zera fusions |
| NO: 43) | | (Llop-Tous et al., |
| | | 2011) |

### Example 6. Production of Single-Domain Antibodies to IL-10

Camelid single-domain antibodies (sdABs; also known as V_{H}H antibodies) with affinity for cIL-10 were generated by immunizing a llama with full-length, purified recombinant cIL-10 (IBI Scientific, Peosta IA). Full-length IL-10 was selected, as opposed to individual cIL-10 peptides, because cIL-10 is only 48% identical to the llama IL-10 homologue. It was previously unknown whether cIL-10 would generate an adequate immune response in llamas, but given the limited sequence identity, cIL-10 was used to test whether llamas would be naïve, and that the full-length molecule could be used to generate an adequate immune response. Furthermore, IL-10 is known to dimerize, thus using the full-length molecule would bias the generated antibodies towards epitopes that are present in the dimerized molecule. That llamas would not otherwise be exposed to cIL-10, except through injection of isolated or recombinant cIL-10, provided a novel process for generating anti-cIL-10 antibodies. Pre-immune serum was collected from a single llama prior to injection with cIL-10. The first immunization was carried out with 200 μg of cIL-10 in the presence of Complete Freund's Adjuvant (CFA). Subsequent booster immunizations were carried out, each with 100 μg cIL-10, in the presence of Incomplete Freund's Adjuvant (IFA) three weeks, seven weeks and eleven weeks after the initial immunization. Blood samples (“bleeds”) were collected from the animal one week after each of the booster immunizations. FIG. 3 illustrates the llama's immune response prior to (pre-immune), and after being dosed with cIL-10.

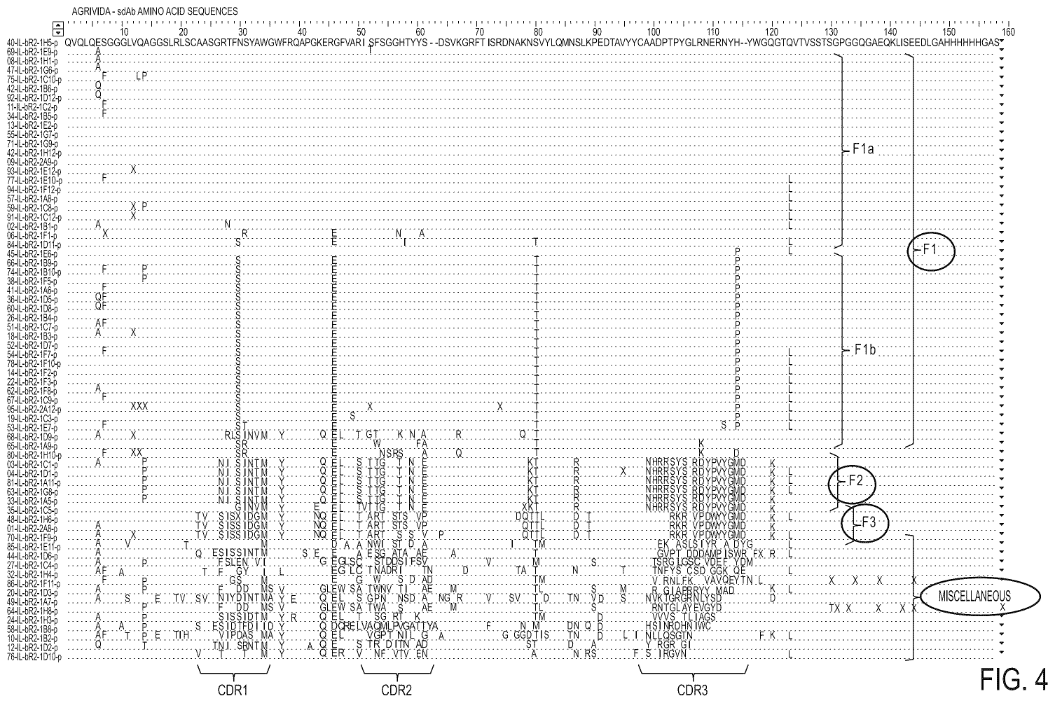

The production of antibodies targeting cIL-10 in the animal during this immunization process was evaluated via ELISA using each of the bleeds as shown in FIG. 3, and the bleeds were then used to develop single-domain antibodies. The alignment of the chicken and llama IL-10 homologs shows 48% identity (68% similarity). The preparation of sdABs has been described elsewhere (Goldman et al. 2006; Arbabi Ghahroudi et al. 1997; Liu et al. 2013). Briefly, peripheral blood mononuclear cells (PBMCs) were isolated from the bleeds that were collected during weeks eight and 12 of the immunization protocol. RNA was purified from the PBMCs and used to create phagemid cDNA libraries for expression and screening of sdABs. Notably, sdABs represent only the heavy chain variable region (VHH) from the llama antibodies produced when the host was injected with recombinant cIL-10, and therefore the RNA-derived, DNA coding sequence of sdABs represents a synthetic nucleotide that is produced through a novel process that does not occur in nature. Two rounds of panning against purified recombinant cIL-10 were used to enrich the library for phage displaying sdAB with affinity for the antigen. From the enriched libraries, individual clones were generated, isolated and sequenced. FIG. 4 illustrates the results of the anti-IL-10 sdAB screening and sequencing. FIG. 4 demonstrates that the isolated sdAB amino acid sequences reside in four distinct groups, with a fifth miscellaneous group. The complementarity-determine regions (CDRs) are designated in FIG. 4.

### Example 7. Anti-IL-10 sdAbEC50 Measurements and Thermal Stability

Candidate sdABs were evaluated for their apparent binding affinity against purified cIL-10, as measured by ELISA. Different concentrations of individual isolated sdABs were incubated on cIL-10 ELISA plates, with increasing signals indicative of higher levels of sdAB binding to cIL-10. Apparent EC50 values were estimated by determining the sdAB concentration at which 50% of the maximum signal was observed. FIG. 5 illustrates the ELISA measurements and apparent binding affinity of selected sdAB candidates to chicken IL-10. Among the candidates tested, chIL10sdAB1A11 (SEQ ID NO: 135), chIL10sdAB1F11 (SEQ ID NO: 146), and chIL10sdAB1B9 (SEQ ID NO: 111), had EC50 values of less than 100 nM, with estimated values of 1 nM, 15 nM, and 35 nM, respectively. Another sdAB, chIL10sdAB1D6 (SEQ ID NO: 143), had an estimated EC50 value of 100 nM. Additional sdABs were evaluated in this way for their EC50 values, including chIL10sdAB1H1 (SEQ ID NO: 89) with an EC50 value of 20 nM. Since higher binding affinity is reflected by lower EC50 values, sdABs with low EC50 values, and more divergent sequences were selected for further assessment and development.

Although sdABs are considered to have high specificity for their antigenic target, and do not bind strongly to non-specific peptides, it is often desirable to demonstrate binding specificity to individual epitopes or peptides. The sdABs were prepared using the full-length chIL-10 protein, however individual peptides could also be used in our method. Likewise, counter selecting sdABs that bind the full length chIL-10, but had little or no affinity to specific peptides was also used to identify sdABs that bound desired antigenic peptides, but not others. Counter selection can be made using several different methods, including an ELISA or dot-blot, where the peptides for counter selection are immobilized on a surface and the anti-cIL-10 sdAB are incubated to allow binding, washed to remove unbound sdAB, then incubated with a labeled anti-llama antibody to detect any bound sdAB. Any sdAB that does not bind the immobilized peptides used for counter selection but still bind the full length IL-10, can be further developed with the confidence that they possess the desired binding criteria. Some peptides that were used for counter selection include: DDELNIQL [peptide 1; SEQ ID NO: 180], VLPRAMQT [peptide 2; SEQ ID NO: 181], EKMDENGI [peptide 3; SEQ ID NO: 182], EPTCLHFS [peptide 4; SEQ ID NO: 183], DQMGDLL [peptide 5; SEQ ID NO: 184], DQLHSLL [peptide 6; SEQ ID NO: 185], VMPKAESD [peptide 7; SEQ ID NO: 186], VMPQAENH [peptide 8; SEQ ID NO: 187], SKLQERGV [peptide 9; SEQ ID NO: 188], SELQERGV [peptide 10; SEQ ID NO; 189], ENSCIHFP [peptide 11; SEQ ID NO: 190], DSSCIHLP [peptide 12; SEQ ID NO: 191], DQLNSML [peptide 13; SEQ ID NO: 192], NMLQERGV [peptide 14; SEQ ID NO: 193], DSSCTHFP [peptide 15; SEQ ID NO: 194], DDLEIGL [peptide 16; SEQ ID NO: 195], VLPTAIADMTEE peptide 17; SEQ ID NO: 196], TQMEGKGP [peptide 18; SEQ ID NO: 197], and NQCCRFV [peptide 19; SEQ ID NO: 198].

Internal screening for thermal stability was performed to determine the heat tolerance of sdABs, which may be important for their use in animal feed processing. In particular, thermal stability is a highly desirable property in animal feed pelleting processes, where the molecules may be exposed to temperatures over 70° C., and up to 125° C., depending on the specific process and pelleting equipment used. In order to evaluate the thermal stability, heat treated sdABs were prepared by incubating the sdABs at 70° C., 75° C., 80° C., 85° C., and 90° C. for 30 seconds, 60 seconds, 90 seconds, 120 seconds, 300 seconds and 600 seconds, and were then allowed to equilibrate to room temperature. Control sdABs were incubated at 37° C. or room temperature, for the same period of time that the heat treated sdABs and also allowed to equilibrate to room temperature. The EC50 values of the sdABs were then compared between the control (37° C. or room temperature treated) and treatment (those heated between 70° C. and 90° C. for various amounts of time) sdABs by ELISA. Thermal stability, as expressed by the ratio of the EC50 values of the heat treated sdABs and control sdABs, ranged between 30% to 90%, with higher thermal stability values correlating to lower temperatures and lower exposure times.

### Example 8. Anti-IL-10 sdAb Gastric Stability

Simulated gastric fluid (SGF) consisted of 0.084 M HCl, 35 mM NaCl, pH 1.2, containing 2630 Units of pepsin per milliliter. Reaction stop solution was 200 mM sodium carbonate. Protein samples, including bovine serum albumin (BSA), chIL10sdAB1A11 (SEQ ID NO: 135), chIL10sdAB1F11 (SEQ ID NO: 146), chIL10sdAB1H1 (SEQ ID NO: 89), chIL10sdAB1B9 (SEQ ID NO: 111), chIL10sdAB1D6 (SEQ ID NO: 143), chIL10sdAB1E11 (SEQ ID NO: 142) chIL10sdAB1F7 (SEQ ID NO: 121) chIL10sdAB1F9 (SEQ ID NO: 141), and chIL10sdAB2A8 (SEQ ID NO: 140), to be tested were brought to a concentration of 5 mg/mL in storage buffer (50 mM MES, 150 mM NaCl, 40% (v/v) glycerol, pH 6.3).

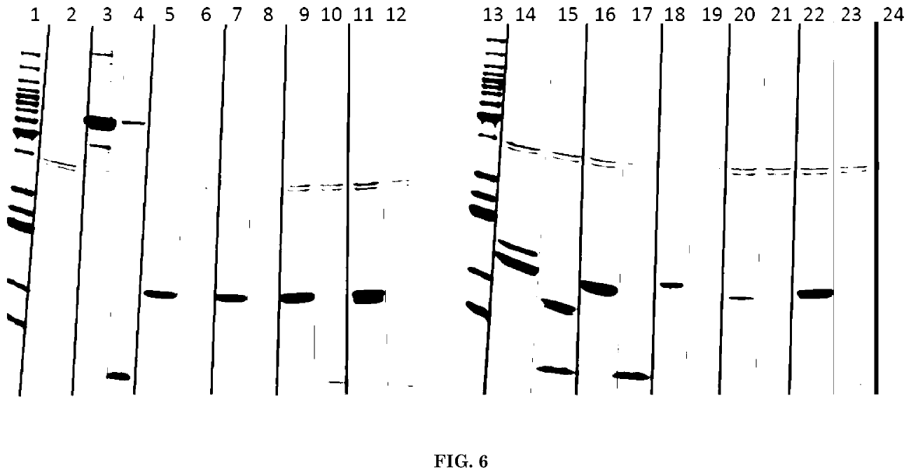

Single domain antibody samples (2.5 μL) were dispensed into 200 μL thin-walled PCR tubes and prewarmed on a PCR thermal cycler set to 37° C. Aliquots (100 μL) of SGF were placed into PCR tubes and also prewarmed. For 0 minute digestion samples, 17.5 μL of stop solution was added to the protein samples before adding SGF. Digestions were initiated by the addition of 47.5 μL of prewarmed SGF to the sdAB samples. After 1 minute, 2 minutes, 5 minutes, 10 minutes, and 30 minutes, reactions were terminated by the addition of 17.5 μL stop solution. SDS-PAGE sample-loading buffer (17.5 μL; ThermoFisher catalog #NP0007, with dithiothreitol added to a concentration of approximately 50 mM) was added to each sample. After heating for 10 minutes at 70° C., 15 μL of each sample was loaded onto a protein electrophoresis gel (ThermoFisher catalog #NP0321 or similar) and electrophoresis was performed as directed by the manufacturer. Gels were then stained with Coomassie Blue dye using standard methods. Results are shown in FIG. 6 for the 0 and 10 minute time points. In this figure, lane 1—molecular weight standards; lane 2—pepsin only; lane 3—BSA 0 min; lane 4—BSA 10 min; lane 5—1A11 0 min; lane 6—1A11 10 min; lane 7—1F11 0 min; lane 8—1F11 10 min; lane 9—1H1 0 min; lane 10—1H1 10 min; lane 11—1B9 0 min; lane 12—1B9 10 min; lane 13—molecular weight standards; lane 14—1D6 0 min; lane 15—1D6 10 min; lane 16—1E11 0 min; lane 17—1E11 10 min; lane 18—1F7 0 min; lane 19—1F7 10 min; lane 20—1F9 0 min; lane 21—1F9 10 min; lane 22—2A8 0 min; lane 23—2A8 10 min; and lane 24—pepsin only. FIG. 6 shows that BSA is significantly degraded within a 10 minute digestion in SGF, as are all of the sdABs tested. Given the inherent rapid digestibility of the sdABs, it is unexpected that the sdABs perform well in controlling Coccidiosis and binding IL-10 when dosed into feed. It may have been anticipated that rapid digestion in SGF would underlie poor performance of sdABs in controlling Coccidiosis as they should be quickly degraded and therefore have a limited ability to bind IL-10 and block IL-10 signaling. Given that the sdABs described herein are effective in controlling Coccidiosis suggests that gastric stability is not a dominating factor in oral antibody administration and is a beneficial trait as proteins that are rapidly degraded in SGF pose a lower allergenicity risk than those that are stable in SGF. Given the increased thermal stability of sdABs (see discussion in Example 1, above), it is unusual that the developed sdABs are readily digestible by pepsin, as it is widely regarded that high thermal stability, as demonstrated by sdABs, correlates with high SGF stability, in contrast to the measured digestibility of the developed anti-IL-10 sdABs. The anti-IL-10 sdABs developed herein have good thermal stability and are readily digested in pepsin, which are attributes that support the product's performance and safety profile, which should aid in its regulatory evaluation and eventual customer acceptance.

### Example 9. Chicken IL-10 Ligand-Receptor Assay and sdAB IC50 Measurement

An assay was developed to measure how the sdABs bind their target cIL-10, and prevent cIL-10 from binding to its receptor. The soluble domain of the cIL-10 receptor was expressed and immobilized on a biacore probe surface. It was then incubated with cIL-10 and different mixtures of sdABs and cIL-10, and the binding of cIL-10 to the soluble domain of the cIL-10 receptor was measured by surface plasmon resonance.

The amino acid sequence of the soluble domain of the chicken interleukin-10 receptor subunit 1 (cIL-10R1) was deduced by alignment of the amino acid sequence of the human IL-10R alpha receptor subunit (UniProtKB/SwissProt accession number Q13651) with the analogous chicken IL-10 receptor subunit 1 (NCBI reference sequence NP_001034686).

For this assay, the gene coding for the chicken receptor soluble domain (residues 22-231) was synthesized as an upstream fusion to the human IgG₁ Fc domain (residues 100-330 of UniProtKB/SwissProt accession number P01857) connected by a linker consisting of IEGRMD [SEQ ID NO: 199] (the final, aggregate expressed molecule comprising the cIL-10R1 fused to IEGRMD [SEQ ID NO: 199] fused to the Fc residues 100-330 will be referred to as “cIL-10R1-Fc”). cIL-10R1-Fc could be directly immobilized to facilitate the surface plasmon resonance binding assay on a biacore instrument. To produce cIL-10R1-Fc, the gene encoding cIL-10R1-Fc was cloned into pGAPZαB (ThermoFisher) via the EcoRI and NotI restriction sites. Pichia pastoris strain GS115 was transformed with the plasmid as directed in the pGAPZαB instruction manual. A high-expressing clone was grown in a 2.5 L fermenter using a fed-batch protocol. Growth medium in the batch phase consisted of 1.5 L of 20 g/L peptone, 10 g/L yeast extract, 13.4 g/L yeast nitrogen base, 10 g/L casamino acids, 10 g/L glycerol, and 100 mM sodium phosphate monobasic. Temperature was maintained at 28° C., pH was maintained at 6.0 by addition of 50% ammonium hydroxide, and dissolved oxygen (pO₂) was maintained at 30%. After glycerol was exhausted, as indicated by a spike in dissolved oxygen, temperature was lowered to 25° C. and feeding of 750 mL of 100 g/L glucose, 50 g/L peptone, 25 g/L yeast extract, 10 g/L casamino acids, 0.5% (v/v) Antifoam 204, and 100 μg/mL zeocin was initiated.

Culture supernatant was isolated by centrifugation and sterile filtered through a 0.22 μm filter. Supernatant was taken to 1M ammonium sulfate by the addition of 0.5 volume of 3M ammonium sulfate, 20 mM Tris·HCl, 1 mM EDTA, pH 8. After filtering, the receptor fusion was purified by hydrophobic interaction chromatography (Phenyl Sepharose), affinity chromatography (Protein G Sepharose), anion-exchange chromatography (MonoQ), and size-exclusion chromatography.

Chicken IL-10 was obtained from Kingfisher Biotech, Inc. Camelid VHH domains fused to C-terminal myc- and his-tags were expressed in E. coli with expression directed to the periplasmic space by an N-terminal signal peptide. Protein was extracted from the periplasm by osmotic shock and then purified by metal chelation chromatography and size-exclusion chromatography. Protein concentrations were determined by measuring the absorbance at 280 nm, using extinction coefficients calculated from the amino acid sequences.

Affinity and inhibition were measured using a BIAcore T200 surface plasmon resonance instrument (GE Healthcare). Approximately 7000 response units of the cIL-10R1-Fc fusion was covalently coupled to a CM5 sensor chip using EDC/NHS chemistry as directed by the manufacturer. All experiments were conducted at 37° C. in HBS-EP buffer (10 mM HEPES, 150 mM NaCl, 3 mM EDTA, 0.005% [v/v] Tween-20, pH 7.4). Binding affinity (K_{d}) for cIL-10 to immobilized cIL-10R1-Fc was determined by injecting five concentrations of cIL-10 over the sensor chip for 120 seconds at 20 μL/second. After 300 seconds of dissociation, the sensor chip was regenerated with a 10 second pulse of 10 mM sodium acetate, 0.5 mM EDTA, pH 4 followed by a 120 second stabilization phase before making the next injection. The BIAcore evaluation software was used to calculate the maximum bound material (Rₘₐₓ) for each cIL-10 concentration. Rₘₐₓ values were plotted against cIL-10 concentration and fit to the equation for a rectangular hyperbola to calculate K_{d}.

Inhibition of cIL-10 binding to cIL-10R1-Fc by the sdABs was measured by preincubating 10 nM cIL-10 with six concentrations of each sdAB in HBS-EP for at least 20 minutes, followed by injection onto the sensor chip as described above. Calculated Rₘₐₓ values for each sdAB concentration were plotted against the sdAB concentration to determine IC₅₀ values.

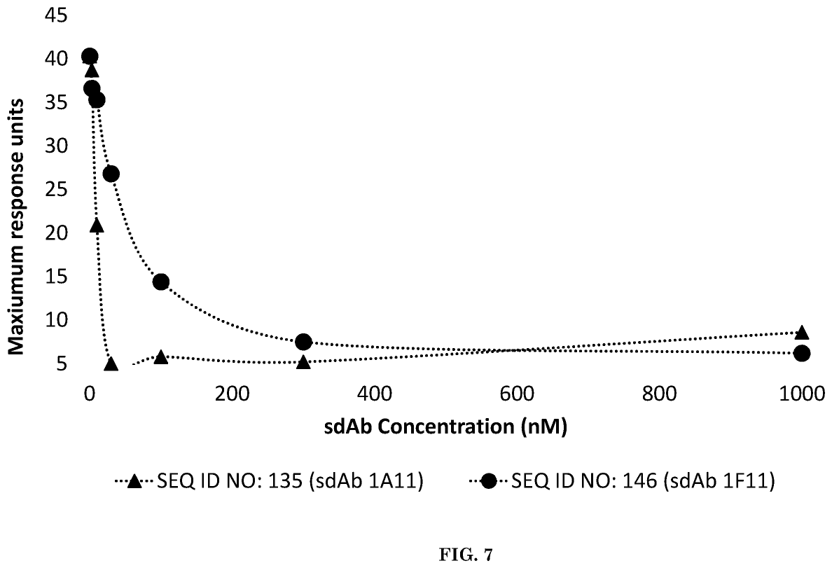

FIG. 7 illustrates anti-IL-10 sdAb IC50 values measured for chIL10sdAB1A11 (SEQ ID NO: 135) and chIL10sdAB1F11 (SEQ ID NO: 146).

### Example 10. Cell-Based Assay to Measure the Biological Activity of cIL-10 on Interferon Gamma (IFN-□) Production and Inhibition of cIL-10 by Anti-IL-10 sdAB in Stimulated Primary Chicken Spleenocytes

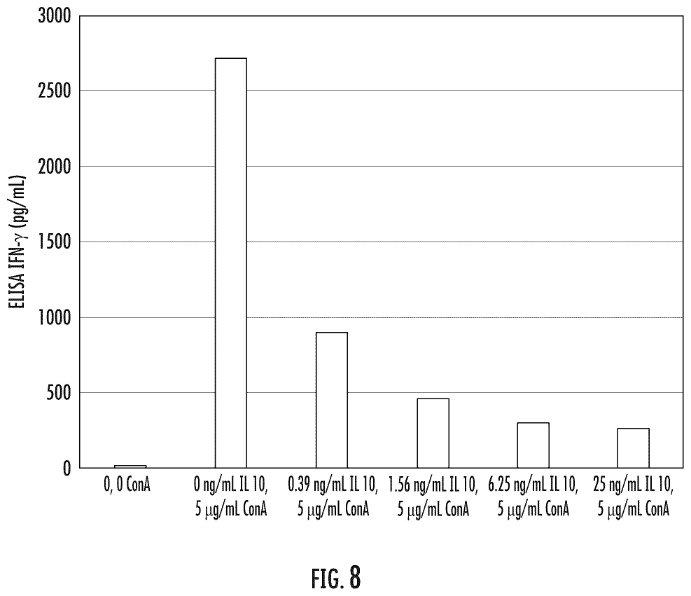

IL-10 is known to be a potent regulator of the immune system that affects many cell types and generally acts to attenuate inflammation and the immune response (Kevin N. Couper, Daniel G. Blount, Eleanor M. Riley, “IL-10: The Master Regulator of Immunity to Infection,” The Journal of Immunology, 180:5771-5777, 2008). Primary chicken spleen cells were used to evaluate the use of sdABs in blocking the biological activity of IL-10 in cellular signaling on a relevant target cell type. A cell-based assay was developed to study the inhibitory effect of cIL-10 on the concanavalin A (ConA) dependent induction (or phytohemagglutinin (PHA) dependent induction, both stimulators work with these cells) of interferon gamma (IFN-□) production in chicken spleen cells (Wu et al. (2016) and Rothwell et al. (2004)). Briefly, lymphocytes and mononuclear cells were isolated from chicken spleens by differential centrifugation on Ficoll-Hypaque. Freshly isolated cells were cultured at 5×10⁶ cells/mL in wells of a 96-well plate for 72 hours in the presence of 1.2 μg/mL ConA (or 12.5 μg/mL PHA) with, or without, cIL-10 at concentrations of 0-25 mg/mL. Levels of IFN-□ in the supernatants of treated cells were determined by ELISA. FIG. 8 shows the IFN-□ response of the cells in the absence of ConA, ConA with no cIL-10, and ConA with 0.39, or 1.56, or 6.25, or 25 ng/mL cIL-10). As seen in FIG. 8, IL-10 suppresses of ConA-induced secretion of IFN-γ in primary chicken spleen cells. FIG. 8 also shows that spleen cells have a dose dependent response in IFN-□ production to chIL-10, as increasing cIL-10 lowers IFN-□ production in a dose-dependent manner.

To test how effective sdABs were in interrupting cIL-10 stimulated production of IFN-□□ primary chicken spleen cells were incubated with and without different concentrations of sdABs ranging from 0.1 nM up to 10 □M, with ConA, and with or without cIL-10. Levels of IFN-□ in the culture supernatants were determined by ELISA. Included in these studies, as control treatments, were spleen cells treated only with 5 μg/mL ConA (positive control for IFN-□ production), cells treated with 5 μg/mL of ConA and 1.5 ng/ml of cIL-10 (positive control for cIL-10 inhibition of ConA-dependent IFN-g production), and cells treated with 5 μg/mL of ConA, 1.5 ng/ml of cIL-10, and either an anti-IL-10 polyclonal antibody (“all10 pAb”, positive control antibody) or a non-specific sdAB that did not bind cIL-10 (“aMOP pAb (NC)”, a negative control antibody to demonstrate that non-specific binding cannot provide the same effect observed with antibodies that specifically bind cIL-10). Experimental treatments contained 5 μg/mL ConA, 1.5 ng/ml of cIL-10, and varying concentrations of anti-IL-10 sdABs. In these experiments, sdABs were dose at 1 nM, 30 nM, and 1000 nM.

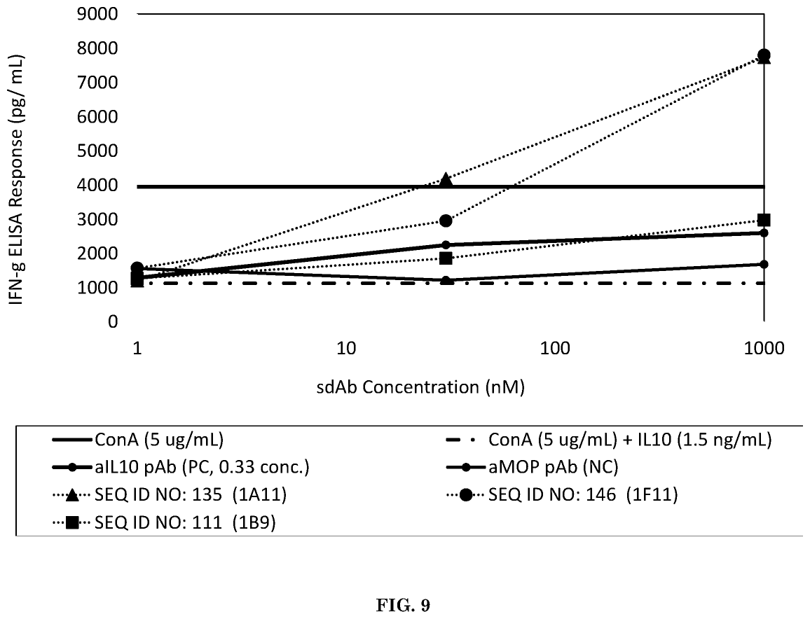

FIG. 9 illustrates the anti-IL-10 antibodies (chIL10sdAB1A11 (SEQ ID NO: 135), chIL10sdAB1B9 (SEQ ID NO: 111), chIL10sdAB1F11 (SEQ ID NO: 146) effect on the IFN-γ secretion in primary chicken spleen cells. Based on these results, the apparent EC50 values for chIL 10sdAB1A11 (SEQ ID NO: 135), chIL10sdAB1B9 (SEQ ID NO: 111), chIL10sdAB1F11 (SEQ ID NO: 146) were measured to be 25 nM, 40 nM, and 60 nM, respectively. Although higher than the EC₅₀ and IC₅₀ values measured for binding of the anti-IL-10 sdABs to cIL-10, they are still in relative agreement with these values and further demonstrate the biological efficacy of the anti-IL-10 sdABs in blocking IL-10 signaling and decrease immune system suppression.

### Example 11. Plant Expression of Anti-IL-10 Single Domain Antibodies

Antibody expression was demonstrated in transient expression using tobacco and in transgenic corn events. Other plant species can be used to express the anti-IL-10 sdABs, including rice, sorghum, soy beans, and canola. Depending on the final product and intended use, a particular plant species may be more suited for production than other species.

Expression cassettes containing the sequences of anti-IL-10 sdAbs for expression in maize are included in Table 6. In Table 6, some vectors contain single expression cassettes, while other vectors contain multiple expression cassettes, which usually helps increase expression of the sdAB. The DNA sequence of each chicken anti-IL-10 sdAB contained in the expression cassettes listed in Table 6 has been codon optimized for maize gene expression, however, the genes may be optimized for other plant (or microbial) species to improve their expression when a different expression host is desired.

TABLE 6
Plant expression vectors for expression
of chicken anti-IL-10 sdABs:

| Vector | Chicken anti-IL-10 sdAb expression cassette(s) |
|---|---|
| pAG4314 | OsGluB4P:xGZein27ss:chIL10sdAB1A11A:KDEL |
| pAG4315 | OsGluB4P:xGZein27ss:chIL10sdAB1B9:KDEL |
| pAG4316 | OsGluB4P:xGZein27ss:chIL10sdAB1F11A:KDEL |
| pAG4317 | OsGluB4P:xGZein27ss:chIL10sdAB1H1A:KDEL |
| pAG4985 | ZmZ27P:xGZein27ss:chIL10sdAB1A11A:KDEL |
| pAG4986 | ZmZ27P:xGZein27ss:chIL10sdAB1B9:KDEL |
| pAG4987 | ZmZ27P:xGZein27ss:chIL10sdAB1F11A:KDEL |
| pAG4988 | ZmZ27P:xGZein27ss:chIL10sdAb1A11A:KDEL + |
| | OsGluB4P:xGZein27ss: chIL10sdAB1A11A:KDEL |
| pAG4989 | ZmZ27P:xGZein27ss:chIL10sdAB1B9:KDEL + |
| | OsGluB4P:xGZein27ss: chIL10sdAB1B9:KDEL |
| pAG4990 | ZmZ27P:xGZein27ss:chIL10sdAB1F11A:KDEL + |
| | OsGluB4P:xGZein27ss: chIL10sdAB1F11A:KDEL |
| pAG4991 | ZmZ27P:xGZein27ss:chIL10sdAB1H1A:KDEL |
| pAG4992 | ZmZ27P:xGZein27ss:chIL10sdAB1H1A:KDEL + |
| | OsGluB4P:xGZein27ss: chIL10sdAB1H1A:KDEL |
| pAG4993 | ZmZ27P:xGZein27ss:chIL10sdAB1A11B:KDEL |
| pAG4994 | ZmZ27P:xGZein27ss:chIL10sdAB1F11B:KDEL |
| pAG4995 | ZmZ27P:xGZein27ss:chIL10sdAB1H1B:KDEL |
| pAG4996 | ZmZ27P:xGZein27ss:chIL10sdAB1A11B:KDEL + |
| | OsGluB4P:xGZein27ss: chIL10sdAB1A11A:KDEL |
| pAG4997 | ZmZ27P:xGZein27ss:chIL10sdAB1F11B:KDEL + |
| | OsGluB4P:xGZein27ss: chIL10sdAB1F11A:KDEL |
| pAG4998 | ZmZ27P:xGZein27ss:chIL10sdAB1H1B:KDEL + |
| | OsGluB4P:xGZein27ss: chIL10sdAB1H1A:KDEL |

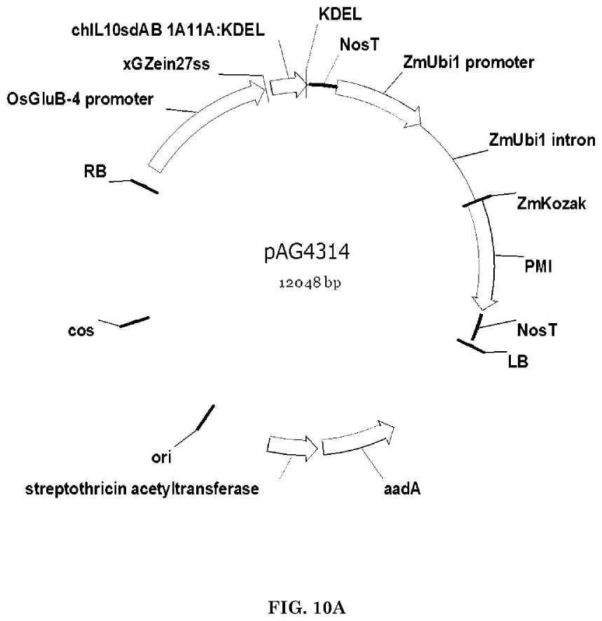

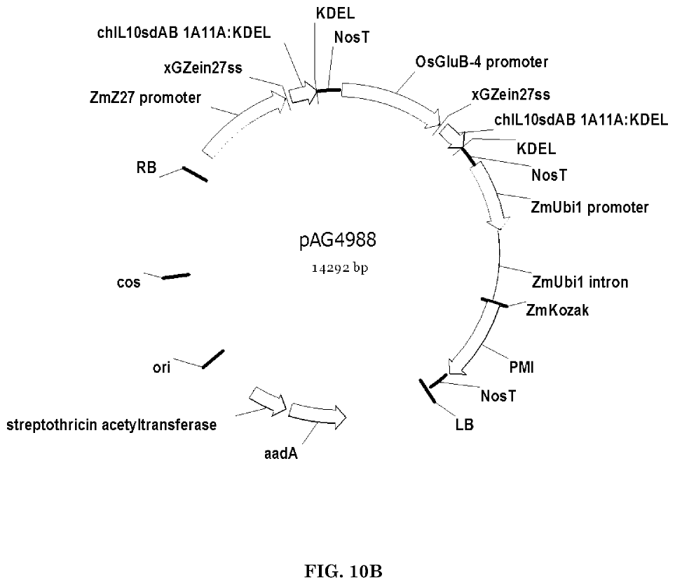

FIG. 10A is a schematic drawing of a vector pAG4314 that includes a single expression cassette for an anti-IL-10 sdAB (OsGluB4P:xGZein27ss:chIL10sdAB1A11A:KDEL) and the PMI expression cassette for selection in plants. FIG. 10B is a schematic drawing of a vector pAG4988 that increases the transgene dosage by including two expression cassettes for the same anti-IL-10 sdAB (ZmZ27P:xGZein27ss:chIL10sdAb1A11A:KDEL+OsGluB4P: xGZein27ss:chIL10sdAB1A11A:KDEL). Vector pAG4988 also includes the PMI expression cassette for selection in plant tissues. The T-DNA sequences for the vectors listed in the table are provided below in such a way that each sequence starts with the right border repeat and ends with the left border repeat.

Deduced protein sequences for selected chicken IL10 sdAb are provided with the maize gamma zein 27 signal sequence and KDEL signal sequence, which are underlined at N-terminal and C-terminal ends of the protein, respectively.

Protein Sequences of chIL 10 sdABs Encoded by Plant Expression Cassettes

>xGZein27ss:chIL10sdAb1A11:KDEL
(SEQ ID NO: 84)
MRVLLVALALLALAASATSQVQLQESGGGLVQPGGSLRLSCASGNIFSIN
TMGWYRQAPGKQRELVASITTGGTTNYEDSVKGRFTISRDNAKKTVYLQM
NRLKPEDTAVYYCNHRRSYSGRDYPVYGMDYWGKGTLVTVSSKDEL
>xGZein27ss:chIL10sdAb1B9:KDEL
(SEQ ID NO: 85)
MRVLLVALALLALAASATSQVQLQESGGGLVQAGGSLRLSCAASGRTFSS
YAWGWFRQAPGKEREFVARISFSGGHTYYSDSVKGRFTISRDNAKNTVYL
QMNSLKPEDTAVYYCAADPTPYGLRNERNYPYWGQGTQVTVSSKDEL
>xGZein27ss:chIL10sdAb1F11:KDEL
(SEQ ID NO: 86)
MRVLLVALALLALAASATSQVQLQEFGGGLVQPGGSLRLSCASGRTGSSY
AMGWFRQAPGKEREFVAAISWSGGSTDYADSVKGRFTISRDNAKNTMYLQ
MNSLKPEDTAVYYCAVDRNLFKLRVAVQEYTNLGQGTQVTVSSKDEL
>xGZein27ss:chIL10sdAb1H1:KDEL
(SEQ ID NO: 179)
MRVLLVALALLALAASATSQVQLQASGGGLVQAGGSLRLSCAASGRTFNS
YAWGWFRQAPGKERGFVARISFSGGHTYYSDSVKGRFTISRDNAKNSVYL
QMNSLKPEDTAVYYCAADPTPYGLRNERNYHYWGQGTQVTVSSKDEL

The nucleotide sequences encoding chIL10sdAB1A11, chIL10sdAB1F11, and chIL10sdAB1H1 antibodies in vectors and nucleotide sequences were named 1A11A (chIL101A11A), 1F11A (chIL101F11A), and 1H1A (chIL101H1A), respectively, to reflect different coding sequences with altered codon useage. This modification was made in order to avoid any possible confusion in the future due to availability of different variants (for example, “variant A” and “variant B”) of the maize codon optimized sequences for maize expression. The deduced protein sequences encoded by the variants “A” and “B” are identical.

Nucleotide sequence alignments of the maize codon optimized variants “A’ and “B” of the selected chIL 10 sdABs:

| CLUSTAL O(1.2.4) multiple sequence alignments | | |
|---|---|---|
| 1A11A | CAGGTTCAGCTGCAGGAAAGCGGTGGCGGACTGGTGCAGCCAGGTGGCAGCCTCAGGCTG | 60 |
| 1A11B | CAGGTGCAGCTCCAGGAGTCCGGCGGCGGCCTCGTGCAGCCGGGCGGCTCCCTCCGCCTG | 60 |
| | ***** ***** ***** *** ***** ** ******** ** *** **** * *** | |
| 1A11A | AGCTGCGCTGCTAGCGGCAATATTTTTAGCATTAACACAATGGGTTGGTATAGACAGGCT | 120 |
| 1A11B | AGCTGCGCCGCGTCCGGCAACATCTTCAGCATCAACACGATGGGCTGGTACAGGCAGGCC | 120 |
| | ******** ** ****** ** ** ***** ***** ***** ***** ** ***** | |
| 1A11A | CCTGGCAAGCAGCGTGAGCTCGTTGCCAGCATTACCACGGGTGGTACAACCAATTATGAA | 180 |
| 1A11B | CCCGGCAAGCAGCGGGAGCTCGTGGCCTCCATCACCACGGGCGGCACCACGAACTACGAG | 180 |
| | ** *********** ******** *** *** ******** ** ** ** ** ** ** | |
| 1A11A | GATAGCGTGAAGGGTCGTTTTACCATTAGCAGGGACAATGCTAAGAAGACCGTTTACCTC | 240 |
| 1A11B | GACAGCGTCAAGGGCCGCTTCACCATCTCCAGGGACAACGCCAAGAAGACGGTGTACCTC | 240 |
| | ** ***** ***** ** ** ***** ********* ** ******** ** ****** | |
| 1A11A | CAGATGAACAGGCTGAAGCCAGAAGATACCGCCGTGTATTACTGCAACCACAGGAGAAGC | 300 |
| 1A11B | CAGATGAACCGCCTGAAGCCGGAGGACACGGCGGTCTACTACTGCAACCACCGCAGGTCC | 300 |
| | ********* * ******** ** ** ** ** ** ** ************ * ** * | |
| 1A11A | TATAGCGGAAGAGATTATCCTGTTTACGGTATGGACTACTGGGGCAAGGGAACCCTGGTT | 360 |
| 1A11B | TACAGCGGCAGGGACTACCCCGTGTACGGCATGGACTACTGGGGCAAGGGCACCCTCGTG | 360 |
| | ** ***** ** ** ** ** ** ***** ******************** ***** ** | |
| 1A11A | ACCGTGAGCAGC 372 [SEQ ID NO: 173] | |
| 1A11B | ACCGTGTCCTCC 372 [SEQ ID NO: 174] | |
| | ****** * * | |
| 1F11A | CAGGTTCAGCTCCAGGAGTTTGGTGGCGGACTGGTGCAGCCAGGTGGCAGCCTCAGGCTG | 60 |
| 1F11B | CAGGTGCAGCTCCAGGAGTTCGGCGGCGGCCTCGTGCAGCCGGGCGGCTCCCTCCGCCTG | 60 |
| | ***** ************** ** ***** ** ******** ** *** **** * *** | |
| 1F11A | AGCTGCGCTGCTAGCGGTAGAACCGGCAGCAGCTATGCTATGGGATGGTTTAGACAGGCT | 120 |
| 1F11B | AGCTGCGCCGCGTCCGGCAGGACGGGCTCCAGCTACGCGATGGGCTGGTTCAGGCAGGCG | 120 |
| | ******** ** *** ** ** *** ****** ** ***** ***** ** ***** | |
| 1F11A. | CCAGGCAAGGAGCGTGAATTTGTTGCTGCCATTAGCTGGAGCGGAGGTAGCACCGATTAT | 180 |
| 1F11B | CCCGGCAAGGAGAGGGAGTTCGTGGCGGCCATCTCGTGGAGCGGCGGCAGCACCGACTAC | 180 |
| | ** ********* * ** ** ** ** ***** ******** ** ******** ** | |
| 1F11A | GCTGACAGCGTGAAGGGCAGGTTTACCATTAGCAGAGATAATGCCAAGAACACCATGTAC | 240 |
| 1F11B | GCTGACTCCGTCAAGGGCCGCTTCACCATCAGCAGGGACAACGCGAAGAACACGATGTAC | 240 |
| | ****** *** ****** * ** ***** ***** ** ** ** ******** ****** | |
| 1F11A | CTCCAGATGAATAGCCTGAAGCCAGAGGATACCGCTGTTTATTACTGCGCCGTGGACCGT | 300 |
| 1F11B | CTCCAGATGAACTCCCTGAAGCCGGAGGACACCGCCGTGTACTACTGCGCGGTCGACCGC | 300 |
| | *********** ********* ***** ***** ** ** ******** ** ***** | |
| 1F11A | AATCTCTTTAAGCTGAGGGTTGCTGTGCAGGAATACACCAACCTCGGCCAGGGAACCCAG | 360 |
| 1F11B | AACCTCTTCAAGCTGAGGGTGGCCGTCCAGGAGTACACCAACCTCGGCCAGGGCACCCAG | 360 |
| | ** ***** *********** ** ** ***** ******************** ****** | |
| 1F11A | GTTACCGTGAGCAGC 373 [SEQ ID NO: 173] | |
| 1F11B | GTGACCGTGTCCTCC 373 [SEQ ID NO: 176] | |
| | ** ****** * * | |
| 1H1A | CAGGTTCAGCTCCAGGCTTCGGGCGGCGGGCTCGTCCAGGCGGGCGGCTCGCTCAGGCTC | 60 |
| 1H1B | CAGGTGCAGCTCCAGGCCTCCGGCGGCGGCCTCGTGCAGGCGGGCGGCTCCCTCCGCCTG | 60 |
| | ***** *********** ** ******** ***** ************** *** * ** | |
| 1H1A | TCGTGCGCGGCGTCGGGGCGGACTTTCAACAGCTACGCTTGGGGCTGGTTCAGGCAGGCG | 120 |
| 1H1B | AGCTGCGCCGCGTCCGGCAGGACCTTCAACAGCTACGCTTGGGGCTGGTTCAGGCAGGCG | 120 |
| | ***** ***** ** **** ************************************ | |
| 1H1A | CCGGGCAAGGAGCGCGGCTTCGTGGCCAGGATCTCCTTCAGCGGCGGCCACACCTACTAC | 180 |
| 1H1B | CCGGGCAAGGAGCGCGGCTTCGTGGCCAGGATCTCCTTCAGCGGCGGCCACACCTACTAC | 180 |
| | ************************************************************ | |
| 1H1A | TCCGACAGCGTCAAGGGCCGCTTCACGATCTCCAGGGACAACGCCAAGAACAGCGTGTAC | 240 |
| 1H1B | TCCGACAGCGTCAAGGGCCGCTTCACGATCAGCAGGGACAACGCCAAGAACTCCGTGTAC | 240 |
| | ****************************** ******************* ******* | |
| 1H1A | CTCCAGATGAACTCCCTGAAGCCCGAGGACACGGCCGTCTACTACTGCGCGGCGGACCCG | 300 |
| 1H1B | CTCCAGATGAACAGCCTGAAGCCCGAGGACACGGCCGTCTACTACTGCGCGGCGGACCCG | 300 |
| | ************ ********************************************** | |
| 1H1A | ACGCCCTACGGCCTCAGGAACGAGCGGAACTACCATTACTGGGGGCAGGGCACGCAGGTC | 360 |
| 1H1B | ACCCCATACGGCCTCCGCAACGAGAGGAACTACCACTACTGGGGCCAGGGCACCCAGGTG | 360 |
| | ** ** ********* * ****** ********** ******** ******** ***** | |
| 1H1A | ACTGTCTCTTCG 372 [SEQ ID NO: 177] | |
| 1H1B | ACCGTGTCCTCC 372 [SEQ ID NO: 178] | |
| | ** ** ** ** | |

TABLE 7
The percentage of nucleotide sequence identity between
“A” and “B” variants

| Sequence | Sequence | Sequence |
|---|---|---|
| “A” | “B” | identity, % |
| 1A11A | 1A11B | 78.2 |
| 1F11A | 1F11B | 79.7 |
| 1H1A | 1H1B | 90.3 |

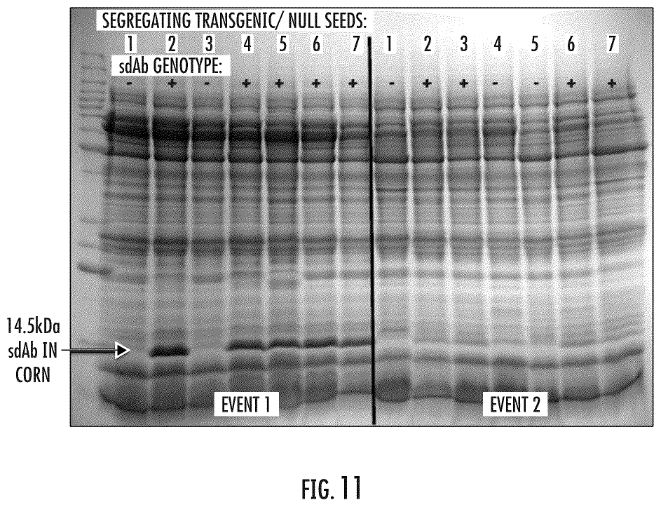

Any polynucleotides encoding anti-IL10 antibodies can be cloned between desirable promoter and terminator sequences in the plant expression vectors described herein (Table 6), in order to generate expression cassettes. In addition, amino terminal (N) signal sequences, such as xGZein27ss in maize expression vectors, can be replaced by other signal sequences in order to modulate specific expression and accumulation of anti-IL-10 antibodies to desired levels. N-terminal signal sequences include, but not limited to, for example by OsGluB4sp (rice GluB-4 glutelin signal peptide), BAASS (barley alpha amylase signal sequence), or PR1 (pathogenesis related protein). The anti-IL-10 antibodies can be expressed to endoplasmic reticulum (ER) for improved accumulation and potential glycosylation using carboxyl terminal (C) retention signal sequences such KDEL (SEQ ID NO: 29), HDEL (SEQ ID NO: 30), or SEKDEL (SEQ ID NO: 31). Furthermore, anti-IL10 antibodies can be expressed and directed to protein storage vacuoles with the help of signal sequences attached to either N-terminal or C-terminal part of the sequence. These storage vacuole signal sequences include HvAle from barley aleurone (thiol protease) or HvVSD from barley polyamine oxidase. If necessary, anti-IL-10 antibodies can be also expressed from expression vectors without signal sequences for accumulating expressed products in apoplast, chloroplast, or cytoplasm. All of the genetic elements mentioned above, including other signal sequences with similar functions, can be added to or removed from the basic plant expression vectors to tailor the expression properties of the anti-IL-10 sdAB. FIG. 11 illustrates that using the genetic elements described herein, sdABs can be expressed at high level in corn grain. In FIG. 11, sdABs expressed in corn were extracted from individual seed from a hemizygous parent, where 50% of the seed contained the expression cassette and 50% of the seed did not contain the expression cassette. In FIG. 11, individual seed from two different transgenic events were genotyped and analyzed for the presence of the expressed sdAB by SDS-PAGE electrophoresis and coomassie staining. As shown in FIG. 11, the presence of the sdAB correlated perfectly with the genotyping result, that is, only seed that tested positive for the gene by PCR produced a protein band at the right size of the sdAB. Further, expression levels of the sdAB in event 1 were estimated at 3 mg per gram of corn grain, which would result in an expression level of 9 mg of sdAB per gram of corn for the fully homozygous event. Expression levels for recombinant proteins, including sdABs, up to 21 mg per gram of corn grain have been observed using the expression cassettes and genetic elements described herein.

### Example 12. Transient Expression of Chicken IL10 sdAb 1A11 in Leaves of Tobacco Nicotiana benthamiana

Transient protein expression in plants has been used by multiple groups for production of therapeutic proteins and vaccine antigens. Among various plant species, tobacco Nicotiana benthamiana, is one of the most suitable production hosts because it can achieve a high level of protein expression in a short timeframe by using a leaf infiltration procedure. Such production attributes are required for economical heterologous protein production.

### Genetic Elements and Construction of Vectors

For expression in N. benthamiana, the chicken IL-10 sdAb 1A11 (referred to herein as Nb1A11; SEQ ID NO: 202) sequence was codon optimized for Nicotiana codon usage and synthesized by GenScript as either an 868 bp NcoI-Avril DNA fragment, which at 5′ end contained 90 bp tobacco PR1a gene sequence [SEQ ID NO: 205] encoding transit peptide, 304 bp first intron of Arabidopsis ubiquitin 10 gene (AtUBQ10i) in Nb1a11 coding region, and at 3′ end myc tag, 6×His, and KDEL sequences [SEQ ID NO: 206], or as 564 bp NcoI-AvrII fragment without the AtUBQ10i intron. The Nb1A11:AtUBQ10i sequence is shown below as SEQ ID NO: 203, and the intron sequence is indicated by the bold characters and is underlined.

[SEQ ID NO: 203]
CAAGTTCAGTTACAGGAAAGCGGGGGAGGTTTAGTTCAGCCTGGGGGTTC
ATTGAGGTTGAGTTGTGCAGCAAGTGGAAATATTTTTTCTATTAATACTA
TGGGATGGTATAGACAAGCTCCAGGTAAATTTCTGTGTTCCTTATTCTCT
CAAAATCTTCGATTTTGTTTTCGTTCGATCCCAATTTCGTATATGTTCTT
TGGTTTAGATTCTGTTAATCTTAGATCGAAGACGATTTTCTGGGTTTGAT
CGTTAGATATCATCTTAATTCTCGATTAGGGTTTCATAGATATCATCCGA
TTTGTTCAAATAATTTGAGTTTTGTCGAATAATTACTCTTCGATTTGTGA
TTTCTATCTAGATCTGGTGTTAGTTTCTAGTTTGTGCGATCGAATTTGTC
GATTAATCTGAGTTTTTCTGATTAACAGGAAAGCAAAGAGAACTTGTTGC
AAGTATTACTACTGGAGGAACTACAAATTACGAAGATAGTGTTAAAGGAA
GATTCACTATTTCAAGAGATAATGCTAAGAAAACAGTTTATCTTCAGATG
AATAGATTGAAGCCAGAAGATACAGCAGTTTACTACTGTAATCATAGAAG
ATCATACTCTGGTAGAGATTATCCTGTTTATGGTATGGATTATTGGGGAA
AAGGGACATTAGTTACAGTTAGCAGC

The AtUBQ10i was inserted into Nb1A11 coding region between nucleotides 124 and 125 for dual purpose: 1) monitoring expression of Nb1A11 from plant cells rather than from Agrobacterium; 2) potentially enhancing expression of Nb1A11 in tobacco, since positive effects of heterologous introns on gene transcription in plants and other species are well documented in the literature (“Introns increase gene expression in cultured maize cells,” J. Callis, M. Fromm, V. Walbot, Genes Dev., 1:1183-1200, 1987; doi: 10.1101/gad. 1.10.1183; “Increased Gene Expression by the First Intron of Maize Shrunken-1 Locus in Grass Species,” V. Vasil, M. Clancy, R. J. Ferl, I. K. Vasil, L. C. Hannah, Plant Physiol. 91:1575-1579, 1989; “Intron-mediated enhancement as a method for increasing transgene expression levels in barley,” J. G. Bartlett, J. W. Snape, W. A. Harwood, Plant Biotechnology Journal, 7:856-866, 2009, all of which are incorporated herein by reference as if fully set forth). The sequences of myc tag and 6×His were included to facilitate Nb1A11 sdAB detection and purification. The KDEL sequence was added for retaining the expressed 1A11 sdAb in endoplasmic reticulum (ER) for improving accumulation levels of the protein. For transient expression of Nb1A11 sdAB in N. benthamiana, a new and previously uncharacterized constitutive ubiquitin 1 gene promoter (prNbUbi1) was used. The sequence of the prNbUbi1 is shown below as SEQ ID NO: 204, wherein the sequence of the intron sequence is indicated by the bold characters and is underlined.

[SEQ ID NO: 204]
CATGAAAGTCCACATCATCAGCTCGTCCCAAACATCACTACTAGACCCAA
CTCGTTCAATCTTCTCGACTACAACAAATGAAATCCGCTCATCAAGGTGT
CTGAGGCTGATCTCAATAAATGGAGGGACTAATTGTATGGATCGAAATCT
GCCCCAAAATATTTAGGGTAAGGTACATTGAAGAAAGAGTCATCGAGGTC
GATCAGGAAACGATCGAGATGTTAACAATGGTCGATGTCGAGCACCGCAT
GTAGAGTTGTAACACCTAGTTTTTAGAATAGGATAATACAAAGAATATTC
TATTGGATATCCTTTACACTTATATTATTAGAGTTTGTTAGGAAAATGAC
CCACATAAATAGGAAAAAAGACAATGAATGGAGACAGGTGACATTTATCT
GATGAGAACAGACTTTTGATAGAAGATATTTTCTCTCTCACTAAGATACA
AACACTACATTTTCATCAAGATTCTTGTTCATATCATTGTACACTTTTCT
ATCAAATCTGAAATAATTTAAATATTCTAGGATTTGTCTGTCACTCATCA
TTGTCAGACGGGATAATCATGTACTCATCCTTTTTTGGCAAACCACTTTT
TCTATTTACTTAAATGCCATTTATTGATATCTATTGCTAGTCATTCCTCC
ACCGTTGCTCATACTTTTTTGCAATAGTATGCATGTTGATATCAATCCAC
CACCAAATCTTCTAACATTAATCATATTTTCACAACTTACATTTATAAAT
ATTATTATTAACTAAGTTTAACTCACTATTATATAAACTCAATTGTTTTA
CTCGAAAGTTACACTATTATATTGAGAATTACGTTTCCAAACTTTTTAAG
CATTTATTGTGTAACCATAAGAGACTTTGATTTTTTAAAAATTATTTAGA
TTTTATTAATGAGAATGGCACAACATTATGGTCAACTATGTATTTCATCA
TTAACTAAATAGTTAGCACTTTGATTCTTTCACATGAATTATGAATTTAT
GATGGGCTCAAATTAAAATTAAATTATTCACAAAAACTTATTTTTATATT
CTACGACACCCACTTTTCTAGCTTTTTCCCGAAGGGGCGTGAGAGTGTCA
CACACGCTCCAAATTTCCCAACCAAACAAGGAAAGGGCAGAGAAAGATAG
CTTTAGCGTGTTGTTTTGGTGCACTACACGTCATTAGGACACGTGTCATG
ATATAATAGGCCAATCCCACGAGGCGGTTTCGTCTTGAGTCGGCCATAGT
GTCCATAAATGAGGGCTCTCCGTCGGTTTCCCCATCATTCATCAGATTTA
TCTTCTATACTTCATCGCCTTCATATTTCTCTCTCAAGGTTTGAGAATTT
CTTCAATTTCTCGCTTTAGCAGTTCTTTTTTATTGAATCAACGATTTCGG
CATCTAAAGTCCTAATTTTGAAGTTCATTGCTTTAATTGTTTGTTGTTGA
TTTTATATTATTACAG

This promoter was identified by screening N. benthamiana Expressed Sequence Tag (EST) database for the most abundant in leaf tissue ubiquitin gene transcript. The database is maintained by the Nicotiana benthamiana Genome and Transcriptome Sequencing Consortium (Nakasugi K, Crowhurst R N, Bally J, Wood C C, Hellens R P, Waterhouse P M (2013) De Novo Transcriptome Sequence Assembly and Analysis of RNA Silencing Genes of Nicotiana benthamiana. PLOS ONE 8 (3): e59534, which is incorporated by reference herein as if fully set forth). The transcript Nbv6.1trP26199, annotated as putative ubiquitin 1, appeared to contain significantly larger number of ESTs (1196) than other ubiquitin related transcripts. The Nbv6.1trP26199 specific 1466 bp upstream genomic sequence, which included 128 bp 3′UTR positioned intron, was identified in N. benthamiana draft genome sequence (v1.0.1) that is available at the Sol Genomics Network at Boyce Thompson Institute for Plant Research (Bombarely, A., H. G. Rosli, J. Vrebalov, P. Moffett, L. A. Mueller, and G. B. Martin (2012). A draft genome sequence of Nicotiana benthamiana to enhance molecular plant-microbe biology research. Molecular Plant-Microbe Interactions 25:1523-1530, which is incorporated by reference herein as if fully set forth).

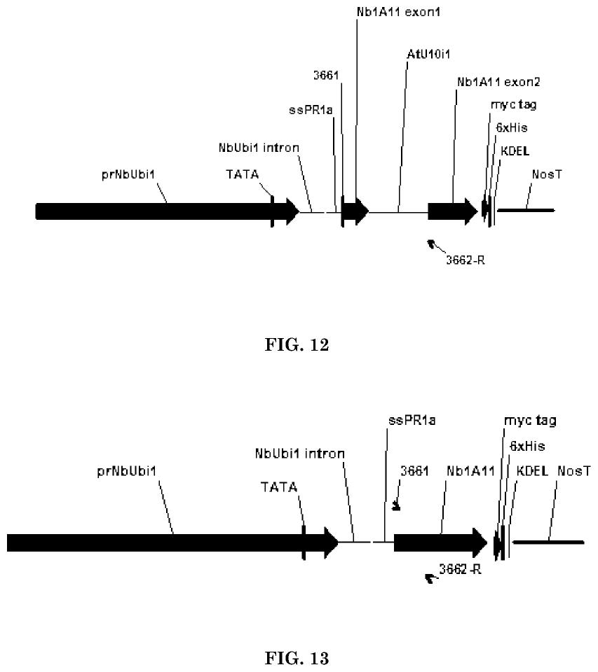

This 1466 bp sequence in N. benthamiana genome has nucleotide coordinates 74703-76169 in the Scaffold No: 5041 and is fused tocoding region of a putative ubiquitin gene that encodes five 76 amino acid long identical ubiquitin monomers. The 1466 bp prNbUbi1 was synthesized by GenScript as NotI-NcoI fragment. The entire Nb1A11+AtUBQ10i or Nb1A11 expression cassettes were cloned into NotI-KpnI sites of the pLH9000 vector, which was kindly provided by Dr. I. Lernomtova (IPK Gatersleben, Germany), and the final constructs were designated as pLH1A11int or pLH1A11 respectively. FIG. 12 illustrates pLH1A11int expression cassette. FIG. 13 illustrates pLH1A11 expression cassette. Subsequently, pLH9000, pLH1A11int and pLH1A11 were electroporated into electrocompetent cells of the Agrobacterium strain GV3101. Agrobacterium colonies carrying pLH9000, pLH1A11int or pLH1A11 constructs were validated by PCR.

N. benthamiana Plant Growing and Inoculation with Agrobacterium

The seeds of N. benthamiana were acquired from The US Nicotiana Germplasm Collection (NC State University). The seeds were sowed into 4″×4′ pots containing ProMix soil. After germination the seedlings and the plants were kept at 16 h day and 8 h night light regime. Five weeks old healthy N. benthamiana plants were used for syringe infiltration with Agrobacterium strains GV3101 harboring either pLH9000 as a negative control, pLH1A11int or pLH1A11 expressing Nb1A11 (chicken IL-10 sdAb1A11 for N. benthamiana expression as described above), or with the mixture of two Agrobacterium strains, such as GV3101 with pLH1A11int and C58C1 with p19. As used herein, 1A11 sdAB is synonomous with anti-chicken IL-10 sdAB and chicken IL-10 sdAb1A11. The p19 is a tomato bushy stunt virus protein, which is involved into suppression of RNA-dependent gene silencing thus improving expression of heterologous proteins. Agrobacterium strains GV3101 and C58C1 with p19 were kindly provided by Dr. I. Lernomtova (IPK Gatersleben, Germany). The Agrobacterium strains containing plasmids were grown from single colonies overnight in LB medium supplemented with corresponding antibiotics, the cells were harvested by centrifugation and resuspended to OD₆₀₀=0.4 in 10 mM MgCl₂, 10 mM MES-K (pH 5.6). Prior to syringe infiltration of N. benthamiana leaves 100 μM Acetosyringone was added to each Agrobacterium strain. The leaf tissues for expression analysis of Nb1A11 were harvested on day 4 post infiltration.

### RNA Analysis of Nb1A11 Expression

The total plant RNA from Agrobacterium infiltrated N. benthamiana leaf tissues was isolated with NucleoSpin RNA Plant Kit (Takara) according to manufacturer's protocol. Subsequently, 1 μg of the total RNA was converted into cDNA using iScript CDNA Synthesis Kit (Bio-Rad) and 1.5 μl of each cDNA was used as template in PCR reactions with the following primers:

forward primer 3661
(5′- CGTGCCCAAGTTCAGTTACA - 3′ [SEQ ID NO: 200 ]),
and
reverse primer 3662
(5′- TTGCAACAAGTTCTCTTTGCTT - 3′ [SEQ ID NO: 201]).

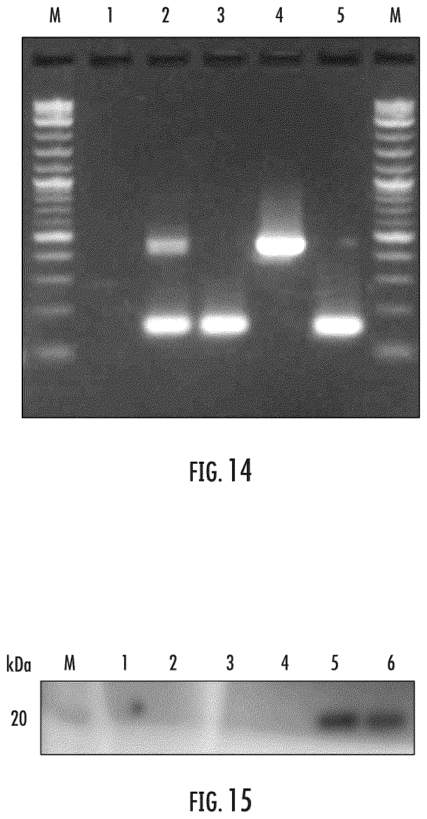

The primers were positioned to flank intron AtUBQ10i within the coding region of Nb1A11 and allow unambiguous identification of the plant cell expressed 1A11 transcript with the fully spliced out intron. The Platinum Taq DNA Polymerase (Invitrogen) was used to amplify PCR products under conditions recommended by manufacturer with 36 cycles of amplification and primer annealing temperature of 55° C. The PCR products were resolved on 2% agarose gel. FIG. 14 illustrates end point RT-PCR analysis of transiently expressed Nb1A11 in N. benthamiana leaves. In this figure, lanes 1-5: lane 1—GV3101+pLH9000 (negative control); lane 2—GV3101+pLH1A11int; lane 3—GV310130 pLH1A11; lane 4—plasmid pLH1A11int; and lane 5—plasmid pLH1A11.

In N. benthamiana leaf tissues infiltrated with the negative control plasmid pLH9000 no 1A11 transcripts were amplified (lane 1). Distinct PCR products of the expected 1A11 transcript sizes were amplified from N. benthamiana leaf tissues infiltrated with either pLH1A11int or pLH1A11 (lanes 2 and 3 respectively).

Amplified PCR products from plasmids pLH1A11int and pLH1A11 were uses as positive controls and run in lanes 4 and 5. The resulting products in lanes 4 and 5 were observed to have identical sizes to the products in lanes 2 and 3. A lower intensity PCR band corresponding in size to the expected 458 bp fragment containing AtUBQ10i was also detected in lane 2. This fragment could have been amplified from either N. benthamiana genomic DNA, which was still lingering in total RNA preparation of the sample despite of its removal by DNase digestion as suggested by manufacturer's instructions, or alternatively, the amplification product is indicative of a fraction of isolated total RNA containing Nb1A11 transcripts with still unspliced AtUBQ10i.

1A11 sdAb Transient Protein Expression Analysis

Based on the results of Nb1A11 RNA transcript analysis in leaves of N. benthamiana, the leaf tissue samples that were infiltrated with pLH1A11int and pLH1A11int+p19 were selected for protein isolation and Western blot analysis. Agrobacterium-infiltrated leaf tissues were ground in liquid nitrogen and the total protein was isolated using extraction buffer composed of 1 M NaCl, 50 mM Sodium Phosphate pH8.0, 10 mM Imidazole. The extraction buffer was supplemented with 1×Halt Protease Inhibitor Cocktail (ThermoFisher) and 2 mM β-mercaptoethanol. Protein extraction was performed at 4° C. for 1 h with agitation, samples were centrifuged twice to remove plant debris. The 1A11 sdAB containing 6×His tag at its C-terminal end was isolated from the cleared supernatant of the total N. benthamiana leaf protein using Ni-NTA spin columns (QIAGEN) according to manufacturer's protocol for native conditions. The 1A11 sdAB was further concentrated using Amicon Ultra-2 centrifugal filters (Millipore-Sigma) and protein concentration was determined by NanoDrop spectrophotometer. Subsequently, 15 μg of 1A11 containing concentrated protein fraction was resolved on 4-12% gradient NuPAGE polyacrylamide gels (ThermoFisher) using 1×MOPS SDS gel running buffer. Biotynylated Protein Ladder (Cell Signalling Technologies) and Precision Plus Protein Kaleidoscope (Bio-Rad) were used as molecular weight standards. The proteins were separated in a polyacrylamide gel and transferred onto PVDF membrane using semi-dry Western blotting procedure. The PVDF membrane bound 1A11 protein was detected using Rabbit Anti-VHH HRP (Invitrogen) as the primary antibody at 1:2500 dilution followed by Anti-Rabbit IgG Peroxidase Goat (Sigma) as the secondary antibody at 1:5000 dilution. Detection of the biotinylated proteins in the protein molecular weight ladder was accomplished by Anti-biotin HRP-liked Ab at 1:15000 dilution (Cell Signaling Technology). The signal detection was achieved using Super Signal West Pico Plus chemiluminescent substrate (ThermoFisher). FIG. 15 illustrates 1A11 protein expression in Agrobacterium infiltrated leaves of N. benthamiana. The Western blot shows detection of 1A11 sdAb in samples 5, 6. It was demonstrated that the lanes 5 and 6 contain a protein of expected molecular weight of 17.4 kDa that is cross reactive with the Anti-VHH primary antibody, indicating the protein is indeed 1A11 sdAB.

### Example 13. Anticoccidial Efficacy of IL-10R Peptide Antagonists and sdABs in Commercial Broiler Chickens Infected with a Mixture of Eimeria acervulina, E. maxima, and E. tenella Field Isolates

The same study design was used to measure the anticoccidial efficacy/sensitivity anti-IL10 antibodies, or IL-10R antagonists against a mixture of Eimeria acervulina, E. maxima, and E. tenella. In these trials, chickens were separated into multiple control groups that were either exposed to Eimeria (Infected, I) or not exposed to Eimeria (Non-Infected, NI), and treatment groups that were exposed to Eimeria and treated with various diets. The control groups included a negative control group receiving normal feed with and without anti-IL-10 sdAB (or with and without IL-10R antagonist) in the feed, and a positive control group receiving a standard chemical Coccidiostat. Treatment groups were fed diets containing from 50 g to four kilograms of milled grain expressing anti-IL-10 sdAB per kilogram of feed (or IL-10R antagonist peptide doses ranging from 1 milligram of peptide per kilogram of feed to 40 milligrams of IL-10R antagonist peptide per kilogram of feed. Anti-IL-10 sdABs, including SEQ ID NO: 87 (chIL10sdAB1H5), SEQ ID NO: 88 (chIL10sdAB1E9), SEQ ID NO: 89 (chIL10sdAB1H1), SEQ ID NO: 90 (chIL10sdAB1G6), SEQ ID NO: 91 (chIL10sdAB1C10), SEQ ID NO: 92 (chIL10sdAB1B6), SEQ ID NO: 93 (chIL10sdAB1D12), SEQ ID NO: 94 (chIL10sdAB1C2), SEQ ID NO: 95 (chIL10sdAB1B5), SEQ ID NO: 96 (chIL10sdAB1E2), SEQ ID NO: 97 (chIL10sdAB1G7), SEQ ID NO: 98 (chIL10sdAB1G9), SEQ ID NO: 99 (chIL10sdAB1H12), SEQ ID NO: 100 (chIL10sdAB2A9), SEQ ID NO: 101 (chIL10sdAB1E12), SEQ ID NO: 102 (chIL10sdAB1E10), SEQ ID NO: 103 (chIL10sdAB1F12), SEQ ID NO: 104 (chIL10sdAB1A8), SEQ ID NO: 105 (chIL10sdAB1C8), SEQ ID NO: 106 (chIL10sdAB1C12), SEQ ID NO: 107 (chIL10sdAB1B1), SEQ ID NO: 108 (chIL10sdAB1F1), SEQ ID NO: 109 (chIL10sdAB1D11), SEQ ID NO: 110 (chIL10sdAB1E6), SEQ ID NO: 111 (chIL10sdAB1B9), SEQ ID NO: 112 (chIL10sdAB1B10), SEQ ID NO: 113 (chIL10sdAB1F5), SEQ ID NO: 114 (chIL10sdAB1A6), SEQ ID NO: 115 (chIL 10sdAB1D5), SEQ ID NO: 116 (chIL 10sdAB1D8), SEQ ID NO: 117 (chIL10sdAB1B4), SEQ ID NO: 118 (chIL 10sdAB1D7), SEQ ID NO: 119 (chIL10sdAB1B3), SEQ ID NO: 120 (chIL10sdAB1D7), SEQ ID NO: 121 (chIL10sdAB1F7), SEQ ID NO: 122 (chIL10sdAB1F10), SEQ ID NO: 123 (chIL10sdAB1F2), SEQ ID NO: 124 (chIL10sdAB1F3), SEQ ID NO:125 (chIL10sdAB1F8), SEQ ID NO: 126 (chIL10sdAB1C9), SEQ ID NO: 127 (chIL10sdAB1A12), SEQ ID NO: 128 (chIL10sdAB1C3), SEQ ID NO: 129 (chIL10sdAB1E7), SEQ ID NO: 130 (chIL10sdAB1D9), SEQ ID NO: 131 (chIL10sdAB1A9), SEQ ID NO: 132 (chIL10sdAB1H10), SEQ ID NO: 133 (chIL10sdAB1C1), SEQ ID NO: 134 (chIL10sdAB1D1), SEQ ID NO: 135 (chIL10sdAB1A11), SEQ ID NO: 136 (chIL10sdAB1G8), SEQ ID NO: 137 (chIL10sdAB1A5), SEQ ID NO: 138 (chIL10sdAB1C5), SEQ ID NO: 139 (chIL10sdAB1H6), SEQ ID NO: 140 (chIL10sdAB2A8), SEQ ID NO: 141 (chIL10sdAB1F9), SEQ ID NO: 142 (chIL10sdAB1E11), SEQ ID NO: 143 (chIL10sdAB1D6), SEQ ID NO: 144 (chIL10sdAB1C4), SEQ ID NO: 145 (chIL10sdAB1H4), SEQ ID NO: 146 (chIL10sdAB1F11), SEQ ID NO: 147 (chIL10sdAB1D3), SEQ ID NO: 148 (chIL10sdAB1A7), SEQ ID NO: 149 (chIL10sdAB1H8), SEQ ID NO: 150 (chIL10sdAB1H3), SEQ ID NO: 151 (chIL10sdAB1B8), SEQ ID NO: 152 (chIL10sdAB1B2), SEQ ID NO: 153 (chIL10sdAB1D2), or SEQ ID NO: 154 (chIL10sdAB1D10), or peptides of SEQ ID NO: 1 [P21], SEQ ID NO: 2 [P22], SEQ ID NO: 3 [P23], SEQ ID NO: 4 [P24], SEQ ID NO: 5 [P25], SEQ ID NO: 6 [P26], SEQ ID NO: 7 [P27], SEQ ID NO: 8 [P28], SEQ ID NO: 9 [P29], SEQ ID NO: 10 [P11], SEQ ID NO: 11 [P30], SEQ ID NO: 12 [P31], SEQ ID NO: 13 [P32] SEQ or concatenated peptides SEQ ID NO: 32 [P2501], SEQ ID NO: 33 [P2502], SEQ ID NO: 34 [P2503 ], SEQ ID NO: 35 [P2504], SEQ ID NO: 36 [P2505], SEQ ID NO: 37 [P2506], SEQ ID NO: 38 [P2507], SEQ ID NO: 39 [P2508], or SEQ ID NO: 40 [P2509] were tested in this manner.

These feeding trials are eight days in length and consist of 96 cages, each starting with 8 male chicks. The treatments will be replicated in 8 blocks, randomized within blocks of 8 cages each. A randomization procedure for pen assignment for treatments and blocks was used by the contracting facility.

TABLE 8
Treatment design to test chIL10sdAB expressing corn grain

| | | Infected/ | Additive | | |
|---|---|---|---|---|---|
| | | Non- | inclusion, g | Cages/ | Birds/ |
| Trt | Description | Infected | additive/kg feed | Trt | Cage |
| T1 | Nonmedicated | NI | 0 | 8 | 8 |
| | (NMNI) | | | | |
| T2 | Nonmedicated | I | 0 | 8 | 8 |
| | (NMI, NC) | | | | |
| T3 | Coccidiostat (PC) | NI | 0.010 | 8 | 8 |
| T4 | Coccidiostat (PC) | I | 0.010 | 8 | 8 |
| T5 | SEQ ID NO: 135 | I | 4000 | 8 | 8 |
| | (chIL10sdAB1A11) | | | | |
| T6 | SEQ ID NO: 135 | I | 4000 | 8 | 8 |
| | (chIL10sdAB1A11) | | | | |
| T7 | SEQ ID NO: 135 | I | 1 | 8 | 8 |
| | (chIL10sdAB1A11) | | | | |
| T8 | SEQ ID NO: 135 | I | 40 | 8 | 8 |
| | (chIL10sdAB1A11) | | | | |
| T9 | SEQ ID NO: 135 | I | 500 | 8 | 8 |
| | (chIL10sdAB1A11) | | | | |
| T10 | SEQ ID NO: 135 | I | 300 | 8 | 8 |
| | (chIL10sdAB1A11) | | | | |
| T11 | SEQ ID NO: 135 | I | 150 | 8 | 8 |
| | (chIL10sdAB1A11) | | | | |
| T12 | SEQ ID NO: 135 | I | 50 | 8 | 8 |
| | (chIL10sdAB1A11) | | | | |

Other sdABs were tested using the same trial design, same grain loadings, but different chIL10sdAB expressing corn grain. In a similar way, the same trial design was used to test IL-10R peptide antagonists as shown in Table 9.

TABLE 9
Treatment design to test IL-10R antagonist peptides

| | | | Additive | | |
|---|---|---|---|---|---|
| | | | inclusion, g | | |
| | | Infected/ | additive/kg | Cages/ | Birds/ |
| Trt | Description | Non-Infected | feed | Trt | Cage |
| T1 | Nonmedicated | NI | 0 | 8 | 8 |
| | (NMNI, NC) | | | | |
| T2 | Nonmedicated | I | 0 | 8 | 8 |
| | (NMI, NC) | | | | |
| T3 | Coccidiostat (PC) | NI | 0.010 | 8 | 8 |
| T4 | Coccidiostat (PC) | I | 0.010 | 8 | 8 |
| T5 | P21 | NI | 0.070 | 8 | 8 |
| T6 | P21 | I | 0.070 | 8 | 8 |
| T7 | P21 | I | 0.050 | 8 | 8 |
| T8 | P21 | I | 0.035 | 8 | 8 |
| T9 | P21 | I | 0.020 | 8 | 8 |
| T10 | P21 | I | 0.015 | 8 | 8 |
| T11 | P21 | I | 0.010 | 8 | 8 |
| T12 | P21 | I | 0.001 | 8 | 8 |

At the start of every trial, the facility was checked to ensure that all cages have water and feed available in each cage, which was provided to animal ad libitum. The building temperature was maintained as appropriate for the age of the birds. Even, continuous illumination was provided by fluorescent lamps hung vertically along the wall. Cages will be checked twice daily, and observations including availability of feed, water, temperature and any unusual conditions were recorded. Mortality birds were removed from cages, and the cage number, date, weight of the bird, sex and probable cause of death were recorded.

As part of the trial, an unmedicated commercial starter ration compounded with basal feedstuffs was formulated. This ration was used to formulate the study's negative and positive control rations, and experimental diets, which were all fed ad libitum from the date of chick arrival until completion of the study. Quantities of all basal feed and test articles used to prepare treatment batches were documented and tested as part of the trial quality control procedures. Treatment diets were mixed to a uniform distribution of test article. The mixer was flushed between control and treatment diets, and in between each treatment diet. Each treatment feed was then distributed among cages of the corresponding treatment.

Day of hatch male chicks (Cobb 500) were used in the study. Upon arrival, chicks will be colony raised in Coccidia free battery cages. At 12 days of age (trial day 0) chicks will grouped into sets of 8, weighed, and placed into an assigned cage. Birds were weighed by cage on day of trial 0 and 8.

On day of trial 2, all non-infected birds received 1 ml of distilled water by oral pipette. All other birds will receive the coccidial inoculum diluted to a 1 ml volume and dosed by oral pipette. The inoculum was a mixture of Eimeria acervulina, E. maxima, and E. tenella field isolates, which produces a mild infection with all species.

Data were collected after starting the study on days 0, 2, 7, and 8. On day 0, birds were weighed and allocated to their cages for the study. On Day 2, designated birds were inoculated with coccidian. On Day 7, dropping pans were cleaned to prepare for droppings collection on Day 8, and subsequent analysis. On Day 8, birds were weighed by cage, along with the remaining feed, and fecal matter. Feces collected from each cage were thoroughly mixed and prepared for fecal floatation, and each sample was examined to determine the number of oocysts per gram of fecal matter. All birds were scored for coccidian lesions on day 8 using the method of Johnson and Reid (1970). During the trial death weights were recorded and clinical observations were recorded twice each day throughout the study.

Feed in-take, body weight gain, feed conversion, opgs, coccidian lesion scores, and mortality were measured for each group and analyzed by standard statistical methods. The effect of sdAB (or peptide) supplementation was compared between groups treated with Eimeria and not treated with Eimeria, between treatment groups treated with Eimeria and antibody (or peptide) and control groups treated and not treated with Eimeria, control groups treated with Eimeria and no antibody (or peptide) or Coccidiostat, and control groups treated with Eimeria and also treated with a Coccidiostat.

Additionally the minimum effective dose was determined by seeing which antibody (or peptide) dose reduced fecal oocyst counts or lesion scores relative to the control birds that were infected but not treated with antibody (or peptide) or Coccidiostat. Using this design the extent of oocyst and lesion scoring reduction were determined as a function of dose.

### REFERENCES

Arbabi Ghahroudi, M. et al., 1997. Selection and identification of single domain antibody fragments from camel heavy-chain antibodies. FEBS Letters, 414 (3), pp.521-526.
Cervantes, H., 2002. Incidence of pathological conditions in clinically normal broilers from different regions of the USA. 51^{st} Western Poultry Disease Conference, May 1-4, Casa Magna Marriott Resort, Puerto Vallart, Jalisco, Mexico, 220-223.
Cervantes, H., 2006. Incidence of subclinical diseases and pathological conditions in clinically normal broilers from 3 production complexes sorted by sex and age. 143^{rd} Annual Convention of the American Veterinary Medical Association and 50^{th} Annual Meeting of the American Association of Avian Pathologists, July 15-19, Hawaii Convention Center, Honolulu, Hawaii.
Cook, M. E., Sand, J. M., McGuirk, S. M., Rieman, J. E., and Raabis, S. M. (2015), U.S. patent application No. US2016/0280778 A1
Diaz-Valdes, N., Manterola, L., Belsue, V., Riezu-Boj, J. I., Larrea, E., Echeverria, I., LLopiz, D., Lopez-Sagaseta, J., Lerat, H., Pawlotsky, J.-M., Prieto, J., Lasarte, J. J., Borras-Cuesta, F., and Sarobe, P. (2011), Hepatology 53, 23-31.
Goldman, E. R. et al., 2006. Facile Generation of Heat Stable Antiviral and Antitoxin Single Domain Antibodies from a Semi-synthetic Llama Library., 78 (24), pp.8245-8255.
Josephson, K., Logsdon, N. J., and Walter, M. W. (2001), Immunity 14, 35-46.
Liu, J. L. et al., 2013. Selection and evaluation of single domain antibodies toward MS2 phage and coat protein. Molecular Immunology, 53 (1-2), pp.118-125.
Naiyer, M. M., Saha, S., Hemke, V., Roy, S., Singh, S., Musti, K. V., and Saha, B. (2013), Human Immunology 74, 28-31.
Ni, G., Chen, S., Yang, Y., Cummins, S. F., Zhan, J., Li, Z., Zhu, B., Mounsey, K., Walton, S., Wei, M. Q., Wang, Y., Zhou, Y., Wang, T., and Liu, X. (2016), PLOSOne, Apr. 21, 2016.
Reineke, U., Sabat, R., Volk, H.-D., and Schneider-Mergener, J. (1998), Protein Sci. 7, 951-960.
Rothwell, L., Young, J. R., Zoorob, R., Whittaker, C. A., Hesketh, P., Archer, A., Smith, A. L., and Kaiser, P. (2004), J. Immunol. 173, 2675-2682.
Sand, J. M, and Cook, M. E. (2014), U.S. Pat. No. 8,652,457 B2.
Wu, Z., Hu, T., Rothwell, L., Vervelde, L., Kaiser, P., Boulton, K., Nolan, M. J., Tomley, F. M., Blake, D. P., and Hume, D. A. (2016), Devel. Comp. Immunol. 63, 206-212.
Yoon, I. L., Jones, B. C., Logsdon, N. J., and Walter, M. R. (2005), Structure 13, 551-564.
Zdanov, A., Schalk-Hihi, C., and Wlodawer, A. (1996), Protein Sci. 5, 1955-1962.

The references cited throughout this application, are incorporated for all purposes apparent herein and in the references themselves as if each reference was fully set forth. For the sake of presentation, specific ones of these references are cited at particular locations herein. A citation of a reference at a particular location indicates a manner(s) in which the teachings of the reference are incorporated. However, a citation of a reference at a particular location does not limit the manner in which all of the teachings of the cited reference are incorporated for all purposes.

It is understood, therefore, that this invention is not limited to the particular embodiments disclosed, but is intended to cover all modifications which are within the spirit and scope of the invention as defined by the appended claims; the above description; and/or shown in the attached drawings.

1. An anti-IL-10 single domain antibody comprising an amino acid sequence with at least 90% identity to the sequence of SEQ ID NO: 135, wherein the anti-IL-10 single domain antibody binds a polypeptide comprising an amino acid sequence of SEQ ID NO: 80 with an EC₅₀ of 30 nM or less, as measured by ELISA.

2. The anti-IL-10 single domain antibody of claim 1, wherein the antibody is stable at a temperature in a range from 70° C. to 90° C.

3. The anti-IL-10 single domain antibody of claim 1, wherein the antibody is digestible in pepsin.

4. The anti-IL-10 single domain antibody of claim 1, wherein the anti-IL-10 single domain antibody is encoded by a synthetic polynucleotide comprising a sequence with at least 90% identity to the sequence of SEQ ID NO: 175 or 176.

5. An animal feed comprising the anti-IL-10 single domain antibody of claim 1.

6. The animal feed of claim 5, wherein the anti-IL-10 single domain antibody is active upon expression in the plant and exposure to a temperature in the range from 25° C. to 130° C.

7. The animal feed of claim 5 further comprising a feed supplement.

8. The animal feed of claim 7, wherein the feed supplement is plant material.

9. The animal feed of claim 8, wherein the plant material is a non-transgenic plant or a transgenic plant.

10. The animal feed of claim 8, wherein the plant material includes at least one component selected from the group consisting of: corn meal, corn pellets, wheat meal, wheat pellets, wheat grain, barley grain, barley pellets, soybean meal, soybean oilcake, sorghum grain and sorghum pellets.

11. The animal feed of claim 7, wherein the feed supplement includes one or more exogenous enzymes.

12. The animal feed of claim 11, wherein the one or more exogenous enzymes includes a hydrolytic enzyme selected from the group consisting of: xylanase, endoglucanase, cellulase, protease, phytase, amylase and mannanase.

13. The animal feed of claim 7, wherein the feed supplement includes at least one component selected from the group consisting of: soluble solids, fat and vermiculite, limestone, plain salt, DL-methionine, L-lysine, L-threonine, monensin, vitamin premix, dicalcium phosphate, selenium premix, choline chloride, sodium chloride, and mineral premix.

14. A method of treating or preventing a gastrointestinal infection in an animal comprising administering to the animal the anti-IL-10 single domain antibody of claim 1 or an animal feed of claim comprising the anti-IL-10 single domain antibody.

15. The method of claim 14, wherein the step of administering is performed by feeding or injecting.

16. The method of claim 14, wherein the gastrointestinal infection is caused by a gastrointestinal pathogen selected from the group consisting of: a bacteria, yeast, fungi, archae, virus, and protozoa.

17. The method of claim 16, wherein the gastrointestinal pathogen belongs to the genus Eimeria.

18. The method of claim 16, wherein the gastrointestinal pathogen is selected from the group consisting of: Eimeria tenella, Eimeria acervulina, and Eimeria maxima.

19. The method of claim 14, wherein treating stimulates the immune system and enhances growth of the animal.

20. The method of claim 18, wherein the animal is selected from the group consisting of: a chicken, a turkey, or a duck.


SEQUENCE LISTING (249 sequences)

SEQ ID NO: 1 - protein, 40 aa, synthetic construct
  REGION 1..40: note = Synthetic construct, P21
PARLRELRVKFEEIKDYFQSRDDELNIQLLSSELLDEFKG

SEQ ID NO: 2 - protein, 27 aa, synthetic construct
  REGION 1..27: note = Synthetic construct, P22
ENGIYKAMGEFDIFINYIEEYLLMRRR

SEQ ID NO: 3 - protein, 47 aa, synthetic construct
  REGION 1..47: note = Synthetic construct, P23
PARLRELRVKFEEIKDYFQGGGSGGGSQQSMGDLGNMLLGLKATMRR

SEQ ID NO: 4 - protein, 50 aa, synthetic construct
  REGION 1..50: note = Synthetic construct, P24
GCQSVSEMLRFYTDEVLPRAMQGGGSGGGSKAMGEFDIFINYIEEYLLMR

SEQ ID NO: 5 - protein, 8 aa, synthetic construct
  REGION 1..8: note = Synthetic construct, P25
PARLRELR

SEQ ID NO: 6 - protein, 11 aa, synthetic construct
  REGION 1..11: note = Synthetic construct, P26
LSSELLDEFKG

SEQ ID NO: 7 - protein, 10 aa, synthetic construct
  REGION 1..10: note = Synthetic construct, P27
GEFDIFNYIE

SEQ ID NO: 8 - protein, 10 aa, synthetic construct
  REGION 1..10: note = Synthetic construct, P28
SLRYYARVRA

SEQ ID NO: 9 - protein, 7 aa, synthetic construct
  REGION 1..7: note = Synthetic construct, P29
TNAFSPQ

SEQ ID NO: 10 - protein, 29 aa, synthetic construct
  REGION 1..29: note = Synthetic construct, P11
YDDIQKHARRYRVYIRRARDNQTYEVWET

SEQ ID NO: 11 - protein, 8 aa, synthetic construct
  REGION 1..8: note = Synthetic construct, P30
IQKHARRY

SEQ ID NO: 12 - protein, 8 aa, synthetic construct
  REGION 1..8: note = Synthetic construct, P31
NQTYEVWE

SEQ ID NO: 13 - protein, 9 aa, synthetic construct
  REGION 1..9: note = Synthetic construct, P32
VASRHIPAM

SEQ ID NO: 14 - protein, 16 aa, synthetic construct
  REGION 1..16: note = Synthetic construct, P9
FFKKFFKKFFKKFFKK

SEQ ID NO: 15 - protein, 16 aa, synthetic construct
  REGION 1..16: note = Synthetic construct, P6
GTELPSPPSVWFEAEF

SEQ ID NO: 16 - DNA, 120 bp, synthetic construct
  misc_feature 1..120: note = Synthetic construct, P21 coding sequence
  source 1..120: mol_type = other DNA
ccggccaggctgagggagctgagggtgaagttcgaggagatcaaggactacttccagagcagggacgacgagctgaacatccagctgctgagcagcgagctgctggacgagttcaagggc

SEQ ID NO: 17 - DNA, 81 bp, synthetic construct
  misc_feature 1..81: note = Synthetic construct, P22 coding sequence
  source 1..81: mol_type = other DNA
gagaacggcatctacaaggccatgggcgagttcgacatcttcatcaactacatcgaggagtacctgctgatgaggaggagg

SEQ ID NO: 18 - DNA, 141 bp, synthetic construct
  misc_feature 1..141: note = Synthetic construct, P23 coding sequence
  source 1..141: mol_type = other DNA
ccggccaggctgagggagctgagggtgaagttcgaggagatcaaggactacttccagggcggcggcagcggcggcggcagccagcagagcatgggcgacctgggcaacatgctgctgggcctgaaggccaccatgaggagg

SEQ ID NO: 19 - DNA, 150 bp, synthetic construct
  misc_feature 1..150: note = Synthetic construct, P24 coding sequence
  source 1..150: mol_type = other DNA
ggctgccagagcgtgagcgagatgctgaggttctacaccgacgaggtgctgccgagggccatgcagggcggcggcagcggcggcggcagcaaggccatgggcgagttcgacatcttcatcaactacatcgaggagtacctgctgatgagg

SEQ ID NO: 20 - DNA, 24 bp, synthetic construct
  misc_feature 1..24: note = Synthetic construct, P25 coding sequence
  source 1..24: mol_type = other DNA
ccggccaggctgagggagctgagg

SEQ ID NO: 21 - DNA, 33 bp, synthetic construct
  misc_feature 1..33: note = Synthetic construct, P26 coding sequence
  source 1..33: mol_type = other DNA
ctgagcagcgagctgctggacgagttcaagggc

SEQ ID NO: 22 - DNA, 30 bp, synthetic construct
  misc_feature 1..30: note = Synthetic construct, P27 coding sequence
  source 1..30: mol_type = other DNA
ggcgagttcgacatcttcaactacatcgag

SEQ ID NO: 23 - DNA, 30 bp, synthetic construct
  misc_feature 1..30: note = Synthetic construct, P28 coding sequence
  source 1..30: mol_type = other DNA
agcctgaggtactacgccagggtgagggcc

SEQ ID NO: 24 - DNA, 21 bp, synthetic construct
  misc_feature 1..21: note = Synthetic construct, P29 coding sequence
  source 1..21: mol_type = other DNA
accaacgccttcagcccgcag

SEQ ID NO: 25 - DNA, 87 bp, synthetic construct
  misc_feature 1..87: note = Synthetic construct, P11 coding sequence
  source 1..87: mol_type = other DNA
tacgacgacatccagaagcacgccaggaggtacagggtgtacatcaggagggccagggacaaccagacctacgaggtgtgggagacc

SEQ ID NO: 26 - DNA, 24 bp, synthetic construct
  misc_feature 1..24: note = Synthetic construct, P30 coding sequence
  source 1..24: mol_type = other DNA
atccagaagcacgccaggaggtac

SEQ ID NO: 27 - DNA, 24 bp, synthetic construct
  misc_feature 1..24: note = Synthetic construct, P31 coding sequence
  source 1..24: mol_type = other DNA
aaccagacctacgaggtgtgggag

SEQ ID NO: 28 - DNA, 27 bp, synthetic construct
  misc_feature 1..27: note = Synthetic construct, P32 coding sequence
  source 1..27: mol_type = other DNA
gtggccagcaggcacatcccggccatg

SEQ ID NO: 29 - protein, 4 aa, synthetic construct
  REGION 1..4: note = Synthetic construct, KDEL
KDEL

SEQ ID NO: 30 - protein, 4 aa, synthetic construct
  REGION 1..4: note = Synthetic construct, HDEL
HDEL

SEQ ID NO: 31 - protein, 6 aa, synthetic construct
  REGION 1..6: note = Synthetic construct, SEKDEL
SEKDEL

SEQ ID NO: 32 - protein, 12 aa, synthetic construct
  REGION 1..12: note = Synthetic construct, P2501
PARLRELRKDEL

SEQ ID NO: 33 - protein, 16 aa, synthetic construct
  REGION 1..16: note = Synthetic construct, P2502
PARLRELRPARLRELR

SEQ ID NO: 34 - protein, 20 aa, synthetic construct
  REGION 1..20: note = Synthetic construct, P2503
PARLRELRPARLRELRKDEL

SEQ ID NO: 35 - protein, 20 aa, synthetic construct
  REGION 1..20: note = Synthetic construct, P2504
PARLRELRAGPAPARLRELR

SEQ ID NO: 36 - protein, 24 aa, synthetic construct
  REGION 1..24: note = Synthetic construct, P2505
PARLRELRAGPAPARLRELRKDEL

SEQ ID NO: 37 - protein, 24 aa, synthetic construct
  REGION 1..24: note = Synthetic construct, P2506
PARLRELRPARLRELRPARLRELR

SEQ ID NO: 38 - protein, 28 aa, synthetic construct
  REGION 1..28: note = Syntheict construct, P2507
PARLRELRPARLRELRPARLRELRKDEL

SEQ ID NO: 39 - protein, 32 aa, synthetic construct
  REGION 1..32: note = Synthetic construct, P2508
PARLRELRAGPAPARLRELRAGPAPARLRELR

SEQ ID NO: 40 - protein, 36 aa, synthetic construct
  REGION 1..36: note = Synthetic construct, P2509
PARLRELRAGPAPARLRELRAGPAPARLRELRKDEL

SEQ ID NO: 41 - protein, 6 aa, synthetic construct
  REGION 1..6: note = Synthetic construct, GSGGSG linker
GSGGSG

SEQ ID NO: 42 - protein, 5 aa, synthetic construct
  REGION 1..5: note = Synthetic construct, zeolin linker
GGGGS

SEQ ID NO: 43 - protein, 5 aa, synthetic construct
  REGION 1..5: note = Synthetic construct, Zera fusion
GGGGG

SEQ ID NO: 44 - protein, 5 aa, synthetic construct
  REGION 1..5: note = Synthetic construct, VPGVG
VPGVG

SEQ ID NO: 45 - DNA, 18 bp, synthetic construct
  misc_feature 1..18: note = Synthetic construct, GSSGGSG coding seq
  source 1..18: mol_type = other DNA
ggcagcggcggcagcggc

SEQ ID NO: 46 - DNA, 15 bp, synthetic construct
  misc_feature 1..15: note = Synthetic construct, zeolin linker coding seq
  source 1..15: mol_type = other DNA
ggcggcggcggcagc

SEQ ID NO: 47 - DNA, 15 bp, synthetic construct
  misc_feature 1..15: note = Synthetic construct, Zera linker coding seq
  source 1..15: mol_type = other DNA
ggcggcggcggcggc

SEQ ID NO: 48 - DNA, 72 bp, synthetic construct
  misc_feature 1..72: note = Synthetic construct, BAAS coding seq
  source 1..72: mol_type = other DNA
atggcgaacaaacatttgtccctctccctcttcctcgtcctccttggcctgtcggccagcttggcctccggg

SEQ ID NO: 49 - DNA, 72 bp, synthetic construct
  misc_feature 1..72: note = Synthetic construct, OsGluB4sp coding seq
  source 1..72: mol_type = other DNA
atggccaccatcgctttctcccgcttgtccatctacttctgcgtgcttctcctgtgccacggctccatggcc

SEQ ID NO: 50 - DNA, 93 bp, synthetic construct
  misc_feature 1..93: note = Synthetic construct, PR1 coding seq
  source 1..93: mol_type = other DNA
atgggcttcgtgctcttctcccagctgccttccttccttcttgtctccaccctgctcttgttcctcgtgatctcccactcctgccgcgcccag

SEQ ID NO: 51 - DNA, 12 bp, synthetic construct
  misc_feature 1..12: note = Synthetic construct, KDEL coding seq
  source 1..12: mol_type = other DNA
aaggacgagctg

SEQ ID NO: 52 - DNA, 12 bp, synthetic construct
  misc_feature 1..12: note = Synthetic construct, HDEL coding seq
  source 1..12: mol_type = other DNA
cacgacgagctg

SEQ ID NO: 53 - DNA, 18 bp, synthetic construct
  misc_feature 1..18: note = Synthetic construct, SEKDEL coding seq
  source 1..18: mol_type = other DNA
tccgagaaggacgagctg

SEQ ID NO: 54 - DNA, 129 bp, synthetic construct
  misc_feature 1..129: note = Synthetic construct, HvAle coding seq
  source 1..129: mol_type = other DNA
atggcccacgcccgcgtcctcctcctggcgctcgccgtcctggccaccgccgccgtcgccgtcgcctcctcctcctccttcgccgactccaacccgatccgcccggtgaccgaccgcgccgcctccacc

SEQ ID NO: 55 - DNA, 24 bp, synthetic construct
  misc_feature 1..24: note = Synthetic construct, HvVSD coding seq
  source 1..24: mol_type = other DNA
gacgagctgaaggccgaggccaag

SEQ ID NO: 56 - DNA, 108 bp, synthetic construct
  misc_feature 1..108: note = Synthetic construct, P2509 coding seq
  source 1..108: mol_type = other DNA
ccggccaggctgagggagctgagggccggcccggccccggccaggctgagggagctgagggccggcccggccccggccaggctgagggagctgaggaaggacgagctg

SEQ ID NO: 57 - protein, 223 aa, synthetic construct
  REGION 1..223: note = Synthetic construct, ZmZ27
MRVLLVALALLALAASATSTHTSGGCGCQPPPPVHLPPPVHLPPPVHLPPPVHLPPPVHLPPPVHLPPPVHVPPPVHLPPPPCHYPTQPPRPQPHPQPHPCPCQQPHPSPCQLQGTCGVGSTPILGQCVEFLRHQCSPTATPYCSPQCQSLRQQCCQQLRQVEPQHRYQAIFGLVLQSILQQQPQSGQVAGLLAAQIAQQLTAMCGLQQPTPCPYAAAGGVPH

SEQ ID NO: 58 - DNA, 672 bp, synthetic construct
  misc_feature 1..672: note = Synthetic construct, ZmZ27 coding seq
  source 1..672: mol_type = other DNA
atgagggtgttgctcgttgccctcgctctcctggctctcgctgcgagcgccacctccacgcatacaagcggcggctgcggctgccagccaccgccgccggttcatctaccgccgccggtgcatctgccacctccggttcacctgccacctccggtgcatctcccaccgccggtccacctgccgccgccggtccacctgccaccgccggtccatgtgccgccgccggttcatctgccgccgccaccatgccactaccctactcaaccgccccggcctcagcctcatccccagccacacccatgcccgtgccaacagccgcatccaagcccgtgccagctgcagggaacctgcggcgttggcagcaccccgatcctgggccagtgcgtcgagttcctgaggcatcagtgcagcccgacggcgacgccctactgctcgcctcagtgccagtcgttgcggcagcagtgttgccagcagctcaggcaggtggagccgcagcaccggtaccaggcgatcttcggcttggtcctccagtccatcctgcagcagcagccgcaaagcggccaggtcgcggggctgttggcggcgcagatagcgcagcaactgacggcgatgtgcggcctgcagcagccgactccatgcccctacgctgctgccggcggtgtcccccactga

SEQ ID NO: 59 - protein, 89 aa, synthetic construct
  REGION 1..89: note = Synthetic construct, ZmZ27_AA24-112
GGCGCQPPPPVHLPPPVHLPPPVHLPPPVHLPPPVHLPPPVHLPPPVHVPPPVHLPPPPCHYPTQPPRPQPHPQPHPCPCQQPHPSPCQ

SEQ ID NO: 60 - DNA, 267 bp, synthetic construct
  misc_feature 1..267: note = Synthetic construct, ZmZ27_AA24-112 coding seq
  source 1..267: mol_type = other DNA
ggcggctgcggctgccagccaccgccgccggttcatctaccgccgccggtgcatctgccacctccggttcacctgccacctccggtgcatctcccaccgccggtccacctgccgccgccggtccacctgccaccgccggtccatgtgccgccgccggttcatctgccgccgccaccatgccactaccctactcaaccgccccggcctcagcctcatccccagccacacccatgcccgtgccaacagccgcatccaagcccgtgccag

SEQ ID NO: 61 - protein, 112 aa, synthetic construct
  REGION 1..112: note = Synthetic construct, Zera_AA1-112
MRVLLVALALLALAASATSTHTSGGCGCQPPPPVHLPPPVHLPPPVHLPPPVHLPPPVHLPPPVHLPPPVHVPPPVHLPPPPCHYPTQPPRPQPHPQPHPCPCQQPHPSPCQ

SEQ ID NO: 62 - DNA, 336 bp, synthetic construct
  misc_feature 1..336: note = Synthetic construct, Zera coding seq
  source 1..336: mol_type = other DNA
atgagggtgttgctcgttgccctcgctctcctggctctcgctgcgagcgccacctccacgcatacaagcggcggctgcggctgccagccaccgccgccggttcatctaccgccgccggtgcatctgccacctccggttcacctgccacctccggtgcatctcccaccgccggtccacctgccgccgccggtccacctgccaccgccggtccatgtgccgccgccggttcatctgccgccgccaccatgccactaccctactcaaccgccccggcctcagcctcatccccagccacacccatgcccgtgccaacagccgcatccaagcccgtgccag

SEQ ID NO: 63 - DNA, 450 bp, synthetic construct
  misc_feature 1..450: note = Synthetic construct, ZeraP2508 coding seq
  source 1..450: mol_type = other DNA
atgagggtgttgctcgttgccctcgctctcctggctctcgctgcgagcgccacctccacgcatacaagcggcggctgcggctgccagccaccgccgccggttcatctaccgccgccggtgcatctgccacctccggttcacctgccacctccggtgcatctcccaccgccggtccacctgccgccgccggtccacctgccaccgccggtccatgtgccgccgccggttcatctgccgccgccaccatgccactaccctactcaaccgccccggcctcagcctcatccccagccacacccatgcccgtgccaacagccgcatccaagcccgtgccagggcagcggcggcagcggcccggccaggctgagggagctgagggccggcccggccccggccaggctgagggagctgagggccggcccggccccggccaggctgagggagctgagg

SEQ ID NO: 64 - DNA, 462 bp, synthetic construct
  misc_feature 1..462: note = Synthetic construct, ZeraP2509KDEL coding seq
  source 1..462: mol_type = other DNA
atgagggtgttgctcgttgccctcgctctcctggctctcgctgcgagcgccacctccacgcatacaagcggcggctgcggctgccagccaccgccgccggttcatctaccgccgccggtgcatctgccacctccggttcacctgccacctccggtgcatctcccaccgccggtccacctgccgccgccggtccacctgccaccgccggtccatgtgccgccgccggttcatctgccgccgccaccatgccactaccctactcaaccgccccggcctcagcctcatccccagccacacccatgcccgtgccaacagccgcatccaagcccgtgccagggcagcggcggcagcggcccggccaggctgagggagctgagggccggcccggccccggccaggctgagggagctgagggccggcccggccccggccaggctgagggagctgaggaaggacgagctg

SEQ ID NO: 65 - protein, 140 aa, synthetic construct
  REGION 1..140: note = Synthetic construct, 28xVPGVG
VPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVG

SEQ ID NO: 66 - DNA, 420 bp, synthetic construct
  misc_feature 1..420: note = Synthetic construct, 28xVPGVG coding seq
  source 1..420: mol_type = other DNA
gtgccgggcgtgggcgtgccgggcgtgggcgtgccgggcgtgggcgtgccgggcgtgggcgtgccgggcgtgggcgtgccgggcgtgggcgtgccgggcgtgggcgtgccgggcgtgggcgtgccgggcgtgggcgtgccgggcgtgggcgtgccgggcgtgggcgtgccgggcgtgggcgtgccgggcgtgggcgtgccgggcgtgggcgtgccgggcgtgggcgtgccgggcgtgggcgtgccgggcgtgggcgtgccgggcgtgggcgtgccgggcgtgggcgtgccgggcgtgggcgtgccgggcgtgggcgtgccgggcgtgggcgtgccgggcgtgggcgtgccgggcgtgggcgtgccgggcgtgggcgtgccgggcgtgggcgtgccgggcgtgggcgtgccgggcgtgggc

SEQ ID NO: 67 - protein, 75 aa, synthetic construct
  REGION 1..75: note = Synthetic construct, HFBI
SNGNGNVCPPGLFSNPQCCATQVLGLIGLDCKVPSQNVYDGTDFRNVCAKTGAQPLCCVAPVAGQALLCQTAVGA

SEQ ID NO: 68 - DNA, 225 bp, synthetic construct
  misc_feature 1..225: note = Synthetic construct, HFBI coding seq
  source 1..225: mol_type = other DNA
agcaacggcaacggcaacgtgtgcccgccgggcctgttcagcaacccgcagtgctgcgccacccaggtgctgggcctgatcggcctggactgcaaggtgccgagccagaacgtgtacgacggcaccgacttcaggaacgtgtgcgccaagaccggcgcccagccgctgtgctgcgtggccccggtggccggccaggccctgctgtgccagaccgccgtgggcgcc

SEQ ID NO: 69 - DNA, 11826 bp, synthetic construct
  misc_feature 1..11826: note = Synthetic construct, pAG4305
  source 1..11826: mol_type = other DNA
ctaggtccccgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattggaattcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcacggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagatccagatctaaaccatgcagaaactcattaactcagtgcaaaactatgcctggggcagcaaaacggcgttgactgaactttatggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggatgattttgccttctcgctgcatgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaagagcttactgaaaaaattaacatctcttgctaagctgggagctctagatccccgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattggcgagctcgaattaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaaatatcatctccctcgcagagatccgaattatcagccttcttattcatttctcgcttaaccgtgacaggctgtcgatcttgagaactatgccgacataataggaaatcgctggataaagccgctgaggaagctgagtggcgctatttctttagaagtgaacgttgacgatcgtcgaccgtaccccgatgaattaattcggacgtacgttctgaacacagctggatacttacttgggcgattgtcatacatgacatcaacaatgtacccgtttgtgtaaccgtctcttggaggttcgtatgacactagtggttcccctcagcttgcgactagatgttgaggcctaacattttattagagagcaggctagttgcttagatacatgatcttcaggccgttatctgtcagggcaagcgaaaattggccatttatgacgaccaatgccccgcagaagctcccatctttgccgccatagacgccgcgccccccttttggggtgtagaacatccttttgccagatgtggaaaagaagttcgttgtcccattgttggcaatgacgtagtagccggcgaaagtgcgagacccatttgcgctatatataagcctacgatttccgttgcgactattgtcgtaattggatgaactattatcgtagttgctctcagagttgtcgtaatttgatggactattgtcgtaattgcttatggagttgtcgtagttgcttggagaaatgtcgtagttggatggggagtagtcatagggaagacgagcttcatccactaaaacaattggcaggtcagcaagtgcctgccccgatgccatcgcaagtacgaggcttagaaccaccttcaacagatcgcgcatagtcttccccagctctctaacgcttgagttaagccgcgccgcgaagcggcgtcggcttgaacgaattgttagacattatttgccgactaccttggtgatctcgcctttcacgtagtgaacaaattcttccaactgatctgcgcgcgaggccaagcgatcttcttgtccaagataagcctgcctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatgatgtttaactcctgaattaagccgcgccgcgaagcggtgtcggcttgaatgaattgttaggcgtcatcctgtgctcccgagaaccagtaccagtacatcgctgtttcgttcgagacttgaggtctagttttatacgtgaacaggtcaatgccgccgagagtaaagccacattttgcgtacaaattgcaggcaggtacattgttcgtttgtgtctctaatcgtatgccaaggagctgtctgcttagtgcccactttttcgcaaattcgatgagactgtgcgcgactcctttgcctcggtgcgtgtgcgacacaacaatgtgttcgatagaggctagatcgttccatgttgagttgagttcaatcttcccgacaagctcttggtcgatgaatgcgccatagcaagcagagtcttcatcagagtcatcatccgagatgtaatccttccggtaggggctcacacttctggtagatagttcaaagccttggtcggataggtgcacatcgaacacttcacgaacaatgaaatggttctcagcatccaatgtttccgccacctgctcagggatcaccgaaatcttcatatgacgcctaacgcctggcacagcggatcgcaaacctggcgcggcttttggcacaaaaggcgtgacaggtttgcgaatccgttgctgccacttgttaacccttttgccagatttggtaactataatttatgttagaggcgaagtcttgggtaaaaactggcctaaaattgctggggatttcaggaaagtaaacatcaccttccggctcgatgtctattgtagatatatgtagtgtatctacttgatcgggggatctgctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggggggggggggggggggttccattgttcattccacggacaaaaacagagaaaggaaacgacagaggccaaaaagctcgctttcagcacctgtcgtttcctttcttttcagagggtattttaaataaaaacattaagttatgacgaagaagaacggaaacgccttaaaccggaaaattttcataaatagcgaaaacccgcgaggtcgccgccccgtaacctgtcggatcaccggaaaggacccgtaaagtgataatgattatcatctacatatcacaacgtgcgtggaggccatcaaaccacgtcaaataatcaattatgacgcaggtatcgtattaattgatctgcatcaacttaacgtaaaaacaacttcagacaatacaaatcagcgacactgaatacggggcaacctcatgtccccccccccccccccctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattggtcgacgatcttgctgcgttcggatattttcgtggagttcccgccacagacccggattgaaggcgagatccagcaactcgcgccagatcatcctgtgacggaactttggcgcgtgatgactggccaggacgtcggccgaaagagcgacaagcagatcacgcttttcgacagcgtcggatttgcgatcgaggatttttcggcgctgcgctacgtccgcgaccgcgttgagggatcaagccacagcagcccactcgaccttctagccgacccagacgagccaagggatctttttggaatgctgctccgtcgtcaggctttccgacgtttgggtggttgaacagaagtcattatcgcacggaatgccaagcactcccgaggggaaccctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaacctgatcatgagcggagaattaagggagtcacgttatgacccccgccgatgacgcgggacaagccgttttacgtttggaactgacagaaccgcaacgttgaaggagccactcagcttaattaagtctaactcgagttactggtacgtatacagggttccttgcgtgaagaagggtggcctgcggttcaccattaacggtcacgactacttccagctagtactggtgaccaacgtcgcggcggcagggtcaatcaagtccatggaggttatgggttccaacacagcggattggatgccgatggcacgtaactggggcgcccaatggcactcactggcctacctcaccggtcaaggtctatcctttagggtcaccaacacagatgaccaaacgctcgtcttcaccaacgtcgtgccaccaggatggaagtttggccagacatttgcaagcaagctgcagttcaagtgagaggagaagcctgaattgataccggagcgtttcttttgggagtaacatctctggttgcctagcaaacatatgattgtatataagtttcgttgtgcgtttattctttcggtgtgtaaaataacatacatgctttcctgatattttcttgtatatatgtacacacacacgacaaatccttccatttctattattattgaacaatttaattgcgagggcgagtacttgtctgtttaccttttttttttcagatggcattttatagtttaacctttcatggaccggcagtagttctaaccatgaatgaaaagaaatcatagtccacaccacgcagggacattgtggtcattttagacaagacgatttgattaatgtcttgtatgatatggtcgacagtgaggactaacaaacatatggcatattttattaccggcgagttaaataaatttatgtcacagtaataaactgcctaataaatgcacgccagaaaatataatgataaaaaaaagaaaagatacataagtccattgcttctacttttttaaaaattaaatccaacattttctattttttggtataaacttggaagtactagttggatatgcaaaatcatctaacctccatatatttcatcaatttgtttactttacatatgggagaggatagtatgtcaaagaaaatgacaacaagcttacaagtttcttattttaaaagttccgctaacttatcaagcatagtgtgccacgcaaaactgacaacaaaccaacaaatttaaggagcgcctaacttatcatctatgacataccgcacaaaatgataacatactagagaaactttattgcacaaaaggaaatttatccataaggcaaaggaacatcttaaggctttggatatacatttaccaacaagcattgtttgtattacccctaaagcgcaagacatgtcatccatgagtcatagtgtgtatatctcaacattgcaaagctaccttttttctattatacttttcgcattataggctagatattatctatacatgtcaacaaactctatccctacgtcatatctgaagattcttttcttcactatataagttggcttccctgtcattgaactcacatcaaccagcccaagtttccaataacatcctcaaatagctggatcctaaaccatgagggtgttgctcgttgccctcgctctcctggctctcgctgcgagcgccacctccggctgccagagcgtgagcgagatgctgaggttctacaccgacgaggtgctgccgagggccatgcagggcggcggcagcggcggcggcagcaaggccatgggcgagttcgacatcttcatcaactacatcgaggagtacctgctgatgaggaaggacgagctgtaac

SEQ ID NO: 70 - DNA, 11772 bp, synthetic construct
  misc_feature 1..11772: note = Synthetic construct, pAG4306
  source 1..11772: mol_type = other DNA
ctaggtccccgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattggaattcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcacggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagatccagatctaaaccatgcagaaactcattaactcagtgcaaaactatgcctggggcagcaaaacggcgttgactgaactttatggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggatgattttgccttctcgctgcatgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaagagcttactgaaaaaattaacatctcttgctaagctgggagctctagatccccgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattggcgagctcgaattaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaaatatcatctccctcgcagagatccgaattatcagccttcttattcatttctcgcttaaccgtgacaggctgtcgatcttgagaactatgccgacataataggaaatcgctggataaagccgctgaggaagctgagtggcgctatttctttagaagtgaacgttgacgatcgtcgaccgtaccccgatgaattaattcggacgtacgttctgaacacagctggatacttacttgggcgattgtcatacatgacatcaacaatgtacccgtttgtgtaaccgtctcttggaggttcgtatgacactagtggttcccctcagcttgcgactagatgttgaggcctaacattttattagagagcaggctagttgcttagatacatgatcttcaggccgttatctgtcagggcaagcgaaaattggccatttatgacgaccaatgccccgcagaagctcccatctttgccgccatagacgccgcgccccccttttggggtgtagaacatccttttgccagatgtggaaaagaagttcgttgtcccattgttggcaatgacgtagtagccggcgaaagtgcgagacccatttgcgctatatataagcctacgatttccgttgcgactattgtcgtaattggatgaactattatcgtagttgctctcagagttgtcgtaatttgatggactattgtcgtaattgcttatggagttgtcgtagttgcttggagaaatgtcgtagttggatggggagtagtcatagggaagacgagcttcatccactaaaacaattggcaggtcagcaagtgcctgccccgatgccatcgcaagtacgaggcttagaaccaccttcaacagatcgcgcatagtcttccccagctctctaacgcttgagttaagccgcgccgcgaagcggcgtcggcttgaacgaattgttagacattatttgccgactaccttggtgatctcgcctttcacgtagtgaacaaattcttccaactgatctgcgcgcgaggccaagcgatcttcttgtccaagataagcctgcctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatgatgtttaactcctgaattaagccgcgccgcgaagcggtgtcggcttgaatgaattgttaggcgtcatcctgtgctcccgagaaccagtaccagtacatcgctgtttcgttcgagacttgaggtctagttttatacgtgaacaggtcaatgccgccgagagtaaagccacattttgcgtacaaattgcaggcaggtacattgttcgtttgtgtctctaatcgtatgccaaggagctgtctgcttagtgcccactttttcgcaaattcgatgagactgtgcgcgactcctttgcctcggtgcgtgtgcgacacaacaatgtgttcgatagaggctagatcgttccatgttgagttgagttcaatcttcccgacaagctcttggtcgatgaatgcgccatagcaagcagagtcttcatcagagtcatcatccgagatgtaatccttccggtaggggctcacacttctggtagatagttcaaagccttggtcggataggtgcacatcgaacacttcacgaacaatgaaatggttctcagcatccaatgtttccgccacctgctcagggatcaccgaaatcttcatatgacgcctaacgcctggcacagcggatcgcaaacctggcgcggcttttggcacaaaaggcgtgacaggtttgcgaatccgttgctgccacttgttaacccttttgccagatttggtaactataatttatgttagaggcgaagtcttgggtaaaaactggcctaaaattgctggggatttcaggaaagtaaacatcaccttccggctcgatgtctattgtagatatatgtagtgtatctacttgatcgggggatctgctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggggggggggggggggggttccattgttcattccacggacaaaaacagagaaaggaaacgacagaggccaaaaagctcgctttcagcacctgtcgtttcctttcttttcagagggtattttaaataaaaacattaagttatgacgaagaagaacggaaacgccttaaaccggaaaattttcataaatagcgaaaacccgcgaggtcgccgccccgtaacctgtcggatcaccggaaaggacccgtaaagtgataatgattatcatctacatatcacaacgtgcgtggaggccatcaaaccacgtcaaataatcaattatgacgcaggtatcgtattaattgatctgcatcaacttaacgtaaaaacaacttcagacaatacaaatcagcgacactgaatacggggcaacctcatgtccccccccccccccccctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattggtcgacgatcttgctgcgttcggatattttcgtggagttcccgccacagacccggattgaaggcgagatccagcaactcgcgccagatcatcctgtgacggaactttggcgcgtgatgactggccaggacgtcggccgaaagagcgacaagcagatcacgcttttcgacagcgtcggatttgcgatcgaggatttttcggcgctgcgctacgtccgcgaccgcgttgagggatcaagccacagcagcccactcgaccttctagccgacccagacgagccaagggatctttttggaatgctgctccgtcgtcaggctttccgacgtttgggtggttgaacagaagtcattatcgcacggaatgccaagcactcccgaggggaaccctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaacctgatcatgagcggagaattaagggagtcacgttatgacccccgccgatgacgcgggacaagccgttttacgtttggaactgacagaaccgcaacgttgaaggagccactcagcttaattaagtctaactcgagttactggtacgtatacagggttccttgcgtgaagaagggtggcctgcggttcaccattaacggtcacgactacttccagctagtactggtgaccaacgtcgcggcggcagggtcaatcaagtccatggaggttatgggttccaacacagcggattggatgccgatggcacgtaactggggcgcccaatggcactcactggcctacctcaccggtcaaggtctatcctttagggtcaccaacacagatgaccaaacgctcgtcttcaccaacgtcgtgccaccaggatggaagtttggccagacatttgcaagcaagctgcagttcaagtgagaggagaagcctgaattgataccggagcgtttcttttgggagtaacatctctggttgcctagcaaacatatgattgtatataagtttcgttgtgcgtttattctttcggtgtgtaaaataacatacatgctttcctgatattttcttgtatatatgtacacacacacgacaaatccttccatttctattattattgaacaatttaattgcgagggcgagtacttgtctgtttaccttttttttttcagatggcattttatagtttaacctttcatggaccggcagtagttctaaccatgaatgaaaagaaatcatagtccacaccacgcagggacattgtggtcattttagacaagacgatttgattaatgtcttgtatgatatggtcgacagtgaggactaacaaacatatggcatattttattaccggcgagttaaataaatttatgtcacagtaataaactgcctaataaatgcacgccagaaaatataatgataaaaaaaagaaaagatacataagtccattgcttctacttttttaaaaattaaatccaacattttctattttttggtataaacttggaagtactagttggatatgcaaaatcatctaacctccatatatttcatcaatttgtttactttacatatgggagaggatagtatgtcaaagaaaatgacaacaagcttacaagtttcttattttaaaagttccgctaacttatcaagcatagtgtgccacgcaaaactgacaacaaaccaacaaatttaaggagcgcctaacttatcatctatgacataccgcacaaaatgataacatactagagaaactttattgcacaaaaggaaatttatccataaggcaaaggaacatcttaaggctttggatatacatttaccaacaagcattgtttgtattacccctaaagcgcaagacatgtcatccatgagtcatagtgtgtatatctcaacattgcaaagctaccttttttctattatacttttcgcattataggctagatattatctatacatgtcaacaaactctatccctacgtcatatctgaagattcttttcttcactatataagttggcttccctgtcattgaactcacatcaaccagcccaagtttccaataacatcctcaaatagctggatcctaaaccatgagggtgttgctcgttgccctcgctctcctggctctcgctgcgagcgccacctccccggccaggctgagggagctgagggccggcccggccccggccaggctgagggagctgagggccggcccggccccggccaggctgagggagctgaggaaggacgagctgtaac

SEQ ID NO: 71 - DNA, 12069 bp, synthetic construct
  misc_feature 1..12069: note = Synthetic construct, pAG4308
  source 1..12069: mol_type = other DNA
ctaggtccccgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattggaattcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcacggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagatccagatctaaaccatgcagaaactcattaactcagtgcaaaactatgcctggggcagcaaaacggcgttgactgaactttatggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggatgattttgccttctcgctgcatgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaagagcttactgaaaaaattaacatctcttgctaagctgggagctctagatccccgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattggcgagctcgaattaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaaatatcatctccctcgcagagatccgaattatcagccttcttattcatttctcgcttaaccgtgacaggctgtcgatcttgagaactatgccgacataataggaaatcgctggataaagccgctgaggaagctgagtggcgctatttctttagaagtgaacgttgacgatcgtcgaccgtaccccgatgaattaattcggacgtacgttctgaacacagctggatacttacttgggcgattgtcatacatgacatcaacaatgtacccgtttgtgtaaccgtctcttggaggttcgtatgacactagtggttcccctcagcttgcgactagatgttgaggcctaacattttattagagagcaggctagttgcttagatacatgatcttcaggccgttatctgtcagggcaagcgaaaattggccatttatgacgaccaatgccccgcagaagctcccatctttgccgccatagacgccgcgccccccttttggggtgtagaacatccttttgccagatgtggaaaagaagttcgttgtcccattgttggcaatgacgtagtagccggcgaaagtgcgagacccatttgcgctatatataagcctacgatttccgttgcgactattgtcgtaattggatgaactattatcgtagttgctctcagagttgtcgtaatttgatggactattgtcgtaattgcttatggagttgtcgtagttgcttggagaaatgtcgtagttggatggggagtagtcatagggaagacgagcttcatccactaaaacaattggcaggtcagcaagtgcctgccccgatgccatcgcaagtacgaggcttagaaccaccttcaacagatcgcgcatagtcttccccagctctctaacgcttgagttaagccgcgccgcgaagcggcgtcggcttgaacgaattgttagacattatttgccgactaccttggtgatctcgcctttcacgtagtgaacaaattcttccaactgatctgcgcgcgaggccaagcgatcttcttgtccaagataagcctgcctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatgatgtttaactcctgaattaagccgcgccgcgaagcggtgtcggcttgaatgaattgttaggcgtcatcctgtgctcccgagaaccagtaccagtacatcgctgtttcgttcgagacttgaggtctagttttatacgtgaacaggtcaatgccgccgagagtaaagccacattttgcgtacaaattgcaggcaggtacattgttcgtttgtgtctctaatcgtatgccaaggagctgtctgcttagtgcccactttttcgcaaattcgatgagactgtgcgcgactcctttgcctcggtgcgtgtgcgacacaacaatgtgttcgatagaggctagatcgttccatgttgagttgagttcaatcttcccgacaagctcttggtcgatgaatgcgccatagcaagcagagtcttcatcagagtcatcatccgagatgtaatccttccggtaggggctcacacttctggtagatagttcaaagccttggtcggataggtgcacatcgaacacttcacgaacaatgaaatggttctcagcatccaatgtttccgccacctgctcagggatcaccgaaatcttcatatgacgcctaacgcctggcacagcggatcgcaaacctggcgcggcttttggcacaaaaggcgtgacaggtttgcgaatccgttgctgccacttgttaacccttttgccagatttggtaactataatttatgttagaggcgaagtcttgggtaaaaactggcctaaaattgctggggatttcaggaaagtaaacatcaccttccggctcgatgtctattgtagatatatgtagtgtatctacttgatcgggggatctgctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggggggggggggggggggttccattgttcattccacggacaaaaacagagaaaggaaacgacagaggccaaaaagctcgctttcagcacctgtcgtttcctttcttttcagagggtattttaaataaaaacattaagttatgacgaagaagaacggaaacgccttaaaccggaaaattttcataaatagcgaaaacccgcgaggtcgccgccccgtaacctgtcggatcaccggaaaggacccgtaaagtgataatgattatcatctacatatcacaacgtgcgtggaggccatcaaaccacgtcaaataatcaattatgacgcaggtatcgtattaattgatctgcatcaacttaacgtaaaaacaacttcagacaatacaaatcagcgacactgaatacggggcaacctcatgtccccccccccccccccctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattggtcgacgatcttgctgcgttcggatattttcgtggagttcccgccacagacccggattgaaggcgagatccagcaactcgcgccagatcatcctgtgacggaactttggcgcgtgatgactggccaggacgtcggccgaaagagcgacaagcagatcacgcttttcgacagcgtcggatttgcgatcgaggatttttcggcgctgcgctacgtccgcgaccgcgttgagggatcaagccacagcagcccactcgaccttctagccgacccagacgagccaagggatctttttggaatgctgctccgtcgtcaggctttccgacgtttgggtggttgaacagaagtcattatcgcacggaatgccaagcactcccgaggggaaccctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaacctgatcatgagcggagaattaagggagtcacgttatgacccccgccgatgacgcgggacaagccgttttacgtttggaactgacagaaccgcaacgttgaaggagccactcagcttaattaagtctaactcgagttactggtacgtatacagggttccttgcgtgaagaagggtggcctgcggttcaccattaacggtcacgactacttccagctagtactggtgaccaacgtcgcggcggcagggtcaatcaagtccatggaggttatgggttccaacacagcggattggatgccgatggcacgtaactggggcgcccaatggcactcactggcctacctcaccggtcaaggtctatcctttagggtcaccaacacagatgaccaaacgctcgtcttcaccaacgtcgtgccaccaggatggaagtttggccagacatttgcaagcaagctgcagttcaagtgagaggagaagcctgaattgataccggagcgtttcttttgggagtaacatctctggttgcctagcaaacatatgattgtatataagtttcgttgtgcgtttattctttcggtgtgtaaaataacatacatgctttcctgatattttcttgtatatatgtacacacacacgacaaatccttccatttctattattattgaacaatttaattgcgagggcgagtacttgtctgtttaccttttttttttcagatggcattttatagtttaacctttcatggaccggcagtagttctaaccatgaatgaaaagaaatcatagtccacaccacgcagggacattgtggtcattttagacaagacgatttgattaatgtcttgtatgatatggtcgacagtgaggactaacaaacatatggcatattttattaccggcgagttaaataaatttatgtcacagtaataaactgcctaataaatgcacgccagaaaatataatgataaaaaaaagaaaagatacataagtccattgcttctacttttttaaaaattaaatccaacattttctattttttggtataaacttggaagtactagttggatatgcaaaatcatctaacctccatatatttcatcaatttgtttactttacatatgggagaggatagtatgtcaaagaaaatgacaacaagcttacaagtttcttattttaaaagttccgctaacttatcaagcatagtgtgccacgcaaaactgacaacaaaccaacaaatttaaggagcgcctaacttatcatctatgacataccgcacaaaatgataacatactagagaaactttattgcacaaaaggaaatttatccataaggcaaaggaacatcttaaggctttggatatacatttaccaacaagcattgtttgtattacccctaaagcgcaagacatgtcatccatgagtcatagtgtgtatatctcaacattgcaaagctaccttttttctattatacttttcgcattataggctagatattatctatacatgtcaacaaactctatccctacgtcatatctgaagattcttttcttcactatataagttggcttccctgtcattgaactcacatcaaccagcccaagtttccaataacatcctcaaatagctggatcctaaaccatgagggtgttgctcgttgccctcgctctcctggctctcgctgcgagcgccacctccacgcatacaagcggcggctgcggctgccagccaccgccgccggttcatctaccgccgccggtgcatctgccacctccggttcacctgccacctccggtgcatctcccaccgccggtccacctgccgccgccggtccacctgccaccgccggtccatgtgccgccgccggttcatctgccgccgccaccatgccactaccctactcaaccgccccggcctcagcctcatccccagccacacccatgcccgtgccaacagccgcatccaagcccgtgccagggcagcggcggcagcggcccggccaggctgagggagctgagggccggcccggccccggccaggctgagggagctgagggccggcccggccccggccaggctgagggagctgaggaaggacgagctgtaac

SEQ ID NO: 72 - DNA, 12057 bp, synthetic construct
  misc_feature 1..12057: note = Synthetic construct, pAG4310
  source 1..12057: mol_type = other DNA
ctaggtccccgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattggaattcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcacggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagatccagatctaaaccatgcagaaactcattaactcagtgcaaaactatgcctggggcagcaaaacggcgttgactgaactttatggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggatgattttgccttctcgctgcatgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaagagcttactgaaaaaattaacatctcttgctaagctgggagctctagatccccgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattggcgagctcgaattaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaaatatcatctccctcgcagagatccgaattatcagccttcttattcatttctcgcttaaccgtgacaggctgtcgatcttgagaactatgccgacataataggaaatcgctggataaagccgctgaggaagctgagtggcgctatttctttagaagtgaacgttgacgatcgtcgaccgtaccccgatgaattaattcggacgtacgttctgaacacagctggatacttacttgggcgattgtcatacatgacatcaacaatgtacccgtttgtgtaaccgtctcttggaggttcgtatgacactagtggttcccctcagcttgcgactagatgttgaggcctaacattttattagagagcaggctagttgcttagatacatgatcttcaggccgttatctgtcagggcaagcgaaaattggccatttatgacgaccaatgccccgcagaagctcccatctttgccgccatagacgccgcgccccccttttggggtgtagaacatccttttgccagatgtggaaaagaagttcgttgtcccattgttggcaatgacgtagtagccggcgaaagtgcgagacccatttgcgctatatataagcctacgatttccgttgcgactattgtcgtaattggatgaactattatcgtagttgctctcagagttgtcgtaatttgatggactattgtcgtaattgcttatggagttgtcgtagttgcttggagaaatgtcgtagttggatggggagtagtcatagggaagacgagcttcatccactaaaacaattggcaggtcagcaagtgcctgccccgatgccatcgcaagtacgaggcttagaaccaccttcaacagatcgcgcatagtcttccccagctctctaacgcttgagttaagccgcgccgcgaagcggcgtcggcttgaacgaattgttagacattatttgccgactaccttggtgatctcgcctttcacgtagtgaacaaattcttccaactgatctgcgcgcgaggccaagcgatcttcttgtccaagataagcctgcctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatgatgtttaactcctgaattaagccgcgccgcgaagcggtgtcggcttgaatgaattgttaggcgtcatcctgtgctcccgagaaccagtaccagtacatcgctgtttcgttcgagacttgaggtctagttttatacgtgaacaggtcaatgccgccgagagtaaagccacattttgcgtacaaattgcaggcaggtacattgttcgtttgtgtctctaatcgtatgccaaggagctgtctgcttagtgcccactttttcgcaaattcgatgagactgtgcgcgactcctttgcctcggtgcgtgtgcgacacaacaatgtgttcgatagaggctagatcgttccatgttgagttgagttcaatcttcccgacaagctcttggtcgatgaatgcgccatagcaagcagagtcttcatcagagtcatcatccgagatgtaatccttccggtaggggctcacacttctggtagatagttcaaagccttggtcggataggtgcacatcgaacacttcacgaacaatgaaatggttctcagcatccaatgtttccgccacctgctcagggatcaccgaaatcttcatatgacgcctaacgcctggcacagcggatcgcaaacctggcgcggcttttggcacaaaaggcgtgacaggtttgcgaatccgttgctgccacttgttaacccttttgccagatttggtaactataatttatgttagaggcgaagtcttgggtaaaaactggcctaaaattgctggggatttcaggaaagtaaacatcaccttccggctcgatgtctattgtagatatatgtagtgtatctacttgatcgggggatctgctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggggggggggggggggggttccattgttcattccacggacaaaaacagagaaaggaaacgacagaggccaaaaagctcgctttcagcacctgtcgtttcctttcttttcagagggtattttaaataaaaacattaagttatgacgaagaagaacggaaacgccttaaaccggaaaattttcataaatagcgaaaacccgcgaggtcgccgccccgtaacctgtcggatcaccggaaaggacccgtaaagtgataatgattatcatctacatatcacaacgtgcgtggaggccatcaaaccacgtcaaataatcaattatgacgcaggtatcgtattaattgatctgcatcaacttaacgtaaaaacaacttcagacaatacaaatcagcgacactgaatacggggcaacctcatgtccccccccccccccccctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattggtcgacgatcttgctgcgttcggatattttcgtggagttcccgccacagacccggattgaaggcgagatccagcaactcgcgccagatcatcctgtgacggaactttggcgcgtgatgactggccaggacgtcggccgaaagagcgacaagcagatcacgcttttcgacagcgtcggatttgcgatcgaggatttttcggcgctgcgctacgtccgcgaccgcgttgagggatcaagccacagcagcccactcgaccttctagccgacccagacgagccaagggatctttttggaatgctgctccgtcgtcaggctttccgacgtttgggtggttgaacagaagtcattatcgcacggaatgccaagcactcccgaggggaaccctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaacctgatcatgagcggagaattaagggagtcacgttatgacccccgccgatgacgcgggacaagccgttttacgtttggaactgacagaaccgcaacgttgaaggagccactcagcttaattaagtctaactcgagttactggtacgtatacagggttccttgcgtgaagaagggtggcctgcggttcaccattaacggtcacgactacttccagctagtactggtgaccaacgtcgcggcggcagggtcaatcaagtccatggaggttatgggttccaacacagcggattggatgccgatggcacgtaactggggcgcccaatggcactcactggcctacctcaccggtcaaggtctatcctttagggtcaccaacacagatgaccaaacgctcgtcttcaccaacgtcgtgccaccaggatggaagtttggccagacatttgcaagcaagctgcagttcaagtgagaggagaagcctgaattgataccggagcgtttcttttgggagtaacatctctggttgcctagcaaacatatgattgtatataagtttcgttgtgcgtttattctttcggtgtgtaaaataacatacatgctttcctgatattttcttgtatatatgtacacacacacgacaaatccttccatttctattattattgaacaatttaattgcgagggcgagtacttgtctgtttaccttttttttttcagatggcattttatagtttaacctttcatggaccggcagtagttctaaccatgaatgaaaagaaatcatagtccacaccacgcagggacattgtggtcattttagacaagacgatttgattaatgtcttgtatgatatggtcgacagtgaggactaacaaacatatggcatattttattaccggcgagttaaataaatttatgtcacagtaataaactgcctaataaatgcacgccagaaaatataatgataaaaaaaagaaaagatacataagtccattgcttctacttttttaaaaattaaatccaacattttctattttttggtataaacttggaagtactagttggatatgcaaaatcatctaacctccatatatttcatcaatttgtttactttacatatgggagaggatagtatgtcaaagaaaatgacaacaagcttacaagtttcttattttaaaagttccgctaacttatcaagcatagtgtgccacgcaaaactgacaacaaaccaacaaatttaaggagcgcctaacttatcatctatgacataccgcacaaaatgataacatactagagaaactttattgcacaaaaggaaatttatccataaggcaaaggaacatcttaaggctttggatatacatttaccaacaagcattgtttgtattacccctaaagcgcaagacatgtcatccatgagtcatagtgtgtatatctcaacattgcaaagctaccttttttctattatacttttcgcattataggctagatattatctatacatgtcaacaaactctatccctacgtcatatctgaagattcttttcttcactatataagttggcttccctgtcattgaactcacatcaaccagcccaagtttccaataacatcctcaaatagctggatcctaaaccatgagggtgttgctcgttgccctcgctctcctggctctcgctgcgagcgccacctccccggccaggctgagggagctgagggccggcccggccccggccaggctgagggagctgagggccggcccggccccggccaggctgagggagctgaggggcagcggcggcagcggcggcggctgcggctgccagccaccgccgccggttcatctaccgccgccggtgcatctgccacctccggttcacctgccacctccggtgcatctcccaccgccggtccacctgccgccgccggtccacctgccaccgccggtccatgtgccgccgccggttcatctgccgccgccaccatgccactaccctactcaaccgccccggcctcagcctcatccccagccacacccatgcccgtgccaacagccgcatccaagcccgtgccagaaggacgagctgtaac

SEQ ID NO: 73 - DNA, 12210 bp, synthetic construct
  misc_feature 1..12210: note = Synthetic construct, pAG4311
  source 1..12210: mol_type = other DNA
ctaggtccccgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattggaattcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcacggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagatccagatctaaaccatgcagaaactcattaactcagtgcaaaactatgcctggggcagcaaaacggcgttgactgaactttatggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggatgattttgccttctcgctgcatgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaagagcttactgaaaaaattaacatctcttgctaagctgggagctctagatccccgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattggcgagctcgaattaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaaatatcatctccctcgcagagatccgaattatcagccttcttattcatttctcgcttaaccgtgacaggctgtcgatcttgagaactatgccgacataataggaaatcgctggataaagccgctgaggaagctgagtggcgctatttctttagaagtgaacgttgacgatcgtcgaccgtaccccgatgaattaattcggacgtacgttctgaacacagctggatacttacttgggcgattgtcatacatgacatcaacaatgtacccgtttgtgtaaccgtctcttggaggttcgtatgacactagtggttcccctcagcttgcgactagatgttgaggcctaacattttattagagagcaggctagttgcttagatacatgatcttcaggccgttatctgtcagggcaagcgaaaattggccatttatgacgaccaatgccccgcagaagctcccatctttgccgccatagacgccgcgccccccttttggggtgtagaacatccttttgccagatgtggaaaagaagttcgttgtcccattgttggcaatgacgtagtagccggcgaaagtgcgagacccatttgcgctatatataagcctacgatttccgttgcgactattgtcgtaattggatgaactattatcgtagttgctctcagagttgtcgtaatttgatggactattgtcgtaattgcttatggagttgtcgtagttgcttggagaaatgtcgtagttggatggggagtagtcatagggaagacgagcttcatccactaaaacaattggcaggtcagcaagtgcctgccccgatgccatcgcaagtacgaggcttagaaccaccttcaacagatcgcgcatagtcttccccagctctctaacgcttgagttaagccgcgccgcgaagcggcgtcggcttgaacgaattgttagacattatttgccgactaccttggtgatctcgcctttcacgtagtgaacaaattcttccaactgatctgcgcgcgaggccaagcgatcttcttgtccaagataagcctgcctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatgatgtttaactcctgaattaagccgcgccgcgaagcggtgtcggcttgaatgaattgttaggcgtcatcctgtgctcccgagaaccagtaccagtacatcgctgtttcgttcgagacttgaggtctagttttatacgtgaacaggtcaatgccgccgagagtaaagccacattttgcgtacaaattgcaggcaggtacattgttcgtttgtgtctctaatcgtatgccaaggagctgtctgcttagtgcccactttttcgcaaattcgatgagactgtgcgcgactcctttgcctcggtgcgtgtgcgacacaacaatgtgttcgatagaggctagatcgttccatgttgagttgagttcaatcttcccgacaagctcttggtcgatgaatgcgccatagcaagcagagtcttcatcagagtcatcatccgagatgtaatccttccggtaggggctcacacttctggtagatagttcaaagccttggtcggataggtgcacatcgaacacttcacgaacaatgaaatggttctcagcatccaatgtttccgccacctgctcagggatcaccgaaatcttcatatgacgcctaacgcctggcacagcggatcgcaaacctggcgcggcttttggcacaaaaggcgtgacaggtttgcgaatccgttgctgccacttgttaacccttttgccagatttggtaactataatttatgttagaggcgaagtcttgggtaaaaactggcctaaaattgctggggatttcaggaaagtaaacatcaccttccggctcgatgtctattgtagatatatgtagtgtatctacttgatcgggggatctgctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggggggggggggggggggttccattgttcattccacggacaaaaacagagaaaggaaacgacagaggccaaaaagctcgctttcagcacctgtcgtttcctttcttttcagagggtattttaaataaaaacattaagttatgacgaagaagaacggaaacgccttaaaccggaaaattttcataaatagcgaaaacccgcgaggtcgccgccccgtaacctgtcggatcaccggaaaggacccgtaaagtgataatgattatcatctacatatcacaacgtgcgtggaggccatcaaaccacgtcaaataatcaattatgacgcaggtatcgtattaattgatctgcatcaacttaacgtaaaaacaacttcagacaatacaaatcagcgacactgaatacggggcaacctcatgtccccccccccccccccctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattggtcgacgatcttgctgcgttcggatattttcgtggagttcccgccacagacccggattgaaggcgagatccagcaactcgcgccagatcatcctgtgacggaactttggcgcgtgatgactggccaggacgtcggccgaaagagcgacaagcagatcacgcttttcgacagcgtcggatttgcgatcgaggatttttcggcgctgcgctacgtccgcgaccgcgttgagggatcaagccacagcagcccactcgaccttctagccgacccagacgagccaagggatctttttggaatgctgctccgtcgtcaggctttccgacgtttgggtggttgaacagaagtcattatcgcacggaatgccaagcactcccgaggggaaccctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaacctgatcatgagcggagaattaagggagtcacgttatgacccccgccgatgacgcgggacaagccgttttacgtttggaactgacagaaccgcaacgttgaaggagccactcagcttaattaagtctaactcgagttactggtacgtatacagggttccttgcgtgaagaagggtggcctgcggttcaccattaacggtcacgactacttccagctagtactggtgaccaacgtcgcggcggcagggtcaatcaagtccatggaggttatgggttccaacacagcggattggatgccgatggcacgtaactggggcgcccaatggcactcactggcctacctcaccggtcaaggtctatcctttagggtcaccaacacagatgaccaaacgctcgtcttcaccaacgtcgtgccaccaggatggaagtttggccagacatttgcaagcaagctgcagttcaagtgagaggagaagcctgaattgataccggagcgtttcttttgggagtaacatctctggttgcctagcaaacatatgattgtatataagtttcgttgtgcgtttattctttcggtgtgtaaaataacatacatgctttcctgatattttcttgtatatatgtacacacacacgacaaatccttccatttctattattattgaacaatttaattgcgagggcgagtacttgtctgtttaccttttttttttcagatggcattttatagtttaacctttcatggaccggcagtagttctaaccatgaatgaaaagaaatcatagtccacaccacgcagggacattgtggtcattttagacaagacgatttgattaatgtcttgtatgatatggtcgacagtgaggactaacaaacatatggcatattttattaccggcgagttaaataaatttatgtcacagtaataaactgcctaataaatgcacgccagaaaatataatgataaaaaaaagaaaagatacataagtccattgcttctacttttttaaaaattaaatccaacattttctattttttggtataaacttggaagtactagttggatatgcaaaatcatctaacctccatatatttcatcaatttgtttactttacatatgggagaggatagtatgtcaaagaaaatgacaacaagcttacaagtttcttattttaaaagttccgctaacttatcaagcatagtgtgccacgcaaaactgacaacaaaccaacaaatttaaggagcgcctaacttatcatctatgacataccgcacaaaatgataacatactagagaaactttattgcacaaaaggaaatttatccataaggcaaaggaacatcttaaggctttggatatacatttaccaacaagcattgtttgtattacccctaaagcgcaagacatgtcatccatgagtcatagtgtgtatatctcaacattgcaaagctaccttttttctattatacttttcgcattataggctagatattatctatacatgtcaacaaactctatccctacgtcatatctgaagattcttttcttcactatataagttggcttccctgtcattgaactcacatcaaccagcccaagtttccaataacatcctcaaatagctggatcctaaaccatgagggtgttgctcgttgccctcgctctcctggctctcgctgcgagcgccacctccccggccaggctgagggagctgagggccggcccggccccggccaggctgagggagctgagggccggcccggccccggccaggctgagggagctgaggggcagcggcggcagcggcgtgccgggcgtgggcgtgccgggcgtgggcgtgccgggcgtgggcgtgccgggcgtgggcgtgccgggcgtgggcgtgccgggcgtgggcgtgccgggcgtgggcgtgccgggcgtgggcgtgccgggcgtgggcgtgccgggcgtgggcgtgccgggcgtgggcgtgccgggcgtgggcgtgccgggcgtgggcgtgccgggcgtgggcgtgccgggcgtgggcgtgccgggcgtgggcgtgccgggcgtgggcgtgccgggcgtgggcgtgccgggcgtgggcgtgccgggcgtgggcgtgccgggcgtgggcgtgccgggcgtgggcgtgccgggcgtgggcgtgccgggcgtgggcgtgccgggcgtgggcgtgccgggcgtgggcgtgccgggcgtgggcgtgccgggcgtgggcaaggacgagctgtaac

SEQ ID NO: 74 - DNA, 12015 bp, synthetic construct
  misc_feature 1..12015: note = Synthetic construct, pAG4312
  source 1..12015: mol_type = other DNA
ctaggtccccgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattggaattcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcacggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagatccagatctaaaccatgcagaaactcattaactcagtgcaaaactatgcctggggcagcaaaacggcgttgactgaactttatggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggatgattttgccttctcgctgcatgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaagagcttactgaaaaaattaacatctcttgctaagctgggagctctagatccccgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattggcgagctcgaattaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaaatatcatctccctcgcagagatccgaattatcagccttcttattcatttctcgcttaaccgtgacaggctgtcgatcttgagaactatgccgacataataggaaatcgctggataaagccgctgaggaagctgagtggcgctatttctttagaagtgaacgttgacgatcgtcgaccgtaccccgatgaattaattcggacgtacgttctgaacacagctggatacttacttgggcgattgtcatacatgacatcaacaatgtacccgtttgtgtaaccgtctcttggaggttcgtatgacactagtggttcccctcagcttgcgactagatgttgaggcctaacattttattagagagcaggctagttgcttagatacatgatcttcaggccgttatctgtcagggcaagcgaaaattggccatttatgacgaccaatgccccgcagaagctcccatctttgccgccatagacgccgcgccccccttttggggtgtagaacatccttttgccagatgtggaaaagaagttcgttgtcccattgttggcaatgacgtagtagccggcgaaagtgcgagacccatttgcgctatatataagcctacgatttccgttgcgactattgtcgtaattggatgaactattatcgtagttgctctcagagttgtcgtaatttgatggactattgtcgtaattgcttatggagttgtcgtagttgcttggagaaatgtcgtagttggatggggagtagtcatagggaagacgagcttcatccactaaaacaattggcaggtcagcaagtgcctgccccgatgccatcgcaagtacgaggcttagaaccaccttcaacagatcgcgcatagtcttccccagctctctaacgcttgagttaagccgcgccgcgaagcggcgtcggcttgaacgaattgttagacattatttgccgactaccttggtgatctcgcctttcacgtagtgaacaaattcttccaactgatctgcgcgcgaggccaagcgatcttcttgtccaagataagcctgcctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatgatgtttaactcctgaattaagccgcgccgcgaagcggtgtcggcttgaatgaattgttaggcgtcatcctgtgctcccgagaaccagtaccagtacatcgctgtttcgttcgagacttgaggtctagttttatacgtgaacaggtcaatgccgccgagagtaaagccacattttgcgtacaaattgcaggcaggtacattgttcgtttgtgtctctaatcgtatgccaaggagctgtctgcttagtgcccactttttcgcaaattcgatgagactgtgcgcgactcctttgcctcggtgcgtgtgcgacacaacaatgtgttcgatagaggctagatcgttccatgttgagttgagttcaatcttcccgacaagctcttggtcgatgaatgcgccatagcaagcagagtcttcatcagagtcatcatccgagatgtaatccttccggtaggggctcacacttctggtagatagttcaaagccttggtcggataggtgcacatcgaacacttcacgaacaatgaaatggttctcagcatccaatgtttccgccacctgctcagggatcaccgaaatcttcatatgacgcctaacgcctggcacagcggatcgcaaacctggcgcggcttttggcacaaaaggcgtgacaggtttgcgaatccgttgctgccacttgttaacccttttgccagatttggtaactataatttatgttagaggcgaagtcttgggtaaaaactggcctaaaattgctggggatttcaggaaagtaaacatcaccttccggctcgatgtctattgtagatatatgtagtgtatctacttgatcgggggatctgctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggggggggggggggggggttccattgttcattccacggacaaaaacagagaaaggaaacgacagaggccaaaaagctcgctttcagcacctgtcgtttcctttcttttcagagggtattttaaataaaaacattaagttatgacgaagaagaacggaaacgccttaaaccggaaaattttcataaatagcgaaaacccgcgaggtcgccgccccgtaacctgtcggatcaccggaaaggacccgtaaagtgataatgattatcatctacatatcacaacgtgcgtggaggccatcaaaccacgtcaaataatcaattatgacgcaggtatcgtattaattgatctgcatcaacttaacgtaaaaacaacttcagacaatacaaatcagcgacactgaatacggggcaacctcatgtccccccccccccccccctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattggtcgacgatcttgctgcgttcggatattttcgtggagttcccgccacagacccggattgaaggcgagatccagcaactcgcgccagatcatcctgtgacggaactttggcgcgtgatgactggccaggacgtcggccgaaagagcgacaagcagatcacgcttttcgacagcgtcggatttgcgatcgaggatttttcggcgctgcgctacgtccgcgaccgcgttgagggatcaagccacagcagcccactcgaccttctagccgacccagacgagccaagggatctttttggaatgctgctccgtcgtcaggctttccgacgtttgggtggttgaacagaagtcattatcgcacggaatgccaagcactcccgaggggaaccctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaacctgatcatgagcggagaattaagggagtcacgttatgacccccgccgatgacgcgggacaagccgttttacgtttggaactgacagaaccgcaacgttgaaggagccactcagcttaattaagtctaactcgagttactggtacgtatacagggttccttgcgtgaagaagggtggcctgcggttcaccattaacggtcacgactacttccagctagtactggtgaccaacgtcgcggcggcagggtcaatcaagtccatggaggttatgggttccaacacagcggattggatgccgatggcacgtaactggggcgcccaatggcactcactggcctacctcaccggtcaaggtctatcctttagggtcaccaacacagatgaccaaacgctcgtcttcaccaacgtcgtgccaccaggatggaagtttggccagacatttgcaagcaagctgcagttcaagtgagaggagaagcctgaattgataccggagcgtttcttttgggagtaacatctctggttgcctagcaaacatatgattgtatataagtttcgttgtgcgtttattctttcggtgtgtaaaataacatacatgctttcctgatattttcttgtatatatgtacacacacacgacaaatccttccatttctattattattgaacaatttaattgcgagggcgagtacttgtctgtttaccttttttttttcagatggcattttatagtttaacctttcatggaccggcagtagttctaaccatgaatgaaaagaaatcatagtccacaccacgcagggacattgtggtcattttagacaagacgatttgattaatgtcttgtatgatatggtcgacagtgaggactaacaaacatatggcatattttattaccggcgagttaaataaatttatgtcacagtaataaactgcctaataaatgcacgccagaaaatataatgataaaaaaaagaaaagatacataagtccattgcttctacttttttaaaaattaaatccaacattttctattttttggtataaacttggaagtactagttggatatgcaaaatcatctaacctccatatatttcatcaatttgtttactttacatatgggagaggatagtatgtcaaagaaaatgacaacaagcttacaagtttcttattttaaaagttccgctaacttatcaagcatagtgtgccacgcaaaactgacaacaaaccaacaaatttaaggagcgcctaacttatcatctatgacataccgcacaaaatgataacatactagagaaactttattgcacaaaaggaaatttatccataaggcaaaggaacatcttaaggctttggatatacatttaccaacaagcattgtttgtattacccctaaagcgcaagacatgtcatccatgagtcatagtgtgtatatctcaacattgcaaagctaccttttttctattatacttttcgcattataggctagatattatctatacatgtcaacaaactctatccctacgtcatatctgaagattcttttcttcactatataagttggcttccctgtcattgaactcacatcaaccagcccaagtttccaataacatcctcaaatagctggatcctaaaccatgagggtgttgctcgttgccctcgctctcctggctctcgctgcgagcgccacctccccggccaggctgagggagctgagggccggcccggccccggccaggctgagggagctgagggccggcccggccccggccaggctgagggagctgaggggcagcggcggcagcggcagcaacggcaacggcaacgtgtgcccgccgggcctgttcagcaacccgcagtgctgcgccacccaggtgctgggcctgatcggcctggactgcaaggtgccgagccagaacgtgtacgacggcaccgacttcaggaacgtgtgcgccaagaccggcgcccagccgctgtgctgcgtggccccggtggccggccaggccctgctgtgccagaccgccgtgggcgccaaggacgagctgtaac

SEQ ID NO: 75 - DNA, 12738 bp, synthetic construct
  misc_feature 1..12738: note = Synthetic construct, pAG4313
  source 1..12738: mol_type = other DNA
ctaggtccccgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattggaattcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcacggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagatccagatctaaaccatgcagaaactcattaactcagtgcaaaactatgcctggggcagcaaaacggcgttgactgaactttatggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggatgattttgccttctcgctgcatgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaagagcttactgaaaaaattaacatctcttgctaagctgggagctctagatccccgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattggcgagctcgaattaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaaatatcatctccctcgcagagatccgaattatcagccttcttattcatttctcgcttaaccgtgacaggctgtcgatcttgagaactatgccgacataataggaaatcgctggataaagccgctgaggaagctgagtggcgctatttctttagaagtgaacgttgacgatcgtcgaccgtaccccgatgaattaattcggacgtacgttctgaacacagctggatacttacttgggcgattgtcatacatgacatcaacaatgtacccgtttgtgtaaccgtctcttggaggttcgtatgacactagtggttcccctcagcttgcgactagatgttgaggcctaacattttattagagagcaggctagttgcttagatacatgatcttcaggccgttatctgtcagggcaagcgaaaattggccatttatgacgaccaatgccccgcagaagctcccatctttgccgccatagacgccgcgccccccttttggggtgtagaacatccttttgccagatgtggaaaagaagttcgttgtcccattgttggcaatgacgtagtagccggcgaaagtgcgagacccatttgcgctatatataagcctacgatttccgttgcgactattgtcgtaattggatgaactattatcgtagttgctctcagagttgtcgtaatttgatggactattgtcgtaattgcttatggagttgtcgtagttgcttggagaaatgtcgtagttggatggggagtagtcatagggaagacgagcttcatccactaaaacaattggcaggtcagcaagtgcctgccccgatgccatcgcaagtacgaggcttagaaccaccttcaacagatcgcgcatagtcttccccagctctctaacgcttgagttaagccgcgccgcgaagcggcgtcggcttgaacgaattgttagacattatttgccgactaccttggtgatctcgcctttcacgtagtgaacaaattcttccaactgatctgcgcgcgaggccaagcgatcttcttgtccaagataagcctgcctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatgatgtttaactcctgaattaagccgcgccgcgaagcggtgtcggcttgaatgaattgttaggcgtcatcctgtgctcccgagaaccagtaccagtacatcgctgtttcgttcgagacttgaggtctagttttatacgtgaacaggtcaatgccgccgagagtaaagccacattttgcgtacaaattgcaggcaggtacattgttcgtttgtgtctctaatcgtatgccaaggagctgtctgcttagtgcccactttttcgcaaattcgatgagactgtgcgcgactcctttgcctcggtgcgtgtgcgacacaacaatgtgttcgatagaggctagatcgttccatgttgagttgagttcaatcttcccgacaagctcttggtcgatgaatgcgccatagcaagcagagtcttcatcagagtcatcatccgagatgtaatccttccggtaggggctcacacttctggtagatagttcaaagccttggtcggataggtgcacatcgaacacttcacgaacaatgaaatggttctcagcatccaatgtttccgccacctgctcagggatcaccgaaatcttcatatgacgcctaacgcctggcacagcggatcgcaaacctggcgcggcttttggcacaaaaggcgtgacaggtttgcgaatccgttgctgccacttgttaacccttttgccagatttggtaactataatttatgttagaggcgaagtcttgggtaaaaactggcctaaaattgctggggatttcaggaaagtaaacatcaccttccggctcgatgtctattgtagatatatgtagtgtatctacttgatcgggggatctgctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggggggggggggggggggttccattgttcattccacggacaaaaacagagaaaggaaacgacagaggccaaaaagctcgctttcagcacctgtcgtttcctttcttttcagagggtattttaaataaaaacattaagttatgacgaagaagaacggaaacgccttaaaccggaaaattttcataaatagcgaaaacccgcgaggtcgccgccccgtaacctgtcggatcaccggaaaggacccgtaaagtgataatgattatcatctacatatcacaacgtgcgtggaggccatcaaaccacgtcaaataatcaattatgacgcaggtatcgtattaattgatctgcatcaacttaacgtaaaaacaacttcagacaatacaaatcagcgacactgaatacggggcaacctcatgtccccccccccccccccctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattggtcgacgatcttgctgcgttcggatattttcgtggagttcccgccacagacccggattgaaggcgagatccagcaactcgcgccagatcatcctgtgacggaactttggcgcgtgatgactggccaggacgtcggccgaaagagcgacaagcagatcacgcttttcgacagcgtcggatttgcgatcgaggatttttcggcgctgcgctacgtccgcgaccgcgttgagggatcaagccacagcagcccactcgaccttctagccgacccagacgagccaagggatctttttggaatgctgctccgtcgtcaggctttccgacgtttgggtggttgaacagaagtcattatcgcacggaatgccaagcactcccgaggggaaccctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaacctgatcatgagcggagaattaagggagtcacgttatgacccccgccgatgacgcgggacaagccgttttacgtttggaactgacagaaccgcaacgttgaaggagccactcagcttaattaagtctaactcgagttactggtacgtatacagggttccttgcgtgaagaagggtggcctgcggttcaccattaacggtcacgactacttccagctagtactggtgaccaacgtcgcggcggcagggtcaatcaagtccatggaggttatgggttccaacacagcggattggatgccgatggcacgtaactggggcgcccaatggcactcactggcctacctcaccggtcaaggtctatcctttagggtcaccaacacagatgaccaaacgctcgtcttcaccaacgtcgtgccaccaggatggaagtttggccagacatttgcaagcaagctgcagttcaagtgagaggagaagcctgaattgataccggagcgtttcttttgggagtaacatctctggttgcctagcaaacatatgattgtatataagtttcgttgtgcgtttattctttcggtgtgtaaaataacatacatgctttcctgatattttcttgtatatatgtacacacacacgacaaatccttccatttctattattattgaacaatttaattgcgagggcgagtacttgtctgtttaccttttttttttcagatggcattttatagtttaacctttcatggaccggcagtagttctaaccatgaatgaaaagaaatcatagtccacaccacgcagggacattgtggtcattttagacaagacgatttgattaatgtcttgtatgatatggtcgacagtgaggactaacaaacatatggcatattttattaccggcgagttaaataaatttatgtcacagtaataaactgcctaataaatgcacgccagaaaatataatgataaaaaaaagaaaagatacataagtccattgcttctacttttttaaaaattaaatccaacattttctattttttggtataaacttggaagtactagttggatatgcaaaatcatctaacctccatatatttcatcaatttgtttactttacatatgggagaggatagtatgtcaaagaaaatgacaacaagcttacaagtttcttattttaaaagttccgctaacttatcaagcatagtgtgccacgcaaaactgacaacaaaccaacaaatttaaggagcgcctaacttatcatctatgacataccgcacaaaatgataacatactagagaaactttattgcacaaaaggaaatttatccataaggcaaaggaacatcttaaggctttggatatacatttaccaacaagcattgtttgtattacccctaaagcgcaagacatgtcatccatgagtcatagtgtgtatatctcaacattgcaaagctaccttttttctattatacttttcgcattataggctagatattatctatacatgtcaacaaactctatccctacgtcatatctgaagattcttttcttcactatataagttggcttccctgtcattgaactcacatcaaccagcccaagtttccaataacatcctcaaatagctggatcctaaaccatgagggtgttgctcgttgccctcgctctcctggctctcgctgcgagcgccacctccccggccaggctgagggagctgagggccggcccggccccggccaggctgagggagctgagggccggcccggccccggccaggctgagggagctgaggggcagcggcggcagcggcggcgtggacccgttcgagaggaacaagatcctgggcaggggcatcaacatcggcaacgccctggaggccccgaacgagggcgactggggcgtggtgatcaaggacgagttcttcgacatcatcaaggaggccggcttcagccacgtgagaatcccgatcaggtggagcacccacgcccaggccttcccgccgtacaagatcgagccgagcttcttcaagagggtggacgaggtgatcaacggcgccctgaagaggggcctggccgtggtgatcaacatccaccactacgaggagctgatgaacgacccggaggagcacaaggagaggttcctggccctgtggaagcagatcgccgacaggtacaaggactacccggagaccctgttcttcgagatcctgaacgagccgcacggcaacctgaccccggagaagtggaacgagctgctggaggaggccctgaaggtgatcaggagcatcgacaagaagcacaccgtgatcatcggcaccgccgagtggggcggcatcagcgccctggagaagctgagggtgccgaagtgggagaagaacgccatcgtgaccatccactactacaacccgttcgagttcacccaccagggcgccgagtgggtgccgggcagcgagaagtggctgggcaggaagtggggcagcccggacgaccagaagcacctgatcgaggagttcaacttcatcgaggagtggagcaagaagaacaagaggccgatctacatcggcgagttcggcgcctacaggaaggccgacctggagagcaggatcaagtggaccagcttcgtggtgagggaggccgagaagaggggctggagctgggcctactgggagttctgcagcggcttcggcgtgtacgacccgctgaggaagcagtggaacaaggacctgctggaggccctgatcggcggcgacagcatcgagaaggacgagctgtaac

SEQ ID NO: 76 - DNA, 11825 bp, synthetic construct
  misc_feature 1..11825: note = Synthetic construct, pAG4981
  source 1..11825: mol_type = other DNA
ctaggtccccgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattggaattcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcacggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagatccagatctaaaccatgcagaaactcattaactcagtgcaaaactatgcctggggcagcaaaacggcgttgactgaactttatggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggatgattttgccttctcgctgcatgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaagagcttactgaaaaaattaacatctcttgctaagctgggagctctagatccccgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattggcgagctcgaattaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaaatatcatctccctcgcagagatccgaattatcagccttcttattcatttctcgcttaaccgtgacaggctgtcgatcttgagaactatgccgacataataggaaatcgctggataaagccgctgaggaagctgagtggcgctatttctttagaagtgaacgttgacgatcgtcgaccgtaccccgatgaattaattcggacgtacgttctgaacacagctggatacttacttgggcgattgtcatacatgacatcaacaatgtacccgtttgtgtaaccgtctcttggaggttcgtatgacactagtggttcccctcagcttgcgactagatgttgaggcctaacattttattagagagcaggctagttgcttagatacatgatcttcaggccgttatctgtcagggcaagcgaaaattggccatttatgacgaccaatgccccgcagaagctcccatctttgccgccatagacgccgcgccccccttttggggtgtagaacatccttttgccagatgtggaaaagaagttcgttgtcccattgttggcaatgacgtagtagccggcgaaagtgcgagacccatttgcgctatatataagcctacgatttccgttgcgactattgtcgtaattggatgaactattatcgtagttgctctcagagttgtcgtaatttgatggactattgtcgtaattgcttatggagttgtcgtagttgcttggagaaatgtcgtagttggatggggagtagtcatagggaagacgagcttcatccactaaaacaattggcaggtcagcaagtgcctgccccgatgccatcgcaagtacgaggcttagaaccaccttcaacagatcgcgcatagtcttccccagctctctaacgcttgagttaagccgcgccgcgaagcggcgtcggcttgaacgaattgttagacattatttgccgactaccttggtgatctcgcctttcacgtagtgaacaaattcttccaactgatctgcgcgcgaggccaagcgatcttcttgtccaagataagcctgcctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatgatgtttaactcctgaattaagccgcgccgcgaagcggtgtcggcttgaatgaattgttaggcgtcatcctgtgctcccgagaaccagtaccagtacatcgctgtttcgttcgagacttgaggtctagttttatacgtgaacaggtcaatgccgccgagagtaaagccacattttgcgtacaaattgcaggcaggtacattgttcgtttgtgtctctaatcgtatgccaaggagctgtctgcttagtgcccactttttcgcaaattcgatgagactgtgcgcgactcctttgcctcggtgcgtgtgcgacacaacaatgtgttcgatagaggctagatcgttccatgttgagttgagttcaatcttcccgacaagctcttggtcgatgaatgcgccatagcaagcagagtcttcatcagagtcatcatccgagatgtaatccttccggtaggggctcacacttctggtagatagttcaaagccttggtcggataggtgcacatcgaacacttcacgaacaatgaaatggttctcagcatccaatgtttccgccacctgctcagggatcaccgaaatcttcatatgacgcctaacgcctggcacagcggatcgcaaacctggcgcggcttttggcacaaaaggcgtgacaggtttgcgaatccgttgctgccacttgttaacccttttgccagatttggtaactataatttatgttagaggcgaagtcttgggtaaaaactggcctaaaattgctggggatttcaggaaagtaaacatcaccttccggctcgatgtctattgtagatatatgtagtgtatctacttgatcgggggatctgctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggggggggggggggggggttccattgttcattccacggacaaaaacagagaaaggaaacgacagaggccaaaaagctcgctttcagcacctgtcgtttcctttcttttcagagggtattttaaataaaaacattaagttatgacgaagaagaacggaaacgccttaaaccggaaaattttcataaatagcgaaaacccgcgaggtcgccgccccgtaacctgtcggatcaccggaaaggacccgtaaagtgataatgattatcatctacatatcacaacgtgcgtggaggccatcaaaccacgtcaaataatcaattatgacgcaggtatcgtattaattgatctgcatcaacttaacgtaaaaacaacttcagacaatacaaatcagcgacactgaatacggggcaacctcatgtccccccccccccccccctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattggtcgacgatcttgctgcgttcggatattttcgtggagttcccgccacagacccggattgaaggcgagatccagcaactcgcgccagatcatcctgtgacggaactttggcgcgtgatgactggccaggacgtcggccgaaagagcgacaagcagatcacgcttttcgacagcgtcggatttgcgatcgaggatttttcggcgctgcgctacgtccgcgaccgcgttgagggatcaagccacagcagcccactcgaccttctagccgacccagacgagccaagggatctttttggaatgctgctccgtcgtcaggctttccgacgtttgggtggttgaacagaagtcattatcgcacggaatgccaagcactcccgaggggaaccctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaacctgatcatgagcggagaattaagggagtcacgttatgacccccgccgatgacgcgggacaagccgttttacgtttggaactgacagaaccgcaacgttgaaggagccactcagcctaagcggccgcattggacttaattaagtgaggccggccaagcgtcgatttaaatgtaccacatggcgcgccaactatcatgcgatcgcttcatgtctaactcgagttactggtacgtaccaaatccatggaatcaaggtaccaaagtaatcatattattttatgtgtgaatcttctttactttttcatttgattatgattatgaaggtatgaccttcataaccttcgtccgaaatccattatatccaaaggaaaataatgcttcgaaggacgaaggattttgatatttaacattttatgttgccttgttcttaattcatagcatttgagaacaagtccccaacaccaatctttatctttactatattaaagcaccagttcaacgatcgtctcgtgtcaatattattaaaaaactcctacatttctttataatcaacccgcactcttataatctcttctcttactactataataagagagtttatgtacaaaataaggtgaaattatgtataagtgttctggaccttggttgttggctcatattcacacaacctaatcaatagaaaacatatgttttattaaaacaaaatttatcatatatatatatatatatatatatatatatatatatatatataatataaaccgtagcaatgcacaggcatatgactagtggcaacttaataccatgtgtgtattaagatgaataagaggtatccaaataaataacttgttcgcttacgtctggatcgaaaggggttggaaacgattaaatctcttcctagtcaaaattaaatagaaggagatttaatcgatttctcccaatccccttcgatccaggtgcaaccgaataagtccttaaatgttgaggaacacgaaacaaccatgcattggcatgtaaagctccaagaattcgttgtatccttaacaactcacagaacatcaaccaaaattgcacgtcaagggtattgggtaagaaacaatcaaacaaatcctctctgtgtgcaaagaaacacggtgagtcatgccgagatcatactcatctgatatacatgcttacagctcacaagacattacaaacaactcatattgcattacaaagatcgtttcatgaaaaataaaataggccggaacaggacaaaaatccttgacgtgtaaagtaaatttacaacaaaaaaaaagccatatgtcaagctaaatctaattcgttttacgtagatcaacaacctgtagaaggcaacaaaactgagccacgcagaagtacagaatgattccagatgaaccatcgacgtgctacgtaaagagagtgacgagtcatatacatttggcaagaaaccatgaagctgcctacagccgtctcggtggcataagaacacaagaaattgtgttaattaatcaaagctataaataacgctcgcatgcctgtgcacttctccatcaccaccactgggtcttcagaccattagctttatctactccagagcgcagaagaacccgatcgacaccggatcctaaaccatgagggtgttgctcgttgccctcgctctcctggctctcgctgcgagcgccacctccggctgccagagcgtgagcgagatgctgaggttctacaccgacgaggtgctgccgagggccatgcagggcggcggcagcggcggcggcagcaaggccatgggcgagttcgacatcttcatcaactacatcgaggagtacctgctgatgaggaaggacgagctgtaac

SEQ ID NO: 77 - DNA, 12533 bp, synthetic construct
  misc_feature 1..12533: note = Synthetic construct, pAG4982
  source 1..12533: mol_type = other DNA
ctaggtccccgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattggaattcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcacggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagatccagatctaaaccatgcagaaactcattaactcagtgcaaaactatgcctggggcagcaaaacggcgttgactgaactttatggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggatgattttgccttctcgctgcatgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaagagcttactgaaaaaattaacatctcttgctaagctgggagctctagatccccgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattggcgagctcgaattaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaaatatcatctccctcgcagagatccgaattatcagccttcttattcatttctcgcttaaccgtgacaggctgtcgatcttgagaactatgccgacataataggaaatcgctggataaagccgctgaggaagctgagtggcgctatttctttagaagtgaacgttgacgatcgtcgaccgtaccccgatgaattaattcggacgtacgttctgaacacagctggatacttacttgggcgattgtcatacatgacatcaacaatgtacccgtttgtgtaaccgtctcttggaggttcgtatgacactagtggttcccctcagcttgcgactagatgttgaggcctaacattttattagagagcaggctagttgcttagatacatgatcttcaggccgttatctgtcagggcaagcgaaaattggccatttatgacgaccaatgccccgcagaagctcccatctttgccgccatagacgccgcgccccccttttggggtgtagaacatccttttgccagatgtggaaaagaagttcgttgtcccattgttggcaatgacgtagtagccggcgaaagtgcgagacccatttgcgctatatataagcctacgatttccgttgcgactattgtcgtaattggatgaactattatcgtagttgctctcagagttgtcgtaatttgatggactattgtcgtaattgcttatggagttgtcgtagttgcttggagaaatgtcgtagttggatggggagtagtcatagggaagacgagcttcatccactaaaacaattggcaggtcagcaagtgcctgccccgatgccatcgcaagtacgaggcttagaaccaccttcaacagatcgcgcatagtcttccccagctctctaacgcttgagttaagccgcgccgcgaagcggcgtcggcttgaacgaattgttagacattatttgccgactaccttggtgatctcgcctttcacgtagtgaacaaattcttccaactgatctgcgcgcgaggccaagcgatcttcttgtccaagataagcctgcctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatgatgtttaactcctgaattaagccgcgccgcgaagcggtgtcggcttgaatgaattgttaggcgtcatcctgtgctcccgagaaccagtaccagtacatcgctgtttcgttcgagacttgaggtctagttttatacgtgaacaggtcaatgccgccgagagtaaagccacattttgcgtacaaattgcaggcaggtacattgttcgtttgtgtctctaatcgtatgccaaggagctgtctgcttagtgcccactttttcgcaaattcgatgagactgtgcgcgactcctttgcctcggtgcgtgtgcgacacaacaatgtgttcgatagaggctagatcgttccatgttgagttgagttcaatcttcccgacaagctcttggtcgatgaatgcgccatagcaagcagagtcttcatcagagtcatcatccgagatgtaatccttccggtaggggctcacacttctggtagatagttcaaagccttggtcggataggtgcacatcgaacacttcacgaacaatgaaatggttctcagcatccaatgtttccgccacctgctcagggatcaccgaaatcttcatatgacgcctaacgcctggcacagcggatcgcaaacctggcgcggcttttggcacaaaaggcgtgacaggtttgcgaatccgttgctgccacttgttaacccttttgccagatttggtaactataatttatgttagaggcgaagtcttgggtaaaaactggcctaaaattgctggggatttcaggaaagtaaacatcaccttccggctcgatgtctattgtagatatatgtagtgtatctacttgatcgggggatctgctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggggggggggggggggggttccattgttcattccacggacaaaaacagagaaaggaaacgacagaggccaaaaagctcgctttcagcacctgtcgtttcctttcttttcagagggtattttaaataaaaacattaagttatgacgaagaagaacggaaacgccttaaaccggaaaattttcataaatagcgaaaacccgcgaggtcgccgccccgtaacctgtcggatcaccggaaaggacccgtaaagtgataatgattatcatctacatatcacaacgtgcgtggaggccatcaaaccacgtcaaataatcaattatgacgcaggtatcgtattaattgatctgcatcaacttaacgtaaaaacaacttcagacaatacaaatcagcgacactgaatacggggcaacctcatgtccccccccccccccccctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattggtcgacgatcttgctgcgttcggatattttcgtggagttcccgccacagacccggattgaaggcgagatccagcaactcgcgccagatcatcctgtgacggaactttggcgcgtgatgactggccaggacgtcggccgaaagagcgacaagcagatcacgcttttcgacagcgtcggatttgcgatcgaggatttttcggcgctgcgctacgtccgcgaccgcgttgagggatcaagccacagcagcccactcgaccttctagccgacccagacgagccaagggatctttttggaatgctgctccgtcgtcaggctttccgacgtttgggtggttgaacagaagtcattatcgcacggaatgccaagcactcccgaggggaaccctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaacctgatcatgagcggagaattaagggagtcacgttatgacccccgccgatgacgcgggacaagccgttttacgtttggaactgacagaaccgcaacgttgaaggagccactcagcctaagcggccgcattggacttaattaagtgaggccggccaagcgtcgatttaaatgtaccacatggcgcgccaactatcatgcgatcgcttcatgtctaactcgagttactggtacgtaccaaatccatggaatcaaggtacctccatgctgtcctactacttgcttcatccccttctacattttgttctggtttttggcctgcatttcggatcatgatgtatgtgatttccaatctgctgcaatatgaatggagactctgtgctaaccatcaacaacatgaaatgcttatgaggcctttgctgagcagccaatcttgcctgtgtttatgtcttcacaggccgaattcctctgttttgtttttcaccctcaatatttggaaacatttatctaggttgtttgtgtccaggcctataaatcatacatgatgttgtcgtattggatgtgaatgtggtggcgtgttcagtgccttggatttgagtttgatgagagttgcttctgggtcaccactcaccattatcgatgctcctcttcagcataaggtaaaagtcttccctgtttacgttattttacccactatggttgcttgggttggttttttcctgattgcttatgccatggaaagtcatttgatatgttgaacttgaattaactgtagaattgtatacatgttccatttgtgttgtacttccttcttttctattagtagcctcagatgagtgtgaaaaaaacagattatataacttgccctataaatcatttgaaaaaaatattgtacagtgagaaattgatatatagtgaatttttaagagcatgttttcctaaagaagtatatattttctatgtacaaaggccattgaagtaattgtagatacaggataatgtagactttttggacttacactgctacctttaagtaacaatcatgagcaatagtgttgcaatgatatttaggctgcattcgtttactctcttgatttccatgagcacgcttcccaaactgttaaactctgtgttttttgccaaaaaaaaatgcataggaaagttgcttttaaaaaatcatatcaatccattttttaagttatagctaatacttaattaatcatgcgctaataagtcactctgtttttcgtactagagagattgttttgaaccagcactcaagaacacagccttaacccagccaaataatgctacaacctaccagtccacacctcttgtaaagcatttgttgcatggaaaagctaagatgacagcaacctgttcaggaaaacaactgacaaggtcatagggagagggagcttttggaaaggtgccgtgcagttcaaacaattagttagcagtagggtgttggtttttgctcacagcaataagaagttaatcatggtgtaggcaacccaaataaaacaccaaaatatgcacaaggcagtttgttgtattctgtagtacagacaaaactaaaagtaatgaaagaagatgtggtgttagaaaaggaaacaatatcatgagtaatgtgtgggcattatgggaccacgaaataaaaagaacattttgatgagtcgtgtatcctcgatgagcctcaaaagttctctcaccccggataagaaacccttaagcaatgtgcaaagtttgcattctccactgacataatgcaaaataagatatcatcgatgacatagcaactcatgcatcatatcatgcctctctcaacctattcattcctactcatctacataagtatcttcagctaaatgttagaacataaacccataagtcacgtttgatgagtattaggcgtgacacatgacaaatcacagactcaagcaagataaagcaaaatgatgtgtacataaaactccagagctatatgtcatattgcaaaaagaggagagcttataagacaaggcatgactcacaaaaattcatttgcctttcgtgtcaaaaagaggagggctttacattatccatgtcatattgcaaaagaaagagagaaagaacaacacaatgctgcgtcaattatacatatctgtatgtccatcattattcatccacctttcgtgtaccacacttcatatatcatgagtcacttcatgtctggacattaacaaactctatcttaacatttagatgcaagagcctttatctcactataaatgcacgatgatttctcattgtttctcacaaaaagcattcagttcattagtcctacaacaacggatcctaaaccatgagggtgttgctcgttgccctcgctctcctggctctcgctgcgagcgccacctccggctgccagagcgtgagcgagatgctgaggttctacaccgacgaggtgctgccgagggccatgcagggcggcggcagcggcggcggcagcaaggccatgggcgagttcgacatcttcatcaactacatcgaggagtacctgctgatgaggaaggacgagctgtaac

SEQ ID NO: 78 - DNA, 13466 bp, synthetic construct
  misc_feature 1..13466: note = Synthetic construct, pAG4983
  source 1..13466: mol_type = other DNA
ctaggtccccgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattggaattcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcacggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagatccagatctaaaccatgcagaaactcattaactcagtgcaaaactatgcctggggcagcaaaacggcgttgactgaactttatggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggatgattttgccttctcgctgcatgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaagagcttactgaaaaaattaacatctcttgctaagctgggagctctagatccccgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattggcgagctcgaattaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaaatatcatctccctcgcagagatccgaattatcagccttcttattcatttctcgcttaaccgtgacaggctgtcgatcttgagaactatgccgacataataggaaatcgctggataaagccgctgaggaagctgagtggcgctatttctttagaagtgaacgttgacgatcgtcgaccgtaccccgatgaattaattcggacgtacgttctgaacacagctggatacttacttgggcgattgtcatacatgacatcaacaatgtacccgtttgtgtaaccgtctcttggaggttcgtatgacactagtggttcccctcagcttgcgactagatgttgaggcctaacattttattagagagcaggctagttgcttagatacatgatcttcaggccgttatctgtcagggcaagcgaaaattggccatttatgacgaccaatgccccgcagaagctcccatctttgccgccatagacgccgcgccccccttttggggtgtagaacatccttttgccagatgtggaaaagaagttcgttgtcccattgttggcaatgacgtagtagccggcgaaagtgcgagacccatttgcgctatatataagcctacgatttccgttgcgactattgtcgtaattggatgaactattatcgtagttgctctcagagttgtcgtaatttgatggactattgtcgtaattgcttatggagttgtcgtagttgcttggagaaatgtcgtagttggatggggagtagtcatagggaagacgagcttcatccactaaaacaattggcaggtcagcaagtgcctgccccgatgccatcgcaagtacgaggcttagaaccaccttcaacagatcgcgcatagtcttccccagctctctaacgcttgagttaagccgcgccgcgaagcggcgtcggcttgaacgaattgttagacattatttgccgactaccttggtgatctcgcctttcacgtagtgaacaaattcttccaactgatctgcgcgcgaggccaagcgatcttcttgtccaagataagcctgcctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatgatgtttaactcctgaattaagccgcgccgcgaagcggtgtcggcttgaatgaattgttaggcgtcatcctgtgctcccgagaaccagtaccagtacatcgctgtttcgttcgagacttgaggtctagttttatacgtgaacaggtcaatgccgccgagagtaaagccacattttgcgtacaaattgcaggcaggtacattgttcgtttgtgtctctaatcgtatgccaaggagctgtctgcttagtgcccactttttcgcaaattcgatgagactgtgcgcgactcctttgcctcggtgcgtgtgcgacacaacaatgtgttcgatagaggctagatcgttccatgttgagttgagttcaatcttcccgacaagctcttggtcgatgaatgcgccatagcaagcagagtcttcatcagagtcatcatccgagatgtaatccttccggtaggggctcacacttctggtagatagttcaaagccttggtcggataggtgcacatcgaacacttcacgaacaatgaaatggttctcagcatccaatgtttccgccacctgctcagggatcaccgaaatcttcatatgacgcctaacgcctggcacagcggatcgcaaacctggcgcggcttttggcacaaaaggcgtgacaggtttgcgaatccgttgctgccacttgttaacccttttgccagatttggtaactataatttatgttagaggcgaagtcttgggtaaaaactggcctaaaattgctggggatttcaggaaagtaaacatcaccttccggctcgatgtctattgtagatatatgtagtgtatctacttgatcgggggatctgctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggggggggggggggggggttccattgttcattccacggacaaaaacagagaaaggaaacgacagaggccaaaaagctcgctttcagcacctgtcgtttcctttcttttcagagggtattttaaataaaaacattaagttatgacgaagaagaacggaaacgccttaaaccggaaaattttcataaatagcgaaaacccgcgaggtcgccgccccgtaacctgtcggatcaccggaaaggacccgtaaagtgataatgattatcatctacatatcacaacgtgcgtggaggccatcaaaccacgtcaaataatcaattatgacgcaggtatcgtattaattgatctgcatcaacttaacgtaaaaacaacttcagacaatacaaatcagcgacactgaatacggggcaacctcatgtccccccccccccccccctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattggtcgacgatcttgctgcgttcggatattttcgtggagttcccgccacagacccggattgaaggcgagatccagcaactcgcgccagatcatcctgtgacggaactttggcgcgtgatgactggccaggacgtcggccgaaagagcgacaagcagatcacgcttttcgacagcgtcggatttgcgatcgaggatttttcggcgctgcgctacgtccgcgaccgcgttgagggatcaagccacagcagcccactcgaccttctagccgacccagacgagccaagggatctttttggaatgctgctccgtcgtcaggctttccgacgtttgggtggttgaacagaagtcattatcgcacggaatgccaagcactcccgaggggaaccctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaacctgatcatgagcggagaattaagggagtcacgttatgacccccgccgatgacgcgggacaagccgttttacgtttggaactgacagaaccgcaacgttgaaggagccactcagcctaagcggccgcattggacttaattaagtgaggccggccaagcgtcgatttaaatgtaccacatggcgcgccaactatcatgcgatcgcttcatgtctaactcgagttactggtacgtaccaaatccatggaatcaaggtacccggtatgaatttggaaacaaattcagtacttttaaaaaaatttgttgtagggagcaaataatacataaaataatttatgcattattttattttttatttgtaataatatgcttgaaacgataattcagtatgcatgttgtgccagtgtactacacgggcggggggaggggattgagtgggccagcgcggtgcgtagggtagatgggctgaaattgataactcaagtccgactaggttctctttttatttcccttccttttctattttcctttcttttaattttcatgctttcaaactaaattcaaattcgagttttgaatttcagcttctaaattgtacactaaaattatatgataaggtaacccctactattacttttaatttttttattctaccccatattgtttacttaggggagaataattgacttaatcacattcttccttaggtttcaattctcaatctttcaaatccacatttttagatttctattttgaatttaaataccagtttggatttagagttcaatttcaaaatacacaaccaaaataccagcatgaatgcaaatatattttatgtttatgtatttacttttcttttatactttgctcaaaatagttattttcatgtatgaaactcaataagcaaggaactcacgttattatataacctaataggaataatttaggtaacataatttatcatcctcttgatttaaaagagatatgcctccagaataagacacatactaaaaataactctaatattgaataactaaagtcgtacaaatctctactattattcctataaaataataaagaactagctacaacttctttaaggcattattcagggtttacagcttgagaggcatgaacccatcctgtatactcctggacttggaagacaaaatgtcaaccaaagtgaaaggttttcttatggttgctgctaagagatagattgaacactagatctctcctaagacgtcagggcatgcgtttagactcctacacatgcgaaaactgcatcttacagttggaagaaactatatctcaccacttcctgcggtgtaactttgcccaaagatgttggctcactgttggaatcactccgccccgaactttggatctaacgcttgcagtgctacatattagagcaagactaacaatgccgtggagaatggaaggtattataaccatgtcatggtgcatatggaaatgtcgaaataactggatattcgaaaacataccgccaacggtggcggcctgcaaggaaatgttcaagactgaaatgaactacatctgctaccaagttaagctcgagacaggagctaaaagtagaaactggatacaacactttgtaacatagtgacactccccttttcctttcttttaccttagaactatacatacaatccacattcaataaaaatttgtaggtacgccatacacactaccggaatccggctctttgccgagtgtgaggcgctttgtcgagtgctttttgtccagcactcggcaaaaaagtctttgccatgtgccgcactcggcaaagtcctgctctcggtaacgaccgcgtttaccgagagcaggactctcgacacagaaatacactcgacaaagaaatctttgccgagagccaaacactcggcgaacggcagcgctcggcaaagggtcgtcagccgccgtctaaagctgacggtcgttatctttgtcgagtgccccctcgtccgacactcagtagagcaagcttgccgagtgccatccttggacactcgataaagtatattttatttttttttattttgccaaccaaactttttgtggtatgttcctacactatgtagatctacatgtaccattttggcacaattacaaaaatgttttctataactattagatttagttcgtttatttgaatttcttcggaaaattcacatatgaactgcaagtcactcgaaacatgaaaaaccgtgcatgcaaaataaatgatatgcatgttatctagcacaagttacgaccgaattcagaagcagaccagaatcttcaagcaccatgctcactaaacatgaccgtgaacttgttatccagttgtttaaaaattgtataaaacacaaataaagtcagaaattaatgaaacttgtccacatgtcatgatatcatatatagaggttgtgataaaaatttgataatgtttcggtaaagttgtgacgtactatgtgtagaaacctaagtgacctacacataaaatcatagagtttcaatgtagttcactcgacaaagactttgtcaagtgtccgataaaaagtattcagcaaagaagccgttgtcgatttactgttcgtcgagatctctttgccgagtgtcacactaggcaaagtctttacggagtgtttttcaggctttgacactcggcaaagcgctcgattccagtagtgacagtaatttgcatcaaaaatagccgagagatttaaaatgagtcaactaatagaccaactaattattagctattagtcgttagcttctttaatctaagctaaaaccaactaatagcttatttgttgaattacaattagctcaacggaattctctgttttttctataaaaaaaagggaaactgcccctcatttacagcaaactgtccgctgcctgtcgtccagatacaatgaacgtacctagtaggaactcttttacacgctcggtcgctcgccgcggatcggagtcccaggaacacgacaccactgtggaacacgacaaagtctgctcagaggcggccacaccctggcgtgcaccgagccggagcccggataagcacggtaaggagagtacggcgggacgtggcgacccgtgtgtctgctgccacgcagccttcctccacgtagccgcgcggccgcgccacgtaccagggcccggcgctggtataaatgcgcgccacctccgctttagttctgcatacagccaacccaacacacacccgagcatatcacagtgacagacactacacgggatcctaaaccatgagggtgttgctcgttgccctcgctctcctggctctcgctgcgagcgccacctccggctgccagagcgtgagcgagatgctgaggttctacaccgacgaggtgctgccgagggccatgcagggcggcggcagcggcggcggcagcaaggccatgggcgagttcgacatcttcatcaactacatcgaggagtacctgctgatgaggaaggacgagctgtaac

SEQ ID NO: 79 - DNA, 12474 bp, synthetic construct
  misc_feature 1..12474: note = Synthetic construct, pAG4984
  source 1..12474: mol_type = other DNA
ctaggtccccgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattggaattcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcacggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagatccagatctaaaccatgcagaaactcattaactcagtgcaaaactatgcctggggcagcaaaacggcgttgactgaactttatggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggatgattttgccttctcgctgcatgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaagagcttactgaaaaaattaacatctcttgctaagctgggagctctagatccccgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattggcgagctcgaattaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaaatatcatctccctcgcagagatccgaattatcagccttcttattcatttctcgcttaaccgtgacaggctgtcgatcttgagaactatgccgacataataggaaatcgctggataaagccgctgaggaagctgagtggcgctatttctttagaagtgaacgttgacgatcgtcgaccgtaccccgatgaattaattcggacgtacgttctgaacacagctggatacttacttgggcgattgtcatacatgacatcaacaatgtacccgtttgtgtaaccgtctcttggaggttcgtatgacactagtggttcccctcagcttgcgactagatgttgaggcctaacattttattagagagcaggctagttgcttagatacatgatcttcaggccgttatctgtcagggcaagcgaaaattggccatttatgacgaccaatgccccgcagaagctcccatctttgccgccatagacgccgcgccccccttttggggtgtagaacatccttttgccagatgtggaaaagaagttcgttgtcccattgttggcaatgacgtagtagccggcgaaagtgcgagacccatttgcgctatatataagcctacgatttccgttgcgactattgtcgtaattggatgaactattatcgtagttgctctcagagttgtcgtaatttgatggactattgtcgtaattgcttatggagttgtcgtagttgcttggagaaatgtcgtagttggatggggagtagtcatagggaagacgagcttcatccactaaaacaattggcaggtcagcaagtgcctgccccgatgccatcgcaagtacgaggcttagaaccaccttcaacagatcgcgcatagtcttccccagctctctaacgcttgagttaagccgcgccgcgaagcggcgtcggcttgaacgaattgttagacattatttgccgactaccttggtgatctcgcctttcacgtagtgaacaaattcttccaactgatctgcgcgcgaggccaagcgatcttcttgtccaagataagcctgcctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatgatgtttaactcctgaattaagccgcgccgcgaagcggtgtcggcttgaatgaattgttaggcgtcatcctgtgctcccgagaaccagtaccagtacatcgctgtttcgttcgagacttgaggtctagttttatacgtgaacaggtcaatgccgccgagagtaaagccacattttgcgtacaaattgcaggcaggtacattgttcgtttgtgtctctaatcgtatgccaaggagctgtctgcttagtgcccactttttcgcaaattcgatgagactgtgcgcgactcctttgcctcggtgcgtgtgcgacacaacaatgtgttcgatagaggctagatcgttccatgttgagttgagttcaatcttcccgacaagctcttggtcgatgaatgcgccatagcaagcagagtcttcatcagagtcatcatccgagatgtaatccttccggtaggggctcacacttctggtagatagttcaaagccttggtcggataggtgcacatcgaacacttcacgaacaatgaaatggttctcagcatccaatgtttccgccacctgctcagggatcaccgaaatcttcatatgacgcctaacgcctggcacagcggatcgcaaacctggcgcggcttttggcacaaaaggcgtgacaggtttgcgaatccgttgctgccacttgttaacccttttgccagatttggtaactataatttatgttagaggcgaagtcttgggtaaaaactggcctaaaattgctggggatttcaggaaagtaaacatcaccttccggctcgatgtctattgtagatatatgtagtgtatctacttgatcgggggatctgctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggggggggggggggggggttccattgttcattccacggacaaaaacagagaaaggaaacgacagaggccaaaaagctcgctttcagcacctgtcgtttcctttcttttcagagggtattttaaataaaaacattaagttatgacgaagaagaacggaaacgccttaaaccggaaaattttcataaatagcgaaaacccgcgaggtcgccgccccgtaacctgtcggatcaccggaaaggacccgtaaagtgataatgattatcatctacatatcacaacgtgcgtggaggccatcaaaccacgtcaaataatcaattatgacgcaggtatcgtattaattgatctgcatcaacttaacgtaaaaacaacttcagacaatacaaatcagcgacactgaatacggggcaacctcatgtccccccccccccccccctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattggtcgacgatcttgctgcgttcggatattttcgtggagttcccgccacagacccggattgaaggcgagatccagcaactcgcgccagatcatcctgtgacggaactttggcgcgtgatgactggccaggacgtcggccgaaagagcgacaagcagatcacgcttttcgacagcgtcggatttgcgatcgaggatttttcggcgctgcgctacgtccgcgaccgcgttgagggatcaagccacagcagcccactcgaccttctagccgacccagacgagccaagggatctttttggaatgctgctccgtcgtcaggctttccgacgtttgggtggttgaacagaagtcattatcgcacggaatgccaagcactcccgaggggaaccctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaacctgatcatgagcggagaattaagggagtcacgttatgacccccgccgatgacgcgggacaagccgttttacgtttggaactgacagaaccgcaacgttgaaggagccactcagcctaagcggccgcattggacttaattaagtgaggccggccaagcgtcgatttaaatgtaccacatggcgcgccaactatcatgcgatcgcttcatgtctaactcgagttactggtacgtaccaaatccatggaatcaaggtaccatcaatcccgggataaaattttagtgaagctaaagcggtgaaagattatagaatttgatgtgccagattaataaatcgattaactcctaaagttcaagccgagactacagacacatgagctacataaatgagccaaggactcgagcaaagacaaatcgacacagacattataattcaagtcattctagaagattcatgagaagagtatcatttatttaaatcaatgacttgatcaaataagacctaggagctactattgataatatatatcatgggtatctagatcaagcattatgaagaagagcctaagtagaaggccccatgggctcgaccacaaacccaaggactcgacaataaagtctaggagggatcccatagctaaaaggactctagaagtgtatgtatggtaaagattttatcgagacaagaaatacgataaagatcttaacagaatcggagtcatacttgtaaaaatagagttggactcgtgtacaacttggtcttcgacttagttcggtcatgaattcagtaaccgactagatatgtaccatggaacccctagggcatgaggctatgagccataggatcatcagatccaaacatacaccaacaaatccatcacacaccgaagatccatattaacaagggattagctactttacaatttcagagtaacaaatagagccaaactcatagcacaggggaacttcatatcacaaatggaggcattgaattgatataaaaagctaaagttctaaaaagtttgaagtgctgaaacttcaaagccgctaactagtgaagcaccgaagccttccggggagagaagacatacacgacacgttagggacgtaaaatgacgaaattatacaactacctctatatgtaacacttatgtaatagaaaagacagaatccatatgaagatgtataatggatcaaccatataaatagataaacaatatatctgctatggggattggcattcttgtatccctacgcctgtatatcccctgtttagagaacctccgaaggtatatgatgctgaagattattgttgtcttgtctttcatcatatatcgagtctttccctaggatattattattcgcaatgtgcattacatggttaatcgattgagagaacatgcatctcacctttagctgataaacgataatccatgttttacacttcgtagctactcatgagtttcgatatacaaatttgttttctggactacgtaccattccatcctcttaggagaggagaggaagtgtcctcgatttaattatgttgtcattttgtagttcttcacaaaatctcaacaggtaccaaacacattgtttccacaagacatattttagtcacaacaaatctatattattattaatcactaaaactatactgaggctcagatgcttttactagctcttgctagtatgtgatgtaggtctctttcgacatcattccatcaaaatcatatgattagcccataccaaacatttctataccattcagagaccagaatagtcttttctaatagaaaaaaggaaaatagagtgggccgacgacgacacaaattactgcgtggaccagaaaatagtgagacacggaagacaaaagaagtaaaagaggcaaggactacggcccacatgagattcggccccgccacctccggcaaccagcggccgatccaacggcagtgcatcctcaacggcgcgcgcgcgcgcgcgcgcgcacaacctcgtatatatcgccgcgcggaagcggcgcgaccgaggaagccttgtcctcgacaccccctacacaggtgtcgcgctgcccccgacacgagtcccgcatgcgtcccacgcggccgcgccagatcccgcctccgcgcgttgccacgccctctataaacacccagctctctccctcgccctcatctatcgcactcgtagtcgtagctcaagcatcagcggcaggagctctgggcagcgtgcgcacgtggggtacctagctcgctctgctagcctaccggatcctaaaccatgagggtgttgctcgttgccctcgctctcctggctctcgctgcgagcgccacctccggctgccagagcgtgagcgagatgctgaggttctacaccgacgaggtgctgccgagggccatgcagggcggcggcagcggcggcggcagcaaggccatgggcgagttcgacatcttcatcaactacatcgaggagtacctgctgatgaggaaggacgagctgtaac

SEQ ID NO: 80 - protein, 153 aa, Gallus gallus
  REGION 1..153: note = MISC_FEATURE - chicken IL-10
LEPTCLHFSELLPARLRELRVKFEEIKDYFQSRDDELNIQLLSSELLDEFKGTFGCQSVSEMLRFYTDEVLPRAMQTSTSHQQSMGDLGNMLLGLKATMRRCHRFFTCEKRSKAIKQIKETFEKMDENGIYKAMGEFDIFINYIEEYLLMRRR

SEQ ID NO: 81 - protein, 160 aa, Homo sapiens
  REGION 1..160: note = MISC_FEATURE - IL-10
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 82 - protein, 233 aa, Homo sapiens
  REGION 1..233: note = MISC_FEATURE - IL-10R1
  REGION 1..233: note = MISC_FEATURE - human IL-10R1
HGTELPSPPSVWFEAEFFHHILHWTPIPNQSESTCYEVALLRYGIESWNSISNCSQTLSYDLTAVTLDLYHSNGYRARVRAVDGSRHSNWTVTNTRFSVDEVTLTVGSVNLEIHNGFILGKIQLPRPKMAPANDTYESIFSHFREYEIAIRKVPGNFTFTHKKVKHENFSLLTSGEVGEFCVQVKPSVASRSNKGMWSKEECISLTRQYFTVTNVIIFFAFVLLLSGALAYCL

SEQ ID NO: 83 - protein, 210 aa, Gallus gallus
  REGION 1..210: note = MISC_FEATURE - huan IL-10R1
  REGION 1..210: note = MISC_FEATURE - chicken IL-10R1
ELRLKPTRVRFVAEMVYHLLQWEPGRDAPSDTRYDVEHKIYGTNSPWTAIPNCMKIHGHSCDLTYYTLDPSLRYYARVRAVVGNHTSDWKRTNAFSPQEASLRLSGHSLAVTDNSIHVQLQLLLRAGNRTVKYDDIQKHARRYRVYIRRARDNQTYEVWETASEFYIRNLFWNTEYCISVEPDVASRHIPAMRTAEQCVTIGHRDESAEL

SEQ ID NO: 84 - protein, 147 aa, synthetic construct
  REGION 1..147: note = Synthetic construct, xGZein27ss:chIL10sdAb1A11:KDEL
MRVLLVALALLALAASATSQVQLQESGGGLVQPGGSLRLSCAASGNIFSINTMGWYRQAPGKQRELVASITTGGTTNYEDSVKGRFTISRDNAKKTVYLQMNRLKPEDTAVYYCNHRRSYSGRDYPVYGMDYWGKGTLVTVSSKDEL

SEQ ID NO: 85 - protein, 147 aa, synthetic construct
  REGION 1..147: note = Synthetic construct, xGZein27ss:chIL10sdAb1B9:KDEL
MRVLLVALALLALAASATSQVQLQESGGGLVQAGGSLRLSCAASGRTFSSYAWGWFRQAPGKEREFVARISFSGGHTYYSDSVKGRFTISRDNAKNTVYLQMNSLKPEDTAVYYCAADPTPYGLRNERNYPYWGQGTQVTVSSKDEL

SEQ ID NO: 86 - protein, 148 aa, synthetic construct
  REGION 1..148: note = Synthetic construct, xGZein27ss:chIL10sdAb1F11:KDEL
MRVLLVALALLALAASATSQVQLQEFGGGLVQPGGSLRLSCAASGRTGSSYAMGWFRQAPGKEREFVAAISWSGGSTDYADSVKGRFTISRDNAKNTMYLQMNSLKPEDTAVYYCAVDRNLFKLRVAVQEYTNLGQGTQVTVSSKDEL

SEQ ID NO: 87 - protein, 125 aa, synthetic construct
  REGION 1..125: note = Synthetic construct, chIL10sdAB1H5
MQVQLQESGGGLVQAGGSLRLSCAASGRTFNSYAWGWFRQAPGKERGFVARISFSGGHTYYSDSVKGRFTISRDNAKNSVYLQMNSLKPEDTAVYYCAADPTPYGLRNERNYHYWGQGTQVTVSS

SEQ ID NO: 88 - protein, 125 aa, synthetic construct
  REGION 1..125: note = Synthetic construct, chIL10sdAB1E9
MQVQLQASGGGLVQAGGSLRLSCAASGRTFNSYAWGWFRQAPGKERGFVARITFSGGHTYYSDSVKGRFTISRDNAKNSVYLQMNSLKPEDTAVYYCAADPTPYGLRNERNYHYWGQGTQVTVSS

SEQ ID NO: 89 - protein, 125 aa, synthetic construct
  REGION 1..125: note = Synthetic construct, chIL10sdAB1H1
MQVQLQASGGGLVQAGGSLRLSCAASGRTFNSYAWGWFRQAPGKERGFVARISFSGGHTYYSDSVKGRFTISRDNAKNSVYLQMNSLKPEDTAVYYCAADPTPYGLRNERNYHYWGQGTQVTVSS

SEQ ID NO: 90 - protein, 125 aa, synthetic construct; identical to SEQ ID NO: 89
  REGION 1..125: note = Synthetic construct, chIL10sdAB1G6

SEQ ID NO: 91 - protein, 125 aa, synthetic construct
  REGION 1..125: note = Synthetic construct, chIL10sdAB1C10
MQVQLQEFGGGLVLPGGSLRLSCAASGRTFNSYAWGWFRQAPGKERGFVARISFSGGHTYYSDSVKGRFTISRDNAKNSVYLQMNSLKPEDTAVYYCAADPTPYGLRNERNYHYWGQGTQVTVSS

SEQ ID NO: 92 - protein, 125 aa, synthetic construct
  REGION 1..125: note = Synthetic construct, chIL10sdAB1B6
MQVQLQQSGGGLVQAGGSLRLSCAASGRTFNSYAWGWFRQAPGKERGFVARISFSGGHTYYSDSVKGRFTISRDNAKNSVYLQMNSLKPEDTAVYYCAADPTPYGLRNERNYHYWGQGTQVTVSS

SEQ ID NO: 93 - protein, 125 aa, synthetic construct; identical to SEQ ID NO: 92
  REGION 1..125: note = Synthetic construct, chIL10sdAB1D12

SEQ ID NO: 94 - protein, 125 aa, synthetic construct
  REGION 1..125: note = Synthetic construct, chIL10sdAB1C2
MQVQLQEFGGGLVQAGGSLRLSCAASGRTFNSYAWGWFRQAPGKERGFVARISFSGGHTYYSDSVKGRFTISRDNAKNSVYLQMNSLKPEDTAVYYCAADPTPYGLRNERNYHYWGQGTQVTVSS

SEQ ID NO: 95 - protein, 125 aa, synthetic construct; identical to SEQ ID NO: 94
  REGION 1..125: note = Synthetic construct, chIL10sdAB1B5

SEQ ID NO: 96 - protein, 125 aa, synthetic construct; identical to SEQ ID NO: 87
  REGION 1..125: note = Synthetic construct, chIL10sdAB1E2

SEQ ID NO: 97 - protein, 125 aa, synthetic construct; identical to SEQ ID NO: 87
  REGION 1..125: note = Synthetic construct, chIL10sdAB1G7

SEQ ID NO: 98 - protein, 125 aa, synthetic construct; identical to SEQ ID NO: 87
  REGION 1..125: note = Synthetic construct, chIL10sdAB1G9

SEQ ID NO: 99 - protein, 125 aa, synthetic construct; identical to SEQ ID NO: 87
  REGION 1..125: note = Synthetic construct, chIL10sdAB1H12

SEQ ID NO: 100 - protein, 125 aa, synthetic construct; identical to SEQ ID NO: 87
  REGION 1..125: note = Synthetic construct, chIL10sdAB2A9

SEQ ID NO: 101 - protein, 125 aa, synthetic construct; identical to SEQ ID NO: 87
  REGION 1..125: note = Synthetic construct, chIL10sdAB1E12

SEQ ID NO: 102 - protein, 125 aa, synthetic construct
  REGION 1..125: note = Synthetic construct, chIL10sdAB1E10
MQVQLQEFGGGLVQAGGSLRLSCAASGRTFNSYAWGWFRQAPGKERGFVARISFSGGHTYYSDSVKGRFTISRDNAKNSVYLQMNSLKPEDTAVYYCAADPTPYGLRNERNYHYWGQGTLVTVSS

SEQ ID NO: 103 - protein, 125 aa, synthetic construct
  REGION 1..125: note = Synthetic construct, chIL10sdAB1F12
MQVQLQESGGGLVQAGGSLRLSCAASGRTFNSYAWGWFRQAPGKERGFVARISFSGGHTYYSDSVKGRFTISRDNAKNSVYLQMNSLKPEDTAVYYCAADPTPYGLRNERNYHYWGQGTLVTVSS

SEQ ID NO: 104 - protein, 125 aa, synthetic construct; identical to SEQ ID NO: 103
  REGION 1..125: note = Synthetic construct, chIL10sdAB1A8

SEQ ID NO: 105 - protein, 125 aa, synthetic construct
  REGION 1..125: note = Synthetic construct, chIL10sdAB1C8
MQVQLQESGGGLVQPGGSLRLSCAASGRTFNSYAWGWFRQAPGKERGFVARISFSGGHTYYSDSVKGRFTISRDNAKNSVYLQMNSLKPEDTAVYYCAADPTPYGLRNERNYHYWGQGTLVTVSS

SEQ ID NO: 106 - protein, 125 aa, synthetic construct; identical to SEQ ID NO: 103
  REGION 1..125: note = Synthetic construct, chIL10sdAB1C12

SEQ ID NO: 107 - protein, 125 aa, synthetic construct
  REGION 1..125: note = Synthetic construct, chIL10sdAB1B1
MQVQLQASGGGLVQAGGSLRLSCAASGRNFNSYAWGWFRQAPGKERGFVARISFSGGHTYYSDSVKGRFTISRDNAKNSVYLQMNSLKPEDTAVYYCAADPTPYGLRNERNYHYWGQGTLVTVSS

SEQ ID NO: 108 - protein, 125 aa, synthetic construct
  REGION 1..125: note = Synthetic construct, chIL10sdAB1F1
MQVQLQESGGGLVQAGGSLRLSCAASGRTFNRYAWGWFRQAPGKEREFVARISFSGGNTYYADSVKGRFTISRDNAKNSVYLQMNSLKPEDTAVYYCAADPTPYGLRNERNYHYWGQGTQVTVSS

SEQ ID NO: 109 - protein, 125 aa, synthetic construct
  REGION 1..125: note = Synthetic construct, chIL10sdAB1D11
MQVQLQESGGGLVQAGGSLRLSCAASGRTFSSYAWGWFRQAPGKEREFVARISFSGGHIYYSDSVKGRFTISRDNAKNTVYLQMNSLKPEDTAVYYCAADPTPYGLRNERNYHYWGQGTLVTVSS

SEQ ID NO: 110 - protein, 125 aa, synthetic construct
  REGION 1..125: note = Synthetic construct, chIL10sdAB1E6
MQVQLQESGGGLVQAGGSLRLSCAASGRTFNSYAWGWFRQAPGKERGFVARISFSGGHTYYSDSVKGRFTISRDNAKNSVYLQMNSLKPEDTAVYYCAADPTPYGLRNERNYPYWGQGTLVTVSS

SEQ ID NO: 111 - protein, 125 aa, synthetic construct
  REGION 1..125: note = Synthetic construct, chIL10sdAB1B9
MQVQLQESGGGLVQAGGSLRLSCAASGRTFSSYAWGWFRQAPGKEREFVARISFSGGHTYYSDSVKGRFTISRDNAKNTVYLQMNSLKPEDTAVYYCAADPTPYGLRNERNYPYWGQGTQVTVSS

SEQ ID NO: 112 - protein, 125 aa, synthetic construct
  REGION 1..125: note = Synthetic construct, chIL10sdAB1B10
MQVQLQEFGGGLVQPGGSLRLSCAASGRTFSSYAWGWFRQAPGKEREFVARISFSGGHTYYSDSVKGRFTISRDNAKNTVYLQMNSLKPEDTAVYYCAADPTPYGLRNERNYPYWGQGTQVTVSS

SEQ ID NO: 113 - protein, 125 aa, synthetic construct
  REGION 1..125: note = Synthetic construct, chIL10sdAB1F5
MQVQLQESGGGLVQPGGSLRLSCAASGRTFSSYAWGWFRQAPGKEREFVARISFSGGHTYYSDSVKGRFTISRDNAKNTVYLQMNSLKPEDTAVYYCAADPTPYGLRNERNYPYWGQGTQVTVSS

SEQ ID NO: 114 - protein, 125 aa, synthetic construct
  REGION 1..125: note = Synthetic construct, chIL10sdAB1A6
MQVQLQEFGGGLVQAGGSLRLSCAASGRTFSSYAWGWFRQAPGKEREFVARISFSGGHTYYSDSVKGRFTISRDNAKNTVYLQMNSLKPEDTAVYYCAADPTPYGLRNERNYPYWGQGTQVTVSS

SEQ ID NO: 115 - protein, 125 aa, synthetic construct
  REGION 1..125: note = Synthetic construct, chIL10sdAB1D5
MQVQLQQFGGGLVQAGGSLRLSCAASGRTFSSYAWGWFRQAPGKEREFVARISFSGGHTYYSDSVKGRFTISRDNAKNTVYLQMNSLKPEDTAVYYCAADPTPYGLRNERNYPYWGQGTQVTVSS

SEQ ID NO: 116 - protein, 125 aa, synthetic construct; identical to SEQ ID NO: 115
  REGION 1..125: note = Synthetic construct, chIL10sdAB1D8

SEQ ID NO: 117 - protein, 125 aa, synthetic construct; identical to SEQ ID NO: 111
  REGION 1..125: note = Synthetic construct, chIL10sdAB1B4

SEQ ID NO: 118 - protein, 125 aa, synthetic construct
  REGION 1..125: note = Synthetic construct, chIL10sdAB1C7
MQVQLQAFGGGLVQAGGSLRLSCAASGRTFSSYAWGWFRQAPGKEREFVARISFSGGHTYYSDSVKGRFTISRDNAKNTVYLQMNSLKPEDTAVYYCAADPTPYGLRNERNYPYWGQGTQVTVSS

SEQ ID NO: 119 - protein, 125 aa, synthetic construct
  REGION 1..125: note = Synthetic construct, chIL10sdAB1B3
MQVQLQASGGGLVQAGGSLRLSCAASGRTFSSYAWGWFRQAPGKEREFVARISFSGGHTYYSDSVKGRFTISRDNAKNTVYLQMNSLKPEDTAVYYCAADPTPYGLRNERNYPYWGQGTQVTVSS

SEQ ID NO: 120 - protein, 125 aa, synthetic construct; identical to SEQ ID NO: 111
  REGION 1..125: note = Synthetic construct, chIL10sdAB1D7

SEQ ID NO: 121 - protein, 125 aa, synthetic construct
  REGION 1..125: note = Synthetic construct, chIL10sdAB1F7
MQVQLQEFGGGLVQAGGSLRLSCAASGRTFSSYAWGWFRQAPGKEREFVARISFSGGHTYYSDSVKGRFTISRDNAKNTVYLQMNSLKPEDTAVYYCAADPTPYGLRNERNYPYWGQGTLVTVSS

SEQ ID NO: 122 - protein, 125 aa, synthetic construct
  REGION 1..125: note = Synthetic construct, chIL10sdAB1F10
MQVQLQESGGGLVQAGGSLRLSCAASGRTFSSYAWGWFRQAPGKEREFVARISFSGGHTYYSDSVKGRFTISRDNAKNTVYLQMNSLKPEDTAVYYCAADPTPYGLRNERNYPYWGQGTLVTVSS

SEQ ID NO: 123 - protein, 125 aa, synthetic construct; identical to SEQ ID NO: 122
  REGION 1..125: note = Synthetic construct, chIL10sdAB1F2

SEQ ID NO: 124 - protein, 125 aa, synthetic construct; identical to SEQ ID NO: 122
  REGION 1..125: note = Synthetic construct, chIL10sdAB1F3

SEQ ID NO: 125 - protein, 125 aa, synthetic construct
  REGION 1..125: note = Synthetic construct, chIL10sdAB1F8
MQVQLQASGGGLVQAGGSLRLSCAASGRTFSSYAWGWFRQAPGKEREFVARISFSGGHTYYSDSVKGRFTISRDNAKNTVYLQMNSLKPEDTAVYYCAADPTPYGLRNERNYPYWGQGTLVTVSS

SEQ ID NO: 126 - protein, 125 aa, synthetic construct; identical to SEQ ID NO: 121
  REGION 1..125: note = Synthetic construct, chIL10sdAB1C9

SEQ ID NO: 127 - protein, 125 aa, synthetic construct; identical to SEQ ID NO: 122
  REGION 1..125: note = Synthetic construct, chIL10sdAB1A12

SEQ ID NO: 128 - protein, 125 aa, synthetic construct
  REGION 1..125: note = Synthetic construct, chIL10sdAB1C3
MQVQLQESGGGLVQAGGSLRLSCAASGRTFSSYAWGWFRQAPGKEREFVSRISFSGGHTYYSDSVKGRFTISRDNAKNTVYLQMNSLKPEDTAVYYCAADPTPYGLRNERNYPYWGQGTLVTVSS

SEQ ID NO: 129 - protein, 125 aa, synthetic construct
  REGION 1..125: note = Synthetic construct, chIL10sdAB1E7
MQVQLQEFGGGLVQAGGSLRLSCAASGRTFSTYAWGWFRQAPGKEREFVARISFSGGHTYYSDSVKGRFTISRDNAKNTVYLQMNSLKPEDTAVYYCAADPTPYGLRNERSYPYWGQGTLVTVSS

SEQ ID NO: 130 - protein, 124 aa, synthetic construct
  REGION 1..124: note = Synthetic construct, chIL10sdAB1D9
MQVQLQASGGGLVQAGGSLRLSCAASGRRLSINVMGWYRQAPGKQRELVATIGTSGKTNYADSVRGRFTISRDNAQNTVYLQMNSLKPEDTAVYYCAADPTPYGLRNERNYHYWGQGTQVTVSS

SEQ ID NO: 131 - protein, 125 aa, synthetic construct
  REGION 1..125: note = Synthetic construct, chIL10sdAB1A9
MQVQLQESGGGLVQAGGSLRLSCAASGRTFSRYAWGWFRQAPGKEREFVARISWSGGHTYFADSVKGRFTISRDNAKNTVYLQMNSLKPEDTAVYYCAADPTPYGLKNERNYHYWGQGTQVTVSS

SEQ ID NO: 132 - protein, 125 aa, synthetic construct
  REGION 1..125: note = Synthetic construct, chIL10sdAB1H10
MQVQLQEFGGGLVQAGGSLRLSCAASGRTFSRYAWGWFRQAPGKEREFVARISFNSRSTYYADSVQGRFTISRDNAKNTVYLQMNSLKPEDTAVYYCAADPTPYGLKNERNYDYWGQGTQVTVSS

SEQ ID NO: 133 - protein, 125 aa, synthetic construct
  REGION 1..125: note = Synthetic construct, chIL10sdAB1C1
MQVQLQASGGGLVQPGGSLRLSCAASGNIFSINTMGWYRQAPGKQRELVASITTGGTTNYEDSVKGRFTISRDNAKKTVYLQMNRLKPEDTAVYYCNHRRSYSGRDYPVYGMDYWGKGTQVTVSS

SEQ ID NO: 134 - protein, 125 aa, synthetic construct
  REGION 1..125: note = Synthetic construct, chIL10sdAB1D1
MQVQLQESGGGLVQPGGSLRLSCAASGNIFSINTMGWYRQAPGKQRELVASITTGGTTNYEDSVKGRFTISRDNAKKTVYLQMNRLKPEDTAVYYCNHRRSYSGRDYPVYGMDYWGKGTLVTVSS

SEQ ID NO: 135 - protein, 125 aa, synthetic construct; identical to SEQ ID NO: 134
  REGION 1..125: note = Synthetic construct, chIL10sdAB1A11

SEQ ID NO: 136 - protein, 125 aa, synthetic construct; identical to SEQ ID NO: 134
  REGION 1..125: note = Synthetic construct, chIL10sdAB1G8

SEQ ID NO: 137 - protein, 125 aa, synthetic construct
  REGION 1..125: note = Synthetic construct, chIL10sdAB1A5
MQVQLQESGGGLVQPGGSLRLSCAASGNIFSINTMGWYRQAPGKQRELVASITTGGTTNYEDSVKGRFTISRDNAKKTVYLQMNRLKPEDTAVYYCNHRRSYSGRDYPVYGMDYWGKGTQVTVSS

SEQ ID NO: 138 - protein, 125 aa, synthetic construct
  REGION 1..125: note = Synthetic construct, chIL10sdAB1C5
MQVQLQESGGGLVQAGGSLRLSCAASGRTFGINVMGWYRQAPGEERELVATVTTGGTTNYEDSVKGRFTISRDNAKKTVYLQMNRLKPEDTAVYYCNHRRSYSGRDYPVYGMDYWGKGTQVTVSS

SEQ ID NO: 139 - protein, 125 aa, synthetic construct
  REGION 1..125: note = Synthetic construct, chIL10sdAB1H6
MQVQLQESGGGLVQAGGSLRLSCTVSGSISSIDGMGWYRQAPGNQRELVATIARTGSTSYVPSVKGRFTISRDNDQTTLYLQMNDLTPEDTAVYYCAADPRKRGVPDWYYGMDYWGKGTLVTVSS

SEQ ID NO: 140 - protein, 125 aa, synthetic construct
  REGION 1..125: note = Synthetic construct, chIL10sdAB2A8
MQVQLQASGGGLVQAGGSLRLSCTVSGSISSIDGMGWYRQAPGNQRELVATIARTGSTSYVPSVKGRFTISRDNAQTTLYLQMNDLTPEDTAVYYCAADPRKRGVPDWYYGMDYWGKGTQVTVSS

SEQ ID NO: 141 - protein, 125 aa, synthetic construct
  REGION 1..125: note = Synthetic construct, chIL10sdAB1F9
MQVQLQASGGGLVQAGGSLRLSCTVSGSISSIDGMGWYRQAPGNQRELVATIARTGSTSYVPSVKGRFTISRDNAQTTLYLQMNDLTPEDTAVYYCAADPRKRGVPDWYYGMDYWGKGTLVTVSS

SEQ ID NO: 142 - protein, 124 aa, synthetic construct
  REGION 1..124: note = Synthetic construct, chIL10sdAB1E11
MQVQLQASGGGVVQAGGSLRLTCAASGRTYAMGWFRQAPGKERDFAAAINWIGSTTDYADSVKGRFTISRDIAKNTMYLQMNSLKPEDTAVYYCAAEKTASLSIYRRAYDYGYWGQGTLVTVSS

SEQ ID NO: 143 - protein, 125 aa, synthetic construct
  REGION 1..125: note = Synthetic construct, chIL10sdAB1D6
MQVQLQASGGGLVQAGGSLRLSCQASESISSINTMGWFRQASGEEREFVAAIESGGATAYAESAKGRFTISRDNAKNTVYLQMNSLKPEDTAVYYCAAGVPTYDDDAMPISWRFWGRGTLVTVSS

SEQ ID NO: 144 - protein, 126 aa, synthetic construct
  REGION 1..126: note = Synthetic construct, chIL10sdAB1C4
MQVQLQASGGGLVQPGGSLRLSCAASGFSLENYVIGWFRQAPGKGREGLSCISSTDDSIFSVDSVKGRFTVSRDNAKNMVYLQMNSLKSEDTAVYYCATSRGLGSCRVDEFYYDMWGQGTQVTVSS

SEQ ID NO: 145 - protein, 125 aa, synthetic construct
  REGION 1..125: note = Synthetic construct, chIL10sdAB1H4
MQVQLQAFGGALVQAGGSLRLSCATSGFTFGYYAIGWLRQAPGKEREGVLCITNADRITYYTNSVKDRFTISRDNTANTVYLQMNNLKPEDTATYYCATNFYSYCSDNGGKYQEWGQGTLVTVSS

SEQ ID NO: 146 - protein, 126 aa, synthetic construct
  REGION 1..126: note = Synthetic construct, chIL10sdAB1F11
MQVQLQEFGGGLVQPGGSLRLSCAASGRTGSSYAMGWFRQAPGKEREFVAAISWSGGSTDYADSVKGRFTISRDNAKNTMYLQMNSLKPEDTAVYYCAVDRNLFKLRVAVQEYTNLGQGTQVTVSS

SEQ ID NO: 147 - protein, 123 aa, synthetic construct
  REGION 1..123: note = Synthetic construct, chIL10sdAB1D3
MQVQLQASGGGLVQPGGSLRLSCAASGFTFDDYAMSWVRQAPGKGLEWVSAITWNVGTIYYAESMKGRFTISRDNAKNTLYLQMNSLKSEDTAMYYCARDGIAPRRYYEMADWGKGTLVTVSS

SEQ ID NO: 148 - protein, 120 aa, synthetic construct
  REGION 1..120: note = Synthetic construct, chIL10sdAB1A7
MQVQLQASGGGSVQAGESLTVSCSVSGNIYDINTMAWYREAPGKQRELVASIGPNGNSDYANGVRGRFTVSRDSVKNTVDLQMTNLKVDDTASYYCNVKTGRGRNLYSDWGDGTQVTVSS

SEQ ID NO: 149 - protein, 122 aa, synthetic construct
  REGION 1..122: note = Synthetic construct, chIL10sdAB1H8
MQVQLQESGGGLVQPGGSLRLSCAASGFTFDDYAMSWVRQAPGKGLEWVSAITWAGGSTYYAESMKGRFTISRDNAKNTLYLQMNSLKSEDTAVYYCARNTGLAYEVGYDYWGQGTQVTVSS

SEQ ID NO: 150 - protein, 119 aa, synthetic construct
  REGION 1..119: note = Synthetic construct, chIL10sdAB1H3
MQVQLQASGGGLVQPGGSLRLSCAASGSISSIDTMGWYRRAPGKQRELVATISSGGRTTYKDSVKGRFTISRDNAKNTMYLQMNSLKPDDTAVYYCAVVVSPTLIAGSWGQGTQVTVSS

SEQ ID NO: 151 - protein, 119 aa, synthetic construct
  REGION 1..119: note = Synthetic construct, chIL10sdAB1B8
MQVQLQASGGALVQPGGSLRLSCSASESIDTFDIIDWYRQAPGKQRDQRELVAQMLPVGATTYADSVKGRFTFSRDNANNMVYLQMDNLQPDDTAVYYCHSINRDHNIWCQGTQVTVSS

SEQ ID NO: 152 - protein, 116 aa, synthetic construct
  REGION 1..116: note = Synthetic construct, chIL10sdAB1B2
MQVQLQAFGGGTVQPGESLTIHCAASGVIPDASAMAWYRQAPGKQRELVARIVGPTNILYGASVKGRFTISRGNGGDTISLQMTNLKPDDTALYICNLLQSGTNYFGKGTLVTVSS

SEQ ID NO: 153 - protein, 116 aa, synthetic construct
  REGION 1..116: note = Synthetic construct, chIL10sdAB1D2
MQVQLQQSGGGLVQTGGSLRLSCAASTNIFSRNTMGWYRQAPAKQREFVASITRSDITNYADSVKGRFTISRDNAKSTVYLQMDSLKAEDTAVYYCYARGRPGIYWGQGTQVTVSS

SEQ ID NO: 154 - protein, 115 aa, synthetic construct
  REGION 1..115: note = Synthetic construct, chIL10sdAB1D10
MQVQLQESGGGLVQAGGSLRLSCVASGITFNTYAMGWYRQAPGKQRERVAVISNFGVTVYENSVKGRFTISRDNAKNAVYLQMNNLRSEDTAVYFCSAIRGVNYWGQGTLVTVSS

SEQ ID NO: 155 - DNA, 6030 bp, synthetic construct
  misc_feature 1..6030: note = Synthetic construct, pAG4314
  source 1..6030: mol_type = other DNA
gtttacccgccaatatatcctgtcaaacactgatagtttaaactgaaggcgggaaacgacaacctgatcatgagcggagaattaagggagtcacgttatgacccccgccgatgacgcgggacaagccgttttacgtttggaactgacagaaccgcaacgttgaaggagccactcagcttaattaagtctaactcgagttactggtacgtatacagggttccttgcgtgaagaagggtggcctgcggttcaccattaacggtcacgactacttccagctagtactggtgaccaacgtcgcggcggcagggtcaatcaagtccatggaggttatgggttccaacacagcggattggatgccgatggcacgtaactggggcgcccaatggcactcactggcctacctcaccggtcaaggtctatcctttagggtcaccaacacagatgaccaaacgctcgtcttcaccaacgtcgtgccaccaggatggaagtttggccagacatttgcaagcaagctgcagttcaagtgagaggagaagcctgaattgataccggagcgtttcttttgggagtaacatctctggttgcctagcaaacatatgattgtatataagtttcgttgtgcgtttattctttcggtgtgtaaaataacatacatgctttcctgatattttcttgtatatatgtacacacacacgacaaatccttccatttctattattattgaacaatttaattgcgagggcgagtacttgtctgtttaccttttttttttcagatggcattttatagtttaacctttcatggaccggcagtagttctaaccatgaatgaaaagaaatcatagtccacaccacgcagggacattgtggtcattttagacaagacgatttgattaatgtcttgtatgatatggtcgacagtgaggactaacaaacatatggcatattttattaccggcgagttaaataaatttatgtcacagtaataaactgcctaataaatgcacgccagaaaatataatgataaaaaaaagaaaagatacataagtccattgcttctacttttttaaaaattaaatccaacattttctattttttggtataaacttggaagtactagttggatatgcaaaatcatctaacctccatatatttcatcaatttgtttactttacatatgggagaggatagtatgtcaaagaaaatgacaacaagcttacaagtttcttattttaaaagttccgctaacttatcaagcatagtgtgccacgcaaaactgacaacaaaccaacaaatttaaggagcgcctaacttatcatctatgacataccgcacaaaatgataacatactagagaaactttattgcacaaaaggaaatttatccataaggcaaaggaacatcttaaggctttggatatacatttaccaacaagcattgtttgtattacccctaaagcgcaagacatgtcatccatgagtcatagtgtgtatatctcaacattgcaaagctaccttttttctattatacttttcgcattataggctagatattatctatacatgtcaacaaactctatccctacgtcatatctgaagattcttttcttcactatataagttggcttccctgtcattgaactcacatcaaccagcccaagtttccaataacatcctcaaatagctggatcctaaaccatgagggtgttgctcgttgccctcgctctcctggctctcgctgcgagcgccacctcccaggttcagctgcaggaaagcggtggcggactggtgcagccaggtggcagcctcaggctgagctgcgctgctagcggcaatatttttagcattaacacaatgggttggtatagacaggctcctggcaagcagcgtgagctcgttgccagcattaccacgggtggtacaaccaattatgaagatagcgtgaagggtcgttttaccattagcagggacaatgctaagaagaccgtttacctccagatgaacaggctgaagccagaagataccgccgtgtattactgcaaccacaggagaagctatagcggaagagattatcctgtttacggtatggactactggggcaagggaaccctggttaccgtgagcagcaaggacgagctgtaacctaggtccccgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattggaattcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcacggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagatccagatctaaaccatgcagaaactcattaactcagtgcaaaactatgcctggggcagcaaaacggcgttgactgaactttatggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggatgattttgccttctcgctgcatgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaagagcttactgaaaaaattaacatctcttgctaagctgggagctctagatccccgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattggcgagctcgaattaattcagtacattaaaaacgtccgcaatgtgttattaagttgtctaagcgtcaatttgtttacaccacaatatatcctgcca

SEQ ID NO: 156 - DNA, 6030 bp, synthetic construct
  misc_feature 1..6030: note = Synthetic construct, pAG4315
  source 1..6030: mol_type = other DNA
gtttacccgccaatatatcctgtcaaacactgatagtttaaactgaaggcgggaaacgacaacctgatcatgagcggagaattaagggagtcacgttatgacccccgccgatgacgcgggacaagccgttttacgtttggaactgacagaaccgcaacgttgaaggagccactcagcttaattaagtctaactcgagttactggtacgtatacagggttccttgcgtgaagaagggtggcctgcggttcaccattaacggtcacgactacttccagctagtactggtgaccaacgtcgcggcggcagggtcaatcaagtccatggaggttatgggttccaacacagcggattggatgccgatggcacgtaactggggcgcccaatggcactcactggcctacctcaccggtcaaggtctatcctttagggtcaccaacacagatgaccaaacgctcgtcttcaccaacgtcgtgccaccaggatggaagtttggccagacatttgcaagcaagctgcagttcaagtgagaggagaagcctgaattgataccggagcgtttcttttgggagtaacatctctggttgcctagcaaacatatgattgtatataagtttcgttgtgcgtttattctttcggtgtgtaaaataacatacatgctttcctgatattttcttgtatatatgtacacacacacgacaaatccttccatttctattattattgaacaatttaattgcgagggcgagtacttgtctgtttaccttttttttttcagatggcattttatagtttaacctttcatggaccggcagtagttctaaccatgaatgaaaagaaatcatagtccacaccacgcagggacattgtggtcattttagacaagacgatttgattaatgtcttgtatgatatggtcgacagtgaggactaacaaacatatggcatattttattaccggcgagttaaataaatttatgtcacagtaataaactgcctaataaatgcacgccagaaaatataatgataaaaaaaagaaaagatacataagtccattgcttctacttttttaaaaattaaatccaacattttctattttttggtataaacttggaagtactagttggatatgcaaaatcatctaacctccatatatttcatcaatttgtttactttacatatgggagaggatagtatgtcaaagaaaatgacaacaagcttacaagtttcttattttaaaagttccgctaacttatcaagcatagtgtgccacgcaaaactgacaacaaaccaacaaatttaaggagcgcctaacttatcatctatgacataccgcacaaaatgataacatactagagaaactttattgcacaaaaggaaatttatccataaggcaaaggaacatcttaaggctttggatatacatttaccaacaagcattgtttgtattacccctaaagcgcaagacatgtcatccatgagtcatagtgtgtatatctcaacattgcaaagctaccttttttctattatacttttcgcattataggctagatattatctatacatgtcaacaaactctatccctacgtcatatctgaagattcttttcttcactatataagttggcttccctgtcattgaactcacatcaaccagcccaagtttccaataacatcctcaaatagctggatcctaaaccatgagggtgttgctcgttgccctcgctctcctggctctcgctgcgagcgccacctcccaggttcagctgcaggaaagcggtggcggactggtgcaggctggtggcagcctcaggctgagctgcgctgctagcggcagaacctttagcagctatgcttggggttggtttaggcaggccccaggcaaggagcgtgaatttgttgccaggattagctttagcggaggtcacacctattacagcgatagcgtgaagggaaggtttaccattagccgtgacaatgctaagaacaccgtttatctccagatgaatagcctgaagccagaggataccgccgtgtattactgcgctgccgacccaaccccttacggtctcaggaatgagagaaactatccttactggggccagggaacccaggttaccgtgagcagcaaggacgagctgtaacctaggtccccgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattggaattcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcacggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagatccagatctaaaccatgcagaaactcattaactcagtgcaaaactatgcctggggcagcaaaacggcgttgactgaactttatggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggatgattttgccttctcgctgcatgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaagagcttactgaaaaaattaacatctcttgctaagctgggagctctagatccccgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattggcgagctcgaattaattcagtacattaaaaacgtccgcaatgtgttattaagttgtctaagcgtcaatttgtttacaccacaatatatcctgcca

SEQ ID NO: 157 - DNA, 6033 bp, synthetic construct
  misc_feature 1..6033: note = Synthetic construct, pAG4316
  source 1..6033: mol_type = other DNA
gtttacccgccaatatatcctgtcaaacactgatagtttaaactgaaggcgggaaacgacaacctgatcatgagcggagaattaagggagtcacgttatgacccccgccgatgacgcgggacaagccgttttacgtttggaactgacagaaccgcaacgttgaaggagccactcagcttaattaagtctaactcgagttactggtacgtatacagggttccttgcgtgaagaagggtggcctgcggttcaccattaacggtcacgactacttccagctagtactggtgaccaacgtcgcggcggcagggtcaatcaagtccatggaggttatgggttccaacacagcggattggatgccgatggcacgtaactggggcgcccaatggcactcactggcctacctcaccggtcaaggtctatcctttagggtcaccaacacagatgaccaaacgctcgtcttcaccaacgtcgtgccaccaggatggaagtttggccagacatttgcaagcaagctgcagttcaagtgagaggagaagcctgaattgataccggagcgtttcttttgggagtaacatctctggttgcctagcaaacatatgattgtatataagtttcgttgtgcgtttattctttcggtgtgtaaaataacatacatgctttcctgatattttcttgtatatatgtacacacacacgacaaatccttccatttctattattattgaacaatttaattgcgagggcgagtacttgtctgtttaccttttttttttcagatggcattttatagtttaacctttcatggaccggcagtagttctaaccatgaatgaaaagaaatcatagtccacaccacgcagggacattgtggtcattttagacaagacgatttgattaatgtcttgtatgatatggtcgacagtgaggactaacaaacatatggcatattttattaccggcgagttaaataaatttatgtcacagtaataaactgcctaataaatgcacgccagaaaatataatgataaaaaaaagaaaagatacataagtccattgcttctacttttttaaaaattaaatccaacattttctattttttggtataaacttggaagtactagttggatatgcaaaatcatctaacctccatatatttcatcaatttgtttactttacatatgggagaggatagtatgtcaaagaaaatgacaacaagcttacaagtttcttattttaaaagttccgctaacttatcaagcatagtgtgccacgcaaaactgacaacaaaccaacaaatttaaggagcgcctaacttatcatctatgacataccgcacaaaatgataacatactagagaaactttattgcacaaaaggaaatttatccataaggcaaaggaacatcttaaggctttggatatacatttaccaacaagcattgtttgtattacccctaaagcgcaagacatgtcatccatgagtcatagtgtgtatatctcaacattgcaaagctaccttttttctattatacttttcgcattataggctagatattatctatacatgtcaacaaactctatccctacgtcatatctgaagattcttttcttcactatataagttggcttccctgtcattgaactcacatcaaccagcccaagtttccaataacatcctcaaatagctggatcctaaaccatgagggtgttgctcgttgccctcgctctcctggctctcgctgcgagcgccacctcccaggttcagctccaggagtttggtggcggactggtgcagccaggtggcagcctcaggctgagctgcgctgctagcggtagaaccggcagcagctatgctatgggatggtttagacaggctccaggcaaggagcgtgaatttgttgctgccattagctggagcggaggtagcaccgattatgctgacagcgtgaagggcaggtttaccattagcagagataatgccaagaacaccatgtacctccagatgaatagcctgaagccagaggataccgctgtttattactgcgccgtggaccgtaatctctttaagctgagggttgctgtgcaggaatacaccaacctcggccagggaacccaggttaccgtgagcagcaaggacgagctgtaacctaggtccccgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattggaattcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcacggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagatccagatctaaaccatgcagaaactcattaactcagtgcaaaactatgcctggggcagcaaaacggcgttgactgaactttatggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggatgattttgccttctcgctgcatgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaagagcttactgaaaaaattaacatctcttgctaagctgggagctctagatccccgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattggcgagctcgaattaattcagtacattaaaaacgtccgcaatgtgttattaagttgtctaagcgtcaatttgtttacaccacaatatatcctgcca

SEQ ID NO: 158 - DNA, 6030 bp, synthetic construct
  misc_feature 1..6030: note = Synthetic construct, pAG4317
  source 1..6030: mol_type = other DNA
gtttacccgccaatatatcctgtcaaacactgatagtttaaactgaaggcgggaaacgacaacctgatcatgagcggagaattaagggagtcacgttatgacccccgccgatgacgcgggacaagccgttttacgtttggaactgacagaaccgcaacgttgaaggagccactcagcttaattaagtctaactcgagttactggtacgtatacagggttccttgcgtgaagaagggtggcctgcggttcaccattaacggtcacgactacttccagctagtactggtgaccaacgtcgcggcggcagggtcaatcaagtccatggaggttatgggttccaacacagcggattggatgccgatggcacgtaactggggcgcccaatggcactcactggcctacctcaccggtcaaggtctatcctttagggtcaccaacacagatgaccaaacgctcgtcttcaccaacgtcgtgccaccaggatggaagtttggccagacatttgcaagcaagctgcagttcaagtgagaggagaagcctgaattgataccggagcgtttcttttgggagtaacatctctggttgcctagcaaacatatgattgtatataagtttcgttgtgcgtttattctttcggtgtgtaaaataacatacatgctttcctgatattttcttgtatatatgtacacacacacgacaaatccttccatttctattattattgaacaatttaattgcgagggcgagtacttgtctgtttaccttttttttttcagatggcattttatagtttaacctttcatggaccggcagtagttctaaccatgaatgaaaagaaatcatagtccacaccacgcagggacattgtggtcattttagacaagacgatttgattaatgtcttgtatgatatggtcgacagtgaggactaacaaacatatggcatattttattaccggcgagttaaataaatttatgtcacagtaataaactgcctaataaatgcacgccagaaaatataatgataaaaaaaagaaaagatacataagtccattgcttctacttttttaaaaattaaatccaacattttctattttttggtataaacttggaagtactagttggatatgcaaaatcatctaacctccatatatttcatcaatttgtttactttacatatgggagaggatagtatgtcaaagaaaatgacaacaagcttacaagtttcttattttaaaagttccgctaacttatcaagcatagtgtgccacgcaaaactgacaacaaaccaacaaatttaaggagcgcctaacttatcatctatgacataccgcacaaaatgataacatactagagaaactttattgcacaaaaggaaatttatccataaggcaaaggaacatcttaaggctttggatatacatttaccaacaagcattgtttgtattacccctaaagcgcaagacatgtcatccatgagtcatagtgtgtatatctcaacattgcaaagctaccttttttctattatacttttcgcattataggctagatattatctatacatgtcaacaaactctatccctacgtcatatctgaagattcttttcttcactatataagttggcttccctgtcattgaactcacatcaaccagcccaagtttccaataacatcctcaaatagctggatcctaaaccatgagggtgttgctcgttgccctcgctctcctggctctcgctgcgagcgccacctcccaggttcagctccaggcttcgggcggcgggctcgtccaggcgggcggctcgctcaggctctcgtgcgcggcgtcggggcggactttcaacagctacgcttggggctggttcaggcaggcgccgggcaaggagcgcggcttcgtggccaggatctccttcagcggcggccacacctactactccgacagcgtcaagggccgcttcacgatctccagggacaacgccaagaacagcgtgtacctccagatgaactccctgaagcccgaggacacggccgtctactactgcgcggcggacccgacgccctacggcctcaggaacgagcggaactaccattactgggggcagggcacgcaggtcactgtctcttcgaaggacgagctgtaacctaggtccccgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattggaattcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcacggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagatccagatctaaaccatgcagaaactcattaactcagtgcaaaactatgcctggggcagcaaaacggcgttgactgaactttatggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggatgattttgccttctcgctgcatgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaagagcttactgaaaaaattaacatctcttgctaagctgggagctctagatccccgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattggcgagctcgaattaattcagtacattaaaaacgtccgcaatgtgttattaagttgtctaagcgtcaatttgtttacaccacaatatatcctgcca

SEQ ID NO: 159 - DNA, 6029 bp, synthetic construct
  misc_feature 1..6029: note = Synthetic construct, pAG4985
  source 1..6029: mol_type = other DNA
gtttacccgccaatatatcctgtcaaacactgatagtttaaactgaaggcgggaaacgacaacctgatcatgagcggagaattaagggagtcacgttatgacccccgccgatgacgcgggacaagccgttttacgtttggaactgacagaaccgcaacgttgaaggagccactcagcctaagcggccgcattggacttaattaagtgaggccggccaagcgtcgatttaaatgtaccacatggcgcgccaactatcatgcgatcgcttcatgtctaactcgagttactggtacgtaccaaatccatggaatcaaggtaccaaagtaatcatattattttatgtgtgaatcttctttactttttcatttgattatgattatgaaggtatgaccttcataaccttcgtccgaaatccattatatccaaaggaaaataatgcttcgaaggacgaaggattttgatatttaacattttatgttgccttgttcttaattcatagcatttgagaacaagtccccaacaccaatctttatctttactatattaaagcaccagttcaacgatcgtctcgtgtcaatattattaaaaaactcctacatttctttataatcaacccgcactcttataatctcttctcttactactataataagagagtttatgtacaaaataaggtgaaattatgtataagtgttctggaccttggttgttggctcatattcacacaacctaatcaatagaaaacatatgttttattaaaacaaaatttatcatatatatatatatatatatatatatatatatatatatatataatataaaccgtagcaatgcacaggcatatgactagtggcaacttaataccatgtgtgtattaagatgaataagaggtatccaaataaataacttgttcgcttacgtctggatcgaaaggggttggaaacgattaaatctcttcctagtcaaaattaaatagaaggagatttaatcgatttctcccaatccccttcgatccaggtgcaaccgaataagtccttaaatgttgaggaacacgaaacaaccatgcattggcatgtaaagctccaagaattcgttgtatccttaacaactcacagaacatcaaccaaaattgcacgtcaagggtattgggtaagaaacaatcaaacaaatcctctctgtgtgcaaagaaacacggtgagtcatgccgagatcatactcatctgatatacatgcttacagctcacaagacattacaaacaactcatattgcattacaaagatcgtttcatgaaaaataaaataggccggaacaggacaaaaatccttgacgtgtaaagtaaatttacaacaaaaaaaaagccatatgtcaagctaaatctaattcgttttacgtagatcaacaacctgtagaaggcaacaaaactgagccacgcagaagtacagaatgattccagatgaaccatcgacgtgctacgtaaagagagtgacgagtcatatacatttggcaagaaaccatgaagctgcctacagccgtctcggtggcataagaacacaagaaattgtgttaattaatcaaagctataaataacgctcgcatgcctgtgcacttctccatcaccaccactgggtcttcagaccattagctttatctactccagagcgcagaagaacccgatcgacaccggatcctaaaccatgagggtgttgctcgttgccctcgctctcctggctctcgctgcgagcgccacctcccaggttcagctgcaggaaagcggtggcggactggtgcagccaggtggcagcctcaggctgagctgcgctgctagcggcaatatttttagcattaacacaatgggttggtatagacaggctcctggcaagcagcgtgagctcgttgccagcattaccacgggtggtacaaccaattatgaagatagcgtgaagggtcgttttaccattagcagggacaatgctaagaagaccgtttacctccagatgaacaggctgaagccagaagataccgccgtgtattactgcaaccacaggagaagctatagcggaagagattatcctgtttacggtatggactactggggcaagggaaccctggttaccgtgagcagcaaggacgagctgtaacctaggtccccgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattggaattcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcacggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagatccagatctaaaccatgcagaaactcattaactcagtgcaaaactatgcctggggcagcaaaacggcgttgactgaactttatggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggatgattttgccttctcgctgcatgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaagagcttactgaaaaaattaacatctcttgctaagctgggagctctagatccccgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattggcgagctcgaattaattcagtacattaaaaacgtccgcaatgtgttattaagttgtctaagcgtcaatttgtttacaccacaatatatcctgcca

SEQ ID NO: 160 - DNA, 6029 bp, synthetic construct
  misc_feature 1..6029: note = Synthetic construct, pAG4986
  source 1..6029: mol_type = other DNA
gtttacccgccaatatatcctgtcaaacactgatagtttaaactgaaggcgggaaacgacaacctgatcatgagcggagaattaagggagtcacgttatgacccccgccgatgacgcgggacaagccgttttacgtttggaactgacagaaccgcaacgttgaaggagccactcagcctaagcggccgcattggacttaattaagtgaggccggccaagcgtcgatttaaatgtaccacatggcgcgccaactatcatgcgatcgcttcatgtctaactcgagttactggtacgtaccaaatccatggaatcaaggtaccaaagtaatcatattattttatgtgtgaatcttctttactttttcatttgattatgattatgaaggtatgaccttcataaccttcgtccgaaatccattatatccaaaggaaaataatgcttcgaaggacgaaggattttgatatttaacattttatgttgccttgttcttaattcatagcatttgagaacaagtccccaacaccaatctttatctttactatattaaagcaccagttcaacgatcgtctcgtgtcaatattattaaaaaactcctacatttctttataatcaacccgcactcttataatctcttctcttactactataataagagagtttatgtacaaaataaggtgaaattatgtataagtgttctggaccttggttgttggctcatattcacacaacctaatcaatagaaaacatatgttttattaaaacaaaatttatcatatatatatatatatatatatatatatatatatatatatataatataaaccgtagcaatgcacaggcatatgactagtggcaacttaataccatgtgtgtattaagatgaataagaggtatccaaataaataacttgttcgcttacgtctggatcgaaaggggttggaaacgattaaatctcttcctagtcaaaattaaatagaaggagatttaatcgatttctcccaatccccttcgatccaggtgcaaccgaataagtccttaaatgttgaggaacacgaaacaaccatgcattggcatgtaaagctccaagaattcgttgtatccttaacaactcacagaacatcaaccaaaattgcacgtcaagggtattgggtaagaaacaatcaaacaaatcctctctgtgtgcaaagaaacacggtgagtcatgccgagatcatactcatctgatatacatgcttacagctcacaagacattacaaacaactcatattgcattacaaagatcgtttcatgaaaaataaaataggccggaacaggacaaaaatccttgacgtgtaaagtaaatttacaacaaaaaaaaagccatatgtcaagctaaatctaattcgttttacgtagatcaacaacctgtagaaggcaacaaaactgagccacgcagaagtacagaatgattccagatgaaccatcgacgtgctacgtaaagagagtgacgagtcatatacatttggcaagaaaccatgaagctgcctacagccgtctcggtggcataagaacacaagaaattgtgttaattaatcaaagctataaataacgctcgcatgcctgtgcacttctccatcaccaccactgggtcttcagaccattagctttatctactccagagcgcagaagaacccgatcgacaccggatcctaaaccatgagggtgttgctcgttgccctcgctctcctggctctcgctgcgagcgccacctcccaggttcagctgcaggaaagcggtggcggactggtgcaggctggtggcagcctcaggctgagctgcgctgctagcggcagaacctttagcagctatgcttggggttggtttaggcaggccccaggcaaggagcgtgaatttgttgccaggattagctttagcggaggtcacacctattacagcgatagcgtgaagggaaggtttaccattagccgtgacaatgctaagaacaccgtttatctccagatgaatagcctgaagccagaggataccgccgtgtattactgcgctgccgacccaaccccttacggtctcaggaatgagagaaactatccttactggggccagggaacccaggttaccgtgagcagcaaggacgagctgtaacctaggtccccgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattggaattcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcacggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagatccagatctaaaccatgcagaaactcattaactcagtgcaaaactatgcctggggcagcaaaacggcgttgactgaactttatggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggatgattttgccttctcgctgcatgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaagagcttactgaaaaaattaacatctcttgctaagctgggagctctagatccccgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattggcgagctcgaattaattcagtacattaaaaacgtccgcaatgtgttattaagttgtctaagcgtcaatttgtttacaccacaatatatcctgcca

SEQ ID NO: 161 - DNA, 6032 bp, synthetic construct
  misc_feature 1..6032: note = Synthetic construct, pAG4987
  source 1..6032: mol_type = other DNA
gtttacccgccaatatatcctgtcaaacactgatagtttaaactgaaggcgggaaacgacaacctgatcatgagcggagaattaagggagtcacgttatgacccccgccgatgacgcgggacaagccgttttacgtttggaactgacagaaccgcaacgttgaaggagccactcagcctaagcggccgcattggacttaattaagtgaggccggccaagcgtcgatttaaatgtaccacatggcgcgccaactatcatgcgatcgcttcatgtctaactcgagttactggtacgtaccaaatccatggaatcaaggtaccaaagtaatcatattattttatgtgtgaatcttctttactttttcatttgattatgattatgaaggtatgaccttcataaccttcgtccgaaatccattatatccaaaggaaaataatgcttcgaaggacgaaggattttgatatttaacattttatgttgccttgttcttaattcatagcatttgagaacaagtccccaacaccaatctttatctttactatattaaagcaccagttcaacgatcgtctcgtgtcaatattattaaaaaactcctacatttctttataatcaacccgcactcttataatctcttctcttactactataataagagagtttatgtacaaaataaggtgaaattatgtataagtgttctggaccttggttgttggctcatattcacacaacctaatcaatagaaaacatatgttttattaaaacaaaatttatcatatatatatatatatatatatatatatatatatatatatataatataaaccgtagcaatgcacaggcatatgactagtggcaacttaataccatgtgtgtattaagatgaataagaggtatccaaataaataacttgttcgcttacgtctggatcgaaaggggttggaaacgattaaatctcttcctagtcaaaattaaatagaaggagatttaatcgatttctcccaatccccttcgatccaggtgcaaccgaataagtccttaaatgttgaggaacacgaaacaaccatgcattggcatgtaaagctccaagaattcgttgtatccttaacaactcacagaacatcaaccaaaattgcacgtcaagggtattgggtaagaaacaatcaaacaaatcctctctgtgtgcaaagaaacacggtgagtcatgccgagatcatactcatctgatatacatgcttacagctcacaagacattacaaacaactcatattgcattacaaagatcgtttcatgaaaaataaaataggccggaacaggacaaaaatccttgacgtgtaaagtaaatttacaacaaaaaaaaagccatatgtcaagctaaatctaattcgttttacgtagatcaacaacctgtagaaggcaacaaaactgagccacgcagaagtacagaatgattccagatgaaccatcgacgtgctacgtaaagagagtgacgagtcatatacatttggcaagaaaccatgaagctgcctacagccgtctcggtggcataagaacacaagaaattgtgttaattaatcaaagctataaataacgctcgcatgcctgtgcacttctccatcaccaccactgggtcttcagaccattagctttatctactccagagcgcagaagaacccgatcgacaccggatcctaaaccatgagggtgttgctcgttgccctcgctctcctggctctcgctgcgagcgccacctcccaggttcagctccaggagtttggtggcggactggtgcagccaggtggcagcctcaggctgagctgcgctgctagcggtagaaccggcagcagctatgctatgggatggtttagacaggctccaggcaaggagcgtgaatttgttgctgccattagctggagcggaggtagcaccgattatgctgacagcgtgaagggcaggtttaccattagcagagataatgccaagaacaccatgtacctccagatgaatagcctgaagccagaggataccgctgtttattactgcgccgtggaccgtaatctctttaagctgagggttgctgtgcaggaatacaccaacctcggccagggaacccaggttaccgtgagcagcaaggacgagctgtaacctaggtccccgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattggaattcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcacggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagatccagatctaaaccatgcagaaactcattaactcagtgcaaaactatgcctggggcagcaaaacggcgttgactgaactttatggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggatgattttgccttctcgctgcatgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaagagcttactgaaaaaattaacatctcttgctaagctgggagctctagatccccgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattggcgagctcgaattaattcagtacattaaaaacgtccgcaatgtgttattaagttgtctaagcgtcaatttgtttacaccacaatatatcctgcca

SEQ ID NO: 162 - DNA, 8274 bp, synthetic construct
  misc_feature 1..8274: note = Synthetic construct, pAG4988
  source 1..8274: mol_type = other DNA
gtttacccgccaatatatcctgtcaaacactgatagtttaaactgaaggcgggaaacgacaacctgatcatgagcggagaattaagggagtcacgttatgacccccgccgatgacgcgggacaagccgttttacgtttggaactgacagaaccgcaacgttgaaggagccactcagcctaagcggccgcattggacttaattaagtgaggccggccaagcgtcgatttaaatgtaccacatggcgcgccaactatcatgcgatcgcttcatgtctaactcgagttactggtacgtaccaaatccatggaatcaaggtaccaaagtaatcatattattttatgtgtgaatcttctttactttttcatttgattatgattatgaaggtatgaccttcataaccttcgtccgaaatccattatatccaaaggaaaataatgcttcgaaggacgaaggattttgatatttaacattttatgttgccttgttcttaattcatagcatttgagaacaagtccccaacaccaatctttatctttactatattaaagcaccagttcaacgatcgtctcgtgtcaatattattaaaaaactcctacatttctttataatcaacccgcactcttataatctcttctcttactactataataagagagtttatgtacaaaataaggtgaaattatgtataagtgttctggaccttggttgttggctcatattcacacaacctaatcaatagaaaacatatgttttattaaaacaaaatttatcatatatatatatatatatatatatatatatatatatatatataatataaaccgtagcaatgcacaggcatatgactagtggcaacttaataccatgtgtgtattaagatgaataagaggtatccaaataaataacttgttcgcttacgtctggatcgaaaggggttggaaacgattaaatctcttcctagtcaaaattaaatagaaggagatttaatcgatttctcccaatccccttcgatccaggtgcaaccgaataagtccttaaatgttgaggaacacgaaacaaccatgcattggcatgtaaagctccaagaattcgttgtatccttaacaactcacagaacatcaaccaaaattgcacgtcaagggtattgggtaagaaacaatcaaacaaatcctctctgtgtgcaaagaaacacggtgagtcatgccgagatcatactcatctgatatacatgcttacagctcacaagacattacaaacaactcatattgcattacaaagatcgtttcatgaaaaataaaataggccggaacaggacaaaaatccttgacgtgtaaagtaaatttacaacaaaaaaaaagccatatgtcaagctaaatctaattcgttttacgtagatcaacaacctgtagaaggcaacaaaactgagccacgcagaagtacagaatgattccagatgaaccatcgacgtgctacgtaaagagagtgacgagtcatatacatttggcaagaaaccatgaagctgcctacagccgtctcggtggcataagaacacaagaaattgtgttaattaatcaaagctataaataacgctcgcatgcctgtgcacttctccatcaccaccactgggtcttcagaccattagctttatctactccagagcgcagaagaacccgatcgacaccggatcctaaaccatgagggtgttgctcgttgccctcgctctcctggctctcgctgcgagcgccacctcccaggttcagctgcaggaaagcggtggcggactggtgcagccaggtggcagcctcaggctgagctgcgctgctagcggcaatatttttagcattaacacaatgggttggtatagacaggctcctggcaagcagcgtgagctcgttgccagcattaccacgggtggtacaaccaattatgaagatagcgtgaagggtcgttttaccattagcagggacaatgctaagaagaccgtttacctccagatgaacaggctgaagccagaagataccgccgtgtattactgcaaccacaggagaagctatagcggaagagattatcctgtttacggtatggactactggggcaagggaaccctggttaccgtgagcagcaaggacgagctgtaacctaggtccccgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattgttaattaagtctaactcgagttactggtacgtatacagggttccttgcgtgaagaagggtggcctgcggttcaccattaacggtcacgactacttccagctagtactggtgaccaacgtcgcggcggcagggtcaatcaagtccatggaggttatgggttccaacacagcggattggatgccgatggcacgtaactggggcgcccaatggcactcactggcctacctcaccggtcaaggtctatcctttagggtcaccaacacagatgaccaaacgctcgtcttcaccaacgtcgtgccaccaggatggaagtttggccagacatttgcaagcaagctgcagttcaagtgagaggagaagcctgaattgataccggagcgtttcttttgggagtaacatctctggttgcctagcaaacatatgattgtatataagtttcgttgtgcgtttattctttcggtgtgtaaaataacatacatgctttcctgatattttcttgtatatatgtacacacacacgacaaatccttccatttctattattattgaacaatttaattgcgagggcgagtacttgtctgtttaccttttttttttcagatggcattttatagtttaacctttcatggaccggcagtagttctaaccatgaatgaaaagaaatcatagtccacaccacgcagggacattgtggtcattttagacaagacgatttgattaatgtcttgtatgatatggtcgacagtgaggactaacaaacatatggcatattttattaccggcgagttaaataaatttatgtcacagtaataaactgcctaataaatgcacgccagaaaatataatgataaaaaaaagaaaagatacataagtccattgcttctacttttttaaaaattaaatccaacattttctattttttggtataaacttggaagtactagttggatatgcaaaatcatctaacctccatatatttcatcaatttgtttactttacatatgggagaggatagtatgtcaaagaaaatgacaacaagcttacaagtttcttattttaaaagttccgctaacttatcaagcatagtgtgccacgcaaaactgacaacaaaccaacaaatttaaggagcgcctaacttatcatctatgacataccgcacaaaatgataacatactagagaaactttattgcacaaaaggaaatttatccataaggcaaaggaacatcttaaggctttggatatacatttaccaacaagcattgtttgtattacccctaaagcgcaagacatgtcatccatgagtcatagtgtgtatatctcaacattgcaaagctaccttttttctattatacttttcgcattataggctagatattatctatacatgtcaacaaactctatccctacgtcatatctgaagattcttttcttcactatataagttggcttccctgtcattgaactcacatcaaccagcccaagtttccaataacatcctcaaatagctggatcctaaaccatgagggtgttgctcgttgccctcgctctcctggctctcgctgcgagcgccacctcccaggttcagctgcaggaaagcggtggcggactggtgcagccaggtggcagcctcaggctgagctgcgctgctagcggcaatatttttagcattaacacaatgggttggtatagacaggctcctggcaagcagcgtgagctcgttgccagcattaccacgggtggtacaaccaattatgaagatagcgtgaagggtcgttttaccattagcagggacaatgctaagaagaccgtttacctccagatgaacaggctgaagccagaagataccgccgtgtattactgcaaccacaggagaagctatagcggaagagattatcctgtttacggtatggactactggggcaagggaaccctggttaccgtgagcagcaaggacgagctgtaacctaggtccccgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattggaattcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcacggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagatccagatctaaaccatgcagaaactcattaactcagtgcaaaactatgcctggggcagcaaaacggcgttgactgaactttatggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggatgattttgccttctcgctgcatgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaagagcttactgaaaaaattaacatctcttgctaagctgggagctctagatccccgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattggcgagctcgaattaattcagtacattaaaaacgtccgcaatgtgttattaagttgtctaagcgtcaatttgtttacaccacaatatatcctgcca

SEQ ID NO: 163 - DNA, 8274 bp, synthetic construct
  misc_feature 1..8274: note = Synthetic construct, pAG4989
  source 1..8274: mol_type = other DNA
gtttacccgccaatatatcctgtcaaacactgatagtttaaactgaaggcgggaaacgacaacctgatcatgagcggagaattaagggagtcacgttatgacccccgccgatgacgcgggacaagccgttttacgtttggaactgacagaaccgcaacgttgaaggagccactcagcctaagcggccgcattggacttaattaagtgaggccggccaagcgtcgatttaaatgtaccacatggcgcgccaactatcatgcgatcgcttcatgtctaactcgagttactggtacgtaccaaatccatggaatcaaggtaccaaagtaatcatattattttatgtgtgaatcttctttactttttcatttgattatgattatgaaggtatgaccttcataaccttcgtccgaaatccattatatccaaaggaaaataatgcttcgaaggacgaaggattttgatatttaacattttatgttgccttgttcttaattcatagcatttgagaacaagtccccaacaccaatctttatctttactatattaaagcaccagttcaacgatcgtctcgtgtcaatattattaaaaaactcctacatttctttataatcaacccgcactcttataatctcttctcttactactataataagagagtttatgtacaaaataaggtgaaattatgtataagtgttctggaccttggttgttggctcatattcacacaacctaatcaatagaaaacatatgttttattaaaacaaaatttatcatatatatatatatatatatatatatatatatatatatatataatataaaccgtagcaatgcacaggcatatgactagtggcaacttaataccatgtgtgtattaagatgaataagaggtatccaaataaataacttgttcgcttacgtctggatcgaaaggggttggaaacgattaaatctcttcctagtcaaaattaaatagaaggagatttaatcgatttctcccaatccccttcgatccaggtgcaaccgaataagtccttaaatgttgaggaacacgaaacaaccatgcattggcatgtaaagctccaagaattcgttgtatccttaacaactcacagaacatcaaccaaaattgcacgtcaagggtattgggtaagaaacaatcaaacaaatcctctctgtgtgcaaagaaacacggtgagtcatgccgagatcatactcatctgatatacatgcttacagctcacaagacattacaaacaactcatattgcattacaaagatcgtttcatgaaaaataaaataggccggaacaggacaaaaatccttgacgtgtaaagtaaatttacaacaaaaaaaaagccatatgtcaagctaaatctaattcgttttacgtagatcaacaacctgtagaaggcaacaaaactgagccacgcagaagtacagaatgattccagatgaaccatcgacgtgctacgtaaagagagtgacgagtcatatacatttggcaagaaaccatgaagctgcctacagccgtctcggtggcataagaacacaagaaattgtgttaattaatcaaagctataaataacgctcgcatgcctgtgcacttctccatcaccaccactgggtcttcagaccattagctttatctactccagagcgcagaagaacccgatcgacaccggatcctaaaccatgagggtgttgctcgttgccctcgctctcctggctctcgctgcgagcgccacctcccaggttcagctgcaggaaagcggtggcggactggtgcaggctggtggcagcctcaggctgagctgcgctgctagcggcagaacctttagcagctatgcttggggttggtttaggcaggccccaggcaaggagcgtgaatttgttgccaggattagctttagcggaggtcacacctattacagcgatagcgtgaagggaaggtttaccattagccgtgacaatgctaagaacaccgtttatctccagatgaatagcctgaagccagaggataccgccgtgtattactgcgctgccgacccaaccccttacggtctcaggaatgagagaaactatccttactggggccagggaacccaggttaccgtgagcagcaaggacgagctgtaacctaggtccccgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattgttaattaagtctaactcgagttactggtacgtatacagggttccttgcgtgaagaagggtggcctgcggttcaccattaacggtcacgactacttccagctagtactggtgaccaacgtcgcggcggcagggtcaatcaagtccatggaggttatgggttccaacacagcggattggatgccgatggcacgtaactggggcgcccaatggcactcactggcctacctcaccggtcaaggtctatcctttagggtcaccaacacagatgaccaaacgctcgtcttcaccaacgtcgtgccaccaggatggaagtttggccagacatttgcaagcaagctgcagttcaagtgagaggagaagcctgaattgataccggagcgtttcttttgggagtaacatctctggttgcctagcaaacatatgattgtatataagtttcgttgtgcgtttattctttcggtgtgtaaaataacatacatgctttcctgatattttcttgtatatatgtacacacacacgacaaatccttccatttctattattattgaacaatttaattgcgagggcgagtacttgtctgtttaccttttttttttcagatggcattttatagtttaacctttcatggaccggcagtagttctaaccatgaatgaaaagaaatcatagtccacaccacgcagggacattgtggtcattttagacaagacgatttgattaatgtcttgtatgatatggtcgacagtgaggactaacaaacatatggcatattttattaccggcgagttaaataaatttatgtcacagtaataaactgcctaataaatgcacgccagaaaatataatgataaaaaaaagaaaagatacataagtccattgcttctacttttttaaaaattaaatccaacattttctattttttggtataaacttggaagtactagttggatatgcaaaatcatctaacctccatatatttcatcaatttgtttactttacatatgggagaggatagtatgtcaaagaaaatgacaacaagcttacaagtttcttattttaaaagttccgctaacttatcaagcatagtgtgccacgcaaaactgacaacaaaccaacaaatttaaggagcgcctaacttatcatctatgacataccgcacaaaatgataacatactagagaaactttattgcacaaaaggaaatttatccataaggcaaaggaacatcttaaggctttggatatacatttaccaacaagcattgtttgtattacccctaaagcgcaagacatgtcatccatgagtcatagtgtgtatatctcaacattgcaaagctaccttttttctattatacttttcgcattataggctagatattatctatacatgtcaacaaactctatccctacgtcatatctgaagattcttttcttcactatataagttggcttccctgtcattgaactcacatcaaccagcccaagtttccaataacatcctcaaatagctggatcctaaaccatgagggtgttgctcgttgccctcgctctcctggctctcgctgcgagcgccacctcccaggttcagctgcaggaaagcggtggcggactggtgcaggctggtggcagcctcaggctgagctgcgctgctagcggcagaacctttagcagctatgcttggggttggtttaggcaggccccaggcaaggagcgtgaatttgttgccaggattagctttagcggaggtcacacctattacagcgatagcgtgaagggaaggtttaccattagccgtgacaatgctaagaacaccgtttatctccagatgaatagcctgaagccagaggataccgccgtgtattactgcgctgccgacccaaccccttacggtctcaggaatgagagaaactatccttactggggccagggaacccaggttaccgtgagcagcaaggacgagctgtaacctaggtccccgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattggaattcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcacggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagatccagatctaaaccatgcagaaactcattaactcagtgcaaaactatgcctggggcagcaaaacggcgttgactgaactttatggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggatgattttgccttctcgctgcatgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaagagcttactgaaaaaattaacatctcttgctaagctgggagctctagatccccgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattggcgagctcgaattaattcagtacattaaaaacgtccgcaatgtgttattaagttgtctaagcgtcaatttgtttacaccacaatatatcctgcca

SEQ ID NO: 164 - DNA, 8280 bp, synthetic construct
  misc_feature 1..8280: note = Synthetic construct, pAG4990
  source 1..8280: mol_type = other DNA
gtttacccgccaatatatcctgtcaaacactgatagtttaaactgaaggcgggaaacgacaacctgatcatgagcggagaattaagggagtcacgttatgacccccgccgatgacgcgggacaagccgttttacgtttggaactgacagaaccgcaacgttgaaggagccactcagcctaagcggccgcattggacttaattaagtgaggccggccaagcgtcgatttaaatgtaccacatggcgcgccaactatcatgcgatcgcttcatgtctaactcgagttactggtacgtaccaaatccatggaatcaaggtaccaaagtaatcatattattttatgtgtgaatcttctttactttttcatttgattatgattatgaaggtatgaccttcataaccttcgtccgaaatccattatatccaaaggaaaataatgcttcgaaggacgaaggattttgatatttaacattttatgttgccttgttcttaattcatagcatttgagaacaagtccccaacaccaatctttatctttactatattaaagcaccagttcaacgatcgtctcgtgtcaatattattaaaaaactcctacatttctttataatcaacccgcactcttataatctcttctcttactactataataagagagtttatgtacaaaataaggtgaaattatgtataagtgttctggaccttggttgttggctcatattcacacaacctaatcaatagaaaacatatgttttattaaaacaaaatttatcatatatatatatatatatatatatatatatatatatatatataatataaaccgtagcaatgcacaggcatatgactagtggcaacttaataccatgtgtgtattaagatgaataagaggtatccaaataaataacttgttcgcttacgtctggatcgaaaggggttggaaacgattaaatctcttcctagtcaaaattaaatagaaggagatttaatcgatttctcccaatccccttcgatccaggtgcaaccgaataagtccttaaatgttgaggaacacgaaacaaccatgcattggcatgtaaagctccaagaattcgttgtatccttaacaactcacagaacatcaaccaaaattgcacgtcaagggtattgggtaagaaacaatcaaacaaatcctctctgtgtgcaaagaaacacggtgagtcatgccgagatcatactcatctgatatacatgcttacagctcacaagacattacaaacaactcatattgcattacaaagatcgtttcatgaaaaataaaataggccggaacaggacaaaaatccttgacgtgtaaagtaaatttacaacaaaaaaaaagccatatgtcaagctaaatctaattcgttttacgtagatcaacaacctgtagaaggcaacaaaactgagccacgcagaagtacagaatgattccagatgaaccatcgacgtgctacgtaaagagagtgacgagtcatatacatttggcaagaaaccatgaagctgcctacagccgtctcggtggcataagaacacaagaaattgtgttaattaatcaaagctataaataacgctcgcatgcctgtgcacttctccatcaccaccactgggtcttcagaccattagctttatctactccagagcgcagaagaacccgatcgacaccggatcctaaaccatgagggtgttgctcgttgccctcgctctcctggctctcgctgcgagcgccacctcccaggttcagctccaggagtttggtggcggactggtgcagccaggtggcagcctcaggctgagctgcgctgctagcggtagaaccggcagcagctatgctatgggatggtttagacaggctccaggcaaggagcgtgaatttgttgctgccattagctggagcggaggtagcaccgattatgctgacagcgtgaagggcaggtttaccattagcagagataatgccaagaacaccatgtacctccagatgaatagcctgaagccagaggataccgctgtttattactgcgccgtggaccgtaatctctttaagctgagggttgctgtgcaggaatacaccaacctcggccagggaacccaggttaccgtgagcagcaaggacgagctgtaacctaggtccccgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattgttaattaagtctaactcgagttactggtacgtatacagggttccttgcgtgaagaagggtggcctgcggttcaccattaacggtcacgactacttccagctagtactggtgaccaacgtcgcggcggcagggtcaatcaagtccatggaggttatgggttccaacacagcggattggatgccgatggcacgtaactggggcgcccaatggcactcactggcctacctcaccggtcaaggtctatcctttagggtcaccaacacagatgaccaaacgctcgtcttcaccaacgtcgtgccaccaggatggaagtttggccagacatttgcaagcaagctgcagttcaagtgagaggagaagcctgaattgataccggagcgtttcttttgggagtaacatctctggttgcctagcaaacatatgattgtatataagtttcgttgtgcgtttattctttcggtgtgtaaaataacatacatgctttcctgatattttcttgtatatatgtacacacacacgacaaatccttccatttctattattattgaacaatttaattgcgagggcgagtacttgtctgtttaccttttttttttcagatggcattttatagtttaacctttcatggaccggcagtagttctaaccatgaatgaaaagaaatcatagtccacaccacgcagggacattgtggtcattttagacaagacgatttgattaatgtcttgtatgatatggtcgacagtgaggactaacaaacatatggcatattttattaccggcgagttaaataaatttatgtcacagtaataaactgcctaataaatgcacgccagaaaatataatgataaaaaaaagaaaagatacataagtccattgcttctacttttttaaaaattaaatccaacattttctattttttggtataaacttggaagtactagttggatatgcaaaatcatctaacctccatatatttcatcaatttgtttactttacatatgggagaggatagtatgtcaaagaaaatgacaacaagcttacaagtttcttattttaaaagttccgctaacttatcaagcatagtgtgccacgcaaaactgacaacaaaccaacaaatttaaggagcgcctaacttatcatctatgacataccgcacaaaatgataacatactagagaaactttattgcacaaaaggaaatttatccataaggcaaaggaacatcttaaggctttggatatacatttaccaacaagcattgtttgtattacccctaaagcgcaagacatgtcatccatgagtcatagtgtgtatatctcaacattgcaaagctaccttttttctattatacttttcgcattataggctagatattatctatacatgtcaacaaactctatccctacgtcatatctgaagattcttttcttcactatataagttggcttccctgtcattgaactcacatcaaccagcccaagtttccaataacatcctcaaatagctggatcctaaaccatgagggtgttgctcgttgccctcgctctcctggctctcgctgcgagcgccacctcccaggttcagctccaggagtttggtggcggactggtgcagccaggtggcagcctcaggctgagctgcgctgctagcggtagaaccggcagcagctatgctatgggatggtttagacaggctccaggcaaggagcgtgaatttgttgctgccattagctggagcggaggtagcaccgattatgctgacagcgtgaagggcaggtttaccattagcagagataatgccaagaacaccatgtacctccagatgaatagcctgaagccagaggataccgctgtttattactgcgccgtggaccgtaatctctttaagctgagggttgctgtgcaggaatacaccaacctcggccagggaacccaggttaccgtgagcagcaaggacgagctgtaacctaggtccccgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattggaattcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcacggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagatccagatctaaaccatgcagaaactcattaactcagtgcaaaactatgcctggggcagcaaaacggcgttgactgaactttatggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggatgattttgccttctcgctgcatgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaagagcttactgaaaaaattaacatctcttgctaagctgggagctctagatccccgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattggcgagctcgaattaattcagtacattaaaaacgtccgcaatgtgttattaagttgtctaagcgtcaatttgtttacaccacaatatatcctgcca

SEQ ID NO: 165 - DNA, 6029 bp, synthetic construct
  misc_feature 1..6029: note = Synthetic construct, pAG4991
  source 1..6029: mol_type = other DNA
gtttacccgccaatatatcctgtcaaacactgatagtttaaactgaaggcgggaaacgacaacctgatcatgagcggagaattaagggagtcacgttatgacccccgccgatgacgcgggacaagccgttttacgtttggaactgacagaaccgcaacgttgaaggagccactcagcctaagcggccgcattggacttaattaagtgaggccggccaagcgtcgatttaaatgtaccacatggcgcgccaactatcatgcgatcgcttcatgtctaactcgagttactggtacgtaccaaatccatggaatcaaggtaccaaagtaatcatattattttatgtgtgaatcttctttactttttcatttgattatgattatgaaggtatgaccttcataaccttcgtccgaaatccattatatccaaaggaaaataatgcttcgaaggacgaaggattttgatatttaacattttatgttgccttgttcttaattcatagcatttgagaacaagtccccaacaccaatctttatctttactatattaaagcaccagttcaacgatcgtctcgtgtcaatattattaaaaaactcctacatttctttataatcaacccgcactcttataatctcttctcttactactataataagagagtttatgtacaaaataaggtgaaattatgtataagtgttctggaccttggttgttggctcatattcacacaacctaatcaatagaaaacatatgttttattaaaacaaaatttatcatatatatatatatatatatatatatatatatatatatatataatataaaccgtagcaatgcacaggcatatgactagtggcaacttaataccatgtgtgtattaagatgaataagaggtatccaaataaataacttgttcgcttacgtctggatcgaaaggggttggaaacgattaaatctcttcctagtcaaaattaaatagaaggagatttaatcgatttctcccaatccccttcgatccaggtgcaaccgaataagtccttaaatgttgaggaacacgaaacaaccatgcattggcatgtaaagctccaagaattcgttgtatccttaacaactcacagaacatcaaccaaaattgcacgtcaagggtattgggtaagaaacaatcaaacaaatcctctctgtgtgcaaagaaacacggtgagtcatgccgagatcatactcatctgatatacatgcttacagctcacaagacattacaaacaactcatattgcattacaaagatcgtttcatgaaaaataaaataggccggaacaggacaaaaatccttgacgtgtaaagtaaatttacaacaaaaaaaaagccatatgtcaagctaaatctaattcgttttacgtagatcaacaacctgtagaaggcaacaaaactgagccacgcagaagtacagaatgattccagatgaaccatcgacgtgctacgtaaagagagtgacgagtcatatacatttggcaagaaaccatgaagctgcctacagccgtctcggtggcataagaacacaagaaattgtgttaattaatcaaagctataaataacgctcgcatgcctgtgcacttctccatcaccaccactgggtcttcagaccattagctttatctactccagagcgcagaagaacccgatcgacaccggatcctaaaccatgagggtgttgctcgttgccctcgctctcctggctctcgctgcgagcgccacctcccaggttcagctccaggcttcgggcggcgggctcgtccaggcgggcggctcgctcaggctctcgtgcgcggcgtcggggcggactttcaacagctacgcttggggctggttcaggcaggcgccgggcaaggagcgcggcttcgtggccaggatctccttcagcggcggccacacctactactccgacagcgtcaagggccgcttcacgatctccagggacaacgccaagaacagcgtgtacctccagatgaactccctgaagcccgaggacacggccgtctactactgcgcggcggacccgacgccctacggcctcaggaacgagcggaactaccattactgggggcagggcacgcaggtcactgtctcttcgaaggacgagctgtaacctaggtccccgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattggaattcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcacggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagatccagatctaaaccatgcagaaactcattaactcagtgcaaaactatgcctggggcagcaaaacggcgttgactgaactttatggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggatgattttgccttctcgctgcatgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaagagcttactgaaaaaattaacatctcttgctaagctgggagctctagatccccgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattggcgagctcgaattaattcagtacattaaaaacgtccgcaatgtgttattaagttgtctaagcgtcaatttgtttacaccacaatatatcctgcca

SEQ ID NO: 166 - DNA, 8274 bp, synthetic construct
  misc_feature 1..8274: note = Synthetic construct, pAG4992
  source 1..8274: mol_type = other DNA
gtttacccgccaatatatcctgtcaaacactgatagtttaaactgaaggcgggaaacgacaacctgatcatgagcggagaattaagggagtcacgttatgacccccgccgatgacgcgggacaagccgttttacgtttggaactgacagaaccgcaacgttgaaggagccactcagcctaagcggccgcattggacttaattaagtgaggccggccaagcgtcgatttaaatgtaccacatggcgcgccaactatcatgcgatcgcttcatgtctaactcgagttactggtacgtaccaaatccatggaatcaaggtaccaaagtaatcatattattttatgtgtgaatcttctttactttttcatttgattatgattatgaaggtatgaccttcataaccttcgtccgaaatccattatatccaaaggaaaataatgcttcgaaggacgaaggattttgatatttaacattttatgttgccttgttcttaattcatagcatttgagaacaagtccccaacaccaatctttatctttactatattaaagcaccagttcaacgatcgtctcgtgtcaatattattaaaaaactcctacatttctttataatcaacccgcactcttataatctcttctcttactactataataagagagtttatgtacaaaataaggtgaaattatgtataagtgttctggaccttggttgttggctcatattcacacaacctaatcaatagaaaacatatgttttattaaaacaaaatttatcatatatatatatatatatatatatatatatatatatatatataatataaaccgtagcaatgcacaggcatatgactagtggcaacttaataccatgtgtgtattaagatgaataagaggtatccaaataaataacttgttcgcttacgtctggatcgaaaggggttggaaacgattaaatctcttcctagtcaaaattaaatagaaggagatttaatcgatttctcccaatccccttcgatccaggtgcaaccgaataagtccttaaatgttgaggaacacgaaacaaccatgcattggcatgtaaagctccaagaattcgttgtatccttaacaactcacagaacatcaaccaaaattgcacgtcaagggtattgggtaagaaacaatcaaacaaatcctctctgtgtgcaaagaaacacggtgagtcatgccgagatcatactcatctgatatacatgcttacagctcacaagacattacaaacaactcatattgcattacaaagatcgtttcatgaaaaataaaataggccggaacaggacaaaaatccttgacgtgtaaagtaaatttacaacaaaaaaaaagccatatgtcaagctaaatctaattcgttttacgtagatcaacaacctgtagaaggcaacaaaactgagccacgcagaagtacagaatgattccagatgaaccatcgacgtgctacgtaaagagagtgacgagtcatatacatttggcaagaaaccatgaagctgcctacagccgtctcggtggcataagaacacaagaaattgtgttaattaatcaaagctataaataacgctcgcatgcctgtgcacttctccatcaccaccactgggtcttcagaccattagctttatctactccagagcgcagaagaacccgatcgacaccggatcctaaaccatgagggtgttgctcgttgccctcgctctcctggctctcgctgcgagcgccacctcccaggttcagctccaggcttcgggcggcgggctcgtccaggcgggcggctcgctcaggctctcgtgcgcggcgtcggggcggactttcaacagctacgcttggggctggttcaggcaggcgccgggcaaggagcgcggcttcgtggccaggatctccttcagcggcggccacacctactactccgacagcgtcaagggccgcttcacgatctccagggacaacgccaagaacagcgtgtacctccagatgaactccctgaagcccgaggacacggccgtctactactgcgcggcggacccgacgccctacggcctcaggaacgagcggaactaccattactgggggcagggcacgcaggtcactgtctcttcgaaggacgagctgtaacctaggtccccgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattgttaattaagtctaactcgagttactggtacgtatacagggttccttgcgtgaagaagggtggcctgcggttcaccattaacggtcacgactacttccagctagtactggtgaccaacgtcgcggcggcagggtcaatcaagtccatggaggttatgggttccaacacagcggattggatgccgatggcacgtaactggggcgcccaatggcactcactggcctacctcaccggtcaaggtctatcctttagggtcaccaacacagatgaccaaacgctcgtcttcaccaacgtcgtgccaccaggatggaagtttggccagacatttgcaagcaagctgcagttcaagtgagaggagaagcctgaattgataccggagcgtttcttttgggagtaacatctctggttgcctagcaaacatatgattgtatataagtttcgttgtgcgtttattctttcggtgtgtaaaataacatacatgctttcctgatattttcttgtatatatgtacacacacacgacaaatccttccatttctattattattgaacaatttaattgcgagggcgagtacttgtctgtttaccttttttttttcagatggcattttatagtttaacctttcatggaccggcagtagttctaaccatgaatgaaaagaaatcatagtccacaccacgcagggacattgtggtcattttagacaagacgatttgattaatgtcttgtatgatatggtcgacagtgaggactaacaaacatatggcatattttattaccggcgagttaaataaatttatgtcacagtaataaactgcctaataaatgcacgccagaaaatataatgataaaaaaaagaaaagatacataagtccattgcttctacttttttaaaaattaaatccaacattttctattttttggtataaacttggaagtactagttggatatgcaaaatcatctaacctccatatatttcatcaatttgtttactttacatatgggagaggatagtatgtcaaagaaaatgacaacaagcttacaagtttcttattttaaaagttccgctaacttatcaagcatagtgtgccacgcaaaactgacaacaaaccaacaaatttaaggagcgcctaacttatcatctatgacataccgcacaaaatgataacatactagagaaactttattgcacaaaaggaaatttatccataaggcaaaggaacatcttaaggctttggatatacatttaccaacaagcattgtttgtattacccctaaagcgcaagacatgtcatccatgagtcatagtgtgtatatctcaacattgcaaagctaccttttttctattatacttttcgcattataggctagatattatctatacatgtcaacaaactctatccctacgtcatatctgaagattcttttcttcactatataagttggcttccctgtcattgaactcacatcaaccagcccaagtttccaataacatcctcaaatagctggatcctaaaccatgagggtgttgctcgttgccctcgctctcctggctctcgctgcgagcgccacctcccaggttcagctccaggcttcgggcggcgggctcgtccaggcgggcggctcgctcaggctctcgtgcgcggcgtcggggcggactttcaacagctacgcttggggctggttcaggcaggcgccgggcaaggagcgcggcttcgtggccaggatctccttcagcggcggccacacctactactccgacagcgtcaagggccgcttcacgatctccagggacaacgccaagaacagcgtgtacctccagatgaactccctgaagcccgaggacacggccgtctactactgcgcggcggacccgacgccctacggcctcaggaacgagcggaactaccattactgggggcagggcacgcaggtcactgtctcttcgaaggacgagctgtaacctaggtccccgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattggaattcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcacggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagatccagatctaaaccatgcagaaactcattaactcagtgcaaaactatgcctggggcagcaaaacggcgttgactgaactttatggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggatgattttgccttctcgctgcatgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaagagcttactgaaaaaattaacatctcttgctaagctgggagctctagatccccgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattggcgagctcgaattaattcagtacattaaaaacgtccgcaatgtgttattaagttgtctaagcgtcaatttgtttacaccacaatatatcctgcca

SEQ ID NO: 167 - DNA, 6029 bp, synthetic construct
  misc_feature 1..6029: note = Synthetic construct, pAG4993
  source 1..6029: mol_type = other DNA
gtttacccgccaatatatcctgtcaaacactgatagtttaaactgaaggcgggaaacgacaacctgatcatgagcggagaattaagggagtcacgttatgacccccgccgatgacgcgggacaagccgttttacgtttggaactgacagaaccgcaacgttgaaggagccactcagcctaagcggccgcattggacttaattaagtgaggccggccaagcgtcgatttaaatgtaccacatggcgcgccaactatcatgcgatcgcttcatgtctaactcgagttactggtacgtaccaaatccatggaatcaaggtaccaaagtaatcatattattttatgtgtgaatcttctttactttttcatttgattatgattatgaaggtatgaccttcataaccttcgtccgaaatccattatatccaaaggaaaataatgcttcgaaggacgaaggattttgatatttaacattttatgttgccttgttcttaattcatagcatttgagaacaagtccccaacaccaatctttatctttactatattaaagcaccagttcaacgatcgtctcgtgtcaatattattaaaaaactcctacatttctttataatcaacccgcactcttataatctcttctcttactactataataagagagtttatgtacaaaataaggtgaaattatgtataagtgttctggaccttggttgttggctcatattcacacaacctaatcaatagaaaacatatgttttattaaaacaaaatttatcatatatatatatatatatatatatatatatatatatatatataatataaaccgtagcaatgcacaggcatatgactagtggcaacttaataccatgtgtgtattaagatgaataagaggtatccaaataaataacttgttcgcttacgtctggatcgaaaggggttggaaacgattaaatctcttcctagtcaaaattaaatagaaggagatttaatcgatttctcccaatccccttcgatccaggtgcaaccgaataagtccttaaatgttgaggaacacgaaacaaccatgcattggcatgtaaagctccaagaattcgttgtatccttaacaactcacagaacatcaaccaaaattgcacgtcaagggtattgggtaagaaacaatcaaacaaatcctctctgtgtgcaaagaaacacggtgagtcatgccgagatcatactcatctgatatacatgcttacagctcacaagacattacaaacaactcatattgcattacaaagatcgtttcatgaaaaataaaataggccggaacaggacaaaaatccttgacgtgtaaagtaaatttacaacaaaaaaaaagccatatgtcaagctaaatctaattcgttttacgtagatcaacaacctgtagaaggcaacaaaactgagccacgcagaagtacagaatgattccagatgaaccatcgacgtgctacgtaaagagagtgacgagtcatatacatttggcaagaaaccatgaagctgcctacagccgtctcggtggcataagaacacaagaaattgtgttaattaatcaaagctataaataacgctcgcatgcctgtgcacttctccatcaccaccactgggtcttcagaccattagctttatctactccagagcgcagaagaacccgatcgacaccggatcctaaaccatgagggtgttgctcgttgccctcgctctcctggctctcgctgcgagcgccacctcccaggtgcagctccaggagtccggcggcggcctcgtgcagccgggcggctccctccgcctgagctgcgccgcgtccggcaacatcttcagcatcaacacgatgggctggtacaggcaggcccccggcaagcagcgggagctcgtggcctccatcaccacgggcggcaccacgaactacgaggacagcgtcaagggccgcttcaccatctccagggacaacgccaagaagacggtgtacctccagatgaaccgcctgaagccggaggacacggcggtctactactgcaaccaccgcaggtcctacagcggcagggactaccccgtgtacggcatggactactggggcaagggcaccctcgtgaccgtgtcctccaaggacgagctgtgacctaggtccccgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattggaattcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcacggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagatccagatctaaaccatgcagaaactcattaactcagtgcaaaactatgcctggggcagcaaaacggcgttgactgaactttatggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggatgattttgccttctcgctgcatgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaagagcttactgaaaaaattaacatctcttgctaagctgggagctctagatccccgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattggcgagctcgaattaattcagtacattaaaaacgtccgcaatgtgttattaagttgtctaagcgtcaatttgtttacaccacaatatatcctgcca

SEQ ID NO: 168 - DNA, 6032 bp, synthetic construct
  misc_feature 1..6032: note = Synthetic construct, pAG4994
  source 1..6032: mol_type = other DNA
gtttacccgccaatatatcctgtcaaacactgatagtttaaactgaaggcgggaaacgacaacctgatcatgagcggagaattaagggagtcacgttatgacccccgccgatgacgcgggacaagccgttttacgtttggaactgacagaaccgcaacgttgaaggagccactcagcctaagcggccgcattggacttaattaagtgaggccggccaagcgtcgatttaaatgtaccacatggcgcgccaactatcatgcgatcgcttcatgtctaactcgagttactggtacgtaccaaatccatggaatcaaggtaccaaagtaatcatattattttatgtgtgaatcttctttactttttcatttgattatgattatgaaggtatgaccttcataaccttcgtccgaaatccattatatccaaaggaaaataatgcttcgaaggacgaaggattttgatatttaacattttatgttgccttgttcttaattcatagcatttgagaacaagtccccaacaccaatctttatctttactatattaaagcaccagttcaacgatcgtctcgtgtcaatattattaaaaaactcctacatttctttataatcaacccgcactcttataatctcttctcttactactataataagagagtttatgtacaaaataaggtgaaattatgtataagtgttctggaccttggttgttggctcatattcacacaacctaatcaatagaaaacatatgttttattaaaacaaaatttatcatatatatatatatatatatatatatatatatatatatatataatataaaccgtagcaatgcacaggcatatgactagtggcaacttaataccatgtgtgtattaagatgaataagaggtatccaaataaataacttgttcgcttacgtctggatcgaaaggggttggaaacgattaaatctcttcctagtcaaaattaaatagaaggagatttaatcgatttctcccaatccccttcgatccaggtgcaaccgaataagtccttaaatgttgaggaacacgaaacaaccatgcattggcatgtaaagctccaagaattcgttgtatccttaacaactcacagaacatcaaccaaaattgcacgtcaagggtattgggtaagaaacaatcaaacaaatcctctctgtgtgcaaagaaacacggtgagtcatgccgagatcatactcatctgatatacatgcttacagctcacaagacattacaaacaactcatattgcattacaaagatcgtttcatgaaaaataaaataggccggaacaggacaaaaatccttgacgtgtaaagtaaatttacaacaaaaaaaaagccatatgtcaagctaaatctaattcgttttacgtagatcaacaacctgtagaaggcaacaaaactgagccacgcagaagtacagaatgattccagatgaaccatcgacgtgctacgtaaagagagtgacgagtcatatacatttggcaagaaaccatgaagctgcctacagccgtctcggtggcataagaacacaagaaattgtgttaattaatcaaagctataaataacgctcgcatgcctgtgcacttctccatcaccaccactgggtcttcagaccattagctttatctactccagagcgcagaagaacccgatcgacaccggatcctaaaccatgagggtgttgctcgttgccctcgctctcctggctctcgctgcgagcgccacctcccaggtgcagctccaggagttcggcggcggcctcgtgcagccgggcggctccctccgcctgagctgcgccgcgtccggcaggacgggctccagctacgcgatgggctggttcaggcaggcgcccggcaaggagagggagttcgtggcggccatctcgtggagcggcggcagcaccgactacgctgactccgtcaagggccgcttcaccatcagcagggacaacgcgaagaacacgatgtacctccagatgaactccctgaagccggaggacaccgccgtgtactactgcgcggtcgaccgcaacctcttcaagctgagggtggccgtccaggagtacaccaacctcggccagggcacccaggtgaccgtgtcctccaaggacgagctgtgacctaggtccccgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattggaattcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcacggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagatccagatctaaaccatgcagaaactcattaactcagtgcaaaactatgcctggggcagcaaaacggcgttgactgaactttatggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggatgattttgccttctcgctgcatgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaagagcttactgaaaaaattaacatctcttgctaagctgggagctctagatccccgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattggcgagctcgaattaattcagtacattaaaaacgtccgcaatgtgttattaagttgtctaagcgtcaatttgtttacaccacaatatatcctgcca

SEQ ID NO: 169 - DNA, 6029 bp, synthetic construct
  misc_feature 1..6029: note = Synthetic construct, pAG4995
  source 1..6029: mol_type = other DNA
gtttacccgccaatatatcctgtcaaacactgatagtttaaactgaaggcgggaaacgacaacctgatcatgagcggagaattaagggagtcacgttatgacccccgccgatgacgcgggacaagccgttttacgtttggaactgacagaaccgcaacgttgaaggagccactcagcctaagcggccgcattggacttaattaagtgaggccggccaagcgtcgatttaaatgtaccacatggcgcgccaactatcatgcgatcgcttcatgtctaactcgagttactggtacgtaccaaatccatggaatcaaggtaccaaagtaatcatattattttatgtgtgaatcttctttactttttcatttgattatgattatgaaggtatgaccttcataaccttcgtccgaaatccattatatccaaaggaaaataatgcttcgaaggacgaaggattttgatatttaacattttatgttgccttgttcttaattcatagcatttgagaacaagtccccaacaccaatctttatctttactatattaaagcaccagttcaacgatcgtctcgtgtcaatattattaaaaaactcctacatttctttataatcaacccgcactcttataatctcttctcttactactataataagagagtttatgtacaaaataaggtgaaattatgtataagtgttctggaccttggttgttggctcatattcacacaacctaatcaatagaaaacatatgttttattaaaacaaaatttatcatatatatatatatatatatatatatatatatatatatatataatataaaccgtagcaatgcacaggcatatgactagtggcaacttaataccatgtgtgtattaagatgaataagaggtatccaaataaataacttgttcgcttacgtctggatcgaaaggggttggaaacgattaaatctcttcctagtcaaaattaaatagaaggagatttaatcgatttctcccaatccccttcgatccaggtgcaaccgaataagtccttaaatgttgaggaacacgaaacaaccatgcattggcatgtaaagctccaagaattcgttgtatccttaacaactcacagaacatcaaccaaaattgcacgtcaagggtattgggtaagaaacaatcaaacaaatcctctctgtgtgcaaagaaacacggtgagtcatgccgagatcatactcatctgatatacatgcttacagctcacaagacattacaaacaactcatattgcattacaaagatcgtttcatgaaaaataaaataggccggaacaggacaaaaatccttgacgtgtaaagtaaatttacaacaaaaaaaaagccatatgtcaagctaaatctaattcgttttacgtagatcaacaacctgtagaaggcaacaaaactgagccacgcagaagtacagaatgattccagatgaaccatcgacgtgctacgtaaagagagtgacgagtcatatacatttggcaagaaaccatgaagctgcctacagccgtctcggtggcataagaacacaagaaattgtgttaattaatcaaagctataaataacgctcgcatgcctgtgcacttctccatcaccaccactgggtcttcagaccattagctttatctactccagagcgcagaagaacccgatcgacaccggatcctaaaccatgagggtgttgctcgttgccctcgctctcctggctctcgctgcgagcgccacctcccaggtgcagctccaggcctccggcggcggcctcgtgcaggcgggcggctccctccgcctgagctgcgccgcgtccggcaggaccttcaacagctacgcttggggctggttcaggcaggcgccgggcaaggagcgcggcttcgtggccaggatctccttcagcggcggccacacctactactccgacagcgtcaagggccgcttcacgatcagcagggacaacgccaagaactccgtgtacctccagatgaacagcctgaagcccgaggacacggccgtctactactgcgcggcggacccgaccccatacggcctccgcaacgagaggaactaccactactggggccagggcacccaggtgaccgtgtcctccaaggacgagctgtgacctaggtccccgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattggaattcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcacggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagatccagatctaaaccatgcagaaactcattaactcagtgcaaaactatgcctggggcagcaaaacggcgttgactgaactttatggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggatgattttgccttctcgctgcatgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaagagcttactgaaaaaattaacatctcttgctaagctgggagctctagatccccgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattggcgagctcgaattaattcagtacattaaaaacgtccgcaatgtgttattaagttgtctaagcgtcaatttgtttacaccacaatatatcctgcca

SEQ ID NO: 170 - DNA, 8274 bp, synthetic construct
  misc_feature 1..8274: note = Synthetic construct, pAG4996
  source 1..8274: mol_type = other DNA
gtttacccgccaatatatcctgtcaaacactgatagtttaaactgaaggcgggaaacgacaacctgatcatgagcggagaattaagggagtcacgttatgacccccgccgatgacgcgggacaagccgttttacgtttggaactgacagaaccgcaacgttgaaggagccactcagcctaagcggccgcattggacttaattaagtgaggccggccaagcgtcgatttaaatgtaccacatggcgcgccaactatcatgcgatcgcttcatgtctaactcgagttactggtacgtaccaaatccatggaatcaaggtaccaaagtaatcatattattttatgtgtgaatcttctttactttttcatttgattatgattatgaaggtatgaccttcataaccttcgtccgaaatccattatatccaaaggaaaataatgcttcgaaggacgaaggattttgatatttaacattttatgttgccttgttcttaattcatagcatttgagaacaagtccccaacaccaatctttatctttactatattaaagcaccagttcaacgatcgtctcgtgtcaatattattaaaaaactcctacatttctttataatcaacccgcactcttataatctcttctcttactactataataagagagtttatgtacaaaataaggtgaaattatgtataagtgttctggaccttggttgttggctcatattcacacaacctaatcaatagaaaacatatgttttattaaaacaaaatttatcatatatatatatatatatatatatatatatatatatatatataatataaaccgtagcaatgcacaggcatatgactagtggcaacttaataccatgtgtgtattaagatgaataagaggtatccaaataaataacttgttcgcttacgtctggatcgaaaggggttggaaacgattaaatctcttcctagtcaaaattaaatagaaggagatttaatcgatttctcccaatccccttcgatccaggtgcaaccgaataagtccttaaatgttgaggaacacgaaacaaccatgcattggcatgtaaagctccaagaattcgttgtatccttaacaactcacagaacatcaaccaaaattgcacgtcaagggtattgggtaagaaacaatcaaacaaatcctctctgtgtgcaaagaaacacggtgagtcatgccgagatcatactcatctgatatacatgcttacagctcacaagacattacaaacaactcatattgcattacaaagatcgtttcatgaaaaataaaataggccggaacaggacaaaaatccttgacgtgtaaagtaaatttacaacaaaaaaaaagccatatgtcaagctaaatctaattcgttttacgtagatcaacaacctgtagaaggcaacaaaactgagccacgcagaagtacagaatgattccagatgaaccatcgacgtgctacgtaaagagagtgacgagtcatatacatttggcaagaaaccatgaagctgcctacagccgtctcggtggcataagaacacaagaaattgtgttaattaatcaaagctataaataacgctcgcatgcctgtgcacttctccatcaccaccactgggtcttcagaccattagctttatctactccagagcgcagaagaacccgatcgacaccggatcctaaaccatgagggtgttgctcgttgccctcgctctcctggctctcgctgcgagcgccacctcccaggtgcagctccaggagtccggcggcggcctcgtgcagccgggcggctccctccgcctgagctgcgccgcgtccggcaacatcttcagcatcaacacgatgggctggtacaggcaggcccccggcaagcagcgggagctcgtggcctccatcaccacgggcggcaccacgaactacgaggacagcgtcaagggccgcttcaccatctccagggacaacgccaagaagacggtgtacctccagatgaaccgcctgaagccggaggacacggcggtctactactgcaaccaccgcaggtcctacagcggcagggactaccccgtgtacggcatggactactggggcaagggcaccctcgtgaccgtgtcctccaaggacgagctgtaacctaggtccccgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattgttaattaagtctaactcgagttactggtacgtatacagggttccttgcgtgaagaagggtggcctgcggttcaccattaacggtcacgactacttccagctagtactggtgaccaacgtcgcggcggcagggtcaatcaagtccatggaggttatgggttccaacacagcggattggatgccgatggcacgtaactggggcgcccaatggcactcactggcctacctcaccggtcaaggtctatcctttagggtcaccaacacagatgaccaaacgctcgtcttcaccaacgtcgtgccaccaggatggaagtttggccagacatttgcaagcaagctgcagttcaagtgagaggagaagcctgaattgataccggagcgtttcttttgggagtaacatctctggttgcctagcaaacatatgattgtatataagtttcgttgtgcgtttattctttcggtgtgtaaaataacatacatgctttcctgatattttcttgtatatatgtacacacacacgacaaatccttccatttctattattattgaacaatttaattgcgagggcgagtacttgtctgtttaccttttttttttcagatggcattttatagtttaacctttcatggaccggcagtagttctaaccatgaatgaaaagaaatcatagtccacaccacgcagggacattgtggtcattttagacaagacgatttgattaatgtcttgtatgatatggtcgacagtgaggactaacaaacatatggcatattttattaccggcgagttaaataaatttatgtcacagtaataaactgcctaataaatgcacgccagaaaatataatgataaaaaaaagaaaagatacataagtccattgcttctacttttttaaaaattaaatccaacattttctattttttggtataaacttggaagtactagttggatatgcaaaatcatctaacctccatatatttcatcaatttgtttactttacatatgggagaggatagtatgtcaaagaaaatgacaacaagcttacaagtttcttattttaaaagttccgctaacttatcaagcatagtgtgccacgcaaaactgacaacaaaccaacaaatttaaggagcgcctaacttatcatctatgacataccgcacaaaatgataacatactagagaaactttattgcacaaaaggaaatttatccataaggcaaaggaacatcttaaggctttggatatacatttaccaacaagcattgtttgtattacccctaaagcgcaagacatgtcatccatgagtcatagtgtgtatatctcaacattgcaaagctaccttttttctattatacttttcgcattataggctagatattatctatacatgtcaacaaactctatccctacgtcatatctgaagattcttttcttcactatataagttggcttccctgtcattgaactcacatcaaccagcccaagtttccaataacatcctcaaatagctggatcctaaaccatgagggtgttgctcgttgccctcgctctcctggctctcgctgcgagcgccacctcccaggttcagctgcaggaaagcggtggcggactggtgcagccaggtggcagcctcaggctgagctgcgctgctagcggcaatatttttagcattaacacaatgggttggtatagacaggctcctggcaagcagcgtgagctcgttgccagcattaccacgggtggtacaaccaattatgaagatagcgtgaagggtcgttttaccattagcagggacaatgctaagaagaccgtttacctccagatgaacaggctgaagccagaagataccgccgtgtattactgcaaccacaggagaagctatagcggaagagattatcctgtttacggtatggactactggggcaagggaaccctggttaccgtgagcagcaaggacgagctgtaacctaggtccccgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattggaattcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcacggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagatccagatctaaaccatgcagaaactcattaactcagtgcaaaactatgcctggggcagcaaaacggcgttgactgaactttatggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggatgattttgccttctcgctgcatgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaagagcttactgaaaaaattaacatctcttgctaagctgggagctctagatccccgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattggcgagctcgaattaattcagtacattaaaaacgtccgcaatgtgttattaagttgtctaagcgtcaatttgtttacaccacaatatatcctgcca

SEQ ID NO: 171 - DNA, 8280 bp, synthetic construct
  misc_feature 1..8280: note = Synthetic construct, pAG4997
  source 1..8280: mol_type = other DNA
gtttacccgccaatatatcctgtcaaacactgatagtttaaactgaaggcgggaaacgacaacctgatcatgagcggagaattaagggagtcacgttatgacccccgccgatgacgcgggacaagccgttttacgtttggaactgacagaaccgcaacgttgaaggagccactcagcctaagcggccgcattggacttaattaagtgaggccggccaagcgtcgatttaaatgtaccacatggcgcgccaactatcatgcgatcgcttcatgtctaactcgagttactggtacgtaccaaatccatggaatcaaggtaccaaagtaatcatattattttatgtgtgaatcttctttactttttcatttgattatgattatgaaggtatgaccttcataaccttcgtccgaaatccattatatccaaaggaaaataatgcttcgaaggacgaaggattttgatatttaacattttatgttgccttgttcttaattcatagcatttgagaacaagtccccaacaccaatctttatctttactatattaaagcaccagttcaacgatcgtctcgtgtcaatattattaaaaaactcctacatttctttataatcaacccgcactcttataatctcttctcttactactataataagagagtttatgtacaaaataaggtgaaattatgtataagtgttctggaccttggttgttggctcatattcacacaacctaatcaatagaaaacatatgttttattaaaacaaaatttatcatatatatatatatatatatatatatatatatatatatatataatataaaccgtagcaatgcacaggcatatgactagtggcaacttaataccatgtgtgtattaagatgaataagaggtatccaaataaataacttgttcgcttacgtctggatcgaaaggggttggaaacgattaaatctcttcctagtcaaaattaaatagaaggagatttaatcgatttctcccaatccccttcgatccaggtgcaaccgaataagtccttaaatgttgaggaacacgaaacaaccatgcattggcatgtaaagctccaagaattcgttgtatccttaacaactcacagaacatcaaccaaaattgcacgtcaagggtattgggtaagaaacaatcaaacaaatcctctctgtgtgcaaagaaacacggtgagtcatgccgagatcatactcatctgatatacatgcttacagctcacaagacattacaaacaactcatattgcattacaaagatcgtttcatgaaaaataaaataggccggaacaggacaaaaatccttgacgtgtaaagtaaatttacaacaaaaaaaaagccatatgtcaagctaaatctaattcgttttacgtagatcaacaacctgtagaaggcaacaaaactgagccacgcagaagtacagaatgattccagatgaaccatcgacgtgctacgtaaagagagtgacgagtcatatacatttggcaagaaaccatgaagctgcctacagccgtctcggtggcataagaacacaagaaattgtgttaattaatcaaagctataaataacgctcgcatgcctgtgcacttctccatcaccaccactgggtcttcagaccattagctttatctactccagagcgcagaagaacccgatcgacaccggatcctaaaccatgagggtgttgctcgttgccctcgctctcctggctctcgctgcgagcgccacctcccaggtgcagctccaggagttcggcggcggcctcgtgcagccgggcggctccctccgcctgagctgcgccgcgtccggcaggacgggctccagctacgcgatgggctggttcaggcaggcgcccggcaaggagagggagttcgtggcggccatctcgtggagcggcggcagcaccgactacgctgactccgtcaagggccgcttcaccatcagcagggacaacgcgaagaacacgatgtacctccagatgaactccctgaagccggaggacaccgccgtgtactactgcgcggtcgaccgcaacctcttcaagctgagggtggccgtccaggagtacaccaacctcggccagggcacccaggtgaccgtgtcctccaaggacgagctgtaacctaggtccccgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattgttaattaagtctaactcgagttactggtacgtatacagggttccttgcgtgaagaagggtggcctgcggttcaccattaacggtcacgactacttccagctagtactggtgaccaacgtcgcggcggcagggtcaatcaagtccatggaggttatgggttccaacacagcggattggatgccgatggcacgtaactggggcgcccaatggcactcactggcctacctcaccggtcaaggtctatcctttagggtcaccaacacagatgaccaaacgctcgtcttcaccaacgtcgtgccaccaggatggaagtttggccagacatttgcaagcaagctgcagttcaagtgagaggagaagcctgaattgataccggagcgtttcttttgggagtaacatctctggttgcctagcaaacatatgattgtatataagtttcgttgtgcgtttattctttcggtgtgtaaaataacatacatgctttcctgatattttcttgtatatatgtacacacacacgacaaatccttccatttctattattattgaacaatttaattgcgagggcgagtacttgtctgtttaccttttttttttcagatggcattttatagtttaacctttcatggaccggcagtagttctaaccatgaatgaaaagaaatcatagtccacaccacgcagggacattgtggtcattttagacaagacgatttgattaatgtcttgtatgatatggtcgacagtgaggactaacaaacatatggcatattttattaccggcgagttaaataaatttatgtcacagtaataaactgcctaataaatgcacgccagaaaatataatgataaaaaaaagaaaagatacataagtccattgcttctacttttttaaaaattaaatccaacattttctattttttggtataaacttggaagtactagttggatatgcaaaatcatctaacctccatatatttcatcaatttgtttactttacatatgggagaggatagtatgtcaaagaaaatgacaacaagcttacaagtttcttattttaaaagttccgctaacttatcaagcatagtgtgccacgcaaaactgacaacaaaccaacaaatttaaggagcgcctaacttatcatctatgacataccgcacaaaatgataacatactagagaaactttattgcacaaaaggaaatttatccataaggcaaaggaacatcttaaggctttggatatacatttaccaacaagcattgtttgtattacccctaaagcgcaagacatgtcatccatgagtcatagtgtgtatatctcaacattgcaaagctaccttttttctattatacttttcgcattataggctagatattatctatacatgtcaacaaactctatccctacgtcatatctgaagattcttttcttcactatataagttggcttccctgtcattgaactcacatcaaccagcccaagtttccaataacatcctcaaatagctggatcctaaaccatgagggtgttgctcgttgccctcgctctcctggctctcgctgcgagcgccacctcccaggttcagctccaggagtttggtggcggactggtgcagccaggtggcagcctcaggctgagctgcgctgctagcggtagaaccggcagcagctatgctatgggatggtttagacaggctccaggcaaggagcgtgaatttgttgctgccattagctggagcggaggtagcaccgattatgctgacagcgtgaagggcaggtttaccattagcagagataatgccaagaacaccatgtacctccagatgaatagcctgaagccagaggataccgctgtttattactgcgccgtggaccgtaatctctttaagctgagggttgctgtgcaggaatacaccaacctcggccagggaacccaggttaccgtgagcagcaaggacgagctgtaacctaggtccccgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattggaattcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcacggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagatccagatctaaaccatgcagaaactcattaactcagtgcaaaactatgcctggggcagcaaaacggcgttgactgaactttatggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggatgattttgccttctcgctgcatgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaagagcttactgaaaaaattaacatctcttgctaagctgggagctctagatccccgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattggcgagctcgaattaattcagtacattaaaaacgtccgcaatgtgttattaagttgtctaagcgtcaatttgtttacaccacaatatatcctgcca

SEQ ID NO: 172 - DNA, 8274 bp, synthetic construct
  misc_feature 1..8274: note = Synthetic construct, pAG4998
  source 1..8274: mol_type = other DNA
gtttacccgccaatatatcctgtcaaacactgatagtttaaactgaaggcgggaaacgacaacctgatcatgagcggagaattaagggagtcacgttatgacccccgccgatgacgcgggacaagccgttttacgtttggaactgacagaaccgcaacgttgaaggagccactcagcctaagcggccgcattggacttaattaagtgaggccggccaagcgtcgatttaaatgtaccacatggcgcgccaactatcatgcgatcgcttcatgtctaactcgagttactggtacgtaccaaatccatggaatcaaggtaccaaagtaatcatattattttatgtgtgaatcttctttactttttcatttgattatgattatgaaggtatgaccttcataaccttcgtccgaaatccattatatccaaaggaaaataatgcttcgaaggacgaaggattttgatatttaacattttatgttgccttgttcttaattcatagcatttgagaacaagtccccaacaccaatctttatctttactatattaaagcaccagttcaacgatcgtctcgtgtcaatattattaaaaaactcctacatttctttataatcaacccgcactcttataatctcttctcttactactataataagagagtttatgtacaaaataaggtgaaattatgtataagtgttctggaccttggttgttggctcatattcacacaacctaatcaatagaaaacatatgttttattaaaacaaaatttatcatatatatatatatatatatatatatatatatatatatatataatataaaccgtagcaatgcacaggcatatgactagtggcaacttaataccatgtgtgtattaagatgaataagaggtatccaaataaataacttgttcgcttacgtctggatcgaaaggggttggaaacgattaaatctcttcctagtcaaaattaaatagaaggagatttaatcgatttctcccaatccccttcgatccaggtgcaaccgaataagtccttaaatgttgaggaacacgaaacaaccatgcattggcatgtaaagctccaagaattcgttgtatccttaacaactcacagaacatcaaccaaaattgcacgtcaagggtattgggtaagaaacaatcaaacaaatcctctctgtgtgcaaagaaacacggtgagtcatgccgagatcatactcatctgatatacatgcttacagctcacaagacattacaaacaactcatattgcattacaaagatcgtttcatgaaaaataaaataggccggaacaggacaaaaatccttgacgtgtaaagtaaatttacaacaaaaaaaaagccatatgtcaagctaaatctaattcgttttacgtagatcaacaacctgtagaaggcaacaaaactgagccacgcagaagtacagaatgattccagatgaaccatcgacgtgctacgtaaagagagtgacgagtcatatacatttggcaagaaaccatgaagctgcctacagccgtctcggtggcataagaacacaagaaattgtgttaattaatcaaagctataaataacgctcgcatgcctgtgcacttctccatcaccaccactgggtcttcagaccattagctttatctactccagagcgcagaagaacccgatcgacaccggatcctaaaccatgagggtgttgctcgttgccctcgctctcctggctctcgctgcgagcgccacctcccaggtgcagctccaggcctccggcggcggcctcgtgcaggcgggcggctccctccgcctgagctgcgccgcgtccggcaggaccttcaacagctacgcttggggctggttcaggcaggcgccgggcaaggagcgcggcttcgtggccaggatctccttcagcggcggccacacctactactccgacagcgtcaagggccgcttcacgatcagcagggacaacgccaagaactccgtgtacctccagatgaacagcctgaagcccgaggacacggccgtctactactgcgcggcggacccgaccccatacggcctccgcaacgagaggaactaccactactggggccagggcacccaggtgaccgtgtcctccaaggacgagctgtaacctaggtccccgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattgttaattaagtctaactcgagttactggtacgtatacagggttccttgcgtgaagaagggtggcctgcggttcaccattaacggtcacgactacttccagctagtactggtgaccaacgtcgcggcggcagggtcaatcaagtccatggaggttatgggttccaacacagcggattggatgccgatggcacgtaactggggcgcccaatggcactcactggcctacctcaccggtcaaggtctatcctttagggtcaccaacacagatgaccaaacgctcgtcttcaccaacgtcgtgccaccaggatggaagtttggccagacatttgcaagcaagctgcagttcaagtgagaggagaagcctgaattgataccggagcgtttcttttgggagtaacatctctggttgcctagcaaacatatgattgtatataagtttcgttgtgcgtttattctttcggtgtgtaaaataacatacatgctttcctgatattttcttgtatatatgtacacacacacgacaaatccttccatttctattattattgaacaatttaattgcgagggcgagtacttgtctgtttaccttttttttttcagatggcattttatagtttaacctttcatggaccggcagtagttctaaccatgaatgaaaagaaatcatagtccacaccacgcagggacattgtggtcattttagacaagacgatttgattaatgtcttgtatgatatggtcgacagtgaggactaacaaacatatggcatattttattaccggcgagttaaataaatttatgtcacagtaataaactgcctaataaatgcacgccagaaaatataatgataaaaaaaagaaaagatacataagtccattgcttctacttttttaaaaattaaatccaacattttctattttttggtataaacttggaagtactagttggatatgcaaaatcatctaacctccatatatttcatcaatttgtttactttacatatgggagaggatagtatgtcaaagaaaatgacaacaagcttacaagtttcttattttaaaagttccgctaacttatcaagcatagtgtgccacgcaaaactgacaacaaaccaacaaatttaaggagcgcctaacttatcatctatgacataccgcacaaaatgataacatactagagaaactttattgcacaaaaggaaatttatccataaggcaaaggaacatcttaaggctttggatatacatttaccaacaagcattgtttgtattacccctaaagcgcaagacatgtcatccatgagtcatagtgtgtatatctcaacattgcaaagctaccttttttctattatacttttcgcattataggctagatattatctatacatgtcaacaaactctatccctacgtcatatctgaagattcttttcttcactatataagttggcttccctgtcattgaactcacatcaaccagcccaagtttccaataacatcctcaaatagctggatcctaaaccatgagggtgttgctcgttgccctcgctctcctggctctcgctgcgagcgccacctcccaggttcagctccaggcttcgggcggcgggctcgtccaggcgggcggctcgctcaggctctcgtgcgcggcgtcggggcggactttcaacagctacgcttggggctggttcaggcaggcgccgggcaaggagcgcggcttcgtggccaggatctccttcagcggcggccacacctactactccgacagcgtcaagggccgcttcacgatctccagggacaacgccaagaacagcgtgtacctccagatgaactccctgaagcccgaggacacggccgtctactactgcgcggcggacccgacgccctacggcctcaggaacgagcggaactaccattactgggggcagggcacgcaggtcactgtctcttcgaaggacgagctgtaacctaggtccccgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattggaattcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcacggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagatccagatctaaaccatgcagaaactcattaactcagtgcaaaactatgcctggggcagcaaaacggcgttgactgaactttatggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggatgattttgccttctcgctgcatgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaagagcttactgaaaaaattaacatctcttgctaagctgggagctctagatccccgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattggcgagctcgaattaattcagtacattaaaaacgtccgcaatgtgttattaagttgtctaagcgtcaatttgtttacaccacaatatatcctgcca

SEQ ID NO: 173 - DNA, 372 bp, synthetic construct
  misc_feature 1..372: note = Synthetic construct, chIL101A11A coding seq
  source 1..372: mol_type = other DNA
caggttcagctgcaggaaagcggtggcggactggtgcagccaggtggcagcctcaggctgagctgcgctgctagcggcaatatttttagcattaacacaatgggttggtatagacaggctcctggcaagcagcgtgagctcgttgccagcattaccacgggtggtacaaccaattatgaagatagcgtgaagggtcgttttaccattagcagggacaatgctaagaagaccgtttacctccagatgaacaggctgaagccagaagataccgccgtgtattactgcaaccacaggagaagctatagcggaagagattatcctgtttacggtatggactactggggcaagggaaccctggttaccgtgagcagc

SEQ ID NO: 174 - DNA, 372 bp, synthetic construct
  misc_feature 1..372: note = Synthetic construct, chIL101A11B coding seq
  source 1..372: mol_type = other DNA
caggtgcagctccaggagtccggcggcggcctcgtgcagccgggcggctccctccgcctgagctgcgccgcgtccggcaacatcttcagcatcaacacgatgggctggtacaggcaggcccccggcaagcagcgggagctcgtggcctccatcaccacgggcggcaccacgaactacgaggacagcgtcaagggccgcttcaccatctccagggacaacgccaagaagacggtgtacctccagatgaaccgcctgaagccggaggacacggcggtctactactgcaaccaccgcaggtcctacagcggcagggactaccccgtgtacggcatggactactggggcaagggcaccctcgtgaccgtgtcctcc

SEQ ID NO: 175 - DNA, 375 bp, synthetic construct
  misc_feature 1..375: note = Synthetic construct, chIL101F11A coding seq
  source 1..375: mol_type = other DNA
caggttcagctccaggagtttggtggcggactggtgcagccaggtggcagcctcaggctgagctgcgctgctagcggtagaaccggcagcagctatgctatgggatggtttagacaggctccaggcaaggagcgtgaatttgttgctgccattagctggagcggaggtagcaccgattatgctgacagcgtgaagggcaggtttaccattagcagagataatgccaagaacaccatgtacctccagatgaatagcctgaagccagaggataccgctgtttattactgcgccgtggaccgtaatctctttaagctgagggttgctgtgcaggaatacaccaacctcggccagggaacccaggttaccgtgagcagc

SEQ ID NO: 176 - DNA, 375 bp, synthetic construct
  misc_feature 1..375: note = Synthetic construct, chIL101F11B coding seq
  source 1..375: mol_type = other DNA
caggtgcagctccaggagttcggcggcggcctcgtgcagccgggcggctccctccgcctgagctgcgccgcgtccggcaggacgggctccagctacgcgatgggctggttcaggcaggcgcccggcaaggagagggagttcgtggcggccatctcgtggagcggcggcagcaccgactacgctgactccgtcaagggccgcttcaccatcagcagggacaacgcgaagaacacgatgtacctccagatgaactccctgaagccggaggacaccgccgtgtactactgcgcggtcgaccgcaacctcttcaagctgagggtggccgtccaggagtacaccaacctcggccagggcacccaggtgaccgtgtcctcc

SEQ ID NO: 177 - DNA, 372 bp, synthetic construct
  misc_feature 1..372: note = Synthetic construct, chIL101H1A coding seq
  source 1..372: mol_type = other DNA
caggttcagctccaggcttcgggcggcgggctcgtccaggcgggcggctcgctcaggctctcgtgcgcggcgtcggggcggactttcaacagctacgcttggggctggttcaggcaggcgccgggcaaggagcgcggcttcgtggccaggatctccttcagcggcggccacacctactactccgacagcgtcaagggccgcttcacgatctccagggacaacgccaagaacagcgtgtacctccagatgaactccctgaagcccgaggacacggccgtctactactgcgcggcggacccgacgccctacggcctcaggaacgagcggaactaccattactgggggcagggcacgcaggtcactgtctcttcg

SEQ ID NO: 178 - DNA, 372 bp, synthetic construct
  misc_feature 1..372: note = Synthetic construct, chIL101H1B coding seq
  source 1..372: mol_type = other DNA
caggtgcagctccaggcctccggcggcggcctcgtgcaggcgggcggctccctccgcctgagctgcgccgcgtccggcaggaccttcaacagctacgcttggggctggttcaggcaggcgccgggcaaggagcgcggcttcgtggccaggatctccttcagcggcggccacacctactactccgacagcgtcaagggccgcttcacgatcagcagggacaacgccaagaactccgtgtacctccagatgaacagcctgaagcccgaggacacggccgtctactactgcgcggcggacccgaccccatacggcctccgcaacgagaggaactaccactactggggccagggcacccaggtgaccgtgtcctcc

SEQ ID NO: 179 - protein, 147 aa, synthetic construct
  REGION 1..147: note = Synthetic construct, xGZein27ss:chIL10sdAb1H1:KDEL
MRVLLVALALLALAASATSQVQLQASGGGLVQAGGSLRLSCAASGRTFNSYAWGWFRQAPGKERGFVARISFSGGHTYYSDSVKGRFTISRDNAKNSVYLQMNSLKPEDTAVYYCAADPTPYGLRNERNYHYWGQGTQVTVSSKDEL

SEQ ID NO: 180 - protein, 8 aa, synthetic construct
  REGION 1..8: note = Synthetic construct, counter selection peptide 1
DDELNIQL

SEQ ID NO: 181 - protein, 8 aa, synthetic construct
  REGION 1..8: note = Synthetic construct, counter selection peptide 2
VLPRAMQT

SEQ ID NO: 182 - protein, 8 aa, synthetic construct
  REGION 1..8: note = Synthetic construct, counter selection peptide 3
EKMDENGI

SEQ ID NO: 183 - protein, 8 aa, synthetic construct
  REGION 1..8: note = Synthetic construct, counter selection peptide 4
EPTCLHFS

SEQ ID NO: 184 - protein, 7 aa, synthetic construct
  REGION 1..7: note = Synthetic construct, counter selection peptide 5
DQMGDLL

SEQ ID NO: 185 - protein, 7 aa, synthetic construct
  REGION 1..7: note = Synthetic construct, counter selection peptide 6
DQLHSLL

SEQ ID NO: 186 - protein, 8 aa, synthetic construct
  REGION 1..8: note = Synthetic construct, counter selection peptide 7
VMPKAESD

SEQ ID NO: 187 - protein, 8 aa, synthetic construct
  REGION 1..8: note = Synthetic construct, counter selection peptide 8
VMPQAENH

SEQ ID NO: 188 - protein, 8 aa, synthetic construct
  REGION 1..8: note = Synthetic construct, counter selection peptide
SKLQERGV

SEQ ID NO: 189 - protein, 8 aa, synthetic construct
  REGION 1..8: note = Synthetic construct, counter selection peptide 10
SELQERGV

SEQ ID NO: 190 - protein, 8 aa, synthetic construct
  REGION 1..8: note = Synthetic construct, counter selection peptide 11
ENSCIHFP

SEQ ID NO: 191 - protein, 8 aa, synthetic construct
  REGION 1..8: note = Synthetic construct, counter selection peptide 12
DSSCIHLP

SEQ ID NO: 192 - protein, 7 aa, synthetic construct
  REGION 1..7: note = Synthetic construct, counter selection peptide 13
DQLNSML

SEQ ID NO: 193 - protein, 8 aa, synthetic construct
  REGION 1..8: note = Synthetic construct, counter selection peptide 14
NMLQERGV

SEQ ID NO: 194 - protein, 8 aa, synthetic construct
  REGION 1..8: note = Synthetic construct, counter selection peptide 15
DSSCTHFP

SEQ ID NO: 195 - protein, 7 aa, synthetic construct
  REGION 1..7: note = Synthetic construct; counter selection peptide 16
DDLEIGL

SEQ ID NO: 196 - protein, 12 aa, synthetic construct
  REGION 1..12: note = Synthetic construct, counter selection peptide 17
VLPTAIADMTEE

SEQ ID NO: 197 - protein, 8 aa, synthetic construct
  REGION 1..8: note = Synthetic construct, counter selection peptide 18
TQMEGKGP

SEQ ID NO: 198 - protein, 7 aa, synthetic construct
  REGION 1..7: note = Synthetic construct, counter selection peptide 19
NQCCRFV

SEQ ID NO: 199 - protein, 6 aa, synthetic construct
  REGION 1..6: note = Synthetic construct, IEGRMD linker
IEGRMD

SEQ ID NO: 200 - DNA, 20 bp, synthetic construct
  misc_feature 1..20: note = Synthetic construct, forward primer 3661
  source 1..20: mol_type = other DNA
cgtgcccaagttcagttaca

SEQ ID NO: 201 - DNA, 22 bp, synthetic construct
  misc_feature 1..22: note = Synthetic construct, [00171], reverse primer 3662
  source 1..22: mol_type = other DNA
ttgcaacaagttctctttgctt

SEQ ID NO: 202 - DNA, 372 bp, synthetic construct
  misc_feature 1..372: note = Synthetic cosntruct, Nb1A11
  source 1..372: mol_type = other DNA
caagttcagttacaggaaagcgggggaggtttagttcagcctgggggttcattgaggttgagttgtgcagcaagtggaaatattttttctattaatactatgggatggtatagacaagctccaggaaagcaaagagaacttgttgcaagtattactactggaggaactacaaattacgaagatagtgttaaaggaagattcactatttcaagagataatgctaagaaaacagtttatcttcagatgaatagattgaagccagaagatacagcagtttactactgtaatcatagaagatcatactctggtagagattatcctgtttatggtatggattattggggaaaagggacattagttacagttagcagc

SEQ ID NO: 203 - DNA, 676 bp, synthetic construct
  misc_feature 1..676: note = Synthetic construct, Nb1A11:AtUBQ10i
  source 1..676: mol_type = other DNA
caagttcagttacaggaaagcgggggaggtttagttcagcctgggggttcattgaggttgagttgtgcagcaagtggaaatattttttctattaatactatgggatggtatagacaagctccaggtaaatttctgtgttccttattctctcaaaatcttcgattttgttttcgttcgatcccaatttcgtatatgttctttggtttagattctgttaatcttagatcgaagacgattttctgggtttgatcgttagatatcatcttaattctcgattagggtttcatagatatcatccgatttgttcaaataatttgagttttgtcgaataattactcttcgatttgtgatttctatctagatctggtgttagtttctagtttgtgcgatcgaatttgtcgattaatctgagtttttctgattaacaggaaagcaaagagaacttgttgcaagtattactactggaggaactacaaattacgaagatagtgttaaaggaagattcactatttcaagagataatgctaagaaaacagtttatcttcagatgaatagattgaagccagaagatacagcagtttactactgtaatcatagaagatcatactctggtagagattatcctgtttatggtatggattattggggaaaagggacattagttacagttagcagc

SEQ ID NO: 204 - DNA, 1466 bp, synthetic construct
  misc_feature 1..1466: note = Synthetic construct, prNbUbi1
  source 1..1466: mol_type = other DNA
catgaaagtccacatcatcagctcgtcccaaacatcactactagacccaactcgttcaatcttctcgactacaacaaatgaaatccgctcatcaaggtgtctgaggctgatctcaataaatggagggactaattgtatggatcgaaatctgccccaaaatatttagggtaaggtacattgaagaaagagtcatcgaggtcgatcaggaaacgatcgagatgttaacaatggtcgatgtcgagcaccgcatgtagagttgtaacacctagtttttagaataggataatacaaagaatattctattggatatcctttacacttatattattagagtttgttaggaaaatgacccacataaataggaaaaaagacaatgaatggagacaggtgacatttatctgatgagaacagacttttgatagaagatattttctctctcactaagatacaaacactacattttcatcaagattcttgttcatatcattgtacacttttctatcaaatctgaaataatttaaatattctaggatttgtctgtcactcatcattgtcagacgggataatcatgtactcatccttttttggcaaaccactttttctatttacttaaatgccatttattgatatctattgctagtcattcctccaccgttgctcatacttttttgcaatagtatgcatgttgatatcaatccaccaccaaatcttctaacattaatcatattttcacaacttacatttataaatattattattaactaagtttaactcactattatataaactcaattgttttactcgaaagttacactattatattgagaattacgtttccaaactttttaagcatttattgtgtaaccataagagactttgattttttaaaaattatttagattttattaatgagaatggcacaacattatggtcaactatgtatttcatcattaactaaatagttagcactttgattctttcacatgaattatgaatttatgatgggctcaaattaaaattaaattattcacaaaaacttatttttatattctacgacacccacttttctagctttttcccgaaggggcgtgagagtgtcacacacgctccaaatttcccaaccaaacaaggaaagggcagagaaagatagctttagcgtgttgttttggtgcactacacgtcattaggacacgtgtcatgatataataggccaatcccacgaggcggtttcgtcttgagtcggccatagtgtccataaatgagggctctccgtcggtttccccatcattcatcagatttatcttctatacttcatcgccttcatatttctctctcaaggtttgagaatttcttcaatttctcgctttagcagttcttttttattgaatcaacgatttcggcatctaaagtcctaattttgaagttcattgctttaattgtttgttgttgattttatattattacag

SEQ ID NO: 205 - DNA, 90 bp, synthetic construct
  misc_feature 1..90: note = Synthetic construct, ssPR1a
  source 1..90: mol_type = other DNA
atgggatttgttctcttttcacaattgccttcatttcttcttgtctctacacttctcttattcctagtaatatcccactcttgccgtgcc

SEQ ID NO: 206 - DNA, 102 bp, synthetic construct
  misc_feature 1..102: note = Synthetic construct, myc:6xHis:KDEL
  source 1..102: mol_type = other DNA
acttctggaccaggaggacaaggagctgaacaaaagttgatttctgaagaggatcttggagctcatcatcatcatcatcatggagcttctaaggatgaactt

SEQ ID NO: 207 - protein, 149 aa, Llama glama
  REGION 1..149: note = MISC_FEATURE - Llama Il-10
ENSCAHFPASLPHMLRELRAAFGRVKTFFQMKDQLDNMLLTRSLLEDFKGYLGCQALSEMIQFYLEEVMPQAENHGPDIKEHVNSLGEKLKTLRLRLRRCHRFLPCENKSKAVEQVRGVFSKLQEKGVYKAMSEFDIFINYIEAYMTMK

SEQ ID NO: 208 - protein, 154 aa, synthetic construct
  REGION 1..154: note = Synthetic construct, 40-IL-bR2-1H5 (FIG.4)
QVQLQESGGGLVQAGGSLRLSCAASGRTFNSYAWGWFRQAPGKERGFVARISFSGGHTYYSDSVKGRFTISRDNAKNSVYLQMNSLKPEDTAVYYCAADPTPYGLRNERNYHYWGQGTQVTVSSTSGPGGQGAEQKLISEEDLGAHHHHHHGAS

SEQ ID NO: 209 - protein, 7 aa, synthetic construct
  REGION 1..7: note = Synthetic construct, 68-IL-bR2-1D9_ AA 28 - 34
RLSINVM

SEQ ID NO: 210 - protein, 4 aa, synthetic construct
  REGION 1..4: note = Synthetic construct, 68-IL-bR2-1D9_AA 54-57
NSRS

SEQ ID NO: 211 - protein, 5 aa, synthetic construct
  REGION 1..5: note = Synthetic construct, 03-IL-bR2-1C1_AA 30-34
SINTM

SEQ ID NO: 212 - protein, 7 aa, synthetic construct
  REGION 1..7: note = Synthetic construct, 03-IL-bR2-1C1 AA 97-103
NHRRSYS

SEQ ID NO: 213 - protein, 9 aa, synthetic construct
  REGION 1..9: note = Synthetic construct, 03-IL-bR2-1C1_AA105-112
RDYPVYGMD

SEQ ID NO: 214 - protein, 5 aa, synthetic construct
  REGION 1..5: note = Synthetic construct, 35-IL-bR2-1C5_AA 30-34
GINVM

SEQ ID NO: 215 - protein, 5 aa, synthetic construct
  REGION 1..5: note = Synthetic construct, 35-IL-bR2-1C5_AA 50 -54
TVTTG

SEQ ID NO: 216 - protein, 8 aa, synthetic construct
  REGION 1..8: note = Synthetic construct, 48-IL-bR2-1H6_AA 27-34
SISSIDGM

SEQ ID NO: 217 - protein, 5 aa, synthetic construct
  REGION 1..5: note = Synthetic construct, 48-IL-bR2-1H6_AA 75-79
DQTTL

SEQ ID NO: 218 - protein, 9 aa, synthetic construct
  REGION 1..9: note = Synthetic construct, 48-IL-bR2-1H6_AA 105 - 112
VPDWYYGMD

SEQ ID NO: 219 - protein, 4 aa, synthetic construct
  REGION 1..4: note = Synthetic construct, 01-IL-bR2-2A8_AA 76-79
QTTL

SEQ ID NO: 220 - protein, 7 aa, synthetic construct
  REGION 1..7: note = Synthetic construct, 85-IL-bR2-1E11_AA 102-108
ASLSIYR

SEQ ID NO: 221 - protein, 9 aa, synthetic construct
  REGION 1..9: note = Synthetic construct, 44-IL-bR2-1D6_AA 26 - 34
ESISSINTM

SEQ ID NO: 222 - protein, 4 aa, synthetic construct
  REGION 1..4: note = Synthetic construct, 44-IL-bR2-1D6_AA 99-102
GVPT

SEQ ID NO: 223 - protein, 11 aa, synthetic construct
  REGION 1..11: note = Synthetic construct, 44-IL-bR2-1D6_AA 104-113
DDDAMPISWRF

SEQ ID NO: 224 - protein, 5 aa, synthetic construct
  REGION 1..5: note = Synthetic construct, 27-IL-bR2-1C4_AA 27 - 31
FSLEN

SEQ ID NO: 225 - protein, 5 aa, synthetic construct
  REGION 1..5: note = Synthetic construct, 27-IL-bR2-1C4_AA 46 - 50
EGLSC

SEQ ID NO: 226 - protein, 9 aa, synthetic construct
  REGION 1..9: note = Synthetic construct, 27-IL-bR2-1C4_AA 53-61
STDDSIFSV

SEQ ID NO: 227 - protein, 8 aa, synthetic construct
  REGION 1..8: note = Synthetic construct, 27-IL-bR2-1C4_AA 98-105
TSRGLGSC

SEQ ID NO: 228 - protein, 6 aa, synthetic construct
  REGION 1..6: note = Synthetic construct, 32-IL-bR2-1H4_AA 52 - 57
TNADRI

SEQ ID NO: 229 - protein, 5 aa, synthetic construct
  REGION 1..5: note = Synthetic construct, 32-IL-bR2-1H4_AA 98 - 102
TNFYS

SEQ ID NO: 230 - protein, 5 aa, synthetic construct
  REGION 1..5: note = Synthetic construct, 86-IL-bR2-1F11_AA 100-104
RNLFK

SEQ ID NO: 231 - protein, 5 aa, synthetic construct
  REGION 1..5: note = Synthetic construct, 86-IL-bR2-1F11_AA 107-110
VAVQE

SEQ ID NO: 232 - protein, 4 aa, synthetic construct
  REGION 1..4: note = Synthetic construct, 20-IL-bR2-1D3_AA 44 - 47
GLEW

SEQ ID NO: 233 - protein, 4 aa, synthetic construct
  REGION 1..4: note = Synthetic construct, 20-IL-bR2-1D3_AA 52-55
TWNV

SEQ ID NO: 234 - protein, 8 aa, synthetic construct
  REGION 1..8: note = Synthetic construct, 20-IL-bR2-1D3_AA 100-107
GIAPRRYY

SEQ ID NO: 235 - protein, 9 aa, synthetic construct
  REGION 1..9: note = Synthetic construct, 49-IL-bR2-1A7_AA 27 - 35
NIYDINTMA

SEQ ID NO: 236 - protein, 13 aa, synthetic construct
  REGION 1..13: note = Synthetic construct, 49-IL-bR2-1A7_AA 97-109
NVKTGRGRNLYSD

SEQ ID NO: 237 - protein, 8 aa, synthetic construct
  REGION 1..8: note = Synthetic construct, 24-IL-bR2-1H3_AA 27 - 34
SISSIDTM

SEQ ID NO: 238 - protein, 4 aa, synthetic construct
  REGION 1..4: note = Synthetic construct, 24-IL-bR2-1H3_AA 98-101
VVVS

SEQ ID NO: 239 - protein, 6 aa, synthetic construct
  REGION 1..6: note = Synthetic construct, 24-IL-bR2-1H3_AA 103 - 108
TLIAGS

SEQ ID NO: 240 - protein, 10 aa, synthetic construct
  REGION 1..10: note = Synthetic construct, 58-IL-bR2-1B8-_AA 26-35
ESIDTFDIID

SEQ ID NO: 241 - protein, 18 aa, synthetic construct
  REGION 1..18: note = Synthetic construct, 58-IL-bR2-1B8_AA 46 - 61
DQRELVAQMLPVGATTYA

SEQ ID NO: 242 - protein, 11 aa, synthetic construct
  REGION 1..11: note = Synthetic construct, 58-IL-bR2-1B8_AA 97-107
HSINRDHNIWC

SEQ ID NO: 243 - protein, 6 aa, synthetic construct
  REGION 1..6: note = Synthetic construct, 10-IL-bR2-1B2_AA 27 - 32
VIPDAS

SEQ ID NO: 244 - protein, 7 aa, synthetic construct
  REGION 1..7: note = Synthetic construct, 10-IL-bR2-1B2_AA 52-59
VGPTNIL

SEQ ID NO: 245 - protein, 6 aa, synthetic construct
  REGION 1..6: note = Synthetic construct, 10-IL-bR2-1B2_AA75-80
GGDTIS

SEQ ID NO: 246 - protein, 8 aa, synthetic construct
  REGION 1..8: note = Synthetic construct, 10-IL-bR2-1B2_AA 97-104
NLLQSGTN

SEQ ID NO: 247 - protein, 5 aa, synthetic construct
  REGION 1..5: note = Synthetic construct, 12-IL-bR2-1D2_AA 30-34
SRNTM

SEQ ID NO: 248 - protein, 4 aa, synthetic construct
  REGION 1..4: note = Synthetic construct, 12-IL-bR2-1D2_AA 55-58
DITN

SEQ ID NO: 249 - protein, 5 aa, synthetic construct
  REGION 1..5: note = Synthetic construct, 76-IL-bR2-1D10_AA 99-103
IRGVN